(19) United States
(12) Patent Application Publication
(10) Pub. No.: US 2011/0178065 A1
(43) Pub. Date: Jul. 21, 2011

(54) NOVEL CYP17 INHIBITORS

(75) Inventors: Daniel Chu, Santa Clara, CA (US); Bing Wang, San Jose, CA (US); Tao Ye, Kowloon (HK)

(73) Assignee: BIOMARIN PHARMACEUTICAL INC., Novato, CA (US)

(21) Appl. No.: 13/005,646

(22) Filed: Jan. 13, 2011

Related U.S. Application Data
(60) Provisional application No. 61/295,472, filed on Jan. 15, 2010.

(51) Int. Cl. A61K 31/55; A61P 35/00; C12N 9/99; A61K 31/366; C07D 335/04; C07D 405/08; C07D 487/04; C07D 333/50; A61K 31/365; A61K 31/443; C07D 401/08; A61K 31/497

(52) U.S. Cl. 514/212.04; 435/184; 514/453; 549/25; 546/282.7; 540/519; 544/336; 549/42; 514/468; 514/337; 546/77; 546/281.7; 514/255.05

(57) ABSTRACT

Provided herein are inhibitors of CYP17 enzyme. Also described herein are pharmaceutical compositions that include at least one compound described herein and the use of a compound or pharmaceutical composition described herein to treat androgen-dependent diseases, disorders and conditions.

### CROSS REFERENCE

This application claims the benefit of U.S. Provisional Application No. 61/295,472, filed Jan. 15, 2010, which is incorporated by reference in its entirety herein.

### FIELD OF THE INVENTION

Described herein are compounds, methods of making such compounds, pharmaceutical compositions and medicaments containing such compounds, and methods of using such compounds to treat androgen-dependent diseases or conditions.

### BACKGROUND OF THE INVENTION

The 17α-hydroxylase/C₁₇₋₂₀ lyase enzyme complex is essential for the biosynthesis of androgens. CYP17 is a bifunctional enzyme which possess both a C_{17,20}-lyase activity and a C₁₇-hydroxylase activity. These two alternative enzymatic activities of CYP17 result in the formation of critically different intermediates in steroid biosynthesis and each activity appear to be differentially and developmentally regulated.

### SUMMARY OF THE INVENTION

Provided herein are compounds, compositions and methods for inhibiting the CYP17 enzyme. Also described herein is the use of such compounds and compositions for the treatment of cancer and/or androgen-dependent diseases, disorders or conditions.

In one aspect, compounds provided herein have the structure of Formula (I), (II), (III), (IA), (IB), (IC), (ID), (IE), (IF), (IG), (IH), (IJ), (IK), (IL), (IM), (IN), (IIA), (IIB) or (IIC), or pharmaceutically acceptable salts, solvates, esters, acids and prodrugs thereof.

In some or any embodiments, isomers and chemically protected forms of compounds having a structure represented by Formula (I), (II), (III), (IA), (IB), (IC), (ID), (IE), (IF), (IG), (IH), (IJ), (IK), (IL), (IM), (IN), (IIA), (IIB) or (IIC) are also provided.

In one aspect is a compound having the structure of Formula (I):

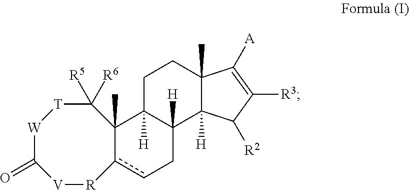

wherein:

R is (CH₂)ₚ wherein p is an integer from 0 to 1;

T is (CH₂)_{q} wherein q is an integer from 0 to 1;

W is O, NR¹, N—COR¹, N—COOR¹ or null;

V is CR⁷, R⁸, O, NR¹, N—COR¹or N—COOR¹;

with the proviso that when W is O, NR¹, N—COR¹, or N—COOR¹ and p and q are each 0, or when W is null and one of p and q is 0, V cannot be CR⁷, R⁸

A is a heteroaryl optionally substituted with 1, 2, 3, or 4 substituents R⁴;

[symbol] is a single bond or double bond;

R¹ is selected from the group consisting of hydrogen, alkyl, cycloalkyl, alkenyl, alkynyl, alkoxyalkyl, hydroxyl, and/or haloalkoxyalkyl; wherein the alkyl, cycloalkyl, alkenyl, alkynyl, alkoxyalkyl, and/or haloalkoxyalkyl groups are optionally substituted with 1, 2, or 3 substituents independently selected from the group consisting of halogen, alkyl, alkenyl, aryl, heteroaryl, alkoxy, alkoxycarbonyl, hydroxyl, hydroxyalkyl, alkynyl, cyano, haloalkoxy, haloalkyl, nitro, NR_{A}R_{B}, and/or (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and/or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and/or optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and/or (NR_{A}R_{B})carbonyl; and

R⁵ and R⁶ are each independently selected from the group consisting of hydrogen, halogen, optionally substituted alkyl, and/or optionally substituted alkoxyalkyl;

R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B}; and

R⁷ and R⁸ are each independently selected from the group consisting of hydrogen, halogen, optionally substituted alkyl, optionally substituted alkoxyalkyl;

or a pharmaceutically acceptable salt, solvate, stereoisomer, tautomer or prodrug thereof. In some or any cases, W is O, NR¹, or N—COR¹. In some or any cases, W is null, O, NR¹, or NCOOR¹. In some or any cases, W is NH or NCH₃. In some or any cases, T is CH₂. In some or any cases, V is CH₂. In some or any cases, p is 0.

In some or any embodiments, disclosed herein is a compound having the structure of Formula (II):

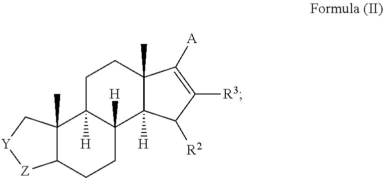

wherein:

Y is (CH₂)ₘ wherein m is an integer from 1 to 3;

Z is O, S(O)ᵤ, NR¹, N—COR¹or N—COOR¹, wherein u is an integer from 0 to 2;

A is a heteroaryl optionally substituted with 1, 2, 3, or 4 R⁴;

R¹ is selected from the group consisting of hydrogen, alkyl, cycloalkyl, alkenyl, alkynyl, alkoxyalkyl, hydroxyl, and/or haloalkoxyalkyl; wherein the alkyl, cycloalkyl, alkenyl, alkynyl, alkoxyalkyl, and/or haloalkoxyalkyl groups are optionally substituted with 1, 2, or 3 substituents independently selected from the group consisting of halogen, alkyl, alkenyl, aryl, heteroaryl, alkoxy, alkoxycarbonyl, hydroxyl, hydroxyalkyl, alkynyl, cyano, haloalkoxy, haloalkyl, nitro, NR_{A}R_{B}, and/or (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and/or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and/or optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and/or (NR_{A}R_{B})carbonyl; and

R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B};

or a pharmaceutically acceptable salt, solvate, stereoisomer, tautomer or prodrug thereof.

In some or any embodiments, disclosed herein is a compound having the structure of Formula (III):

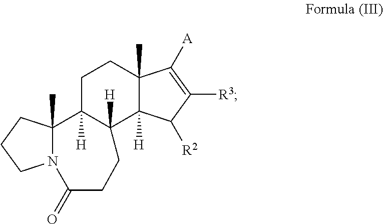

wherein:

A is a heteroaryl optionally substituted with 1, 2, 3, or 4 R⁴;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and/or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and/or optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and/or (NR_{A}R_{B})carbonyl; and

R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B};

or a pharmaceutically acceptable salt, solvate, stereoisomer, tautomer or prodrug thereof.

In some or any embodiments, disclosed herein is a compound having the structure of Formula (IA):

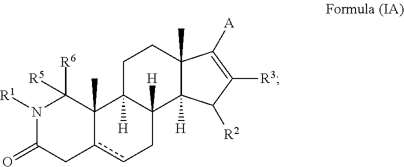

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (IB):

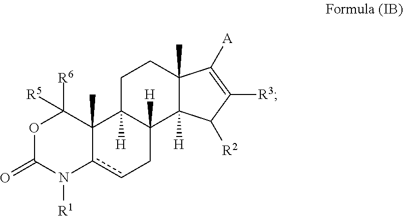

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (IC):

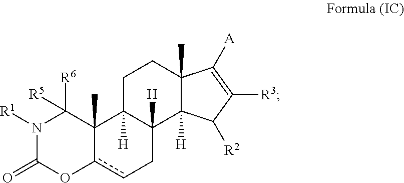

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (ID):

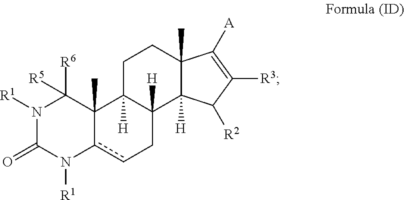

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (IE):

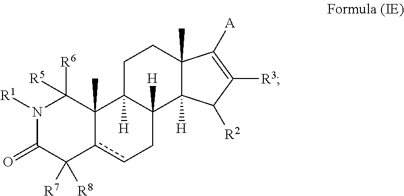

or a pharmaceutically acceptable salt, solvate, stereoisomer, tautomer or prodrug thereof, wherein A, R¹, R², R³, R⁵, R⁶, R⁷, and R⁸ are as defined for Formula (I).

In some or any embodiments, disclosed herein is a compound having the structure of Formula (IF):

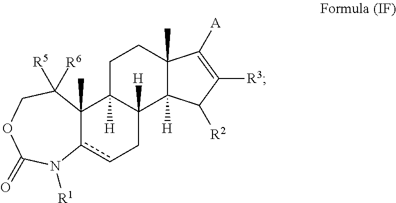

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (IG):

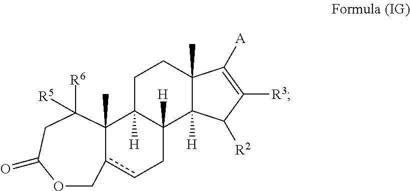

or a pharmaceutically acceptable salt, solvate, stereoisomer, tautomer or prodrug thereof, wherein A, R², R³, R⁵, and R⁶ are as defined for Formula (I).

In some or any embodiments, disclosed herein is a compound having the structure of Formula (IH):

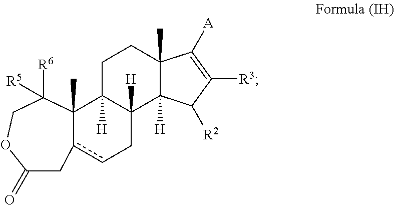

or a pharmaceutically acceptable salt, solvate, stereoisomer, tautomer or prodrug thereof, wherein A, R², R³, R⁵, and R⁶ are as defined for Formula (I).

In some or any embodiments, disclosed herein is a compound having the structure of Formula (IJ):

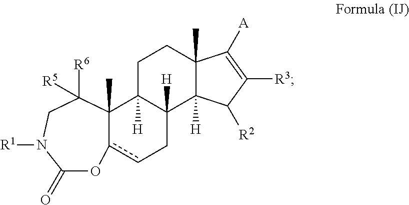

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (IK):

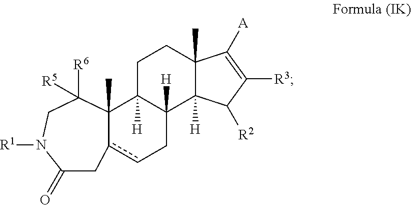

or a pharmaceutically acceptable salt, solvate, stereoisomer, tautomer or prodrug thereof, wherein A, R¹, R², R³, R⁵, and R⁶ are as defined for Formula (I).

In some or any embodiment, disclosed herein is a compound having the structure of Formula (IL):

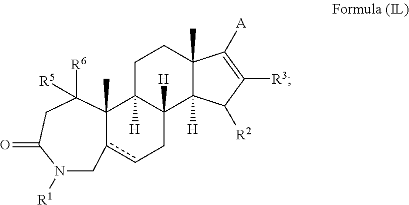

or a pharmaceutically acceptable salt, solvate, stereoisomer, tautomer or prodrug thereof, wherein A, R¹, R², R³, R⁵, and R⁶ are as defined for Formula (I).

In some or any embodiments, disclosed herein is a compound having the structure of Formula (IM):

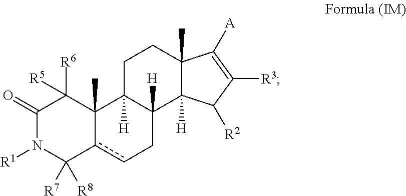

or a pharmaceutically acceptable salt, solvate, stereoisomer, tautomer or prodrug thereof, wherein A, R¹, R², R³, R⁵, R⁶, R⁷, and R⁸ are as defined for Formula (I).

In some or any embodiments, disclosed herein is a compound having the structure of Formula (IN):

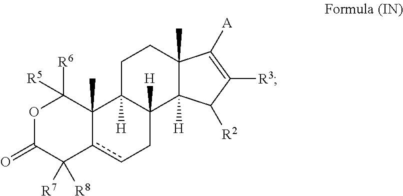

or a pharmaceutically acceptable salt, solvate, stereoisomer, tautomer or prodrug thereof, wherein A, R², R³, R⁵, R⁶, R⁷ and R⁸ are as defined for Formula (I).

In some or any embodiments, disclosed herein is a compound having the structure of Formula (IIA):

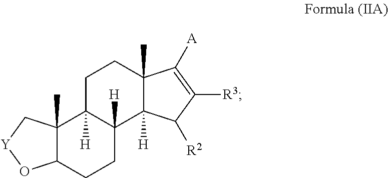

or a pharmaceutically acceptable salt, solvate, stereoisomer, tautomer or prodrug thereof, wherein A, Y, R², and R³ are as defined for Formula (II).

In some or any embodiments, disclosed herein is a compound having the structure of Formula (IIB):

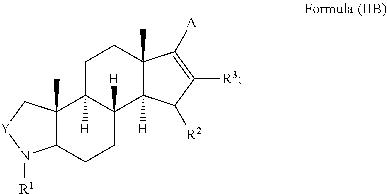

or a pharmaceutically acceptable salt, solvate, stereoisomer, tautomer or prodrug thereof, wherein A, Y, R¹, R², and R³ are as defined for Formula (II).

In some or any embodiments, disclosed herein is a compound having the structure of Formula (IIC):

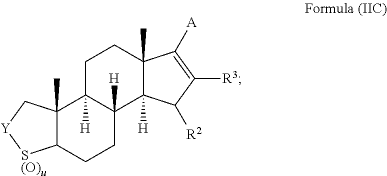

or a pharmaceutically acceptable salt, solvate, stereoisomer, tautomer or prodrug thereof, wherein A, Y, R², R³, and u are as defined for Formula (II).

In some or any embodiments, disclosed herein is a compound having the structure of Formula (I), (II), (III), (IA), (IB), (IC), (ID), (IE), (IF), (IG), (IH), (II), (IK), (IL), (IM), (IN), (IIA), (IIB) or (IIC), wherein A is an optionally substituted heteroaryl.

In some or any embodiments, disclosed herein is a compound having the structure of Formula (I), (II), (III), (IA), (IB), (IC), (ID), (IE), (IF), (IG), (IH), (II), (IK), (IL), (IM), (IN), (IIA), (IIB) or (IIC), wherein A is a heteroaryl and is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and/or pteridine. In some or any embodiments, A is 3-pyridyl, imidazole, triazole, or pyrazine, and optionally substituted with one to four R⁴ substituents. In some or any cases disclosed herein, R⁴ is hydrogen, C₁₋₄alkyl, C₁₋₄alkoxy, or halogen. In some or any cases, at least one of R², R³, R⁵, R⁶, R⁷, and R⁸ is hydrogen. In some or any cases, each of R², R³, R⁵, R⁶, R⁷, and R⁸ is hydrogen. In some or any cases, R₁ is hydrogen or methyl.

In some or any embodiments, disclosed herein is a compound having the structure of Formula (I), (II), (III), (IA), (IB), (IC), (ID), (IE), (IF), (IG), (IH), (H), (IK), (IL), (IM), (IN), (IIA), (IIB) or (IIC) wherein A is a heteroaryl and is selected from pyridine, imidazole, triazole, benzimidazole, pyrrole, pyrazole, pyrimidine, pyrazine, and/or pyridazine.

In some or any embodiment, disclosed herein is a compound having the structure of Formula (I), (II), (III), (IA), (IB), (IC), (ID), (IE), (IF), (IG), (IH), (H), (IK), (IL), (IM), (IN), (IIA), (IIB) or (IIC), wherein A is pyridine.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC), wherein A is benzimidazole.

In some or any embodiments, disclosed herein is a compound having the structure of Formula (I), (II) or (III), wherein A is imidazole.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is selected from the group consisting of pyrazine and/or pyrimidine.

In some or any embodiments, disclosed herein is a compound having the structure of Formula (I), (II), (III), (IA), (IB), (IC), (ID), (IE), (IF), (IG), (IH), (H), (IK), (IL), (IM), (IN), (IIA), (IIB) or (IIC) wherein A is triazole. In some or any embodiments, the triazole is a 1,2,3-triazole. In a further embodiment, the triazole is a 1,2,4-triazole. In yet a further embodiment, the 1,2,3-triazole and/or 1,2,4-triazole is substituted with at least one substituent selected from alkyl, cycloalkyl, alkenyl, alkynyl, alkoxyalkyl, hydroxyl, and/or haloalkoxyalkyl.

In some or any embodiments, disclosed herein is a compound having the structure of Formula (I), (II), (III), (IA), (IB), (IC), (ID), (IE), (IF), (IG), (IH), (H), (IK), (IL), (IM), (IN), (IIA), (IIB) or (IIC) wherein R¹ is hydrogen, alkyl, and/or cycloalkyl and wherein the alkyl and cycloalkyl groups are optionally substituted with 1, 2, or 3 substituents independently selected from the group consisting of halogen, alkyl, alkenyl, alkoxy, alkoxycarbonyl, hydroxyl, hydroxyalkyl, alkynyl, cyano, haloalkoxy, haloalkyl, nitro, NR_{A}R_{B}, and/or (NR_{A}R_{B})carbonyl.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R¹ is hydrogen or C₁-C₆ alkyl.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R² is selected from the group consisting of hydrogen, halogen, hydroxyl, optionally substituted alkyl, optionally substituted cycloalkyl, cyano, and/or nitro.

In some or any embodiment, disclosed herein is a compound having the structure of Formula (I), (II), (III), (IA), (IB), (IC), (ID), (IE), (IF), (IG), (IH), (II), (IK), (IL), (IM), (IN), (IIA), (IIB) or (IIC) wherein R² is hydrogen or C₁-C₆ alkyl.

In some or any embodiment, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R³ is selected from the group consisting of hydrogen, halogen, optionally substituted alkyl, optionally substituted cycloalkyl, cyano, hydroxyl, and/or nitro.

In some or any embodiments, disclosed herein is a compound having the structure of Formula (I), (II), (III), (IA), (IB), (IC), (ID), (IE), (IF), (IG), (IH), (II), (IK), (IL), (IM), (IN), (IIA), (IIB) or (IIC) wherein R³ is hydrogen or C₁-C₆ alkyl.

In some or any embodiments, disclosed herein is a compound having the structure of Formula (I), (IA), (IB), (IC), (ID), (IE), (IF), (IG), (IH), (IJ), (IK), (IL), (IM) or (IN), wherein is a double bond.

In some or any embodiments, disclosed herein is a compound having the structure of Formula (I), (IA), (IB), (IC), (ID), (IE), (IF), (IG), (IH), (IJ), (IK), (IL), (IM) or (IN), wherein is a single bond.

Also described herein is a pharmaceutical composition comprising a compound having a structure of Formula (I), (II), (III), (IA), (IB), (IC), (ID), (IE), (IF), (IG), (IH), (IJ), (IK), (IL), (IM), (IN), (IIA), (IIB) or (IIC) and a pharmaceutically acceptable carrier, excipient or binder.

In one aspect, provided is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof.

In some or any embodiments, disclosed herein is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof wherein the cancer is selected from the group consisting of bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, thoracic cancer, and/or combinations thereof.

In some or any embodiments, disclosed herein is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof wherein the cancer is prostate cancer.

In some or any embodiments, the method of treating cancer further comprises providing to the subject in need an additional therapy selected from the group consisting of surgery, radiation therapy, chemotherapy, gene therapy, immunotherapy, and/or a combination thereof. In some or any embodiments, disclosed herein is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof wherein the cancer is breast cancer.

In some or any embodiments, disclosed herein are methods wherein the additional therapy is surgery.

In some or any embodiments, disclosed herein are methods wherein providing chemotherapy to the subject in need comprises administering a therapeutically effective amount of at least one anti-androgenic agent.

In some or any embodiments, the at least one anti-androgenic agent is selected from the group consisting of flutamide, nicalutamide, bicalutamide, inhibitors of 17α-hydroxylase/C17-20 lyase, luteinizing hormone-releasing hormone agonists, luteinizing hormone-releasing hormone antagonists, and 5α-reductase type 1 and/or type 2 and/or combinations thereof.

Also disclosed herein is a method of inhibiting CYP17 enzyme comprising contacting a compound having the structure of Formula (I), (II), (III), (IA), (IB), (IC), (ID), (IE), (IF), (IG), (IH), (IA (IK), (IL), (IM), (IN), (IIA), (IIB) or (IIC) or a pharmaceutically acceptable salt or solvate thereof with a CYP17 enzyme.

In some or any embodiments, the contacting step is in vivo.

Also described herein is a method of treating an androgen-dependent disorder in a subject comprising administering to a subject in need a therapeutically effective amount of a compound having the structure of Formula (I), (II), (III), (IA), (IB), (IC), (ID), (IE), (IF), (IG), (IH), (IA (IK), (IL), (IM), (IN), (IIA), (IIB) or (IIC) or a pharmaceutically acceptable salt or solvate thereof.

In some or any embodiments, the androgen-dependent disorder is selected from the group consisting of prostate cancer, benign prostatic hyperplasia, prostatic intraepithelial neoplasia, hirsutism, acne, androgenic alopecia, and/or polycystic ovary syndrome.

In some or any embodiments, the androgen-dependent disorder is prostate cancer.

Presented herein is a method of treating a proliferative disease comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof.

In some or any embodiments, the method further comprises administering a therapeutically effective amount of at least one agent or therapy selected from the group consisting of a chemotherapeutic agent, a biological agent, surgery, and/or radiation therapy.

In some or any embodiments, the administration of the at least one agent or therapy is performed concurrently or sequentially.

In one aspect, disclosed herein is an article of manufacture, comprising packaging material, a compound having the structure of Formula (I), (II), (III), (IA), (IB), (IC), (ID), (IE), (IF), (IG), (IH), (IA (IK), (IL), (IM), (IN), (IIA), (IIB) or (IIC) and a label, wherein the compound is effective for the treatment of an androgen dependent disorder, wherein the compound is packaged within the packaging material, and wherein the label indicates that the compound, or pharmaceutically acceptable salt or solvate thereof is used for the treatment of an androgen dependent disorder.

In one aspect, disclosed herein is a use of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof in the manufacture of a medicament for the treatment of prostate cancer. Further disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof for treating an androgen dependent disorder as disclosed herein, such as, for example, prostate cancer.

### DETAILED DESCRIPTION OF THE INVENTION

In the testes and adrenal glands, the last step in the biosynthesis of testosterone involves two key reactions, which act sequentially and are both catalyzed by a single enzyme, the cytochrome P450 monooxygenase 17α-hydroxylase/C_{17,20}-lyase (P450₁₇ or CYP17). CYP17 is a key enzyme in the biosynthesis of androgens, and converts the C₂₁ steroids (pregnenolone and progesterone) to the C₁₉ androgens, dehydroepiandrosterone (DHEA), androstenediol (A-diol), testosterone, and androstenedione in the testes and adrenals. Both DHEA and androstenedione lyase products are key intermediates in the synthesis of not only the androgens testosterone and dihydrotestosterone (DHT), but also the estrogens 17β-estradiol and estrone. Adrenal and ovarian estrogens are the main sources of estrogens in postmenopausal women. The C₁₇-hydroxylase activity of CYP17 catalyzes the conversion of the common intermediate progesterone to 17-hydroxyprogesterone, a precursor of cortisol. Thus, the C₁₇-hydroxylase activity promotes the formation of glucocorticoids while the C_{17,20}-lyase activity promotes the formation of sex hormones—particularly androgens including testosterone as well as estrogens.

Prostate cancer is the most common malignancy and age-related cause of cancer death worldwide. Apart from lung cancer, prostate cancer is the most common form of cancer in men and the second leading cause of death in American men. During the period of 1992 to 1999, the average annual incidence of prostate cancer among African American men was 59% higher than among Caucasian men, and the average annual death rate was more than twice that of Caucasian men (American Cancer Society—Cancer Facts and Figures 2003).

Androgens play an important role in the development, growth, and progression of prostate cancer. Two important androgens in this regard are testosterone and dihydrotestosterone (DHT). The testes synthesize about 90% of testosterone and the rest (10%) is synthesized by the adrenal glands. Testosterone is further converted to the more potent androgen DHT by the enzyme steroid 5α-reductase that is localized primarily in the prostate.

Since prostate cancer is typically androgen-dependent, the reduction of androgen production via surgical or pharmacological castration is the major treatment option for this indication. Androgen deprivation has been used as therapy for advanced and metastatic prostate cancer. Androgen ablation therapy has been shown to produce the most beneficial responses in multiple settings in prostate cancer patients. However, orchidectomy remains the standard treatment option for most prostate cancer patients.

Medical and surgical orchidectomy reduces or eliminates androgen production by the testes but does not affect androgen synthesis in the adrenal glands. Several studies have reported that orchidectomy therapy and treatment with anti-androgens to inhibit the action of adrenal androgens significantly prolongs the survival of prostate cancer patients. Further, it has been shown that testosterone and DHT occur in recurrent prostate cancer tissues at levels sufficient to activate androgen receptor. In addition, the use of microarray-based profiling of isogenic prostate cancer xenograft models showed that a modest increase in androgen receptor mRNA was the only change consistently associated with the development of resistance to anti-androgen therapy. Since CYP17 is implicated in the synthesis of key intermediates of androgens, the pharmacological inhibition of CYP17 is a promising treatment in that testicular, adrenal, and peripheral androgen biosynthesis would be reduced rather than only testicular androgen production. (Njar, V. et al., J. Med. Chem. 1998, 41, 902).

Inhibitors of CYP17 have been previously described. For example, ketoconazole, an active imidazole fungicide has been used to reduce testosterone biosynthesis in the treatment of patients with advanced prostatic cancer. However, there are side-effects including liver damage, inhibition of several other cytochrome P₄₅₀ steroidogenic enzymes, and reduction of cortisol production.

Potent and selective inhibitors of CYP17 as potential prostate cancer treatments have been the subject of previous studies. Finasteride, a 5α-reductatse inhibitor, is an approved treatment for benign prostatic hyperplasia (BPH), although it is only effective with patients exhibiting minimal disease. While finasteride reduces serum DHT levels, it increases testosterone levels and may therefore be insufficient for prostate cancer treatment.

In addition to the use of CYP17 inhibitors in the treatment of prostate cancer, CYP17 inhibitors will find utility for the indication of breast cancer, more particularly, estrogen-dependent breast cancer. In post-menopausal patients with advanced breast cancer, treatment with high doses of ketoconazole resulted in suppression of both testosterone and estradiol levels, implicating CYP17 as a potential target for hormone therapy. (Harris, A. L. et al., Br. J. Cancer 1988, 58, 493).

Provided herein are compounds having the structure of Formulas (I), (II), (III), (IA), (IB), (IC), (ID), (IE), (IF), (IG), (IH), (IJ), (IK), (IL), (IM), (IN), (IIA), (IIB) or (IIC) or pharmaceutically acceptable salts or solvates thereof, in the treatment of cancer, in the inhibition of CYP17, and/or in the treatment of androgen-dependent diseases.

In one aspect, disclosed herein is a compound having the structure of Formula (I):

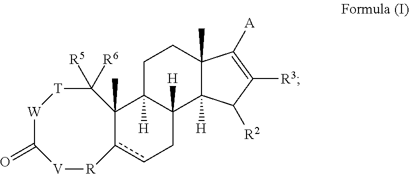

wherein:

R is (CH₂)ₚ wherein p is an integer from 0 to 1;

T is (CH₂)_{q} wherein q is an integer from 0 to 1;

W is O, NR¹, N—COR¹, N—COOR¹or null;

V is CR⁷, R⁸, O, NR¹, N—COR¹ or N—COOR¹;

with the proviso that when W is O, NR¹, N—COR¹, or N—COOR¹ and p and q are each 0, or when W is null and one of p and q is 0, V cannot be CR⁷, R⁸

A is a heteroaryl optionally substituted with 1, 2, 3, or 4 R⁴;

[symbol] is a single bond or double bond; R¹ is selected from the group consisting of hydrogen, alkyl, cycloalkyl, alkenyl, alkynyl, alkoxyalkyl, hydroxyl, and/or haloalkoxyalkyl; wherein the alkyl, cycloalkyl, alkenyl, alkynyl, alkoxyalkyl, and/or haloalkoxyalkyl groups are optionally substituted with 1, 2, or 3 substituents independently selected from the group consisting of halogen, alkyl, alkenyl, aryl, heteroaryl, alkoxy, alkoxycarbonyl, hydroxyl, hydroxyalkyl, alkynyl, cyano, haloalkoxy, haloalkyl, nitro, NR_{A}R_{B}, and/or (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and/or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and/or optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and/or (NR_{A}R_{B})carbonyl; and

R⁵ and R⁶ are each independently selected from the group consisting of hydrogen, halogen, optionally substituted alkyl, and/or optionally substituted alkoxyalkyl;

R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B}; and

R⁷ and R⁸ are each independently selected from the group consisting of hydrogen, halogen, optionally substituted alkyl, and/or optionally substituted alkoxyalkyl;

or a pharmaceutically acceptable salt, solvate, stereoisomer, tautomer or prodrug thereof. In some or any cases, W is O or NR¹. In some or any cases, W is null, O, NR¹, or NCOOR¹. In some or any cases, W is NH or NCH₃. In some or any cases, T is CH₂. In some or any cases, V is CH₂. In some or any cases, p is 0.

In some or any embodiments, disclosed herein is a compound having the structure of Formula (II):

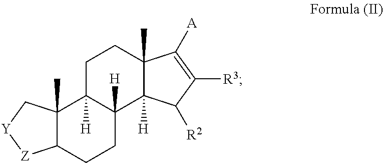

wherein:

Y is (CH₂)ₘ wherein m is an integer from 1 to 3;

Z is O, S(O)ᵤ, NR¹, N—COR¹or N—COOR¹, wherein u is an integer from 0 to 2;

A is a heteroaryl optionally substituted with 1, 2, 3, or 4 R⁴;

R¹ is selected from the group consisting of hydrogen, alkyl, cycloalkyl, alkenyl, alkynyl, hydroxyl, alkoxyalkyl, and/or haloalkoxyalkyl; wherein the alkyl, cycloalkyl, alkenyl, alkynyl, alkoxyalkyl, and/or haloalkoxyalkyl groups are optionally substituted with 1, 2, or 3 substituents independently selected from the group consisting of halogen, alkyl, alkenyl, aryl, heteroaryl, alkoxy, alkoxycarbonyl, hydroxyl, hydroxyalkyl, alkynyl, cyano, haloalkoxy, haloalkyl, nitro, NR_{A}R_{B}, and/or (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and/or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and/or optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and/or (NR_{A}R_{B})carbonyl; and

R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B};

or a pharmaceutically acceptable salt, solvate, stereoisomer, tautomer or prodrug thereof.

In some or any embodiments, disclosed herein is a compound having the structure of Formula (III):

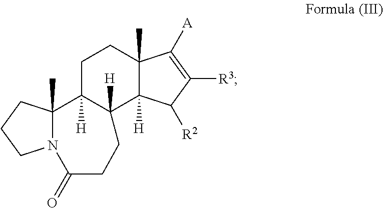

wherein:

A is a heteroaryl optionally substituted with 1, 2, 3, or 4 R⁴;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and/or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and/or optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and/or (NR_{A}R_{B})carbonyl; and

R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B};

or a pharmaceutically acceptable salt, solvate, stereoisomer, tautomer or prodrug thereof.

In some or any embodiments, disclosed herein is a compound having the structure of Formula (IA):

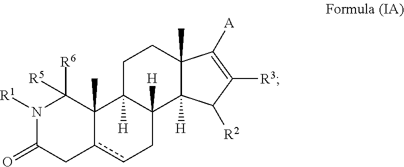

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IB):

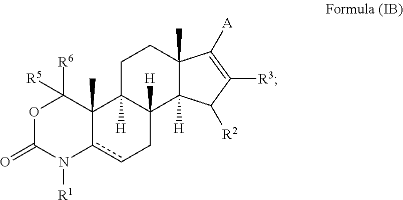

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IC):

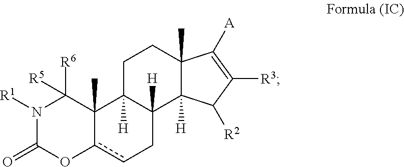

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (ID):

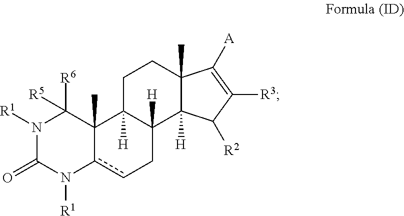

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IE):

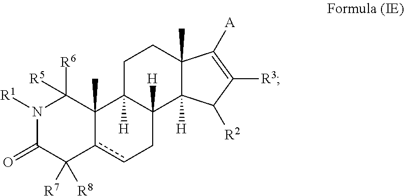

or a pharmaceutically acceptable salt, solvate, stereoisomer, tautomer or prodrug thereof, wherein A, R¹, R², R³, R⁵, R⁶, R⁷, and R⁸ are as defined for compounds of Formula (I).

In some or any embodiments, disclosed herein is a compound having the structure of Formula (IF):

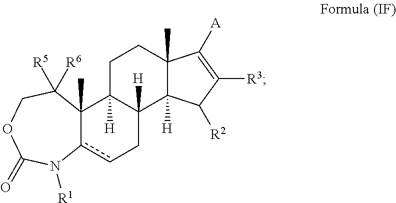

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IG):

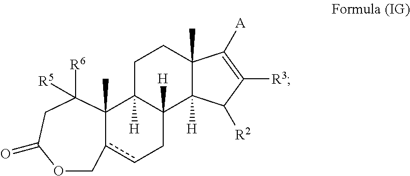

or a pharmaceutically acceptable salt, solvate, stereoisomer, tautomer or prodrug thereof, wherein A, R², R³, R⁵, and R⁶ are as defined for compounds of Formula (I).

In some or any embodiments, disclosed herein is a compound having the structure of Formula (IH):

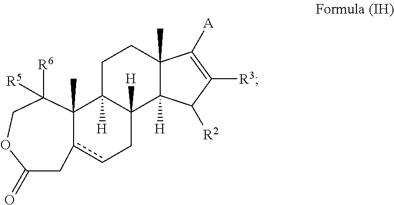

or a pharmaceutically acceptable salt, solvate, stereoisomer, tautomer or prodrug thereof, wherein A, R², R³, R⁵, and R⁶ are as defined for compounds of Formula (I).

In some or any embodiments, disclosed herein is a compound having the structure of Formula (IJ):

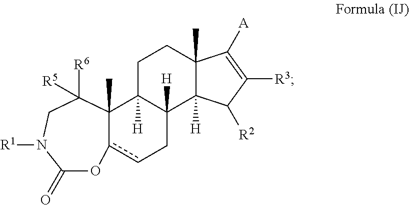

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IK):

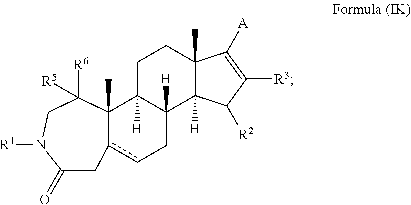

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IL):

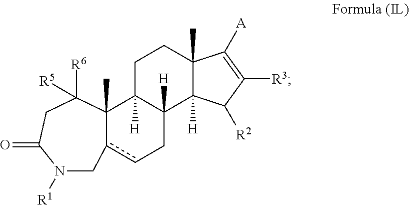

or a pharmaceutically acceptable salt, solvate, stereoisomer, tautomer or prodrug thereof, wherein A, R¹, R², R³, R⁵, and R⁶ are as defined for compounds of Formula (I).

In some or any embodiments, disclosed herein is a compound having the structure of Formula (IM):

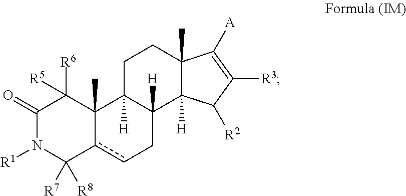

or a pharmaceutically acceptable salt, solvate, stereoisomer, tautomer or prodrug thereof, wherein A, R¹, R², R³, R⁵, R⁶, R⁷ and R⁸ are as defined for compounds of Formula (I).

In some or any embodiments, disclosed herein is a compound having the structure of Formula (IN):

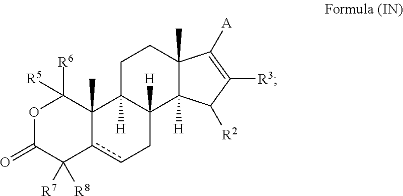

or a pharmaceutically acceptable salt, solvate, stereoisomer, tautomer or prodrug thereof, wherein A, R², R³, R⁵, R⁶, R⁷, and R⁸ are as defined for compounds of Formula (I).

In some or any embodiment, disclosed herein is a compound having the structure of Formula (IIA):

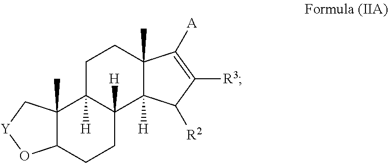

or a pharmaceutically acceptable salt, solvate, stereoisomer, tautomer or prodrug thereof, wherein A, R², R³, and Y are as defined for compounds of Formula (II).

In some or any embodiments, disclosed herein is a compound having the structure of Formula (IIB):

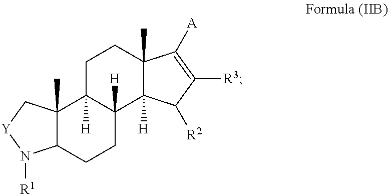

or a pharmaceutically acceptable salt, solvate, stereoisomer, tautomer or prodrug thereof, wherein A, R¹, R², R³, and Y are as defined for compounds of Formula (II).

In some or any embodiments, disclosed herein is a compound having the structure of Formula (IIC):

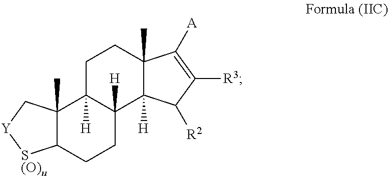

or a pharmaceutically acceptable salt, solvate, stereoisomer, tautomer or prodrug thereof, wherein A, R², R³, Y, and u are as defined for compounds of Formula (II).

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is an optionally substituted heteroaryl.

In some or any embodiments, disclosed herein is a compound having the structure of Formula (I), (II), (III), (IA), (IB), (IC), (ID), (IE), (IF), (IG), (IH), (IJ), (IK), (IL), (IIA), (IIB) or (IIC) wherein A is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and/or pteridine. In some or any embodiments, A is 3-pyridyl, imidazole, triazole, or pyrazine, and optionally substituted with one to four R⁴ substituents. In some or any cases disclosed herein, R⁴ is hydrogen, C₁₋₄alkyl, C₁₋₄alkoxy, or halogen. In some or any cases, at least one of R², R³, R⁵, R⁶, R⁷, and R⁸ is hydrogen. In some or any cases, each of R², R³, R⁵, R⁶, R⁷, and R⁸ is hydrogen. In some or any cases, R¹ is hydrogen or methyl.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is selected from pyridine, imidazole, thiazole, benzimidazole, pyrrole, pyrazole, pyrimidine, pyrazine, and/or pyridazine.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is pyridine.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is benzimidazole.

In some or any embodiments, disclosed herein is a compound having the structure of Formula (I), (II), (III), (IA), (IB), (IC), (ID), (IE), (IF), (IG), (IH), (IJ), (IK), (IL), (IIA), (IIB) or (IIC) wherein A is imidazole.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is pyrazine or pyrimidine.

In some or any embodiments, disclosed herein is a compound having the structure of Formula (I), (II), (III), (IA), (IB), (IC), (ID), (IE), (IF), (IG), (IH), (IJ), (IK), (IL), (IIA), (IIB) or (IIC) wherein A is triazole. In some or any embodiments, the triazole is a 1,2,3-triazole or a 1,2,4-triazole.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R¹ is hydrogen, alkyl, or cycloalkyl and wherein the alkyl and cycloalkyl groups are optionally substituted with 1, 2, or 3 substituents independently selected from the group consisting of halogen, alkyl, alkenyl, alkoxy, alkoxycarbonyl, hydroxyl, hydroxyalkyl, alkynyl, cyano, haloalkoxy, haloalkyl, nitro, NR_{A}R_{B}, and/or (NR_{A}R_{B})carbonyl.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R¹ is hydrogen or C₁-C₆ alkyl.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R² is selected from the group consisting of hydrogen, halogen, hydroxyl, optionally substituted alkyl, optionally substituted cycloalkyl, cyano, and/or nitro.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R² is hydrogen or C₁-C₆ alkyl.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein R³ is selected from the group consisting of hydrogen, halogen, optionally substituted alkyl, optionally substituted cycloalkyl, cyano, hydroxyl, and/or nitro.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) and/or (IIC) wherein R³ is hydrogen or C₁-C₆ alkyl.

In some or any embodiments, disclosed herein is a compound having the structure of Formula (I), (IA), (IB), (IC), (ID), (IE), (IF), (IG), (IH), (IJ), (IK), (IL), (IM) or (IN), wherein is a double bond.

In some or any embodiments, disclosed herein is a compound having the structure of Formula (I), (IA), (IB), (IC), (ID), (IE), (IF), (IG), (IH), (IJ), (IK), (IL), (IM) or (IN), wherein the is a single bond.

Also described herein is a pharmaceutical composition comprising a compound having a structure of Formula (I), (II), (III), (IA), (IB), (IC), (ID), (IE), (IF), (IG), (IH), (IJ), (IK), (IL), (IM), (IN), (IIA), (IIB) or (IIC) and a pharmaceutically acceptable carrier, excipient or binder.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is an optionally substituted heteroaryl. In some or any embodiments, A is an optionally substituted heteroaryl group. In some or any embodiments, the heteroaryl group consists of one, two, three, or four heteroatoms selected from N, S, and O. In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and/or pteridine. In some or any embodiments, A is pyridine, imidazole, triazole, benzimidazole, pyrrole, pyrazole, pyrimidine, pyrazine, or pyridazine. In some or any embodiments, A is pyridine. In some or any embodiments, A is benzimidazole. In some or any embodiments, A is imidazole. In various embodiments, A is pyrazine.

In some or any embodiments, disclosed herein is a compound having the structure of Formula (I), (II), (III), (IA), (IB), (IC), (ID), (IE), (IF), (IG), (IH), (IJ), (IK), (IL), (IM), (IN), (IIA), (IIB) or (IIC) wherein A is a heteroaryl wherein the heteroaryl is substituted by 1 to 4 R⁴ substituents each independently selected from a group consisting of hydrogen, halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/or NR_{A}R_{B}. In some or any embodiments, each R⁴ is independently hydrogen. In some or any embodiments, at least one R⁴ is halogen. In some or any embodiments, at least one R⁴ is selected from Cl, Br, and/or F. In some or any embodiments, at least one R⁴ is C₁-C₆ alkoxy. In some or any embodiments, R⁴ is selected from methoxy, ethoxy, n-propoxy, and/or iso-propoxy. In some or any embodiments, at least one R⁴ is C₁-C₆ alkyl. In some or any embodiments, R⁴ is selected from methyl, ethyl, n-propyl, iso-propyl, n-butyl, iso-butyl, and/or tert-butyl.

In some or any embodiment, disclosed herein is a compound of Formula (I), (II), (III), (IA), (IB), (IC), (ID), (IE), (IF), (IG), (IH), (IJ), (IK), (IL), (IM), (IN), (IIA), (IIB) or (IIC) wherein A is an optionally substituted heteroaryl attached to the rest of core multi-ring scaffold at a heteroatom of the heteroaryl group. By way of example only, A is an optionally substituted imidazole group,

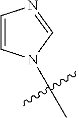

wherein the imidazole group is attached to rest of core multi-ringscaffold at a nitrogen atom,

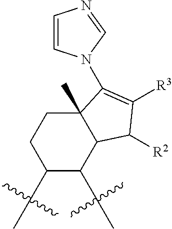

where the wavy lines indicate the attachment to the rest of the core multi-ring scaffold. In various embodiments, disclosed herein is a compound of Formula (I), (II), (III), (IA), (IB), (IC), (ID), (IE), (IF), (IG), (IH), (IJ), (IK), (IL), (IM), (IN), (IIA), (IIB) or (IIC), wherein A is an optionally substituted heteroaryl attached to the rest of the core multi-ring scaffold at a carbon atom of the heteroaryl group. Also by way of example only, A is an optionally substituted pyridine group,

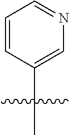

wherein the pyridine group is attached to rest of the core multi-ring scaffold at a carbon atom,

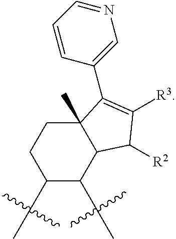

Also described herein is a compound selected from the group consisting of: Compound (1), Compound (2a), Compound (2b), Compound (3a), Compound (3b), Compound (4a), Compound (4b), Compound (5a), Compound (5b), Compound (6a), Compound (6b), Compound (7a), Compound (7b), Compound (8a), Compound (8b), Compound (9), Compound (10), Compound (Ha), Compound (11b), Compound (12), Compound (13), Compound (14), Compound (15), Compound (16), Compound (17), Compound (17g), Compound (18), Compound (19), Compound (20), Compound (20A), Compound (20B), Compound (20C), Compound (21), Compound (22), Compound (22A), Compound (22B), Compound (23), Compound (23A), Compound (23B), Compound (23C), Compound (24), Compound (25), Compound (26), Compound (26f), Compound (27), Compound (28), Compound (29), Compound (29A), Compound (29B), Compound (29C), Compound (29D), Compound (30), Compound (31), Compound (31A), Compound (31B), Compound (32), Compound (33), Compound (33A), Compound (33B), Compound (34), Compound (34A), Compound (34B), Compound (35), Compound (36), Compound (37), Compound (38), Compound (39), Compound (40), Compound (41), Compound (42), Compound (42A), Compound (42B), Compound (43), Compound (44), Compound (45), Compound (46), Compound (47), Compound (48), Compound (49), Compound (50), Compound (51), Compound (52), Compound (53), Compound (54), Compound (55), Compound (56), Compound (57), Compound (58), Compound (59), Compound (60), Compound (61), Compound (62), Compound (63), Compound (64), Compound (65), Compound (66), Compound (67), Compound (68), Compound (69), Compound (70), Compound (71), Compound (72A), Compound (72B), Compound (73A), Compound (73B), Compound (73C), Compound (73D), Compound (73E), Compound (73F), Compound (73G), Compound (73H), Compound (74A), Compound (74B), Compound (75A), Compound (75B), Compound (76), Compound (77), Compound (78A), Compound (78B), Compound (79), Compound (80), Compound (81), Compound (82), Compound (83), Compound (84), Compound (85), Compound (86), Compound (87), Compound (88), Compound (89), Compound (90), Compound (91), Compound (92), Compound (93), Compound (94), Compound (95), Compound (96A), and Compound (96B), or a pharmaceutically acceptable salt, solvate, stereoisomer, tautomer, or prodrug thereof.

Provided herein are pharmaceutical compositions comprising of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt, a pharmaceutically acceptable solvate, pharmaceutically acceptable prodrug thereof in combination with a pharmaceutically acceptable carrier, excipient, binder or diluent.

Also provided herein are methods of treating an androgen-dependent disease in a subject in need of such treatment comprising administering to the subject a therapeutically effective amount of a compound having a structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof.

In one aspect is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof.

In some or any embodiments, provided herein are methods and compositions for the treatment of CYP17-associated diseases and disorders. Examples include, but are not limited to, sex steroid hormone dependent cancers, such as androgen-dependent prostate cancer, which in some or any embodiments is treated by inhibiting CYP17-mediated androgen synthesis, and estrogen-dependent breast cancer or ovarian cancer, which in some or any embodiments is treated by inhibiting CYP17-mediated estrogen synthesis.

For example, adenocarcinoma of the prostate is a common disease that causes significant morbidity and mortality in the adult male population (see Han and Nelson, Expert Opin. Pharmacother. 2000, 1, 443-9). Hormonal therapy for prostate cancer is considered when a patient fails with initial curative therapy, such as radical prostatectomy or definitive radiation therapy, or if he is found with an advanced disease. Hormonal agents have been developed to exploit the fact that prostate cancer growth is dependent on androgen. Non-steroidal anti-androgens (NSAAs) block androgen at the cellular level. Castration is another, albeit drastic means of decreasing androgens levels in order to treat or prevent prostate cancer. The methods and compositions described herein are useful in inhibiting the C_{17,20}-lyase activity of CYP17 and thereby decreasing levels of androgen production and the associated growth of androgen-dependent cancers such as prostate cancer.

In some or any embodiments, breast cancer, such as, by way of example only, breast cancer in postmenopausal women, is treated by administration of a CYP17 inhibitor described herein since adrenal and ovarian androgens are the main precursors of the estrogens which stimulate the growth of hormone dependent breast cancer. In further embodiments, breast cancer is treated with one or more CYP17 inhibitors disclosed herein that inhibit interconversion of estrogens and adrenal and ovarian androgens. It has been shown that patients failing to respond to aromatase inhibitors show elevated levels of androgens in response to aromatase inhibitor treatment (see Harris et al., Bi. J. Cancer 1988, 58, 493-6). Accordingly, in some or any embodiments, sequential blockade to inhibit androgen production as well as inhibit aromatase produces greater estrogen suppression and enhanced therapeutic effects in treating breast and other estrogen hormone-dependent forms of cancer is disclosed herein. Therefore, in some or any embodiments, the inhibitors described herein are used alone or in combination with other drugs to treat and/or prevent hormone-dependent cancers such as breast and prostate cancer.

Furthermore, susceptibility to prostate cancer and breast cancer has been associated with particular polymorphic alleles of the CYP17 gene (see e.g. McKean-Cowdin, Cancer Res. 2001, 61, 848-9; Haiman et al., Cancer Epidmeiol. Biomarkers 2001,10,743-8; Huang et al., Cancer Res. 2001, 59, 4870-5). Accordingly, in some or any embodiments, the compositions described herein are suited to treating or preventing hormone-dependent cancers in individuals genetically predisposed to such cancers, particularly those predisposed due to an alteration in the CYP17 gene.

In some or any embodiments, disclosed herein is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof wherein the cancer is selected from the group consisting of bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and/or thoracic cancer.

In some or any embodiments, disclosed herein is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof wherein the cancer is prostate cancer.

In some or any embodiments, disclosed herein is a method for treating cancer in a subject comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof wherein the cancer is breast cancer.

In some or any embodiments, the method of treating cancer further comprises providing to the subject in need an additional therapy selected from the group consisting of surgery, radiation therapy, chemotherapy, gene therapy, immunotherapy, and/or a combination thereof.

In some or any embodiments, the additional therapy is surgery.

In some or any embodiments, providing chemotherapy to the subject in need comprises administering a therapeutically effective amount of at least one anti-androgenic agent.

In some or any embodiments, the at least one anti-androgenic agent is selected from the group consisting of flutamide, nicalutamide, bicalutamide, inhibitors of 17α-hydroxylase/C17-20 lyase, luteinizing hormone-releasing hormone agonists, luteinizing hormone-releasing hormone antagonists, and 5α-reductase type 1 and/or type 2 and/or combinations thereof.

Also disclosed herein is a method of inhibiting CYP17 enzyme comprising contacting a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof with a CYP17 enzyme.

In some or any embodiments, the contacting step is in vivo.

Also described herein is a method of treating an androgen-dependent disorder in a subject comprising administering to a subject in need a therapeutically effective amount of a compound having the structure of Formula (I), (II), (III), (IA), (IB), (IC), (ID), (IE), (IF), (IG), (IH), (Ti), (IK), (IL), (IM), (IN), (IIA), (IIB) or (IIC) or a pharmaceutically acceptable salt or solvate thereof.

In some or any embodiments, the androgen-dependent disorder is selected from the group consisting of prostate cancer, benign prostatic hyperplasia, prostatic intraepithelial neoplasia, hirsutism, acne, androgenic alopecia, and/or polycystic ovary syndrome.

In some or any embodiments, the androgen-dependent disorder is prostate cancer.

Presented herein is a method of treating a proliferative disease comprising administering to a subject in need a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof.

In some or any embodiments, the method further comprises administering a therapeutically effective amount of at least one agent or therapy selected from the group consisting of a chemotherapeutic agent, a biological agent, surgery, and radiation therapy.

In some or any embodiments, the administration is performed concurrently or sequentially.

In some or any embodiments, provided herein is a method of treating a disease associated with cancer ameliorated by the inhibition of CYP17 enzyme comprising administering to a subject in need of treatment a therapeutically effective amount of a compound having the structure of Formula (I), (II), (III), (IA), (IB), (IC), (ID), (IE), (IF), (IG), (IH), (Ti), (IK), (IL), (IM), (IN), (IIA), (IIB) or (IIC) or a pharmaceutically acceptable salt or solvate thereof.

In some or any embodiments, disclosed herein is a method for the treatment of or prevention of a disease such as prostate or breast cancer comprising administering to a subject in need of treatment a therapeutically effective amount of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof.

It is generally contemplated that a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or pharmaceutically acceptable salt or solvate thereof can be employed in the treatment of and in some embodiments inhibits especially the inhibition of the CYP17 enzyme.

Another group of CYP17-associated diseases or disorders amenable to treatment with the compounds, compositions, and methods of the present disclosure include those associated with mineralocorticoid excess such as hypertension caused by sodium retention at renal tubules. In some or any embodiments, a decrease in CYP17 activity results in an alteration in mineralocorticoid (e.g. aldosterone) biosynthesis. Accordingly, in some or any embodiments, the CYP17-associated diseases include those associated with altered levels of aldosterone production (e.g. hypertension, primary adrenal hyperplasia).

Still other examples of CYP17-associated diseases or disorders contemplated for treatment using a compound having the structure of Formula (I), (II), (III), (IA), (IB), (IC), (ID), (IE), (IF), (IG), (IH), (IJ), (IK), (IL), (IM), (IN), (IIA), (IIB) or (IIC) are Cushing's disease, prostatic hyperplasia, glucocorticoid deficiency, and/or endometrial cancer.

Some or any embodiments provide a use of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof in combination with other agents for treatment of various diseases or conditions. Combination therapies according to the present disclosure comprise the administration of at least one compound disclosed herein and at least one other pharmaceutically active ingredient. In some or any embodiments, second pharmaceutically active agents for combination therapy include anti-cancer agents. In some or any embodiments, the active ingredient(s) and pharmaceutically active agents are administered separately or together. In further embodiments, separate administration occurs simultaneously or separately in any order. The amounts of the active ingredients(s) and pharmaceutically active agent(s) and the relative timings of administration is selected in order to achieve the desired combined therapeutic effect.

Some or any embodiments provide a use of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) or a pharmaceutically acceptable salt or solvate thereof, in the preparation of a medicament for treating diseases associated with the CYP17 enzyme.

### Certain Chemical Terminology

Unless defined otherwise, all technical and scientific terms used herein have the standard meaning pertaining to the claimed subject matter belongs. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless otherwise indicated, conventional methods of mass spectroscopy, NMR, HPLC, protein chemistry, biochemistry, recombinant DNA techniques and pharmacology are employed. Unless specific definitions are provided, the standard nomenclature employed in connection with, and the standard laboratory procedures and techniques of, analytical chemistry, synthetic organic chemistry, and medicinal and pharmaceutical chemistry are employed. In certain instances, standard techniques are used for chemical syntheses, chemical analyses, pharmaceutical preparation, formulation, and delivery, and treatment of patients. In certain embodiments, standard techniques are used for recombinant DNA, oligonucleotide synthesis, and tissue culture and transformation (e.g., electroporation, lipofection). In some embodiments, reactions and purification techniques are performed e.g., using kits of manufacturer's specifications or as commonly accomplished or as described herein.

As used throughout this application and the appended claims, the following terms have the following meanings:

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. Depending on the structure, an alkenyl group includes a monoradical or a diradical (i.e., an alkenylene group). Alkenyl groups include optionally substituted groups. Illustrative examples of alkenyl are ethenyl, 2-propenyl, 2-methyl-2-propenyl, 3-butenyl, 4-pentenyl, 5-hexenyl, 2-heptenyl, 2-methyl-1-heptenyl, and 3-cecenyl.

The term “alkoxy” as used herein, means an alkyl group, as defined herein, appended to the parent molecular moiety through an oxygen atom. Illustrative examples of alkoxy are methoxy, ethoxy, propoxy, 2-propoxy, butoxy, tert-butoxy, pentyloxy, and hexyloxy.

The term “alkyl” as used herein, means a straight, branched chain, or cyclic (in this case, it would also be known as “cycloalkyl”) hydrocarbon containing from 1-10 carbon atoms. Illustrative examples of alkyl are methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

The term “cycloalkyl” as used herein, means a monocyclic or polycyclic radical that contains only carbon and hydrogen, and includes those that are saturated, partially unsaturated, or fully unsaturated. Cycloalkenyl refers to a cycloalkyl group having one or more double bonds. Cycloalkyl and cycloalkenyl groups include groups having from 3 to 10 ring atoms. Illustrative examples of cycloalkyl and cycloalkenyl groups are the following moieties:

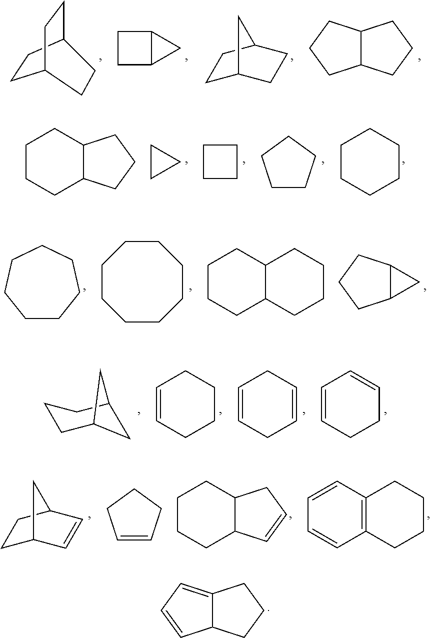

Depending on the structure, a cycloalkyl group includes a monoradical or a diradical (e.g., a cycloalkylene group).

Cycloalkyl and cycloalkenyl groups are optionally substituted with 1, 2, 3, or 4 substituents selected from alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxyl, hydroxyalkyl, mercapto, oxo, —NR_{A}R_{A}, and (NR_{A}R_{B})carbonyl.

The term “cycloalkylalkyl” as used herein, means a cycloalkyl group, as defined herein, appended to the parent molecular moiety through an alkyl group, as defined herein. Illustrative examples of cycloalkylalkyl are cyclopropylmethyl, 2-cyclobutylethyl, cyclopentylmethyl, cyclohexylmethyl, and 4-cycloheptylbutyl.

The term “carbocycle” as used herein, refers to a ring, wherein each of the atoms forming the ring is a carbon atom. Carbocyclic rings include those formed by three, four, five, six, seven, eight, nine, or more than nine carbon atoms. Carbocycles are optionally substituted.

The term “alkoxyalkyl” as used herein, means at least one alkoxy group, as defined herein, appended to the parent molecular moiety through an alkyl group, as defined herein. Illustrative examples of alkoxyalkyl are 2-methoxyethyl, 2-ethoxyethyl, tert-butoxyethyl and methoxymethyl.

The term “alkoxycarbonyl” as used herein, means an alkoxy group, as defined herein, appended to the parent molecular moiety through a carbonyl group, as defined herein. Illustrative examples of alkoxycarbonyl are methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein.

The term “alkylcarbonyl” as used herein, means an alkyl group, as defined herein, appended to the parent molecular moiety through a carbonyl group, as defined herein. Illustrative examples of alkylcarbonyl are acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Illustrative examples of alkylcarbonyloxy are acetyloxy, ethylcarbonyloxy, and tert-butylcarbonyloxy.

The term “alkylthio” or “thioalkoxy” as used herein, means an alkyl group, as defined herein, appended to the parent molecular moiety through a sulfur atom. Illustrative examples of alkylthio are methylthio, ethylthio, butylthio, tert-butylthio, and hexylthio.

The term “alkylthioalkyl” as used herein, means an alkylthio group, as defined herein, appended to the parent molecular moiety through an alkyl group, as defined herein. Illustrative examples of alkylthioalkyl are methylthiomethyl, 2-(ethylthio)ethyl, butylthiomethyl, and hexylthioethyl.

The term “alkynyl” as used herein, means a straight, branched chain hydrocarbon containing from 2-10 carbons and containing at least one carbon-carbon triple bond. Alkynyl groups are optionally substituted. Illustrative examples of alkynyl are acetylenyl, 1-propynyl, 2-propynyl, 3-butynyl, 2-pentynyl, and 1-butynyl.

The term “aromatic” as used herein, refers to a planar ring having a delocalized π-electron system containing 4n+2 π electrons, where n is an integer. Aromatic rings include those formed by five, six, seven, eight, nine, or more than nine atoms. Aromatics are be optionally substituted. The term includes monocyclic or fused-ring polycyclic (i.e., rings which share adjacent pairs of carbon atoms) groups.

The term “aryl” as used herein, refers to an aromatic ring wherein each of the atoms forming the ring is a carbon atom. Aryl rings include those formed by five, six, seven, eight, nine, or more than nine carbon atoms. Illustrative examples of aryl groups are phenyl, naphthalenyl, phenanthrenyl, anthracenyl, fluorenyl, and indenyl.

An aryl group is optionally substituted with one, two, three, four or five substituents independently selected from the group consisting of alkenyl, alkoxy, alkoxyalkyl, alkoxycarbonyl, alkyl, alkylcarbonyl, alkylcarbonyloxy, alkylthio, alkylthioalkyl, alkynyl, carbonyl, cyano, formyl, haloalkoxy, haloalkyl, halogen, hydroxyl, hydroxyalkyl, mercapto, nitro, —NR_{A}R_{A}, and (NR_{A}R_{B})carbonyl.

The term “arylalkyl” as used herein, means an aryl group, as defined herein, appended to the parent molecular moiety through an alkyl group, as defined herein. Illustrative examples of arylalkyl are benzyl, 2-phenylethyl, -phenylpropyl, 1-methyl-3-phenylpropyl, and 2-naphth-2-ylethyl.

The term “carbonyl” as used herein, means a —C(O)— group.

The term “carboxy” as used herein, means a —COOH group.

The term “cyano” as used herein, means a —CN group.

The term “formyl” as used herein, means a —C(O)H group.

The term “halo” or “halogen” as used herein, means a —Cl, —Br, —I or —F.

The term “mercapto” as used herein, means a —SH group.

The term “nitro” as used herein, means a —NO₂ group.

The term “hydroxy” as used herein, means a —OH group.

The term “oxo” as used herein, means a ═O group.

The term “bond” or “single bond” as used herein, refers to a chemical bond between two atoms, or two moieties when the atoms joined by the bond are considered to be part of larger substructure.

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In some embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “alkylamine” refers to the —N(alkyl)ₓH_{y} group, where x and y are selected from among x=1, y=1 and x=2, y=0. When x=2, the alkyl groups, taken together with the N atom to which they are attached, optionally form a cyclic ring system.

The term “amide” as used herein, is a chemical moiety with the formula —C(O)NHR or —NHC(O)R, where R is selected from among hydrogen, alkyl, cycloalkyl, aryl, heteroaryl (bonded through a ring carbon) and heteroalicyclic (bonded through a ring carbon). An amide moiety includes a linkage between an amino acid or a peptide molecule and a compound described herein, e.g., in a prodrug. Any amine, or carboxyl side chain on the compounds described herein is optionally amidified.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein is optionally esterified.

The terms “heteroalkyl” “heteroalkenyl” and “heteroalkynyl” as used herein, include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” as used herein refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms are the same as one another, or some or all of the two or more heteroatoms are different from the other or others.

The term “ring” as used herein, refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings are optionally substituted. In some instances, rings form part of a ring system.

As used herein, the term “ring system” refers to two or more rings, wherein two or more of the rings are fused. The term “fused” refers to structures in which two or more rings share one or more bonds.

The terms “heteroaryl” or, alternatively, “heteroaromatic” refers to an aromatic group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group includes both fused and non-fused groups. Illustrative of heteroaryl groups are the following moieties:

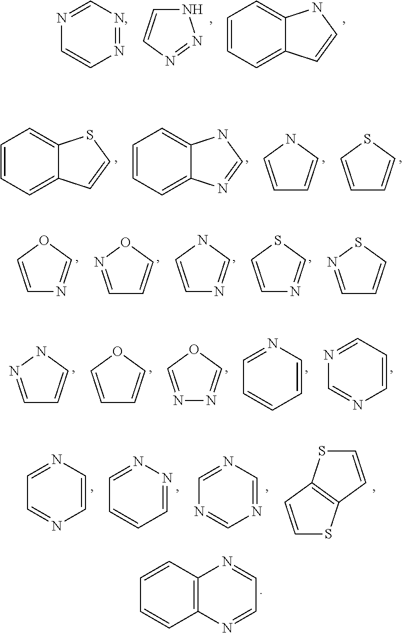

Depending on the structure, a heteroaryl group includes a monoradical or a diradical (i.e., a heteroarylene group).

The term “substituted heteroaryl” (or its equivalent) means heteroaryl groups that are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkyl, mercapto, nitro, —NR_{A}R_{B}, and —(NR_{A}R_{B})carbonyl.

The term “heteroarylalkyl” as used herein, means a heteroaryl, as defined herein, appended to the parent molecular moiety through an alkyl group, as defined herein. An illustrative example of heteroarylalkyl is pyridinylmethyl.

The term “non-aromatic heterocycle”, “non-aromatic heterocyclic”, “heterocycloalkyl” or “heteroalicyclic” as used herein, refers to a non-aromatic ring wherein one or more atoms forming the ring is a heteroatom. A “non-aromatic heterocycle” or “non-aromatic heterocyclic”, “heterocycloalkyl” or “heteroalicyclic” group refers to a cycloalkyl group that includes at least one heteroatom selected from nitrogen, oxygen and sulfur. The radicals include those fused with an aryl or heteroaryl. Non-aromatic heterocycle rings include those formed by three, four, five, six, seven, eight, nine, or more than nine atoms. Heterocycloalkyl rings are optionally substituted. In certain embodiments, non-aromatic heterocycles contain one or more carbonyl or thiocarbonyl groups such as, for example, oxo- and thio-containing groups. Illustrative examples of heterocycloalkyls are lactams, lactones, cyclic imides, cyclic thioamides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine , maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidone, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles are

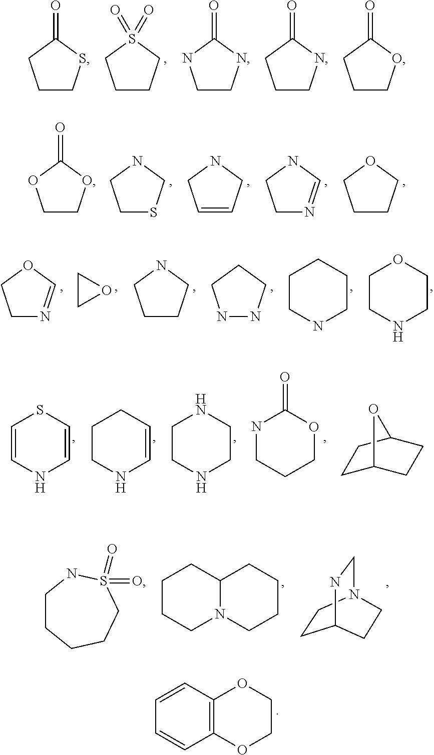

The term heteroalicyclic also includes all ring forms of the carbohydrates, including but not limited to the monosaccharides, the disaccharides and the oligosaccharides.

The term “heterocycle” refers to heteroaromatic and heteroalicyclic used herein, refers to groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocyclic ring optionally has additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms are the same or different from one another. Heterocycles are optionally substituted. Binding to a heterocycle is at a heteroatom or at a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thietanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, include those that are C-attached or N-attached where such is possible. For instance, a group derived from pyrrole includes pyrrol-1-yl groups (N-attached) or pyrrol-3-yl groups (C-attached). Further, a group derived from imidazole includes imidazol-1-yl or imidazol-3-yl (both N-attached) or imidazol-2-yl, imidazol-4-yl or imidazol-S-yl (all C-attached) groups. The heterocyclic groups include benzo-fused ring systems and ring systems substituted with one or two oxo (═O) moieties such as pyrrolidin-2-one. Depending on the structure, a heterocycle group includes a monoradical or a diradical (i.e., a heterocyclene group).

The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkyl, mercapto, nitro, —NR_{A}R_{B}, and —(NR_{A}R_{B})carbonyl.

The term “heterocycloalkylalkyl” as used herein, means a heterocycloalkyl, as defined herein, appended to the parent molecular moiety through an alkyl group, as defined herein.

The term “membered ring” embraces any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “non-aromatic 5, 6, 7, 8, 9, 10, 11 or 12-bicyclic heterocycle” as used herein, means a non-aromatic heterocycle, as defined herein, consisting of two carbocyclic rings, fused together at the same carbon atom (forming a spiro structure) or different carbon atoms (in which two rings share one or more bonds), having 5 to 12 atoms in its overall ring system, wherein one or more atoms forming the ring is a heteroatom. Illustrative examples of non-aromatic 5, 6, 7, 8, 9, 10, 11, or 12-bicyclic heterocycle ring are 2-azabicyclo[2.2.1]heptanyl, 7-azabicyclo[2.2.1]heptanyl, 2-azabicyclo[3.2.0]heptanyl, 3-azabicyclo[3.2.0]heptanyl, 4-azaspiro[2.4]heptanyl, 5-azaspiro[2.4]heptanyl, 2-oxa-S-azabicyclo[2.2.1]heptanyl, 4-azaspiro[2.5]octanyl, 5-azaspiro[2.5]octanyl, 5-azaspiro[3.4]octanyl, 6-azaspiro[3.4]octanyl, 4-oxa-7-azaspiro[2.5]octanyl, 2-azabicyclo[2.2.2]octanyl, 1,3-diazabicyclo[2.2.2]octanyl, 5-azaspiro[3.5]nonanyl, 6-azaspiro[3.5]nonanyl, 5-oxo-8-azaspiro[3.5]nonanyl, octahydrocyclopenta[c]pyrrolyl, octahydro-1H-quinolizinyl, 2,3,4,6,7,9a-hexahydro-1H-quinolizinyl, decahydropyrido[1,2-a]azepinyl, decahydro-1H-pyrido[1,2-a]azocinyl, 1-azabicyclo[2.2.1]heptanyl, 1-azabicyclo[3.3.1]nonanyl, quinuclidinyl, and 1-azabicyclo[4.4.0]decanyl.

The term hydroxylalkyl” as used herein, means at least one hydroxyl group, as defined herein, is appended to the parent molecular moiety through an alkyl group, as defined herein. Illustrative examples of hydroxyalkyl are hydroxymethyl, 2-hydroxy-ethyl, 3-hydroxypropyl and 4-hydroxyheptyl.

The term “NR_{A}R_{B}” as used herein, means two group, R_{A} and R_{B}, as defined herein, which are appended to the parent molecular moiety through a nitrogen atom. Illustrative examples of NR_{A}R_{B} are amino, methylamino, acetylamino, and acetylmethylamino.

The term “(NR_{A}R_{B})carbonyl” as used herein, means a NR_{A}R_{B}, group, as defined herein, which are appended to the parent molecular moiety through a carbonyl group, as defined herein. Illustrative examples of (NR_{A}R_{B})carbonyl are aminocarbonyl, (methylamino)carbonyl, (dimethylamino)carbonyl, and (ethylmethylamino)carbonyl.

The term “NR_{C}R_{D}” as used herein, means two group, R_{C} and R_{D}, as defined herein which are appended to the parent molecular moiety through a nitrogen atom. Illustrative examples of NR_{C}R_{D} are amino, methylamino, acetylamino, and acetylmethylamino.

The term “(NR_{C}R_{D})carbonyl” as used herein, means a NR_{C}R_{D}, group, as defined herein, appended to the parent molecular moiety through a carbonyl group, as defined herein. Illustrative examples of (NR_{C}R_{D})carbonyl are aminocarbonyl, (methylamino)carbonyl, (dimethylamino)carbonyl, and (ethylmethylamino)carbonyl.

As used herein, the term “mercaptyl” refers to a (alkyl)S— group.

As used herein, the term “moiety” refers to a specific segment or functional group of a molecule. Chemical moieties are often recognized chemical entities embedded in or appended to a molecule.

As used herein, the term “sulfinyl” refers to a —S(═O)—R, where R is selected from the group consisting of alkyl, cycloalkyl, aryl, heteroaryl (bonded through a ring carbon) and heteroalicyclic (bonded through a ring carbon).

As used herein, the term “sulfonyl” refers to a —S(═O)₂—R, where R is selected from the group consisting of alkyl, cycloalkyl, aryl, heteroaryl (bonded through a ring carbon) and heteroalicyclic (bonded through a ring carbon).

As used herein, the term “O carboxy” refers to a group of formula RC(═O)O—.

As used herein, the term “C carboxy” refers to a group of formula —C(═O)OR.

As used herein, the term “acetyl” refers to a group of formula —C(═O)CH₃.

As used herein, the term “acyl” refers to a group or radical of formula —C(═O)R where R is an organic group (an example of acyl group is the acetyl group).

As used herein, the term “arylacyl” refers to a group or radical of formula —C(═O)R where R is an aryl group wherein aryl is as defined.

As used herein, the term “heteroarylacyl” refers to a group or radical of formula —C(═O)R where R is a heteroaryl group wherein heteroaryl is as defined.

As used herein, the term “substituted arylacyl” refers to a group or radical of formula —C(═O)R where R is a substituted aryl group wherein substituted aryl is as defined.

As used herein, the term “substituted heteroarylacyl” refers to a group or radical of formula —C(═O)R where R is a substituted heteroaryl group wherein substituted heteroaryl is as defined.

As used herein, the term “trihalomethanesulfonyl” refers to a group of formula X₃CS(═O)₂— where X is a halogen.

As used herein, the term “isocyanato” refers to a group of formula —NCO.

As used herein, the term “thiocyanato” refers to a group of formula —CNS.

As used herein, the term “isothiocyanato” refers to a group of formula —NCS.

As used herein, the term “S sulfonamido” refers to a group of formula —S(═O)₂NR₂.

As used herein, the term “N sulfonamido” refers to a group of formula RS(═O)₂NH—.

As used herein, the term “trihalomethanesulfonamido” refers to a group of formula X₃CS(═O)₂NR—.

As used herein, the term “O carbamyl” refers to a group of formula —OC(═O)NR₂.

As used herein, the term “N carbamyl” refers to a group of formula ROC(═O)NH—. As used herein, the term “O thiocarbamyl” refers to a group of formula —OC(═S)NR₂.

As used herein, the term “N thiocarbamyl” refers to a group of formula ROC(═S)NH—.

As used herein, the term “C amido” refers to a group of formula —C(═O)NR₂.

As used herein, the term “N amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “substituted” means that the referenced group is optionally substituted (substituted or unsubstituted) with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents is LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl.

The term “optionally substituted” as defined herein, means the referenced group is substituted with one or more substituents as defined herein.

The term “protected-hydroxy” refers to a hydroxy group protected with a hydroxy protecting group. The protecting groups that can form the protected hydroxy group are known to those of skill in the art and can be found in references such as Greene and Wuts, Protective Groups in Organic Synthesis; 3rd Edition, John Wiley and Sons: New York, 2006.

In some embodiments, the compounds described herein exist as stereoisomers, wherein asymmetric or chiral centers are present. Stereoisomers are designated (R) or (S) depending on the configuration of substituents around the chiral carbon atom. The term (R) and (S) used herein are configurations as defined in IUPAC 1974 Recommendations for Section E, Fundamental Stereochemistry, Pure Appl. Chem., (1976), 45:13-30, hereby incorporated by reference for this purpose. The embodiments described herein specifically includes the various stereoisomers and mixtures thereof. Stereoisomers include enantiomers, diastereomers, and mixtures of enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary or (2) direct separation of the mixture of optical enantiomers on chiral chromatographic column.

The methods and formulations described herein include the use of N-oxides, crystalline forms (also known as polymorphs), or pharmaceutically acceptable salts of compounds described herein, as well as active metabolites of these compounds having the same type of activity. In some situations, compounds exist as tautomers. All tautomers are included within the scope of the compounds presented herein. In some embodiments, the compounds described herein exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof are chosen, in certain embodiments, to provide stable moieties and compounds.

### Preparation of Compounds

In certain embodiments, the compounds described herein are synthesized using any synthetic techniques including standard synthetic techniques and the synthetic processes described herein. In specific embodiments, the following synthetic processes are utilized.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof.” Precursor functional groups are shown as electrophilic groups and nucleophilic groups. In certain embodiments, a functional group on an organic substance is attached directly, or attached via any useful spacer or linker as defined below.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl , aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a heteroatom, e.g., oxygen or nitrogen.

### Use of Protecting Groups

The term “protecting group” refers to chemical moieties that block some or all reactive moieties and prevent such groups from participating in chemical reactions until the protective group is removed. In some or any specific embodiments, more than one protecting group is utilized. In more specific embodiments, each protective group is removable by a different process. Protective groups that are cleaved under totally disparate reaction conditions fulfill the requirement of differential removal. In some or any embodiments, protective groups are removed by acid, base, or hydrogenolysis. Groups such as trityl, dimethoxytrityl, acetal and t-butyldimethylsilyl are acid labile and are, in some embodiments, used to protect carboxy and hydroxy reactive moieties in the presence of amino groups protected with Cbz groups, which are removable by hydrogenolysis, and Fmoc groups, which are base labile. In some or any embodiments, carboxylic acid and hydroxy reactive moieties are blocked with base labile groups such as, without limitation, methyl, ethyl, and acetyl in the presence of amines blocked with acid labile groups such as t-butyl carbamate or with carbamates that are both acid and base stable but hydrolytically removable.

In some or any embodiments, carboxylic acid and hydroxy reactive moieties are blocked with hydrolytically removable protective groups such as the benzyl group, while, in some or any embodiments, amine groups capable of hydrogen bonding with acids are blocked with base labile groups such as Fmoc. In some or any embodiments, carboxylic acid reactive moieties are protected by conversion to simple ester derivatives as exemplified herein, or they are blocked with oxidatively-removable protective groups such as 2,4-dimethoxybenzyl, while, in some embodiments, co-existing amino groups are blocked with fluoride labile silyl carbamates.

In some or any instances, allyl blocking groups are useful in the presence of acid- and base-protecting groups since the former are stable. In some or any embodiments, such groups are subsequently removed by metal or pi-acid catalysts. For example, in some or any embodiments, an allyl-blocked carboxylic acid is deprotected with a Pd⁰-catalyzed reaction in the presence of acid labile t-butyl carbamate or base-labile acetate amine protecting groups. In some or any embodiments, a protecting group is a resin to which a compound or intermediate is attached. As long as the residue is attached to the resin, that functional group is blocked and cannot react. Once released from the resin, the functional group is available to react.

In some or any embodiments, blocking/protecting groups are selected from, by way of non-limiting example:

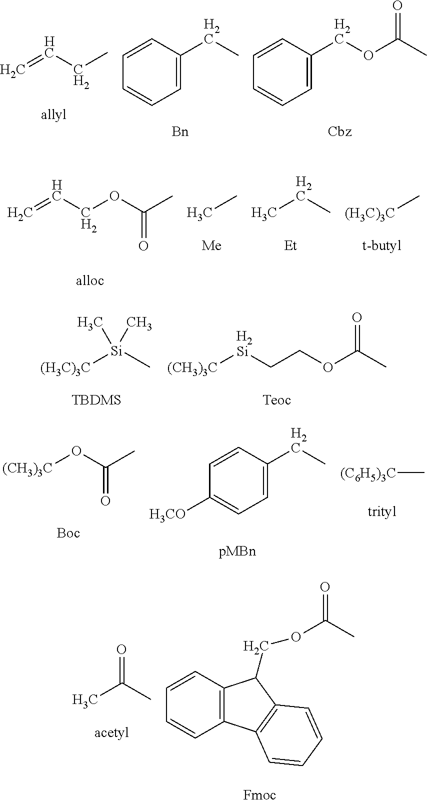

Other protecting groups are described in Greene and Wuts, Protective Groups in Organic Synthesis, 3rd Ed., John Wiley & Sons, New York, N.Y., 1999.

### Compounds of Formula (I), (II) or (III).

In some or any embodiments, compounds of Formula (I), (II) and (III) are prepared by various methods, as outlined in Synthetic Schemes I-XI. In each scheme, the variables (e.g., R¹, R², R³, etc) are represented by hydrogen. In some embodiments, compounds are synthesized using methodologies analogous to those described below by the use of appropriate alternative starting materials. When R² and R³ are other than hydrogen, the appropriate starting material is obtained before subsequent synthetic steps are performed.

In some or any embodiments, compounds of Formula (IIA) are synthesized according to Synthetic Scheme I wherein by way of example, the A is represented by a 3-pyridinyl group, Y is (CH₂)ₘ wherein m is 2 and Z is oxygen. Compounds with A as other heteroaryl groups are prepared using similar synthetic route with the use of appropriate boronic acid derivatives in step 4.

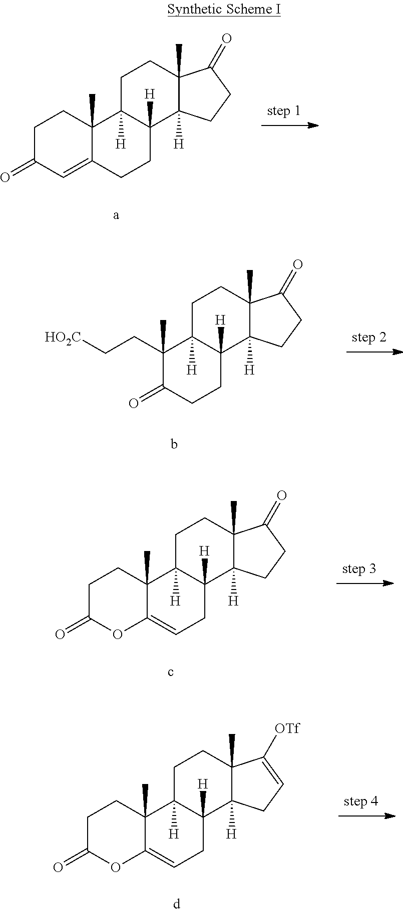

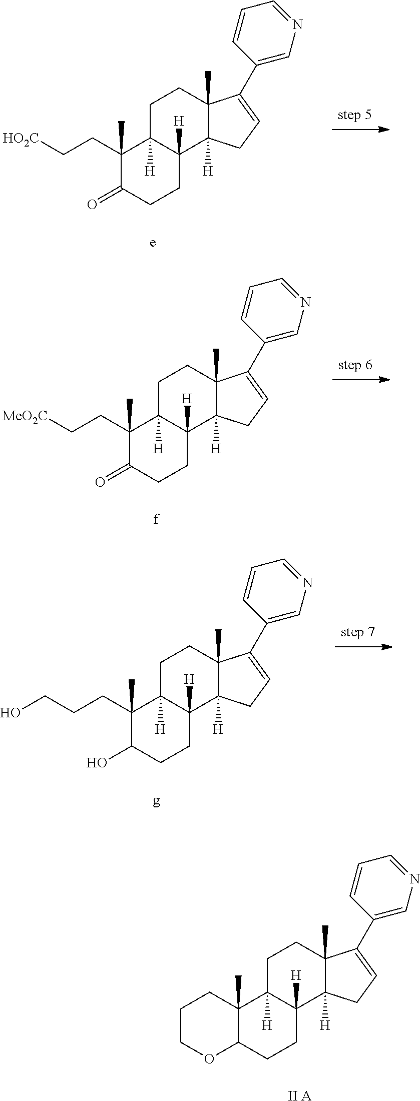

Compounds of structure of Formula (IIA) having A as 3-pyridinyl group and Y being (CH₂)ₘ wherein m as 2 and Z being oxygen are synthesized from commercially available starting material 1. Oxidation of compound (a) with sodium periodate, potassium permanganate and potassium carbonate in water and t-butanol solution at refluxing temperature and subsequent treatment with dilute hydrochloric acid gives the ring opening product compound (b) (step 1). Treatment of compound (2) with acetic anhydride in the presence of sodium acetate yields the compound (3) (step 2). Conversion of compound (c) to compound (d) is achieved by using condition as in Step 3 involving the enol triflate formation by the use of triflic anhydride (trifluoromethanesulfonic anhydride) in the presence of base such as triethylamine and the like. Suzuki coupling reaction on compound (d) with 3-(diethylboryl)pyridine (or pyridine-3-yl boronic acid), and (Ph₃P)₂PdCl₂ in THF-water or dioxane-water in the presence of a base such as sodium carbonate followed by acidification yields compound (e) (step 4). Esterification of compound (e) with thionyl chloride in methanol gives the methyl ester compound (f) (step 5). Treatment of compound (f) with diisobutylaluminium hydride (DIBAL-H) in tetrahydrofuran (THF) yields compound (g) (step 6). Reaction of compound (g) with to p-toluenesulfonyl chloride in pyridine yields compound of Formula (IIA) where the A is a 3 pyridinyl group and Y being (CH₂)ₘ wherein m as 2 and Z being oxygen (step 7). Compounds of Formula (IIA) having different A are made similarly by using appropriate boronic acid instead of pyridine-3-yl boronic acid in step 4.

In some or any embodiments, compounds of Formula (IIA) wherein the A is represented by a 3-pyridinyl group, Y is (CH₂)ₘ wherein m is 1 and Z is oxygen are synthesized according to Synthetic Scheme II . Compounds with A as other heteroaryl groups are prepared using similar synthetic route with the use of appropriate boronic acid derivatives in step 10.

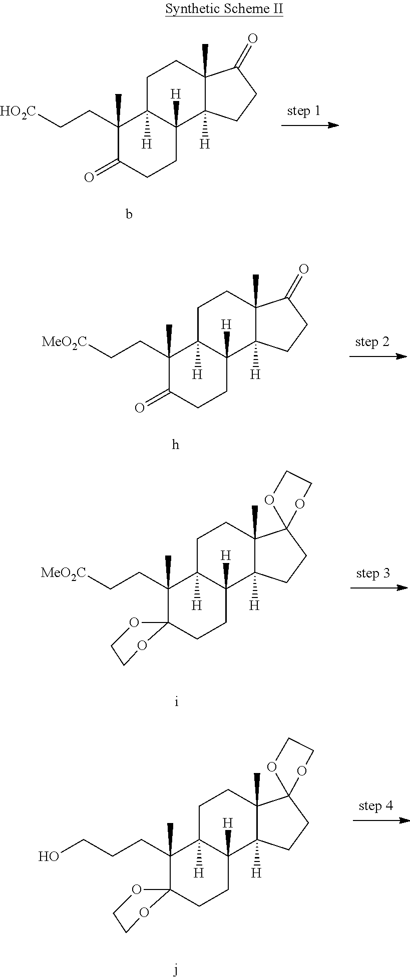

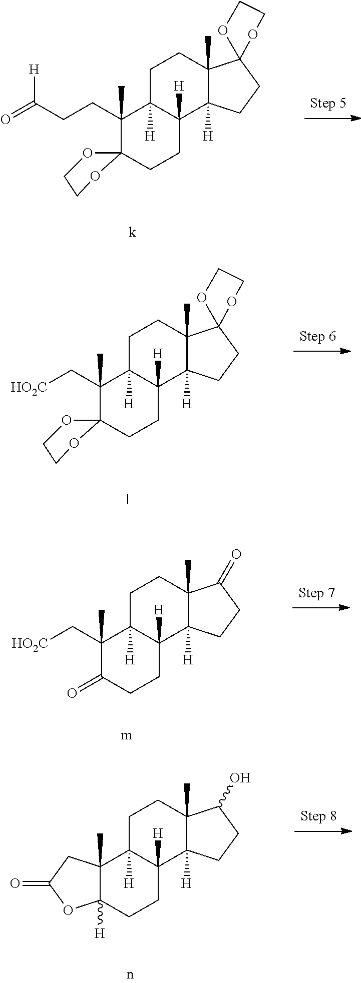

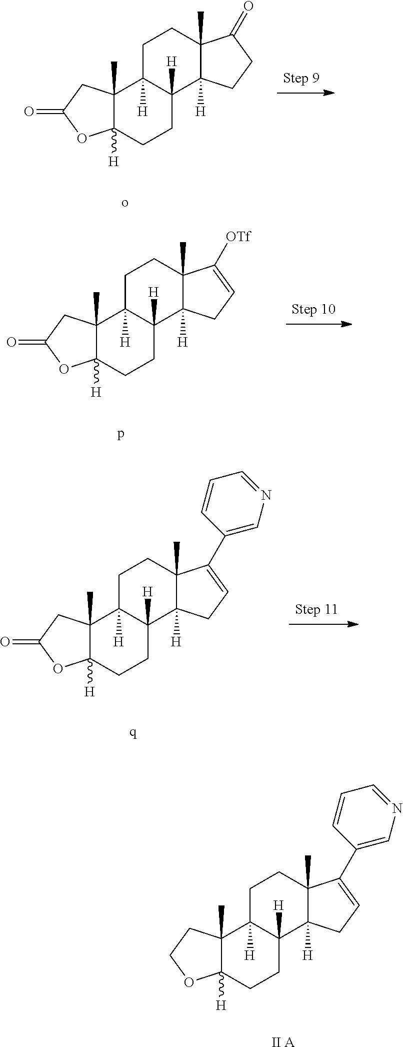

Compounds having the structure of Formula (IIA) wherein the A is represented by a 3-pyridinyl group, Y is (CH₂)ₘ wherein m is 1 and Z is oxygen are synthesized from compound (b) which is synthesized by a route mentioned above. Esterification of compound (b) with thionyl chloride in methanol gives the methyl ester compound (h) (step 1). Step 2 of the synthesis requires the ketalization of the keto groups with p-toluenesulfonic acid and ethylene glycol in organic solution such as toluene and the like at reflux with removal of water to give compound (i). Treatment of compound (i) with lithium aluminium hydride (LIAlH₄) in tetrahydrofuran (THF) yields compound (j) (step 3). Oxidation of compound (j) with trichloroisocyanuric acid (TCCA) and catalytic amount of 2,2,6,6-tetramethyl-piperidinyloxy (TEMPO) in methylene chloride or other organic solvent at room temperature yields the corresponding aldehyde compound (k) (step 4). Step 5 involves the use of N-methylmorpholine, N-methylmorpholine N-oxide and Osmium tetraoxide for the oxidation of compound (k) to the acid with the removal of one carbon atom compound (1). Alternatively, compound (1) is prepared by stepwise by reating compound (k) with sodium periodate in acetone-water to yield the aldehyde followed by further oxidation with m-chloroperbenzoic zcid and sodium bicarbonate (step 5a). Acid hydrolysis of the ketal groups of compound (1) with aqueous hydrochloric acid and the like in tetrahydrofuran yields compound (m) (step 6). Sodium borohydride reduction followed by acidic work up with refluxing the product in toluene in the presence of p-toluenesulfonic acid gives the lactone alcohol compound (n) (step 7). Step 8 involves the use of trichloroisocyanuric acid (TCCA) and 2,2,6,6-tetramethyl-piperidinyloxy (TEMPO) in methylene chloride for the oxidation of compound (n) to the keto derivative compound (o). Step 9 involves the triflate formation by the use of triflic anhydride (trifluoromethanesulfonic anhydride) in the presence of base such as triethylamine and the like to give compound (p). Step 10 requires the Suzuki coupling reaction on compound (p) with 3-(diethylboryl)pyridine (or pyridine-3-yl boronic acid), and (Ph₃P)₂PdCl₂ in THF in the presence of a base such as sodium carbonate yields compound (q). Lithium aluminium hydride reduction of compound (q) in tetrahydrofuran or other organic solvent gives the diol which is immediately treated with tosyl chloride in pyridine to give compound of Formula (IIA) wherein the A is a 3 pyridinyl group and Y being (CH₂)ₘ wherein m as 1 and Z being oxygen (step 11). Compounds of Formula (IVA) wherein the A is a heteroaryl group and Y being (CH₂)ₘ wherein m as 1 and Z being oxygen having different A are made similarly by using appropriate boronic acid instead of pyridine-3-yl boronic acid in step 10.

In some or any embodiments, compounds of Formula (IIB) are synthesized according to Synthetic Scheme III wherein by way of example, the A is represented by a 3-pyridinyl group, Y is (CH₂)ₘ wherein m is 2 and Z is nitrogen. Compounds with A as other heteroaryl groups are prepared using similar synthetic route with the use of appropriate boronic acid derivatives in step 9.

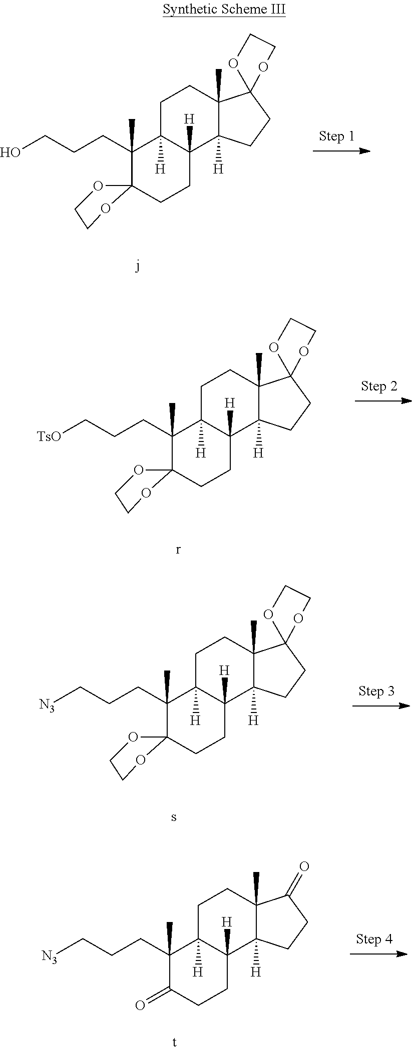

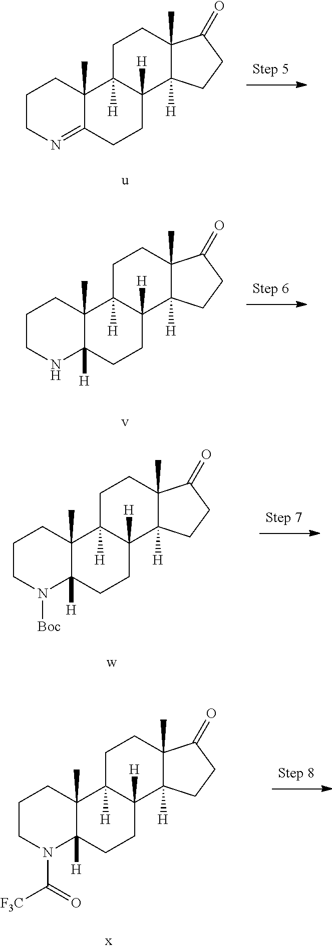

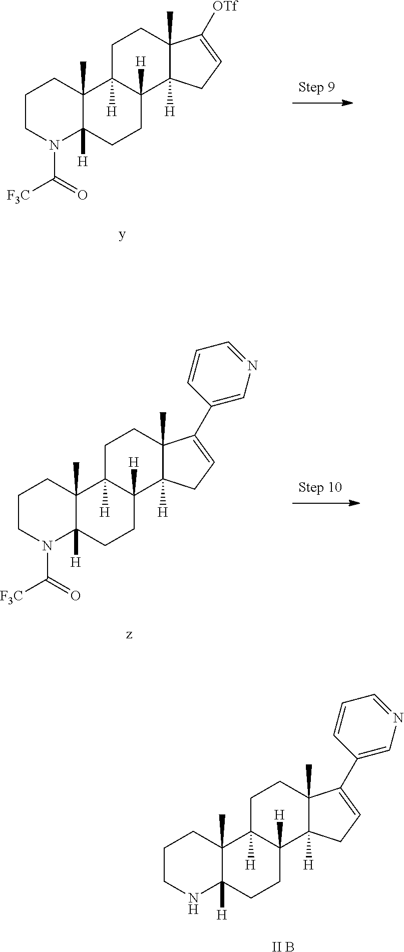

Compounds of structure of Formula (IIB) having A as 3-pyridinyl group and Y being (CH₂)ₘ wherein m as 2 and Z being nitrogen are synthesized from compound (j) which is synthesized by a route mentioned above. Reaction of compound (j) with tosyl chloride in the presence of an organic base such as pyridine, triethylamine and the like in an organic solvent such as methylene chloride yields the tosylate compound (r) (step 1). Treatment of compound (r) with sodium azide in dimethylformamide or similar organic solvent yields the azide compound (s) (step 2). Conversion of compound (s) to compound (t) is achieved by hydrolyzing the ketals with hydrochloric acid solution in tetrahydrofuran (Step 3). Compound (t) is subjected to catalytic hydrogen under hydrogen atmosphere using catalytic amount of palladium on charcoal in methanol (step 4). The azide is reduced and it cyclized to give the imine compound (u) which is reduced immediately with sodium cyanoborohydride in acetic acid at low temperature to give compound (v) (step 5). Compound (v) is converted to the Boc-derivative compound (w) upon treatment with di-tert-butyl dicarbonate (step 6). Conversion of compound (w) to compound (x) involves the hydrolysis of the Boc group with trifluoroacetic acid followed upon worked up by treatment with trifluoroacetic anhydride in dichloromethane at low temperature (step 7). Reaction of compound (x) with trifluoromethanesulfonic anhydride in dichloromethane in the presence of triethylamine and the like gives compound (y) (step 8). Suzuki coupling reaction on compound (y) with 3-(diethylboryl)pyridine (or pyridine-3-yl boronic acid), and (Ph₃P)₂PdCl₂ in THF-water or dioxane—water in the presence of a base such as sodium carbonate yields compound (z) (step 9). Treatment of compound (z) with aqueous potassium carbonate solution in methanol at high temperature yields compound (II B) wherein the A is a 3 pyridinyl group and Y being (CH₂)ₘ wherein m as 2 and Z being NR¹ where R¹ is H (step 10). Reductive amination of this compound with various aldehydes in the presence of sodium cyanoborohydride will give compound with Z being NR¹ where R¹ is alkyl or other R¹ substituents. Acylation of compound (II B) wherein the A is a 3 pyridinyl group and Y being (CH₂)ₘ wherein m as 2 and Z being NR¹ where R¹ is H with various acyl chlorides will give compound (II B) wherein the A is a 3 pyridinyl group and Y being (CH₂)ₘ wherein m as 2 and Z being NCOR¹. Compounds of Formula (IIB) having different A are made similarly by using appropriate boronic acid instead of pyridine-3-yl boronic acid in step 9.

In some or any embodiments, compounds of Formula (IIB) wherein the A is represented by a 3-pyridinyl group, Y is (CH₂)ₘ wherein m is 1 and Z is nitrogen are synthesized according to Synthetic Scheme IV . Compounds with A as other heteroaryl groups are prepared using similar synthetic route with the use of appropriate boronic acid derivatives in step 8.

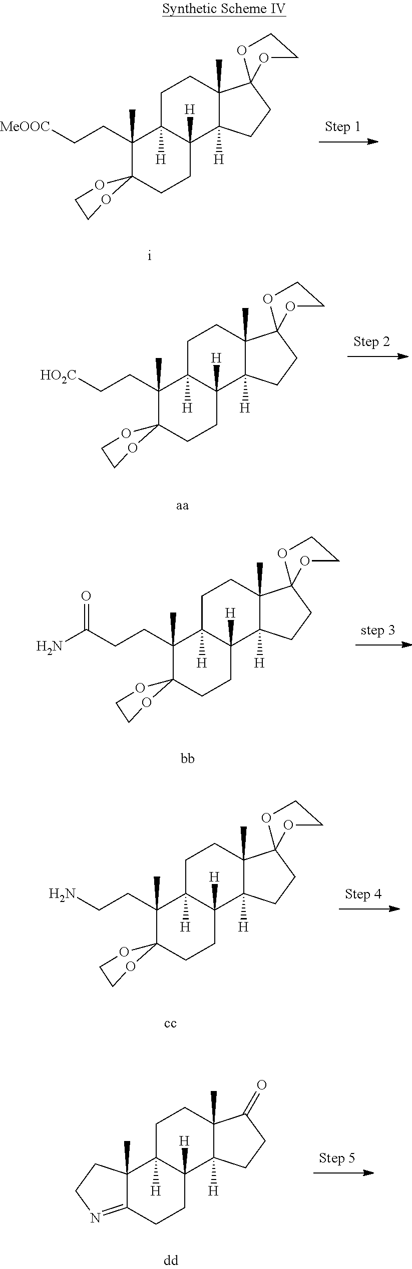

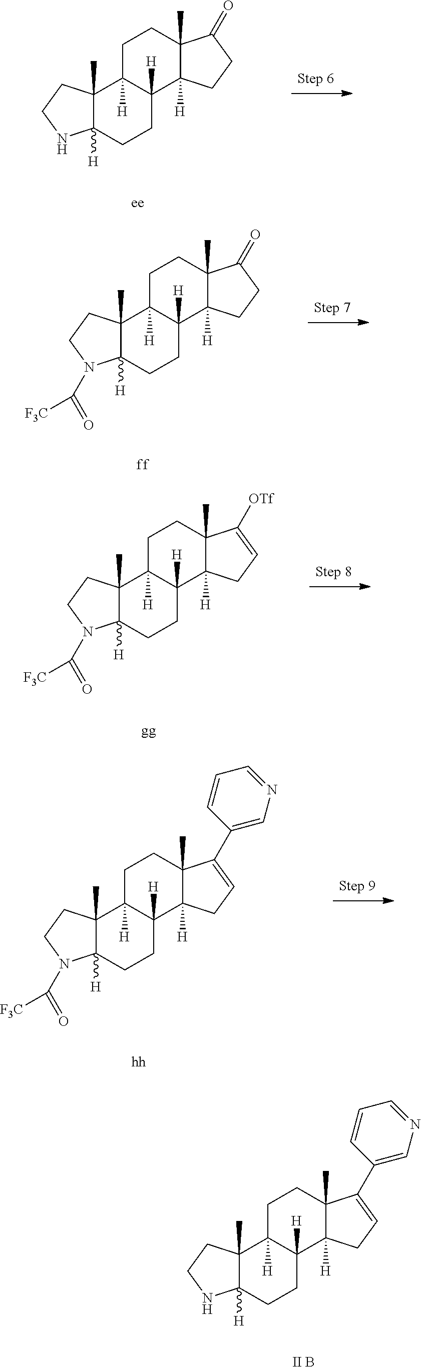

Compounds of structure of Formula (IIB) having A as 3-pyridinyl group and Y being (CH₂)ₘ wherein m as 1 and Z being nitrogen are synthesized from compound (i) which is synthesized by a route mentioned above. Reaction of compound (9) with aqueous sodium hydroxide in methanol gives compound (aa) (step 1). Treatment of compound (aa) with di-tert-butyl dicarbonate and pyridine in acetonitrile followed by ammonium bicarbonate yields amide compound (bb) (step 2). Hofmann rearrangement reaction on compound (bb) by treatment of it with phenyl iododiacetate in acetonitrile-water gives compound (cc) (step 3). Conversion of compound (cc) to compound (dd) is achieved by hydrolyzing the ketals with hydrochloric acid solution in tetrahydrofuran (step 4). Compound (dd) is subjected to sodium cyanoborohydride in acetic acid at low temperature to give compound (ee) (step 5). Compound (ee) is converted to compound (ff) through the preparation of Boc-derivative upon treatment with di-tert-butyl dicarbonate. This boc-derivative is then subjected to the hydrolysis of the Boc group with trifluoroacetic acid followed upon worked up by treatment with trifluoroacetic anhydride in dichloromethane at low temperature (step 6). Reaction of compound (ff) with trifluoromethanesulfonic anhydride in dichloromethane in the presence of triethylamine and the like gives compound (gg) (step 7). Suzuki coupling reaction on compound (gg) with 3-(diethylboryl)pyridine (or pyridine-3-yl boronic acid), and (Ph₃P)₂PdCl₂ in THF-water or dioxane-water in the presence of a base such as sodium carbonate yields compound (hh) (step 8). Treatment of compound (hh) with aqueous potassium carbonate solution in methanol at high temperature yields compound (II B) wherein the A is a 3 pyridinyl group and Y being (CH₂)ₘ wherein m as 1 and Z being NR¹ where R¹ is H (step 9). Reductive amination of this compound with various aldehydes in the presence of sodium cyanoborohydride will give compound with Z being NR¹ where R¹ is alkyl or other R¹ substituents. Acylation of compound (II B) wherein the A is a 3 pyridinyl group and Y being (CH₂)ₘ wherein m as 1 and Z being NR¹ where R¹ is H with various acyl chlorides will give compound (II B) wherein the A is a heteroaryl and Y being (CH₂)ₘ wherein m as 1 and Z being NCOR¹. Compounds of Formula (IIB) having different A are made similarly by using appropriate boronic acid instead of pyridine-3-yl boronic acid in step 8.

In some or any embodiments, compounds of Formula (IG) and (IH) are synthesized according to Synthetic Scheme V wherein by way of example, A is represented by a 3-pyridinyl group. Compounds with A as other heteroaryl groups are prepared using similar synthetic route with the use of appropriate boronic acid derivatives in step 6.

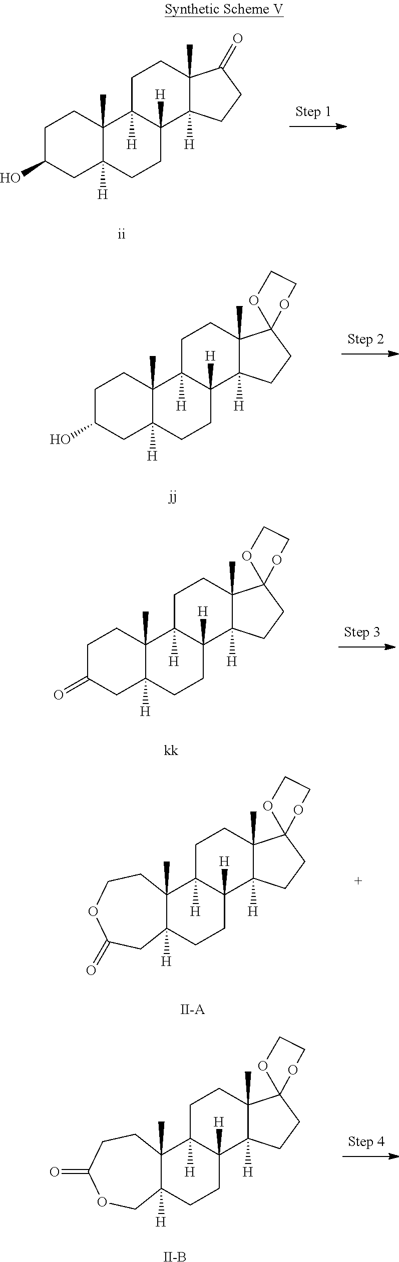

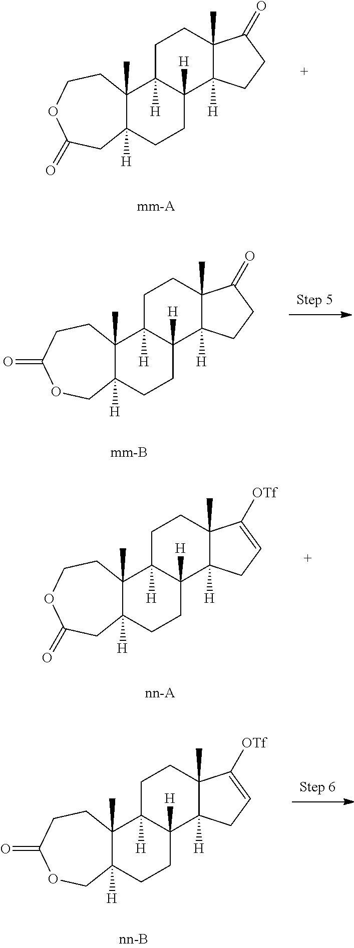

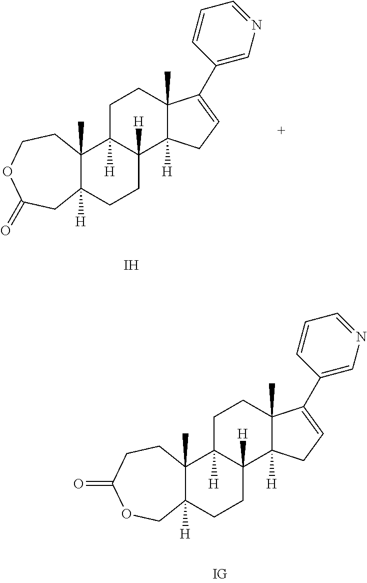

Compounds of structure of Formula (IG and IH) having A as 3-pyridinyl group are synthesized from commercially available starting material epiandrosterone (ii). Ketalization of the keto group of compound (ii) with p-toluenesulfonic acid and ethylene glycol in organic solution such as toluene and the like at reflux with removal of water gives compound (jj) (step 1). Oxidation of compound (jj) with Dess-Martin Periodinane in dichloromethane gives the keto product compound (kk) (step 2). Treatment of compound (kk) with m-chloroperbenzoic acid in dichloromethane yields a mixture of compounds (ll-A and ll-B) (step 3). These two compounds are separated by flash column chromatography. Treatment of the mixture of compounds (ll-A) and (ll-B) with p-toluenesulfonic acid in water-acetone solution yields a mixture of the keto compounds (mm-A) and (mm-B) which are purified and separated by flash column chromatography (step 4). Conversion of compounds (mm-A) and (mm-B) to compounds (nn-A) and (nn-B) is achieved by using condition involving the enol triflate formation by the use of triflic anhydride (trifluoromethanesulfonic anhydride) in the presence of base such as triethylamine and the like (step 5). Suzuki coupling reaction on compounds (nn-A) and (nn-B) with 3-(diethylboryl)pyridine (or pyridine-3-yl boronic acid), and (Ph₃P)₂PdCl₂ in THF-water or dioxane-water in the presence of a base such as sodium carbonate followed by acidification yields compounds of formula (IH) and (IG) wherein the A is a 3 pyridinyl group (step 6). Compounds of Formula (IH) and (IG) having different heteroaryl A are made similarly by using appropriate boronic acid instead of pyridine-3-yl boronic acid in step 6.

In some or any embodiments, compounds of Formula (IK) are synthesized according to Synthetic Scheme VI wherein by way of example the A is represented by a 3-pyridinyl group. Compounds with A as other heteroaryl groups are prepared using similar synthetic route with the use of appropriate boronic acid derivatives in step 1.

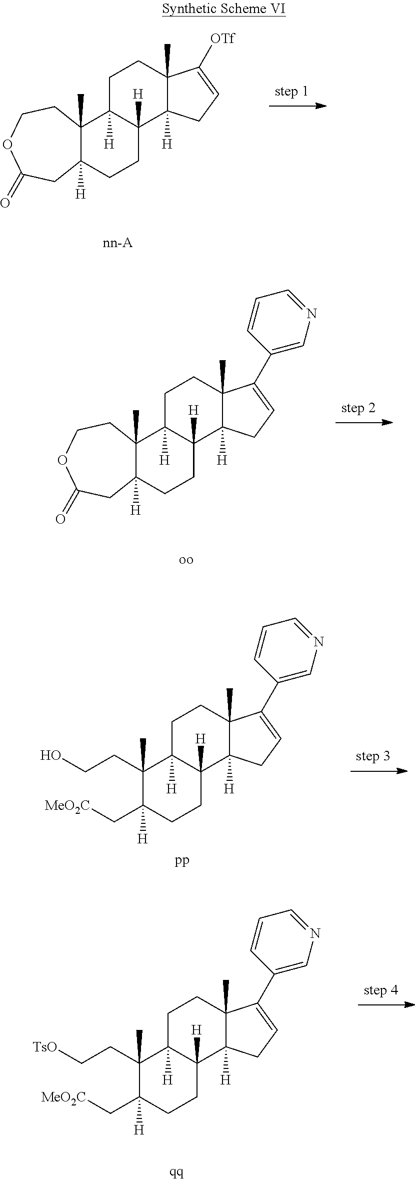

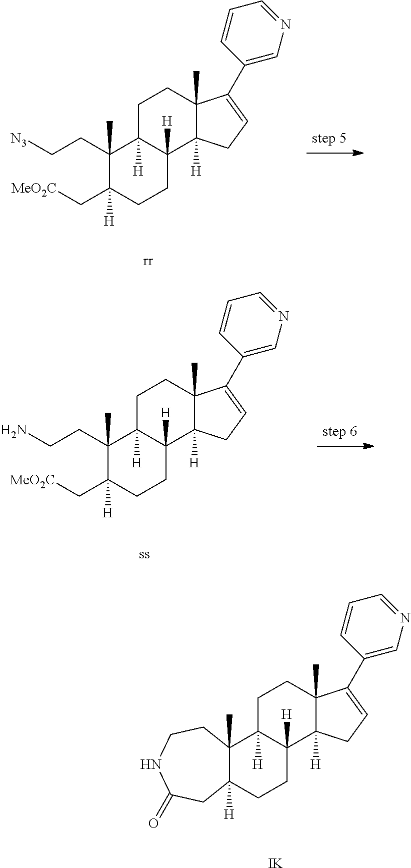

Compounds of structure of Formula (IK) having A as 3-pyridinyl group are synthesized from compound (nn-A) which is prepared from method described above. Suzuki coupling reaction on compounds (nn-A) with 3-(diethylboryl)pyridine (or pyridine-3-yl boronic acid), and (Ph₃P)₂PdCl₂ in THF-water or dioxane-water in the presence of a base such as sodium carbonate followed by acidification yields compound (oo) (step 1). Hydrolysis the lactone ring with the use of p-toluenesulfonic acid in methanol yields the ester-alcohol compound (pp) (step 2). Treatment of compound (pp) with p-toluenesulfonyl chloride in the presence of pyridine or triethyl amine and the like in dichloromethane or other organic solvent yields the tosyl compound (qq) (step 3). Treatment of compound (qq) with sodium azide in dimethylformamide or similar organic solvent yields the azide compound (rr) (step 4). The azide group of compound (rr) is reduced by the use of tributylphosphine in THF-water to give the amino compound (ss) (step 5). Cyclization of compound (ss) with the use of strong base yields compound of formula (IK) wherein the A is a 3 pyridinyl group (step 6). Compounds of Formula (IK) having different heteroaryl A are made similarly by using appropriate boronic acid instead of pyridine-3-yl boronic acid in step 1.

In some or any embodiments, compounds of Formula (IL) are synthesized according to Synthetic Scheme VII wherein by way of example the A is represented by a 3-pyridinyl group. Compounds with A as other heteroaryl groups are prepared using similar synthetic route with the use of appropriate boronic acid derivatives in step 1.

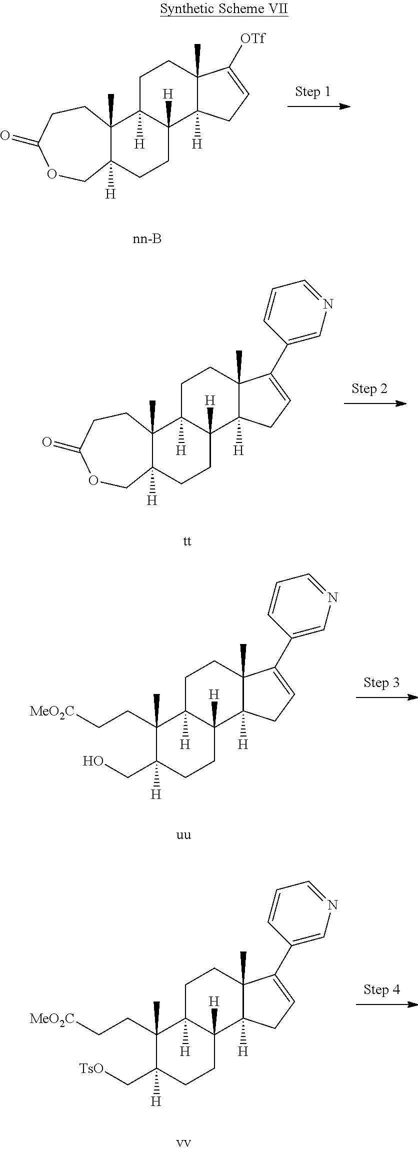

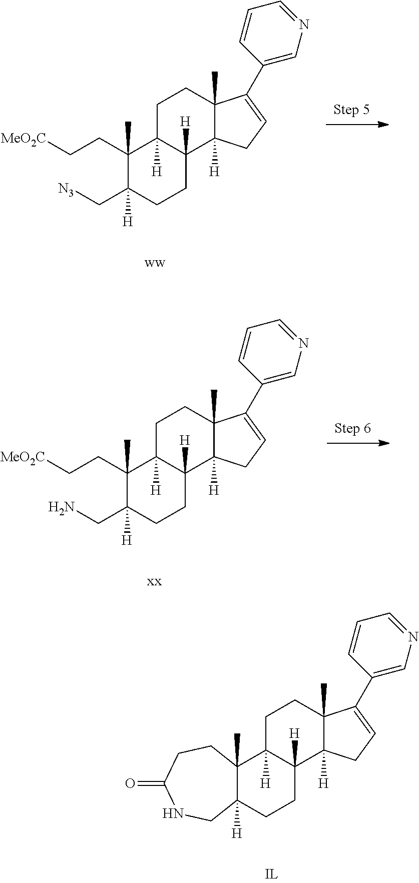

Compounds of structure of Formula (IL) having A as 3-pyridinyl group are synthesized from compound (nn-B) which is prepared from method described above. Suzuki coupling reaction on compounds (nn-B) with 3-(diethylboryl)pyridine (or pyridine-3-yl boronic acid), and (Ph₃P)₂PdCl₂ in THF-water or dioxane-water in the presence of a base such as sodium carbonate followed by acidification yields compound (tt) (step 1). Hydrolysis the lactone ring with the use of p-toluenesulfonic acid in methanol yields the ester-alcohol compound (uu) (step 2). Treatment of compound (uu) with p-toluenesulfonyl chloride in the presence of pyridine or triethyl amine and the like in dichloromethane or other organic solvent yields the tosyl compound (vv) (step 3). Treatment of compound (vv) with sodium azide in dimethylformamide or similar organic solvent yields the azide compound (ww) (step 4). The azide group of compound (ww) is reduced by the use of tributylphosphine in THF-water to give the amino compound (xx) (step 5). Cyclization of compound (xx) with the use of strong base yields compound of formula (IL) wherein the A is a 3 pyridinyl group (step 6). Compounds of Formula (IL) having different heteroaryl A are made similarly by using appropriate boronic acid instead of pyridine-3-yl boronic acid in step 1.

In some or any embodiments, compounds of Formula (III) are synthesized according to Synthetic Scheme VIII wherein by way of example the A is represented by a 3-pyridinyl group. Compounds with A as other heteroaryl groups are prepared using similar synthetic route with the use of appropriate boronic acid derivatives in step 3.

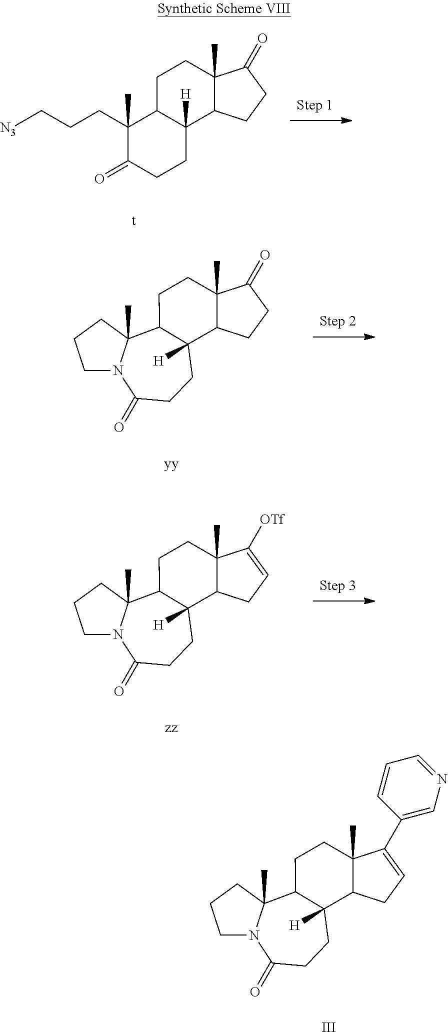

Compounds of structure of Formula (III) having A as 3-pyridinyl group are synthesized from compound (t) which is prepared from method described above. Treatment of compound (t) with titanium(IV) chloride in dichloromethane gives compound (yy) (step 1). Conversion of compound (yy) to compound (zz) is achieved by using condition involving the enol triflate formation by the use of triflic anhydride (trifluoromethanesulfonic anhydride) in dichloromethane in the presence of base such as triethylamine and the like (step 2). Suzuki coupling reaction on compounds (zz) with 3-(diethylboryl)pyridine (or pyridine-3-yl boronic acid), and (Ph₃P)₂PdCl₂ in THF-water or dioxane—water in the presence of a base such as sodium carbonate yields compound of formula (III) (step 3) wherein the A is a 3 pyridinyl group. Compounds of Formula (III) having different heteroaryl A are made similarly by using appropriate boronic acid instead of pyridine-3-yl boronic acid in step 3.

In some or any embodiments, compounds of Formula (I J) are synthesized according to Synthetic Scheme IX wherein by way of example the A is represented by a 3-pyridinyl group. Compounds with A as other heteroaryl groups are prepared using similar synthetic route with the use of appropriate boronic acid derivatives in step 1.

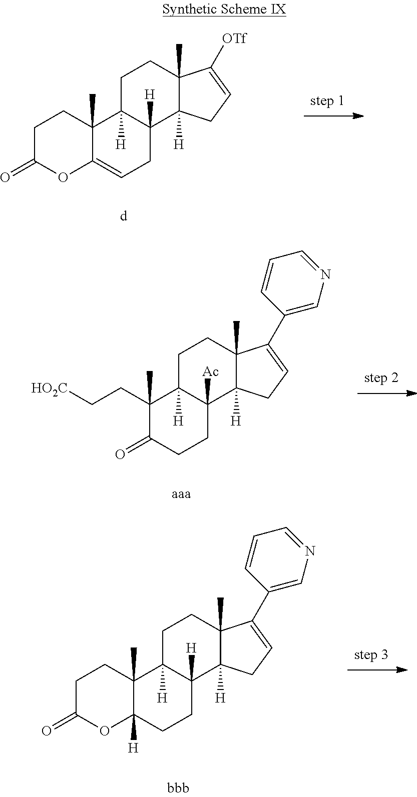

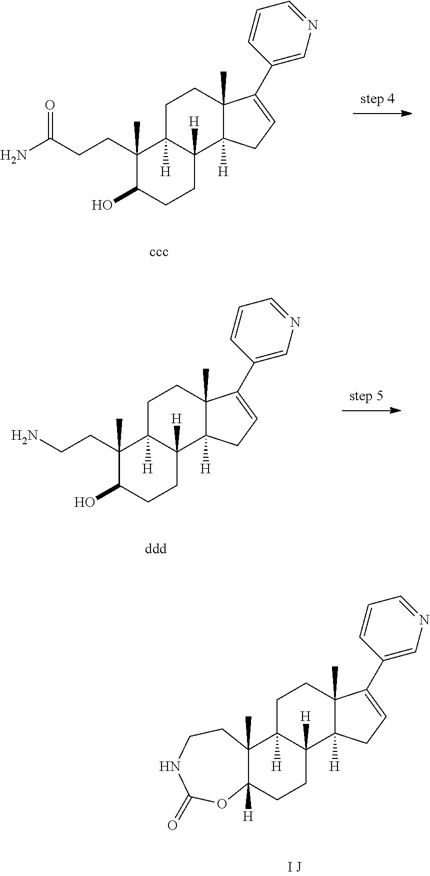

Compounds of structure of Formula (I J) having A as 3-pyridinyl group are synthesized from compound (d) which is prepared from method described above. Suzuki coupling reaction on compounds (d) with 3-(diethylboryl)pyridine (or pyridine-3-yl boronic acid), and (Ph₃P)₂PdCl₂ in THF-water or dioxane-water in the presence of a base such as sodium carbonate followed by acidification yields compound (aaa) (step 1). Sodium borohydride reduction followed by acidic work up with refluxing the product in toluene in the presence of p-toluenesulfonic acid gives the lactone compound (bbb) (step 2). Aminoloysis of the lactone ring with the use of ammonia in methanol yields the amide alcohol compound (ccc) (step 3). Hofmann rearrangement reaction on compound (ccc) with phenyl iododiacetate in acetonitrile-water yields the amine alcohol compound (ddd) (step 4). Treatment of compound (ddd) with phosgene and triethyl amine in tetrahydrofuran gives compound of formula (I J) wherein the A is a 3 pyridinyl group (step 5). Compounds of Formula (I J) having different heteroaryl A are made similarly by using appropriate boronic acid instead of pyridine-3-yl boronic acid in step 1.

In some or any embodiments, compounds of Formula (IN) are synthesized according to Synthetic Scheme X wherein by way of example the A is represented by a 3-pyridinyl group. Compounds with A as other heteroaryl groups are prepared using similar synthetic route with the use of appropriate boronic acid derivatives in step 9.

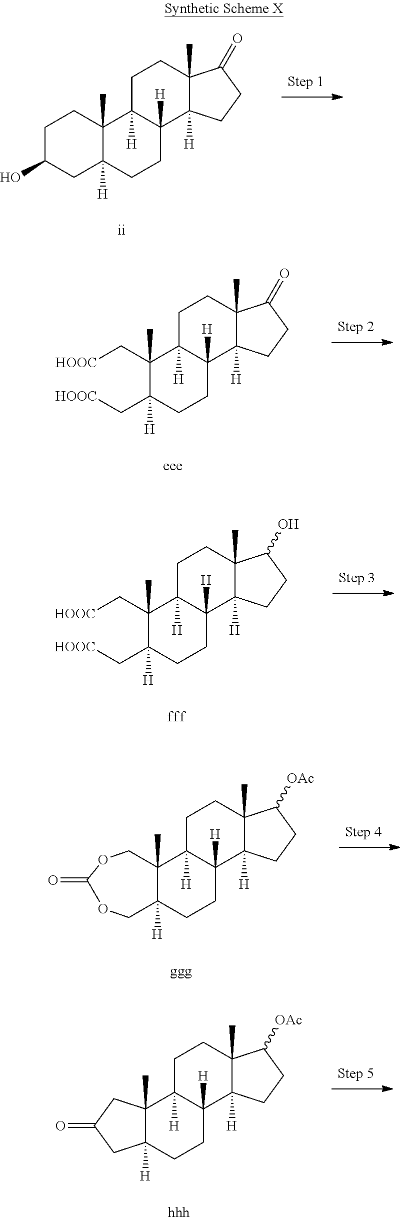

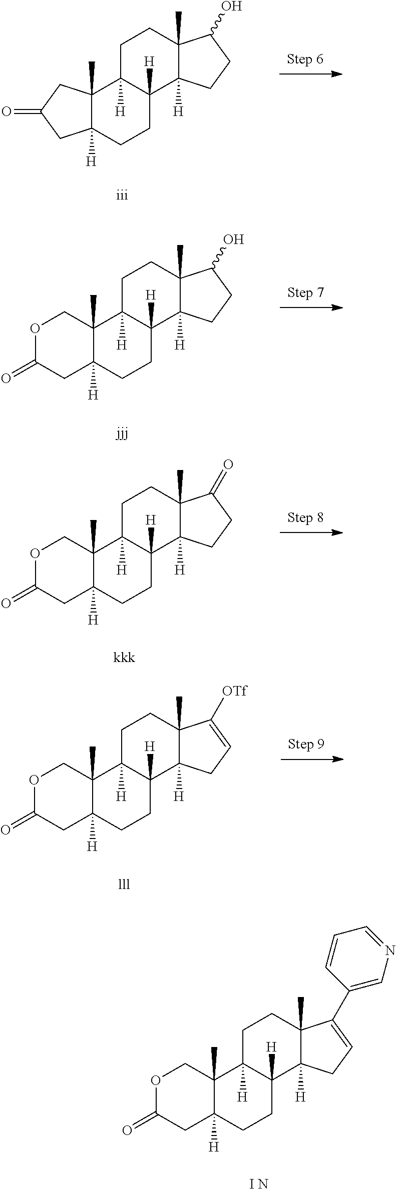

Compounds of structure of Formula (IN) having A as 3-pyridinyl group are synthesized from epiandrosterone compound (ii) which is commericial available. Oxidation of compound (ii) with chromium (VI) oxide in the presence of sulfuric acid and acetic acid-water yields the diacid compound (eee) (step 1). Reduction the diacid compound (eee) with sodium borohydride gives the alcohol compound (fff) (step 2). Treatment of compound (eee) with sodium acetate in acetic anhydride yields the anhydride compound (ggg) (step 3). Compound (ggg) upon heating gives the ketone acetate (hhh) (step 4). Hydrolysis of the O-acetyl group with sodium hydroxide solution yields the ketone alcohol compound (iii) (step 5). Treatment of compound (iii) with m-chloroperbenzoic acid in dichloromethane yields compounds (hhh) together with the other regioisomer (step 6). Step 7 involves the use of trichloroisocyanuric acid (TCCA) and 2,2,6,6-tetramethyl-piperidinyloxy (TEMPO) in methylene chloride for the oxidation of compound (hhh) to the keto derivative compound (kkk) Conversion of compound (kkk) to compound (lll) is achieved by using condition involving the enol triflate formation by the use of triflic anhydride (trifluoromethanesulfonic anhydride) in the presence of base such as triethylamine and the like (step 8). Suzuki coupling reaction on compounds (lll) with 3-(diethylboryl)pyridine (or pyridine-3-yl boronic acid), and (Ph₃P)₂PdCl₂ in THF-water or dioxane-water in the presence of a base such as sodium carbonate followed by acidification yields compounds of Formula (IN) (step 9). Preparation of compounds of Formula (IN) having different heteroaryl A are made similarly by using appropriate boronic acid instead of pyridine-3-yl boronic acid in step 9.

In some or any embodiments, compounds of Formula (IE) and (I M) are synthesized according to Synthetic Scheme XI wherein by way of example the A is represented by a 3-pyridinyl group. Compounds with A as other heteroaryl groups are prepared using similar synthetic route with the use of appropriate boronic acid derivatives in step 3.

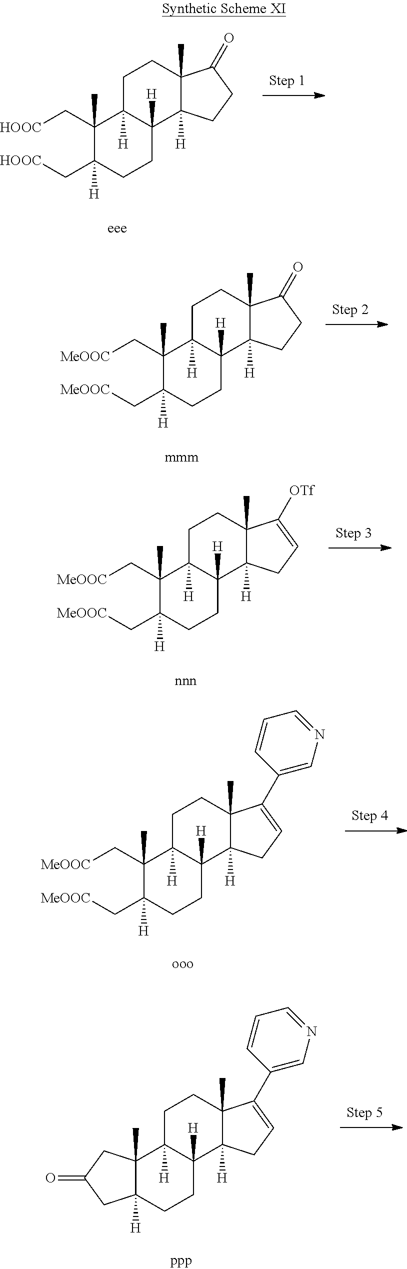

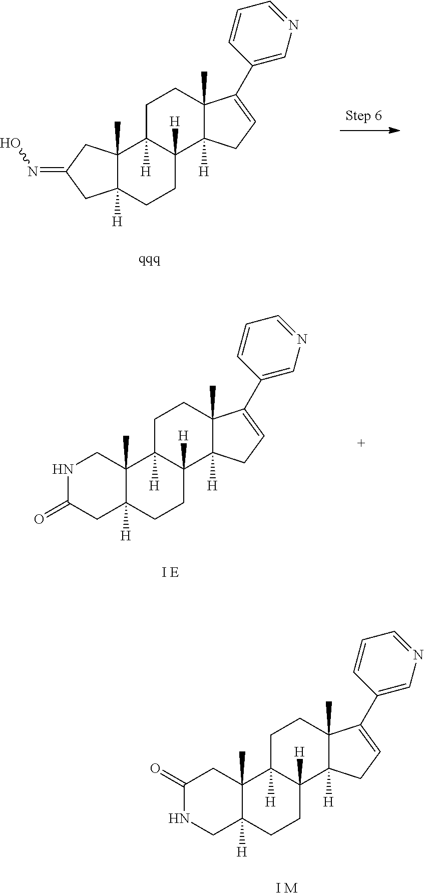

Compounds of structure of Formula (IE) and (I M) having A as 3-pyridinyl group are synthesized from the diacid compound (eee) prepared as above. Esterification of compound (eee) by first converting it to acid chloride with the treatment of thionyl and the treated with in methanol to give the methyl ester compound (mmm) (step 1). Conversion of compound (mmm) to the triflate compound (nnn) is achieved by using condition involving the enol triflate formation by the use of triflic anhydride (trifluoromethanesulfonic anhydride) in the presence of base such as triethylamine and the like (step 2). Suzuki coupling reaction on compounds (nnn) with 3-(diethylboryl)pyridine (or pyridine-3-yl boronic acid), and (Ph₃P)₂PdCl₂ in THF-water or dioxane-water in the presence of a base such as sodium carbonate followed by acidification yields compound (ooo) (step 3). Preparation of compound (ppp) from compound (ooo) is achieved by first hydrolysis of the ester to the corresponding diacid followed by treatment of it with sodium acetate in acetic anhydride and subsequence heating (step 4). Reaction of compound (ppp) hydroxylamine hydrochloride in the presence of triethylamine and organic solvent yields the oxime compound (qqq) (step 5). Beckmann rearrangement reaction on the oxime (qqq) in the presence of polyphosphoric acid or sulfuric acid yields a mixture of compounds of formula (IE) and (I M). Preparation of compounds of Formula (I E and I M) having different heteroaryl A are made similarly by using appropriate boronic acid instead of pyridine-3-yl boronic acid in step 3.

### Certain Pharmaceutical Terminology

The term “acceptable” with respect to a formulation, composition or ingredient, as used herein, means having no persistent detrimental effect on the general health of the subject being treated.

As used herein, the term “selective binding compound” refers to a compound that selectively binds to any portion of one or more target proteins.

As used herein, the term “selectively binds” refers to the ability of a selective binding compound to bind to a target protein, such as, for example, CYP17 enzyme, with greater affinity than it binds to a non-target protein. In certain embodiments, specific binding refers to binding to a target with an affinity that is at least about 10, about 50, about 100, about 250, about 500, about 1000 or more times greater than the affinity for a non-target.

As used herein, the term “target protein” refers to a molecule or a portion of a protein capable of being bound by a selective binding compound. In certain embodiments, a target protein is the enzyme CYP17.

As used herein, the terms “treating” or “treatment” encompass either or both responsive and prophylaxis measures, e.g., designed to inhibit, slow or delay the onset of a symptom of a disease or disorder, achieve a full or partial reduction of a symptom or disease state, and/or to alleviate, ameliorate, lessen, or cure a disease or disorder and/or its symptoms.

As used herein, amelioration of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

As used herein, the term inhibitor refers to a compound that decreases in the magnitude of a certain activity of a target protein or molecule compared to the magnitude of the activity in the absence of the inhibitor.

As used herein, the term “selective inhibitor” refers to a compound that selectively inhibits a target activity.

As used herein, the IC₅₀ refers to an amount, concentration or dosage of a particular test compound that achieves a 50% inhibition of a maximal response, such as modulation of CYP17, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

In some or any embodiments, toxicity and therapeutic efficacy of the compounds is determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and is expressed as the ratio LD₅₀/ED₅₀. Compounds which exhibit large therapeutic indices are contemplated herein. While in some or any embodiments, compounds that exhibit toxic side effects are used, care should be taken to design a delivery system that targets such reagents to the site of affected tissue in order to minimize potential damage to normal cells and, thereby, reduce side effects.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “CYP17 substrate” includes any of the various steroid hormones acted upon by a CYP17 or a CYP17-like P₄₅₀ enzyme. Examples include pregnenolone, progesterone and their 17α-hydroxylated forms. Pregnenolone is converted to DHEA via a CYP17 C_{17,20}-lyase reaction, but is also subject to C17α-hydroxylation via the C_{17,20}-lyase activity. Progesterone is converted to δ 4-androstenedione via a CYP17 C_{17,20}-lyase reaction, but is also subject to C17α-hydroxylation via the C17-hydroxylase activity to form 17-hydroxy-progesterone, a precursor to hydrocortisone (i.e. cortisol).

The term “CYP17 metabolite-associated disease or disorder” refers to a disease or disorder which in some embodiments is treated by alteration of the level of one or more CYP17 metabolites. Examples include a hormone dependent cancer, such as an androgen-dependent prostate cancer, which in other embodiments is treated by inhibiting CYP17-mediated androgen synthesis, and an estrogen-dependent breast cancer or ovarian cancer, which in further embodiments is treated by inhibiting CYP17-mediated estrogen synthesis.

The term “diluent” refers to chemical compounds that are used to dilute the compound of interest prior to delivery. Diluents include chemicals used to stabilize compounds because they provide a more stable environment. Salts dissolved in buffered solutions (which also can provide pH control or maintenance) are utilized as diluents in certain embodiments, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “enzymatically cleavable linker,” as used herein refers to unstable or degradable linkages which are degraded by one or more enzymes.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in certain instances, enzymes produce specific structural alterations to a compound. In some embodiments, metabolites of the compounds disclosed herein are identified either by administration of compounds to a host and analysis of tissue samples from the host, or by incubation of compounds with hepatic cells in vitro and analysis of the resulting compounds.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

By “pharmaceutically acceptable” or “therapeutically acceptable”, as used herein, refers a material, such as a carrier or diluent, which does not abrogate the biological activity or properties of the compound, and is relatively nontoxic. In certain instances, nontoxic and non-abrogative materials includes materials that when administered to an individual do not cause substantial, undesirable biological effects and/or do not interact in a deleterious manner with any of the components of the composition in which it is contained.

The term “pharmaceutically acceptable salt” or “therapeutically acceptable salt”, refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

A “prodrug” refers to an agent that is converted into the parent drug in vivo. Prodrugs are often useful because, in some situations, they are easier to administer than the parent drug. In certain instances, a prodrug is bioavailable by oral administration whereas the parent is not. In some instances, a prodrug has improved solubility in pharmaceutical compositions over the parent drug. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid or amino group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In some embodiments, the prodrug is designed to alter the metabolic stability or the transport characteristics of a drug, to mask side effects or toxicity, to improve the flavor of a drug or to alter other characteristics or properties of a drug.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Composition/Formulation

In certain embodiments, pharmaceutical compositions are formulated in any manner, including using one or more physiologically acceptable carriers comprising excipients and/or auxiliaries which facilitate processing of the active compounds into pharmaceutical preparations. In some or any embodiments, proper formulation is dependent upon the route of administration chosen. In some or any embodiments, any techniques, carriers, and excipients are used as suitable.

Provided herein are pharmaceutical compositions that include a compound described herein and a pharmaceutically acceptable diluent(s), excipient(s), and/or carrier(s). In addition, in some or any embodiments, the compounds described herein are administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents, and/or excipients. In certain embodiments, a pharmaceutical composition facilitates administration of the compound to an organism. In some or any embodiments, practicing the methods of treatment or use provided herein, includes administering or using a pharmaceutical composition comprising a therapeutically effective amount of a compound provided herein. In specific embodiments, the methods of treatment provided for herein include administering such a pharmaceutical composition to a mammal having a disease or condition to be treated. In some or any embodiments, the mammal is a human. In some or any embodiments, the therapeutically effective amount varies widely depending on the severity of the disease, the age and relative health of the subject, the potency of the compound used and other factors. In some or any embodiments, the compounds described herein are used singly or in combination with one or more therapeutic agents as components of mixtures.

In some or any embodiments, the pharmaceutical compositions provided herein are formulated for intravenous injections. In some or any aspects, the intravenous injection formulations provided herein are formulated as aqueous solutions, and, in some or any embodiments, in physiologically compatible buffers such as Hank's solution, Ringer's solution, or physiological saline buffer. In some or any embodiments, the pharmaceutical compositions provided herein are formulated for transmucosal administration. In some or any aspects, transmucosal formulations include penetrants appropriate to the barrier to be permeated. In some or any embodiments, the pharmaceutical compositions provided herein are formulated for other parenteral injections, appropriate formulations include aqueous or nonaqueous solutions, and in some or any embodiments, with physiologically compatible buffers or excipients.

In some or any embodiments, the pharmaceutical compositions provided herein are formulated for oral administration. In some or any aspects, the oral formulations provided herein comprise compounds described herein that are formulated with pharmaceutically acceptable carriers or excipients. Such carriers enable the compounds described herein to be formulated as tablets, powders, pills, dragees, capsules, liquids, gels, syrups, elixirs, slurries, suspensions and the like, for oral ingestion by a patient to be treated.

In some or any embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

In some or any embodiments, provided herein is a pharmaceutical composition formulated as dragee cores with suitable coatings. In certain embodiments, concentrated sugar solutions are used in forming the suitable coating, and optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs and/or pigments are added to tablets, dragees and/or the coatings thereof for, e.g., identification or to characterize different combinations of active compound doses.

In some or any embodiments, pharmaceutical preparations which are used include orally include push-fit capsules made of gelatin, as well as soft, sealed capsules made of gelatin and a plasticizer, such as glycerol or sorbitol. In some or any embodiments, the push-fit capsules contain the active ingredients in admixture with filler such as lactose, binders such as starches, and/or lubricants such as talc or magnesium stearate and, optionally, stabilizers. In some or any embodiments, in soft capsules, the active compounds are dissolved or suspended in suitable liquids, such as fatty oils, liquid paraffin, or liquid polyethylene glycols. In addition, stabilizers are optionally added. In some or any embodiments, the formulations for oral administration are in dosages suitable for such administration.

In some or any embodiments, the pharmaceutical compositions provided herein are formulated for buccal or sublingual administration. In some or any embodiments, buccal or sublingual compositions take the form of tablets, lozenges, or gels formulated in a conventional manner. In some or any embodiments, parenteral injections involve bolus injection or continuous infusion. In some or any embodiments, formulations for injection are presented in unit dosage form, e.g., in ampoules or in multi-dose containers, with an added preservative. In some or any embodiments, the pharmaceutical composition described herein is in a form suitable for parenteral injection as a sterile suspensions, solutions or emulsions in oily or aqueous vehicles, and optionally contains formulatory agents such as suspending, stabilizing and/or dispersing agents. Pharmaceutical formulations for parenteral administration include aqueous solutions of the active compounds in water-soluble form. In some or any embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In some or any embodiments, aqueous injection suspensions contain substances which increase the viscosity of the suspension, such as sodium carboxymethyl cellulose, sorbitol, or dextran. Optionally, the suspensions also contain suitable stabilizers or agents which increase the solubility of the compounds to allow for the preparation of highly concentrated solutions. In some or any embodiments, the active ingredient is in powder form for constitution with a suitable vehicle, e.g., sterile pyrogen-free water, before use.

In some or any embodiments, the compounds described herein are administered topically. In some or any embodiments, the compounds described herein are formulated into a variety of topically administrable compositions, such as solutions, suspensions, lotions, gels, pastes, medicated sticks, balms, creams or ointments. Such pharmaceutical compounds optionally contain solubilizers, stabilizers, tonicity enhancing agents, buffers and/or preservatives.

In some or any embodiments, the pharmaceutical compositions provided herein are formulated for transdermal administration of compounds described herein. In some or any embodiments, administration of such compositions employs transdermal delivery devices and transdermal delivery patches. In some or any embodiments, the compositions are lipophilic emulsions or buffered, aqueous solutions, dissolved and/or dispersed in a polymer or an adhesive. Such patches include those constructed for continuous, pulsatile, or on demand delivery of pharmaceutical agents. In some or any embodiments, transdermal delivery of the compounds described herein is accomplished by use of iontophoretic patches and the like. In some or any embodiments, transdermal patches provide controlled delivery of the compounds provided herein, such as, for example, compounds of Formula (I), (II), or (III). In some or any embodiments, the rate of absorption is slowed by using rate-controlling membranes or by trapping the compound within a polymer matrix or gel. Conversely, absorption enhancers are optionally used to increase absorption. Absorption enhancer and carrier include absorbable pharmaceutically acceptable solvents that assist in passage of the compound through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In some or any embodiments, the pharmaceutical compositions provided herein are formulated for administration by inhalation. In some or any embodiments, in such pharmaceutical compositions formulated for inhalation, the compounds described herein are in a form as an aerosol, a mist or a powder. In some or any embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In some or any aspects of a pressurized aerosol, the dosage unit is determined by providing a valve to deliver a metered amount. In certain embodiments, capsules and cartridges of, such as, by way of example only, gelatin for use in an inhaler or insufflator is formulated containing a powder mix of the compound described herein and a suitable powder base such as lactose or starch.

In some or any embodiments, the compounds described herein are formulated in rectal compositions such as enemas, rectal gels, rectal foams, rectal aerosols, suppositories, jelly suppositories, or retention enemas. In some or any embodiments, rectal compositions optionally contain conventional suppository bases such as cocoa butter or other glycerides, as well as synthetic polymers such as polyvinylpyrrolidone, PEG, and the like. In certain suppository forms of the compositions, a low-melting wax such as, but not limited to, a mixture of fatty acid glycerides, optionally in combination with cocoa butter is first melted.

In some or any embodiments provided herein, the pharmaceutical compositions are formulated in a conventional manner using one or more physiologically acceptable carriers comprising excipients and auxiliaries which facilitate processing of the active compounds into pharmaceutically acceptable preparations. In some or any embodiments, proper formulation is dependent upon the route of administration chosen. In various embodiments, any of the techniques, carriers, and excipients is used as suitable. In some or any embodiments, pharmaceutical compositions comprising a compound described herein are manufactured in a conventional manner, such as, by way of example only, by means of conventional mixing, dissolving, granulating, dragee-making, levigating, emulsifying, encapsulating, entrapping or compression processes.

In some or any embodiments, the pharmaceutical compositions include at least one pharmaceutically acceptable carrier, diluent or excipient and a compound described herein described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds described herein exist as tautomers. All tautomers are included within the scope of the compounds presented herein. Additionally, included herein are the solvated and unsolvated forms of the compounds described herein. Solvated compounds include those that are solvated with pharmaceutically acceptable solvents such as water, ethanol, and the like. The solvated forms of the compounds presented herein are also considered to be disclosed herein. In some or any embodiments, the pharmaceutical compositions described herein include other medicinal or pharmaceutical agents, carriers, adjuvants, such as preserving, stabilizing, wetting or emulsifying agents, solution promoters, salts for regulating the osmotic pressure, and/or buffers. In some or any embodiments, the pharmaceutical compositions described herein also contain other therapeutically valuable substances.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In some or any embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents, and so forth.

In some or any embodiments, a composition comprising a compound described herein takes the form of a liquid where the agents are present in solution, in suspension or both. In some or any embodiments, when the composition is administered as a solution or suspension a first portion of the agent is present in solution and a second portion of the agent is present in particulate form, in suspension in a liquid matrix. In certain embodiments, a liquid composition includes a gel formulation. In other embodiments, the liquid composition is aqueous.

Useful aqueous suspension optionally contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions optionally comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions optionally include solubilizing agents to aid in the solubility of a compound described herein. The term “solubilizing agent” generally includes agents that result in formation of a micellar solution or a true solution of the agent. Solubilizing agents include certain acceptable nonionic surfactants, for example polysorbate 80, and ophthalmically acceptable glycols, polyglycols, e.g., polyethylene glycol 400, and glycol ethers.

Useful compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Certain useful compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Some useful compositions optionally include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Certain useful compositions optionally one or more antioxidants to enhance chemical stability where required. Suitable antioxidants include, by way of example only, ascorbic acid and sodium metabisulfite.

In some or any embodiments, aqueous suspension compositions are packaged in single-dose non-reclosable containers. In alternative embodiments, multiple-dose reclosable containers are used, in which case it is typical to include a preservative in the composition.

In various embodiments, any delivery system for hydrophobic pharmaceutical compounds is employed. Liposomes and emulsions are examples of delivery vehicles or carriers for hydrophobic drugs. In some or any embodiments, certain organic solvents such as N-methylpyrrolidone are employed. In some or any embodiments, the compounds are delivered using a sustained-release system, such as semipermeable matrices of solid hydrophobic polymers containing the therapeutic agent. Various sustained-release materials are utilized in the embodiments herein. In certain embodiments, sustained-release capsules release the compounds for a few weeks up to over 100 days. In some or any embodiments, depending on the chemical nature and the biological stability of the therapeutic reagent, additional strategies for protein stabilization are employed.

In some or any embodiments, the formulations or compositions described herein benefit from and/or optionally comprise antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

In some or any embodiments, the compounds described herein are used in the preparation or manufacture of medicaments for the treatment of diseases or conditions that are mediated by the CYP17 enzyme. Inhibition of the enzymes ameliorates the disease or condition associated with CYP17. In some or any embodiments, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In some or any embodiments, the compositions containing the compound(s) described herein are administered for prophylactic and/or therapeutic treatments. In some or any therapeutic applications, the compositions are administered to a patient already suffering from a disease or condition, in an amount sufficient to cure or at least partially arrest the symptoms of the disease or condition. In some or any embodiments, amounts effective for this use will depend on the severity and course of the disease or condition, previous therapy, the patient's health status, weight, and response to the drugs, and the judgment of the treating physician.

In some or any prophylactic applications, compositions containing the compounds described herein are administered to a patient susceptible to or otherwise at risk of a particular disease, disorder or condition. In some or any embodiments, the amount administered is defined to be a “prophylactically effective amount or dose.” In some or any embodiments of this use, the precise amounts of compound administered depend on the patient's state of health, weight, and the like. In some or any embodiments, when used in a patient, effective amounts for this use will depend on the severity and course of the disease, disorder or condition, previous therapy, the patient's health status and response to the drugs, and the judgment of the treating physician.

In some or any embodiments, a patient's condition does not improve or does not significantly improve following administration of a compound or composition described herein and, upon the doctor's discretion the administration of the compounds is optionally administered chronically, that is, for an extended period of time, including throughout the duration of the patient's life in order to ameliorate or otherwise control or limit the symptoms of the patient's disease or condition.

In some or any embodiments, once improvement of the patient's conditions has occurred, a maintenance dose is administered if necessary. In some or any embodiments, the dosage, e.g., of the maintenance dose, or the frequency of administration, or both, are reduced, as a function of the symptoms, to a level at which the improved disease, disorder or condition is retained. In some or any embodiments, however, patients are optionally given intermittent treatment on a long-term basis upon any recurrence of symptoms.

In some or any embodiments, the amount of a given agent that corresponds to an effective amount varies depending upon factors such as the particular compound, disease or condition and its severity, the identity (e.g., weight) of the subject or host in need of treatment. In some or any embodiments, the effective amount is, nevertheless, determined according to the particular circumstances surrounding the case, including, e.g., the specific agent that is administered, the route of administration, the condition being treated, and the subject or host being treated. In some or any embodiments, however, doses employed for adult human treatment is in the range of about 0.02 to about 5000 mg per day. In some or any embodiments, dose employment for adult human treatment is about 1 to about 1500 mg per day. In various embodiments, the desired dose is conveniently presented in a single dose or as divided doses administered simultaneously (or over a short period of time) or at appropriate intervals, for example as two, three, four or more sub-doses per day.

In some or any embodiments, while the dose varies depending on age, body weight, symptom, treatment effect, administration method and the like, the pharmaceutical compositions described herein are given at a dose from about 0.01 mg to about 1 g per administration for an adult given once or several times a day orally or in a dosage form of an injection such as intravenous injection and the like. An anti-cancer agent is generally required to sustain its effect for a long time, so that can be effective not only for temporary suppression but also for prohibition on a long term basis. In some or any embodiments, the compounds described herein are administered on a long term basis.

In some or any embodiments, the pharmaceutical compositions described herein are in a unit dosage form suitable for single administration of precise dosages. In some or any instances, in unit dosage form, the formulation is divided into unit doses containing appropriate quantities of one or more compound. In some or any embodiments, the unit dosage is in the form of a package containing discrete quantities of the formulation. Non-limiting examples are packaged tablets or capsules, and powders in vials or ampoules. In some or any embodiments, aqueous suspension compositions are packaged in single-dose non-reclosable containers. In alternative embodiments, multiple-dose reclosable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

In some or any embodiments, the daily dosages appropriate for the compounds described herein are from about 0.01 to about 5 mg/kg per body weight. In some or any embodiments, an indicated daily dosage in the larger subject, including, but not limited to, humans, is in the range from about 0.5 mg to about 1000 mg, conveniently administered in divided doses, including, but not limited to, up to four times a day or in extended release form. In certain embodiments, suitable unit dosage forms for oral administration comprise from about 1 to about 500 mg active ingredient. The foregoing ranges are merely suggestive, as the number of variables in regard to an individual treatment regime is large, and considerable excursions from these recommended values are not uncommon. In some or any embodiments, the dosages are altered depending on a number of variables, not limited to the activity of the compound used, the disease or condition to be treated, the mode of administration, the requirements of the individual subject, the severity of the disease or condition being treated, and the judgment of the practitioner.

In some or any embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In some or any embodiments, compounds exhibiting high therapeutic indices are preferred. In some or any embodiments, the data obtained from cell culture assays and animal studies is used in formulating a range of dosage for use in human. In some or any embodiments, the dosage of such compounds lies within a range of circulating concentrations that include the ED₅₀ with minimal toxicity. In some or any embodiments, the dosage varies within this range depending upon the dosage form employed and the route of administration utilized.

### Combination Treatments

Presented herein are compounds having the structure of Formula (I), (II), (III), (IA), (IB), (IC), (ID), (IE), (IF), (IG), (IH), (IJ), (IK), (IL), (IM), (IN), (IIA), (IIB) or (IIC) in combination with a second therapeutic agent for the treatment of an androgen dependent disease, disorder or condition. In some or any embodiments, the compounds described herein are administered in combination with a second active agent which is effective against cancer.

Suitable compounds used in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC) include anti-cancer agents, such as for example, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, and/or anti-androgens. The amount of the additional anti-cancer agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC) Below are lists of examples of some of the classes of anti-cancer agents. The examples are not all inclusive and are for purposes of illustration and not for purposes of limitation. Many of the examples below are not restricted in any way to the class in which they are listed in and in some embodiments are listed in multiple classes of anti-cancer agents.

Suitable hormonal ablation agents include, but are not limited to, androgen ablation agents and estrogen ablation agents. In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered with a hormonal ablation agent, such as deslorelin, leuprolide, goserelin or triptorelin. The amount of the hormonal ablation agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC).

Suitable anti-androgen agents include but are not limited to bicalutamide, flutamide and nilutamide. The amount of the anti-androgen agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered with a differentiating agent. Suitable differentiating agents include, but are not limited to, polyamine inhibitors; vitamin D and its analogs, such as, calcitriol, doxercalciferol and seocalcitol; metabolites of vitamin A, such as, ATRA, retinoic acid, retinoids; short-chain fatty acids; phenylbutyrate; and nonsteroidal anti-inflammatory agents. The amount of the differentiating agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered with an anti-neoplastic agent, including, but not limited to, tubulin interacting agents, topoisomerase inhibitors and agents, acitretin, alstonine, amonafide, amphethinile, amsacrine, ankinomycin, anti-neoplaston, aphidicolin glycinate, asparaginase, baccharin, batracylin, benfluoron, benzotript, bromofosfamide, caracemide, carmethizole hydrochloride, chlorsulfaquinoxalone, clanfenur, claviridenone, crisnatol, curaderm, cytarabine, cytocytin, dacarbazine, datelliptinium, dihaematoporphyrin ether, dihydrolenperone, dinaline, distamycin, docetaxel, elliprabin, elliptinium acetate, epothilones, ergotamine, etoposide, etretinate, fenretinide, gallium nitrate, genkwadaphnin, hexadecylphosphocholine, homoharringtonine, hydroxyurea, ilmofosine, isoglutamine, isotretinoin, leukoregulin, lonidamine, merbarone, merocyanlne derivatives, methylanilinoacridine, minactivin, mitonafide, mitoquidone, mitoxantrone, mopidamol, motretinide, N-(retinoyl)amino acids, N-acylated-dehydroalanines, nafazatrom, nocodazole derivative, ocreotide, oquizanocine, paclitaxel, pancratistatin, pazelliptine, piroxantrone, polyhaematoporphyrin, polypreic acid, probimane, procarbazine, proglumide, razoxane, retelliptine, spatol, spirocyclopropane derivatives, spirogermanium, strypoldinone, superoxide dismutase, teniposide, thaliblastine, tocotrienol, topotecan, ukrain, vinblastine sulfate, vincristine, vindesine, vinestramide, vinorelbine, vintriptol, vinzolidine, and withanolides. The amount of the anti-neoplastic agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), JR (IK), (IL), (IM), (IN), (IIA), (IIB) or (IIC).

In some or any embodiments, the compounds described herein, such as for example, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is used with a kinase inhibitor including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, SOD mimics or αᵥβ₃ inhibitors. The amount of the kinase inhibitor administered to a mammal having cancer is an amount that is sufficient to treat the cancer whether administered alone or in combination with a compound having the structure of Formula (I), (II) or (III) or (I), (II), (III), (IA), (IB), (IC), (ID), (IE), (IF), (IG), (IH), (IJ), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered with an anti-metabolite agent. In some or any embodiments, suitable anti-metabolite agents are selected from, but not limited to, 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, and uricytin. The amount of the anti-metabolite agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered with an alkylating agent. In some or any embodiments, suitable alkylating agents are selected from, but not limited to, aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, elmustine, estramustine phosphate sodium, fotemustine, hepsul-fam, Ifosfamide, iproplatin, lomustine, mafosfamide, mitolactol, oxaliplatin, prednimustine, ranimustine, semustine, spiromustine, tauromustine, temozolomide, teroxirone, tetraplatin and trimelamol. The amount of the alkylating agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (Ti), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered with an antibiotic agent. In some or any embodiments, suitable antibiotic agents are selected from, but not limited to, aclarubicin, actinomycin D, actinoplanone, adriamycin, aeroplysinin derivative, amrubicin, anthracycline, azino-mycin-A, bisucaberin, bleomycin sulfate, bryostatin-1, calichemycin, chromoximycin, dactinomycin, daunorubicin, ditrisarubicin B, dexamethasone, doxorubicin, doxorubicin-fibrinogen, elsamicin-A, epirubicin, erbstatin, esorubicin, esperamicin-Al, esperamicin-Alb, fostriecin, glidobactin, gregatin-A, grincamycin, herbimycin, corticosteroids such as hydrocortisone, idarubicin, illudins, kazusamycin, kesarirhodins, menogaril, mitomycin, neoenactin, oxalysine, oxaunomycin, peplomycin, pilatin, pirarubicin, porothramycin, prednisone, prednisolone, pyrindanycin A, rapamycin, rhizoxin, rodorubicin, sibanomicin, siwenimycin, sorangicin-A, sparsomycin, talisomycin, terpentecin, thrazine, tricrozarin A, and zorubicin. The amount of the antibiotic agent administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is used with other anti-cancer agents, including but not limited to, acemannan, aclarubicin, aldesleukin, alemtuzumab, alitretinoin, altretamine, amifostine, amsacrine, anagrelide, anastrozole, ancestim, bexarotene, broxuridine, capecitabine, celmoleukin, cetrorelix, cladribine, clotrimazole, daclizumab, dexrazoxane, dilazep, docosanol, doxifluridine, bromocriptine, carmustine, cytarabine, diclofenac, edelfosine, edrecolomab, eflornithine, emitefur, exemestane, exisulind, fadrozole, filgrastim, finasteride, fludarabine phosphate, formestane, fotemustine, gallium nitrate, gemcitabine, glycopine, heptaplatin, ibandronic acid, imiquimod, iobenguane, irinotecan, irsogladine, lanreotide, leflunomide, lenograstim, lentinan sulfate, letrozole, liarozole, lobaplatin, lonidamine, masoprocol, melarsoprol, metoclopramide, mifepristone, miltefosine, mirimostim, mitoguazone, mitolactol, molgramostim, nafarelin, nartograstim, nedaplatin, nilutamide, noscapine, oprelvekin, osaterone, oxaliplatin, pamidronic acid, pegaspargase, pentosan polysulfate sodium, pentostatin, picibanil, pirarubicin, porfimer sodium, raloxifene, raltitrexed, rasburicase, rituximab, romurtide, sargramostim, sizofuran, sobuzoxane, sonermin, suramin, tasonermin, tazarotene, tegafur, temoporfin, temozolomide, teniposide, tetrachlorodecaoxide, thalidomide, thymalfasin, thyrotropin alfa, topotecan, toremifene, trastuzumab, treosulfan, tretinoin, trilostane, trimetrexate, ubenimex, valrubicin, verteporfin, vinorelbine. The amount of the anti-cancer agent administered to a mammal having cancer is an amount that is sufficient to treat the cancer whether administered alone or in combination with a compound having the structure of Formula (I), (II) or (III) or (I), (II), (III), (IA), (IB), (IC), (ID), (IE), (IF), (IG), (IH), (IJ), (IK), (IL), (IM), (IN), (IIA), (IIB) or (IIC).

In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is administered or combined with steroids, such as corticosteroids or glucocorticoids. In some or any embodiments, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) and the steroid are administered in the same or in different compositions. Non-limiting examples of suitable steroids include hydrocortisone, prednisone, or dexamethasone. The amount of the steroid administered to a mammal having cancer is an amount that is sufficient to treat the cancer whether administered alone or in combination with a compound having the structure of Formula (I), (II), (III), (IA), (IB), (IC), (ID), (IE), (IF), (IG), (IH), (IJ), (IK), (IL), (IM), (IN), (IIA), (IIB) or (IIC).

In some or any embodiments, if one of the side effects experienced by a patient upon receiving one of the compounds herein is inflammation, then, in some or any embodiments, it is appropriate to administer an anti-inflammatory agent in combination with the initial therapeutic agent. In some or any embodiments, the therapeutic effectiveness of one of the compounds described herein is enhanced by administration of an adjuvant (i.e., by itself the adjuvant may have minimal therapeutic benefit, but in combination with another therapeutic agent, the overall therapeutic benefit to the patient is enhanced). In some or any embodiments, the benefit experienced by a patient is increased by administering one of the compounds described herein with another therapeutic agent (which also includes a therapeutic regimen that also has same therapeutic benefit (e.g. anti-cancer agent against the same enzyme as the compound described herein but of different mode of action) so as to reduce the chance of enzyme resistant development. In some or any embodiments, regardless of the disease, disorder or condition being treated, the overall benefit experienced by the patient as a result of a combination treatment is additive or synergistic.

In some or any embodiments, therapeutically-effective dosages vary when the drugs are used in treatment combinations. In some or any embodiments, therapeutically-effective dosages of drugs and other agents for use in combination treatment regimens is determined in any suitable manner, e.g., through the use of metronomic dosing, i.e., providing more frequent, lower doses in order to minimize toxic side effects. In some or any embodiments, combination treatment regimen described herein encompass treatment regimens in which administration of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) described herein is initiated prior to, during, or after treatment with a second agent described above, and continues until any time during treatment with the second agent or after termination of treatment with the second agent. It also includes treatments in which a compound having the structure of Formula (I), (II), (III), (IA), (IB), (IC), (ID), (IE), (IF), (IG), (IH), (IJ), (IK), (IL), (IM), (IN), (IIA), (IIB) or (IIC) described herein and the second agent being used in combination are administered simultaneously or at different times and/or at decreasing or increasing intervals during the treatment period.

In some or any embodiments, compositions and methods for combination therapy are provided herein. In accordance with some or any aspects, the pharmaceutical compositions disclosed herein are used to in a method of treating a CYP17 mediated condition or a disease or condition that is ameliorated by inhibition of these enzymes.

In some or any embodiments, combination therapies described herein are used as part of a specific treatment regimen intended to provide a beneficial effect from the co-action of a compound having the structure of Formula (I), (II), (III), (IA), (IB), (IC), (ID), (IE), (IF), (IG), (IH), (IA (IK), (IL), (IM), (IN), (IIA), (IIB) or (IIC) described herein and a concurrent treatment. It is understood that the dosage regimen to treat, prevent, or ameliorate the condition(s) for which relief is sought, is optionally modified in accordance with a variety of factors.

In some or any combination therapies described herein, dosages of the co-administered compounds vary depending on the type of co-drug employed, on the specific drug employed, on the disease or condition being treated and so forth. In some embodiments, when co-administered with one or more biologically active agents, the compound provided herein is administered either simultaneously with the biologically active agent(s), or sequentially. In certain aspects wherein the agents are administered sequentially, the attending physician will decide on the appropriate sequence of administering protein in combination with the biologically active agent(s).

In some or any embodiments, the multiple therapeutic agents (one of which is one of the compounds described herein) are administered in any order or even simultaneously. In some or any instances, administration is simultaneous and the multiple therapeutic agents are, optionally, provided in a single, unified form, or in multiple forms (by way of example only, either as a single pill or as two separate pills). In some or any embodiments, one of the therapeutic agents is given in multiple doses, or both are given as multiple doses. In some or any instances, administration is not simultaneous and the timing between the multiple doses varies, by way of non-limiting example, from more than zero weeks to less than four weeks. In addition, the combination methods, compositions and formulations are not to be limited to the use of only two agents; the use of multiple therapeutic combinations is also contemplated herein.

In some or any embodiments, the compounds described herein and combination therapies are administered before, during or after the occurrence of a disease or condition. In some or any embodiments, the timing of administering the composition containing a compound varies. Thus, for example, in some or any embodiments, the compounds are used as a prophylactic and are administered continuously to subjects with a propensity to develop conditions or diseases in order to prevent the occurrence of the disease or condition. In some or any embodiments, the compounds and compositions are administered to a subject during or as soon as possible after the onset of the symptoms. The initial administration is achieved via any route practical, such as, for example, an intravenous injection, a bolus injection, infusion over 5 minutes to about 5 hours, a pill, a capsule, transdermal patch, buccal delivery, and the like, or combination thereof.

### Kits/Articles of Manufacture

For use in the therapeutic applications described herein, kits and articles of manufacture are also described herein. In some or any embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some or any embodiments, the containers are formed from a variety of materials such as glass or plastic.

In some or any embodiments, the articles of manufacture provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

In some or any embodiments, the container(s) described herein comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

In some or any embodiments, a kit will comprises one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions is optionally included.

In some or any embodiments, a label is on or associated with the container. In some or any embodiments, a label is on a container when letters, numbers or other characters forming the label are attached, molded or etched into the container itself; a label can be associated with a container when it is present within a receptacle or carrier that also holds the container, e.g., as a package insert. In some or any embodiments, a label indicates that the contents are to be used for a specific therapeutic application. In some or any embodiments, the label indicates directions for use of the contents, such as in the methods described herein.

In some or any embodiments, the pharmaceutical compositions are presented in a pack or dispenser device which contains one or more unit dosage forms containing a compound provided herein. In some or any embodiments, the pack contains a metal or plastic foil, such as a blister pack. The pack or dispenser device is optionally accompanied by instructions for administration. In some or any embodiments, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. In some or any embodiments, such notice is, for example, the labeling approved by the U.S. Food and Drug Administration for prescription drugs, or the approved product insert. In some or any embodiments, compositions containing a compound provided herein are formulated in a compatible pharmaceutical carrier and are placed in an appropriate container labeled for treatment of an indicated condition.

### EXAMPLES

### Biological Studies

### Human and Murine C_{17,20}-Lyase Biochemical Assays:

Recombinant human C_{17,20}-lyase (hLyase) is expressed in baculovirus-infected Sf9 cells and hLyase enriched microsomes are prepared from cultures as described (Barnes H. J.; Jenlins, C. M.; Waterman, M. R. Archives of Biochemistry and Biophysics 1994, 315 (2), 489-494). Recombinant murine C_{17,20}-lyase (mLyase) is prepared in a similar manner. hlyase and mLyase preparations are titrated using assay conditions to determine protein concentrations to be used for assays. Both mLyase and hLyase assays are run in an identical manner except that cytochrome b5 is omitted in the murine assay.

Test compound solutions (20 mM in DMSO) are diluted 1:4 with DMSO and put into the top well of a 96-well mother plate. These solutions are then diluted serially in six steps (1:4 each step) with DMSO to obtain 800 μM to 51.2 nM concentrations on a mother plate (columns 3-12) for subsequent use in the assay. These compound solutions are further diluted twenty-fold in water to obtain a daughter plate containing compound concentrations ranging from 40 μM to 2.56 nM in 5% DMSO. The first 2 columns (of wells) on each 96-well mother plate are used for the DHEA (dehydroepiandrosterone) standard curve. DHEA standards are serially diluted (in half-logs) in DMSO to obtain 400 μM to 120 nM standards, then diluted (1:19) in water to obtain 20 μM to 6 nM solutions in 5% DMSO on the daughter plate. These 5% DMSO solutions (5 μL each) from the daughter plate are transferred to the SPA assay plate prior to adding the reaction mixture.

To prepare the reaction mixture, clear-bottomed opaque 96-well assay plates are loaded with 50 μL of assay buffer (50 mM Na₃PO₄, pH 7.5), 5 mL of the diluted compounds (or standards), and 30 mL of substrate solutions (7 mM NADPH, 3.35 μM 17-OH-pregnenolone, 3.35 μg/mL human cytochrome b5 in 50 mM Na₃PO₄). Reactions are initiated with the addition of hLyase or mLyase in assay buffer (10 μL). Enzymatic reactions are incubated at room temperature for 2 hours with gentle agitation. Reactions are terminated with the addition of 5 μL of 1 mM (50 μM final concentration) YM116, a potent C_{17,20}-lyase inhibitor.

The concentration of DHEA generated by hLyase (or mLyase) is determined by radioimmunoassay (RIA). RIA will utilize a ³H-DHEA (0.08 μCi) tracer in 50 μL of scintillation proximity assay (SPA) buffer (100 mM Tris-HCl, pH 7.5, 50 mM NaCl, 0.5% BSA, 0.2% Tween 20) which is added to each well. DHEA antiserum from rabbit (50 μL) with anti-rabbit SPA beads in SPA buffer is added to all wells. Mixtures are allowed to equilibrate with gentle agitation for 1 hour followed by overnight equilibration with no agitation. H-DHEA bound to the SPA beads is determined by scintillation counting with a Wallac microbeta counter. The concentration of DHEA generated is calculated from raw data (CPM) and the standard curve. The concentration of DHEA formed in the presence of test compounds is then expressed as a percent inhibition compared to the DHEA concentration in the absence of test compounds: [1-(nM DHEA formed in the presence of test compound/nM DHEA formed in the absence of test compounds)]×100. Determination of IC₅₀ for each compound will be performed using the Analyze 5 program.

### Human C_{17,20}-Lyase Cellular Assay:

Human HEK 293-lyase stable transfectant cells are seeded in a 96-well plate at 10,000 cells/well/100 μL in DMEM plus 10% FBS (supplemented with 1% glutamine, 0.8 mg/mL G418) and allowed to attach overnight. The next day, the media is removed from the cell plate and replaced with 100 μL RPMI without phenol red. Test compounds, DMSO vehicle, or DHEA standards of 5 mL each are added to the cell plate and incubated for 10 min. at room temperature. The reaction is initiated with 10 μL of 5 μM 17-OH-pregnenolone added to all the wells of the cell plate, then incubated for 1 hour at 37° C. Following the incubation, 90 μL of media (containing DHEA product) is removed from the cell plate and transferred to the SPA assay plate. The subsequent SPA procedure for the detection of DHEA product is performed in the same manner as described for the enzyme assay (see above). The mother plate of test compounds is also prepared in the same manner as the enzyme assay.

Reagents (including catalog #) for the SPA assay are obtained from the following sources: ³H-DHEA: NEN (NET814), Anti-DHEA: Endocrine Sciences (D7-421), Anti-Rabbit SPA Beads: Amersham (RPNQ 0016), 17-OH-pregnenolone: Steraloids (Q4710), NADPH: Sigma (N1630), Cytochrome b5: Panvera (P2252), DHEA (500 μM stock in 100% EtOH), BSA: Sigma (A9647).

Evaluation of a Compound Having the Structure of Formula (I), (II) or (III) as Inhibitors of Testicular Human and Rat 17α-Hydroxylase/C_{17,20}-Lyase (17α-Lyase) in vitro

The potency as inhibitors of P450₁₇α of the compounds described herein are evaluated in human and rat testicular microsomes.

Human testicular microsomes are prepared from human testes (obtained from untreated prostatic cancer patients undergoing orchidectomy), as described in Li et al., The Prostate, 26:140-150 (1995).

Rat testicular microsomes are prepared from the testes of adult Sprague-Dawley rats, as described by Li et al., J. Med. Chem., 39:4335-4339 (1996).

The microsomes are stored at −70° C. until assayed. Just before use, the thawed microsomes are diluted with 0.1 M phosphate buffer (pH 7.4) to appropriate concentrations.

The protein concentration of the microsomes used in the assay are determined by the method of Lowry et al., J. Biol. Chem., 193:265-275 (1951).

The enzyme reaction (activity) is monitored by determination of the release of C³H³COOH from [21-³H³]-17α-hydroxypregnenolone during cleavage of the C-21 side-chain in the conversion to dehydroepiandrosterone (DHEA) as described by Njar et al., Steroids, 62:468-473 (1997). This assay measures only the lyase activity of the P450₁₇α enzyme. This assay is comparable to the HPLC assay procedure (which utilizes [7-³H]-pregnenolone as substrate), and measures both the hydroxylase and lyase activities of the enzyme.

IC₅₀ values for inhibitors are calculated from the linear regression line in the plot of logit of lyase activity versus log of inhibitor concentration. Kᵢ values are also determined from assays as described by Njar et al., (1997), supra. Each inhibitor is examined at three concentrations. Data from the various assays are used to obtain Lineweaver-Burk plots and from replots of slopes versus inhibitor concentration, Kᵢ values are obtained and the Kₘ for 17α-hydroxypregnenolone (substrate) is also determined.

Human C_{17,20}-lyase enzymatic assays were conducted in 200 μL volume in Eppendorf tubes, using microsomal fraction from human testis (Celsis Cat #S00110) as the enzyme source. Total protein concentration of the microsomal fraction is estimated to be 20 mg/ml. Prior to adding the microsomal fractions, reaction mixtures containing 50 mM NaPO₄ buffer (pH 7.4), 1 mM MgCl₂, 0.1 mM EDTA, 0.1 mM dithiothreitol, 0.5 mM NADPH, 4 μM 17□-hydroxypregnenolone, 1 μL of [21-³H]− 17α-hydroxypregnenolone (American Radiolabeled Chemicals, ART #1663, Specific activity=50-60 Ci/mmol), and the appropriate testing compounds were incubated for 5 minutes in a 37° C. shaking water bath (150 rpm). Following the 5-minute pre-incubation, 5 μL of human testis microsome was added to each of the reaction mixtures (except for the Negative Controls, which received 5 μL, of H₂O). After 30-minute incubation at 37° C. in shaking water bath (150 rpm), reactions were stopped by addition of 200 μL of cold chloroform and vigorous shaking for 30 minutes. Tubes were centrifuged at 1,500×g for 15 minutes at 4° C., and the aqueous phase was transferred to fresh Eppendorf tubes. Forty microliters (40 μL) of 8.5% charcoal (Sigma Cat # C6241) suspension was added to each tube, mixed well and incubated at 4° C. for 30 minutes. Tubes were centrifuged at 1,500×g for 15 minutes at 4° C., and 100 μL upper layer from each tube was transferred into each well of a 96-well isoplate (PerkinElmer Cat # 6005040). Finally, 1004, of Optiphase supermix scintillation fluid (PerkinElmer Cat # 1200430) was added to each well, mixed by pippeting up and down 3 times. Radioactivity was measured with MicroBeta Trilux Counter using tritium program. All testing compounds were dissolved and diluted in methanol. Two microliters (2 μL) of the properly diluted test compound was added to each reaction to reach the desired concentration. In Negative control (no enzyme activity) and Activity control (100% enzyme activity), 2 μL of methanol was added. Each data point was tested in duplicate. Inhibition of human C_{17,20}-lyase activity was calculated either by inhibition rate at 100 nM concentration and the inhibition rate was calculated as following;

$$Inhibition Rate ( \% ) = 100 - ( 1 - \frac{Test compound cpm - Negative control cpm}{Activity control cpm - Negative control cpm} )$$

or by IC₅₀ value which was generated using Prism software under “non-linear regression analysis”. Most of the compounds tested have an inhibition rate over 50% when tested at 100 nM concentration.
In vivo Antitumor Studies (LAPC-4 Prostate Cancer Xenografts)

All animal studies are performed according to the guidelines and approval of the Animal Care Committee of the testing facility.

Male sever combined immunodeficient (SCID) mice 4-6 weeks of age are purchased, for example, from the National Cancer Institute-Frederick Cancer Research and Development Center and housed in a pathogen-free environment under controlled conditions of light and humidity and allowed free access to food and water. Tumors are developed from LAPC-4 cells inoculated subcutaneously (s.c.) in the mice. LAPC-4 cells are grown in IMEM with 15% FBS plus 1% PS and 10 nm DHT until 80% confluent. Cells are scraped into DPBS, collected by centrifugation, and resuspended in Matrigel (10 mg/ml) at 3×10⁷ cells/ml. Mice are injected s.c. with 100 μl of the cell suspension at one site on each flank. Tumors will be measured weekly with calipers, and tumor volumes will be calculated by the formula: 4/3Π×r₁²×r₂ (r₁<r₂).

LAPC-4 tumors are allowed to grow for 8-10 weeks following inoculation. Groups of 5 mice with comparable total tumor volumes are either castrated under methoxyfluorane anesthesia or treated with a compound having the structure of Formula (I), (II), (III), (IA), (IB), (IC), (ID), (IE), (IF), (IG), (IH), (IJ), (IK), (IL), (IM), (IN), (IIA), (IIB) and (IIC) (about 0.15 mmol/kg once-daily and 0.15 mmol/kg twice-daily). A compound having the structure of Formula (I), (II), (III), (IA), (IB), (IC), (ID), (IE), (IF), (IG), (IH), (H), (IK), (IL), (IM), (IN), (IIA), (IIB) and (IIC)) is prepared at about 17 mg/ml in about a 0.3% solution of hydroxypropyl cellulose in saline, and mice receive s.c. injections daily. Control and castrated mice are treated with vehicle only. Tumors are measured weekly for the 4 weeks of treatment and tumor volumes are calculated. At the end of the treatment period, the mice are sacrificed under halothane anesthesia; the tumors are excised, weighed and stored at −80° C. The mice are also weighed weekly and monitored for general health status and signs of possible toxicity due to treatment.

### Human Clinical Trial of the Safety and Efficacy of Compounds as Disclosed Herein

Objective: To evaluate the safety, pharmacokinetics, pharmacodynamics, and anti-tumor activities of an oral CYP17 inhibitor, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) administered to patients with hormone refractory prostate cancer (HRPC).

Patients: Eligible subjects will be men 18 years and older.

Inclusion criteria for Phase I will include:
Histologically confirmed adenocarcinoma of the prostate;
No prior therapy with chemotherapy for prostate cancer;
Ongoing gonadal androgen deprivation therapy with LHRH analogues or orchiectomy. Patients, who have not had an orchiectomy will be maintained on effective LHRH analogue therapy for the duration of the trial;
Testosterone <50 ng/dL;
Progressive disease after androgen deprivation: PSA evidence for progressive prostate cancer consists of a PSA level of at least 5 ng/ml which has risen on at least 2 successive occasions, at least 2 weeks apart. If the confirmatory PSA value is less than the screening PSA value, then an additional test for rising PSA will be required to document progression;
Antiandrogen Withdrawal Patients who are receiving an antiandrogen as part of primary androgen ablation must demonstrate disease progression following discontinuation of antiandrogen. Disease progression after antiandrogen withdrawal is defined as 2 consecutive rising PSA values, obtained at least 2 weeks apart, or documented osseous or soft tissue progression.
   For patients receiving flutamide, at least one of the PSA values must be obtained 4 weeks or more after flutamide discontinuation;
   For patients receiving bicalutamide or nilutamide, at least one of the PSA values must be obtained 6 weeks or more after antiandrogen discontinuation;
ECOG Performance Status 0-1;
Serum Creatinine ≦1.5×ULN;
K⁺ more than or equal to 3.5 mmol/L;
Bilirubin ≦1.5×ULN;
AST and ALT ≦2.5×ULN;
Systolic blood pressure <160 mmHg and diastolic blood pressure <110 mmHg documented on at least 3 different days;
Baseline ACTH stimulation test demonstrating a peak cortisol >18 μg/dL; and
Life expectancy of more than or equal to 12 weeks.
   Exclusion criteria for Phase I will include:
Therapy with other hormonal therapy, including any dose of megestrol acetate (Megace), finasteride (Proscar), dutasteride (Avodart) any herbal product known to decrease PSA levels (e.g., Saw Palmetto and PC-SPES), or any systemic corticosteroid within 4 weeks prior to first dose of study drug;
Initiation of bisphosphonate therapy within 4 weeks prior to first dose of study drug. Patients on stable doses of bisphosphonates that show subsequent tumor progression may continue on the medication; however, patients will not be allowed to initiate bisphosphonate therapy during the study;
Therapy with supplements or complementary medicines/botanicals within 4 weeks of first dose of study drug, except for any combination of the following:
   conventional multivitamin supplements;
   selenium;
   lycopene;
   soy supplements;
Prior radiation therapy completed <4 weeks prior to enrollment;
Prior chemotherapy for hormone refractory prostate cancer;
Hemoglobin less than or equal to 9.0 g/dL;
ANC less than or equal to 1.5×109/L;
Platelets less than or equal to 100×109/L;
Any “currently active” second malignancy, other than non-melanoma skin cancer. Patients will not considered to have a “currently active” malignancy, if they have completed therapy and are considered by their physician to be at least less than 30% risk of relapse over next 3 months;
Systolic blood pressure more than or equal to 160 mmHg or diastolic blood pressure more than or equal to 110 mmHg measured on at least 2 occasions;
Serum K+<3.5 mmoL/L;
NYHA Class III or IV Congestive Heart Failure;
Myocardial infarction within the 6 months prior to the first dose of study drug;
Serious intercurrent infections or nonmalignant medical illnesses that are uncontrolled;
Active psychiatric illnesses/social situations that would limit compliance with protocol requirements; and
Active or uncontrolled autoimmune disease that may require corticosteroid therapy during study.

Inclusion criteria for Phase II will include the same criteria for Phase I with the following additions:
Neoadjuvant or adjuvant chemotherapy is only allowed if the last dose is >1 year from Cycle 1 Day 1;
Target or Non-Target abnormalities must be present either on screening bone scan, CT or MRI; and
No prior treatment with ketoconazole for the management of androgen independent prostate cancer.

Exclusion criteria for Phase II will include the same criteria as Phase I with the following addition:
Abnormal electrocardiogram, including any finding which would interfere with assessment of intervals (patients with long QT syndrome, bundle branch blocks or hemiblocks will be prohibited).

Study Design: This will be a Phase I/II, non-randomized, open label dose escalation, single group assignment clinical trial of an oral compound of Formula (I), (II), (III), (IA), (IB), (IC), (ID), (IE), (IF), (IG), (IH), (IJ), (IK), (IL), (IM), (IN), (IIA), (IIB) or (IIC).

Primary Outcome Measures: Phase I: To determine maximum tolerated dose of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) administered orally on a continuous once-daily schedule in patients with HRPC. Phase II: To assess proportion of patients achieving a >50% PSA decline during therapy with concurrent prednisone.

Secondary Outcome Measures: Phase I: 1. Safety/tolerability; 2. Pharmacokinetics; 3. Pharmacodynamics; 4. Need for steroids; 5. Preliminary anti-tumor activities. Phase II: 1. To assess safety and tolerability of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) with concurrent prednisone; 2. Additional parameters for anti-tumor activity and clinical benefits.

Arms: Experimental—Phase I: A compound having the structure of Formula (I), (II), (III), (IA), (IB), (IC), (ID), (IE), (IF), (IG), (IH), (IJ), (IK), (IL), (IM), (IN), (IIA), (IIB) or (IIC); Phase II: A compound having the structure of Formula (I), (II), (I), (II), (III), (IA), (IB), (IC), (ID), (IE), (IF), (IG), (IH), (IJ), (IK), (IL), (IM), (IN), (IIA), (IIB) or (IIC) and prednisone.

Assigned Interventions: Drug: A compound having the structure of Formula (I), (II), (III), (IA), (IB), (IC), (ID), (IE), (IF), (IG), (IH), (IJ), (IK), (IL), (IM), (IN), (IIA), (IIB) or (IIC)—Phase I: Dose escalating; Phase II: 1000 mg of a compound having the structure of Formula (I), (II), (III), (IA), (IB), (IC), (ID), (IE), (IF), (IG), (IH), (IJ), (IK), (IL), (IIA), (IIB) or (IIC) PO daily and 5 mg of prednisone PO bid.

The following Examples are intended as an illustration of the various embodiments as defined in appended claims. In some embodiments, the compounds are prepared by a variety of synthetic routes.

### Example 1

### Example 1a

### Parenteral Composition

To prepare a parenteral pharmaceutical composition suitable for administration by injection, 100 mg of a water-soluble salt of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is mixed with 2-hydroxypropyl-β-cyclodextrin and then dissolved in 10 mL of 0.9% sterile saline. The mixture is incorporated into a dosage unit form suitable for administration by injection.

### Example 1b

### Oral Composition

To prepare a capsule suitable for oral administration, a water-soluble salt of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) (20 mg) is mixed with lactose (180 mg), microcrystalline cellulose (140 mg) and magnesium stearate (20 mg). The mixture is granulated and the remaining 10 mg of magnesium stearate is added. The content is then sealed in a gelation capsule.

To prepare a tablet suitable for oral administration, a water-soluble salt of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) (20 mg) is mixed with lactose (70 mg), corn starch (300 mg), microcrystalline cellulose (60 mg) and magnesium stearate (10 mg). The mixture is granulated and the remaining 10 mg of microcrystalline cellulose and 2.5 mg of magnesium stearate is added. The mixture is compression formed to give a suitable tablet.

To prepare a syrup suitable for oral administration, a compound having the structure of Formula (I), (II), (III), (IA), (IB), (IC), (ID), (IE), (IF), (IG), (IH), (IJ), (IK), (IL), (IM), (IN), (IIA), (IIB) or (IIC) (15 mg per 5 ml of syrup) is added to a solution of 0.1% benzoic acid, 5% alcohol, citric acid, edetate disodium, ethyl maltol, flavors, glycerin, ammoniated glycyrrhizin, propylene glycol, purified water, sodium saccharin, sucrose, FD&C blue #1 and FD&C red #40.

### Example 1c

### Sublingual (Hard Lozenge) Composition

To prepare a pharmaceutical composition for buccal delivery, such as a hard lozenge, mix 100 mg of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 1d

### Inhalation Composition

To prepare a pharmaceutical composition for inhalation delivery, 20 mg of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is mixed with 50 mg of anhydrous citric acid and 100 mL of 0.9% sodium chloride solution. The mixture is incorporated into an inhalation delivery unit, such as a nebulizer, which is suitable for inhalation administration.

### Example 1e

### Rectal Gel Composition

To prepare a pharmaceutical composition for rectal delivery, 100 mg of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 1f

### Topical Gel Composition

To prepare a pharmaceutical topical gel composition, 100 mg of a compound having the structure of Formula (I), (II), (III), (IA), (IB), (IC), (ID), (IE), (IF), (IG), (IH), (IJ), (IK), (IL), (IM), (IN), (IIA), (IIB) or (IIC) is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 2

### Preparation of Compound (1)

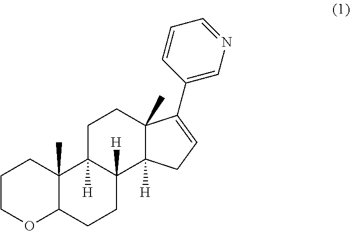

### Example 2A

### Preparation of Compound (1a)

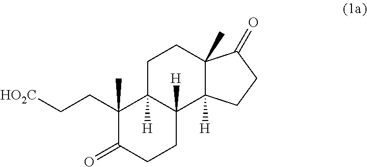

To a mixture of (8R,9S,10R,13S,14S)-10,13-dimethyl-7,8,9,10, 11, 12, 13, 14, 15,16-decahydro-1H-cyclopenta[α]phenanthrene-3,17(2H, 6H)-dione (androstenedione, 5 g, 17.5 mmol) suspended in t-BuOH (200 mL) was added K₂CO₃ (2.9 g, 20.9 mmol, 1.2 equiv) in water (15 mL). After the mixture was heated to 80° C., a solution of KMnO₄ (166 mg, 1.05 mmol, 0.06 equiv) and NaIO₄ (21 g, 99.8 mmol, 5.7 equiv) in water (150 mL) was added dropwise over 1.5 hours. The mixture was heated to 80-90° C. for 5 hours, cooled to room temperature, and filtered. The solid was washed with water (3×). The filtrate was concentrated to remove most of t-BuOH, adjusted pH to 1.5 with 1N HCl, extracted with dichloromethane (DCM) (3×), dried (Na₂SO₄), concentrated to dryness to give compound (1a) as a colorless gum. MS calcd for (C₁₈H₂₆O₄)⁺: 306.2; MS found (electrospray): (M−H)⁻=305.0; ¹H NMR (CDCl₃, 300 MHz) major characteristic peaks: δ 1.15 (s, 3H), 0.90 (s, 3H).

### Example 2B

### Preparation of Compound (1b)

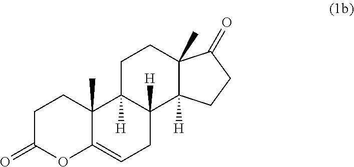

To a solution of Compound (1a) 3-((3aS,5aS,6R, 9aR, 9bS)-3a,6-dimethyl-3,7-dioxododecahydro-1H-cyclopenta[α]naphthalen-6-yl)propanoic acid (5 g) in Ac₂O (60 ml) was added solid NaOAc (1.34 g). The reaction mixture was refluxed for 5 h. The mixture was cooled to room temperature and filtered. The solid was washed with 25% EtOAc in hexanes. The solution was concentrated under vacuum. The residue was purified by column chromatography on silica gel (hexanes:EtOAc=8:1 then 4:1) to provide (4aR,4bS,6aS,9aS,9bR)-4a,6a-dimethyl-4,4a,4b,5,6,6a,9,9a,9b,10-decahydroindeno[5,4-f]chromene-2,7 (3H, 8H)-dione, compound (1b) (3.8 g, 80%). MS calcd for (C₁₈H₂₄O3) [2M+Na]+599.76 Found: 599.9; [2M−H]− 575.76 Found: 576.0. ¹H NMR (CDCl₃, 300 MHz): δ 5.35 (1H), 1.11 (3H) 0.88 (3H).

### Example 2C

### Preparation of Compound (1c)

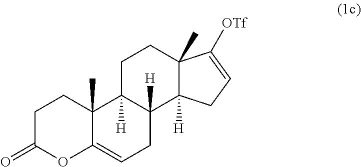

To a solution of (4aR,4bS,6aS,9aS,9bR)-4a,6a-dimethyl-4,4a,4b,5,6,6a,9, 9a,9b,10-decahydroindeno[5,4-f]chromene-2,7 (3H, 8H)-dione (2.0 g, 6.9 mmol) in DCM (35 mL) was added trifluoromethane sulfonic anhydride (0.75 mL, 10.4 mmol, 1.5 equiv) at room temperature. The solution was stirred over 10 min and TEA (1 mL, 6.9 mmol, 1 eq.) in dichloromethane (DCM) (10 mL) was added dropwise within 30 min. The mixture was stirred for 5 h. The reaction was monitored by TLC (EtOAc:hexanes=1:3) and the starting material was completely consumed. Water (20 mL) was added and the layers were separated. The aqueous layer was extracted with DCM (3×50 mL). The organic layers were combined, washed with 2N HCl, brine, dried (MgSO₄). The solution was concentrated and purified by column chromatography on silica gel (hexanes/EA=1:1,1% HOAc) to give a mixture of (4aR,4bS,6aS,9aS,9bR)-4a,6a-dimethyl-2-oxo-2,3,4,4a,4b,5,6,6a,9,9a,9b,10-decahydroindeno[5,4-f]chromene-7-yl trifluoromethanesulfonate compound (1c) and 3-((3aS,5aS,6R, 9aS,9bS)-3a,6-dimethyl-7-oxo-3-(trifluoromethylsulfonyloxy)-3a,4,5,5a,6, 7, 8, 9, 9a,9b-decahydro-1H-cyclopenta[α]naphthalen-6-yl)propanoic acid (2 g, 66%), which was used in the next step without further purification. MS calcd for compound (1c) (C₁₉H₂₅F₃O₆S), [M+H]+439.46 Found: 439.0; [2M−H]− 437.46 Found: 437.1.

### Example 2D

### Preparation of Compound (1d)

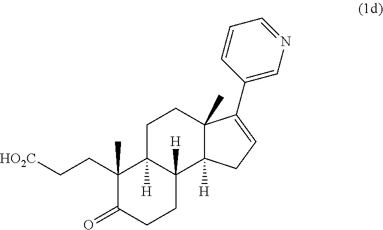

To a solution of (4aR,4bS,6aS,9aS,9bR)-4a,6a-dimethyl-2-oxo-2,3,4,4a,4b, 5,6,6a,9,9a,9b,10-decahydroindeno[5,4-f]chromene-7-yltrifluoromethanesulfonate compound (1c) and 3-((3aS,5aS,6R, 9aS,9bS)-3a,6-dimethyl-7-oxo-3-(trifluoromethylsulfonyloxy)-3a,4,5, 5a,6, 7, 8, 9, 9a,9b-decahydro-1H-cyclopenta[α]naphthalen-6-yl)propanoic acid (2.3 g) in THF (350 mL) was added pyridin-3-yl boronic acid (1.5 g, 2.5 equiv), (Ph₃P)₂PdCl₂ (160 mg, 0.05 equiv) and 2 N aqueous Na₂CO₃ (12 mL). The mixture was degassed and refilled with Argon three times. And, the mixture was heated at 80° C. overnight. The reaction was monitored by TLC. The mixture was cool to room temperature and extracted with DCM (2×30 mL). The organic layers were combined, washed with brine (2×20 mL) dried (Na₂SO₄). The solution was concentrated and purified by column chromatography on silica gel (EtOAc/Hexanes=1:1, 0.5% HOAc) to give 3-((3aS,5aS,6R, 9aS,9bS)-3a,6-dimethyl-7-oxo-3-(pyridin-3-yl)-3a,4,5,5a,6, 7, 8, 9, 9a,9b-decahydro-1H-cyclopenta[α]naphthalen-6-yl)propanoic acid compound (1d), (1.1 g, 65%) as a pale yellow solid. MS calcd for (C₂₃H₂₉NO₃) [2M+H]+ 735.96 Found: 735.5; [2M−H]− 733.96 Found: 733.6. ¹H NMR (CDCl3,300 MHz): δ 8.619 (s, 1H), 8.45 (brs, 1H), 7.68 (d, 1H), 7.29 (m, 1H), 6.00 (s, 1H), 1.163 (s, 3H), 1.062 (s, 3H).

### Example 2E

### Preparation of Compound (1e)

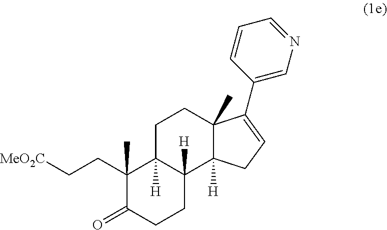

A solution of 3-((3aS,5aS,6R, 9aS,9bS)-3a,6-dimethyl-7-oxo-3-(pyridin-3-yl)-3a,4,5,5a,6, 7, 8, 9, 9a,9b-decahydro-1H-cyclopenta[α]naphthalen-6-yl)propanoic acid, compound (1d) (300 mg, 0.82 mmol) in MeOH (30 mL) was treated with SOCl₂ (0.6 mL, 8.2 mmol) at room temperature. The resulting solution was stirred at reflux for 10 min and all the starting material has been consumed (TLC: EtOAc:Hexane=2:1 1% HOAc). The reaction solution was concentrated under reduce pressure. The residue was then dissolved in 30 mL EtOAc, and washed saturated NaHCO₃ and brine, dried over anhydrous sodium sulfate and was filtered, the solvent was evaporated to yield 320 mg (100%) of the product compound (1e) as a yellowish powder. which was used in the next step without further purification. A small sample of the power was purified through HPLC giving pure compound (1e). 1H NMR (CDCl₃, 300 MHz): δ 8.7 (s, 1H), 8.5 (brs, 1H), 7.7 (d, 1H), 7.3 (m, 1H), 6.0 (s, 1H), 3.7 (s, 3H), 1.2 (s, 3H), 1.1 (s, 3H).

### Example 2F

### Preparation of Compound (1f)

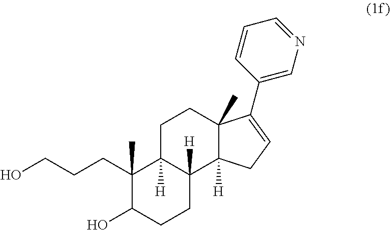

A solution of compound (1e) (1.1 g, 2.88 mmol) in tetrahydrofuran (THF) (80 ml) was treated with LiAlH₄ (0.43 g, 11.5 mmol) at room temperature. The resulting mixture was stirred at room temperature until all the starting material has been consumed (ILC: EtOAc:Hexane=2:1). The reaction mixture was concentrated under reduce pressure. The residue was dissolved in a solution of dichloromethane-methanol (1/1,50 mL) and filtered though a pad of silica gel. The filtrate was evaporated to afford 0.8 g of compound (1f) as off-white powder. MS calculated for (C₂₃H₃₃NO₂) [M+H]+ 356.51. Found: 357.2; [M+Na]+378.51 Found: 379.2; ¹H NMR (CDCl₃, 300 MHz): δ 8.65 (s, 1H), 8.55 (brs, 1H), 7.75 (d, 1H), 7.25 (m, 1H), 5.98 (s, 1H), 3.75 (m, 2H), 3.55-3.64 (m, 1H) 1.05 (s, 3H), 0.98 (s, 3H). ¹³C NMR (CDCl3, 300 MHz): 151.23, 147.11, 147.03, 134.26, 133.36, 129.57, 123.32, 72.34, 62.76, 57.24, 47.39, 46.03, 40.52, 16.50, 15.02

### Example 2G

### Preparation of Compound (1)

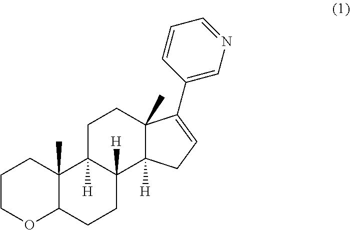

A solution of compound (1f) (400 mg, 1.12 mmol) in pyridine (5 mL) was treated with tosyl chloride (1 mL) was at 70° C. The reaction mixture was stirred until all the starting material was consumed (ILC: EtOAc:Hexane=2:1). The resulting mixture was diluted with EtOAc (50 mL) and water (20 mL). The organic layer was separated. The aqueous layer was extracted with EtOAc. The combined organic layer was washed with water (3×20 mL) and brine (2×20 mL), dried over anhydrous sodium sulfate, filtered, the solvent was evaporated to give 350 mg (89%) of isomeric mixture of 5α-epimer and 5β-epimer of compound (1). The mixture was applied to pre-HPLC to isolate 5α-epimer and 5β-epimer. Analytical data of 5α-epimer of compound (1): Retention time at HPLC: 11.83 minutes (min) [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. MS calculated for (C₂₃H₃₁NO) [M+H]+ 337.50 Found: 338.1; ¹H NMR (CDCl3, 300 MHz): δ 8.67 (s, 1H), 8.52 (brs, 1H), 7.65 (d, 1H), 7.21 (m, 1H), 5.98 (s, 1H), 3.96-4.01 (m, 1H), 3.55 (m, 1H), 2.98 (m, 1H), 1.09 (s, 3H), 1.05 (s, 3H); ¹³C NMR (CDCl3, 300 MHz): 152.00, 147.94, 147.86, 133.67, 133.04, 129.15, 123.00, 84.95, 69.08, 57.11, 52.08, 47.69 Analytical data of 5β-epimer of compound (1): Retention time at HPLC: 12.54 min [Mobile phase: B %=10-100 (gradient 20 min); B=MeCN, A=H2O (0.1% TFA); Flow rate: 0.8 mL/min; UV=266 nm column: zorbax Eclipse XDB-C8 5u, (150×4.6 mmID)]; MS calculated for (C₂₃H₃₁NO) [M+H]+ 337.50 Found: 338.2; 1H NMR (CDCl₃, 300 MHz): δ 8.62 (s, 1H), 8.55 (brs, 1H), 7.65 (d, 1H), 7.21 (m, 1H), 5.98 (s, 1H), 4.01 (m, 1H), 3.45 (m, 1H), 3.15 (s, 1H), 1.09 (s, 3H), 0.98 (s, 3H); ¹³C NMR (CDCl₃, 300 MHz): 151.70, 147.92, 147.77, 133.74, 133.07, 129.35, 123.00, 82.55, 68.91, 57.14, 47.62, 40.64

### Example 3

### Preparation of Compound (2a) and Compound (1b)

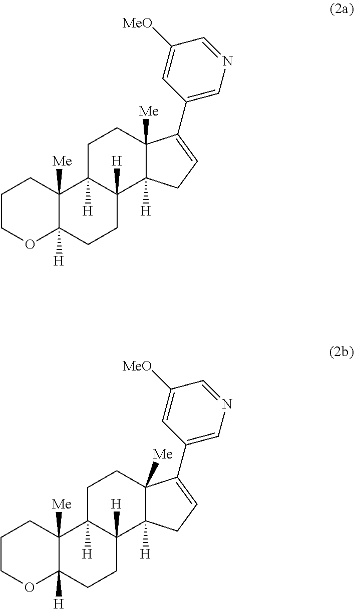

Using a synthetic procedure and conditions similar to Examples 2A -2G in the preparation of compound (1), replacing pyridin-3-yl boronic acid with 5-methoxypyridin-3-ylboronic acid in Example 2D, 5α-epimer compound (2a) and 5β-epimer compound (2b) were made.

Compound (2a): Ret time at HPLC: 13.142 min [Mobile phase: B %=10-100 (gradient 20 min); B=MeCN, A=H2O (0.1% TFA); Flow rate: 0.8 mL/min; UV=266 nm; column: zorbax Eclipse XDB-C8 5u, (150*4.6 mmID)]; ¹H NMR (CDCl₃, 400 MHz): 8.4 (1H), 8.2 (1H), 7.12 (1H), 5.9 (1H), 3.92 (1H), 3.83 (3H), 3.65 (1H), 2.92 (1H), 1.01 (6H); ¹³C NMR (CDCl3, 400 MHz): 155.22, 151.51, 140.50, 135.18, 133.52, 129.41, 84.90, 69.05, 57.07, 55.49, 52.05, 47.70, 36.54, 36.18, 35.15, 33.43, 31.61, 29.60, 27.38, 22.73, 20.39, 16.73, 12.62; ESI-MS: m/z calcd for C₂₄H₃₃NO₂: [M+H]+ 368.52. Found: 368.8; [2M+H]+ 736.04 Found: 735.9.

Compound (2b): Ret time: 13.797 min [Mobile phase: B %=10-100 (gradient 20 min); B=MeCN, A=H2O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.24 (s, 1H), 8.15 (s, 1H), 7.12 (s, 1H), 5.97 (s, 1H), 4.02 (m, 1H), 3.87 (s, 3H), 3.40 (m, 1H), 3.15 (s, 1H), 2.25 (m, 1H), 2.02 (m, 2H), 1.60-1.9 (m, 8H), 1.2-1.6 (m, 7H), 1.1 (m, 1H), 1.01 (s, 3H), 0.91 (s, 3H); ¹³C NMR (400 MHz, CDCl₃): 154.22, 150.48, 139.48, 134.15, 132.61, 128.61, 117.65, 81.51, 67.88, 56.11, 54.48, 46.63, 39.62, 39.62, 34.54, 34.38, 33.36, 32.66, 30.71, 26.27, 24.81, 21.09, 20.35, 19.64, 15.66; ESI-MS: m/z calcd for C₂₄H₃₃NO₂: [M+H]+ 368.5. Found: 368.3.

### Example 4

### Preparation of Compound (3a) and Compound (3b)

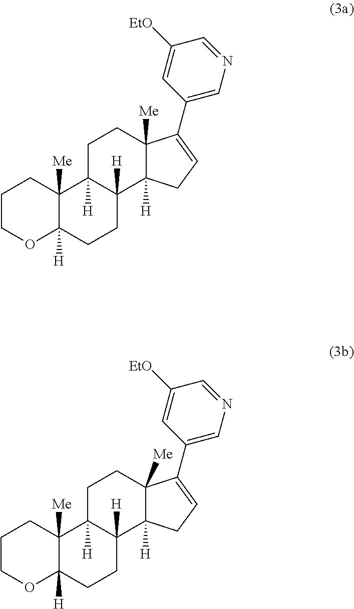

Using a synthetic procedure and conditions similar to Examples 2A -2G in the preparation of compound (1), replacing pyridin-3-yl boronic acid with 5-ethoxypyridin-3-ylboronic acid in Example 2D, 5α-epimer compound (3a) and 5β-epimer compound (3b) were made.

Compound (3a): Ret time at HPLC: 14.037 min [Mobile phase: B %=10-100 (gradient 20 min); B=MeCN, A=H2O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.2 (1H), 8.12 (1H), 7.12 (1H), 5.95 (1H), 4.12 (2H), 3.96 (1H), 3.45 (1H), 2.97 (1H), 1.45 (3H), 0.95 (6H); ¹³C NMR (CDCl₃, 400 MHz): 154.60, 151.53, 140.37, 135.54, 133.51, 129.29, 119.25, 84.90, 69.05, 63.81, 57.07, 52.05, 47.68, 36.54, 36.18, 35.15, 33.42, 31.60, 29.60, 27.38, 22.74, 20.39, 16.73, 14.77, 12.62; ESI-MS: m/z calcd for C₂₅H₃₅NO₂: [M+H]+ 382.55. Found: 382.5

Compound (3b): Ret time: 14.601 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.23 (s, 1H), 8.15 (s, 1H), 7.139 (s, 1H), 5.96 (s, 1H), 4.12 (m, 2H), 4.02 (m, 1H), 3.40 (m, 1H), 3.13 (s, 1H), 2.25 (m, 1H), 2.01 (m, 3H), 1.60-1.95 (m, 9H), 1.30-1.60 (m, 9H), 1.25 (m, 1H), 1.10 (m, 2H), 1.01 (s, 3H), 0.91 (s, 3H); ¹³C NMR (400 MHz, CDCl₃): 153.61, 150.51, 139.35, 134.50, 132.64, 128.51, 118.31, 81.52, 67.88, 62.81, 56.10, 46.62, 39.62, 34.53, 34.38, 33.37, 32.66, 30.70, 26.27, 24.81, 21.08, 20.36, 19.63, 15.66, 13.76; ESI-MS: m/z calcd for C₂₅H₃₅NO₂: [M+H]+ 382.6. Found: 382.6.

### Example 5

### Preparation of Compound (4a) and Compound (4b)

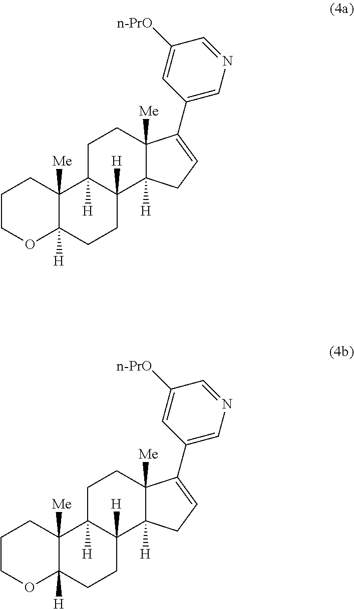

Using a synthetic procedure and conditions similar to Examples 2A -2G in the preparation of compound (1), replacing pyridin-3-yl boronic acid with 5-propoxypyridin-3-ylboronic acid in Example 2D, 5α-epimer compound (4a) and 5β-epimer compound (4b) were made.

Compound (4a): Ret time at HPLC: 15.255 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.21 (1H), 8.12 (1H), 7.12 (1H), 5.95 (1H), 3.91-4.05 (3H), 3.44 (1H), 2.89 (1H), 0.95-1.1 (12H); ¹³C NMR (CDCl₃, 400 MHz): 154.80, 151.54, 140.24, 135.59, 133.49, 129.26, 119.21, 84.88, 69.75, 69.02, 57.05, 52.04, 47.67, 12.60, 10.46; ESI-MS: m/z calcd for C₂₆H₃₇NO₂: [M+H]+ 396.58. Found: 396.3.

Compound (4b): Ret time at HPLC: 16.098 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.209 (1H, s), 8.148 (1H, s), 7.142 (1H, s), 5.96 (1H, s), 4.01 (1H, m), 3.95 (2H, m), 3.45 (1H, m), 3.14 (1H, m), 2.22 (1H, m), 2.01 (2H, m), 1.03 (3H, m), 1.0 (3H, s), 0.95 (3H, s); ¹³C NMR (CDCl₃, 400 MHz): 153.81, 150.54, 139.26, 134.59, 132.62, 128.49, 118.30, 81.52, 68.78, 67.89, 56.11, 46.63, 39.63, 15.67, 9.46; ESI-MS: m/z calcd for C₂₆H₃₇NO₂; [M+H]+ 396.58. Found: 396.3.

### Example 6

### Preparation of Compound (5a) and Compound (5b)

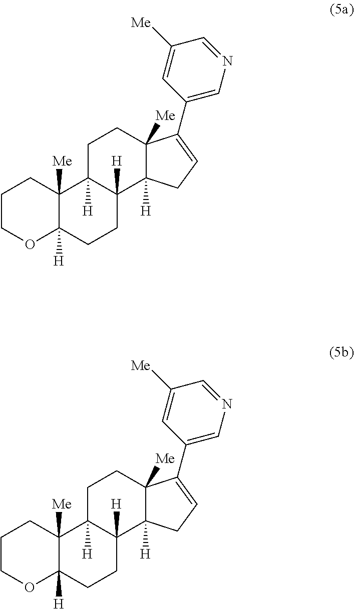

Using a synthetic procedure and conditions similar to Examples 2A -2G in the preparation of compound (1), replacing pyridin-3-yl boronic acid with 5-methylypyridin-3-ylboronic acid in Example 2D, 5α-epimer compound (5a) and 5β-epimer compound (5b) were made.

Compound (5a): Ret time: 12.358 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.392 (s, 1H), 8.269 (s, 1H), 8.42 (s, 1H), 5.92 (s, 1H), 3.85 (m, 1H), 3.41 (m, 1H), 2.94 (m, 1H), 2.31 (s, 3H), 2.20 (m, 1H), 2.01 (m, 1H), 1.7-1.9 (m, 3H), 1.3-1.7 (m, 9H), 1.0-1.15 (m, 2H), 0.98 (d, 6H); ¹³C NMR (400 MHz, CDCl₃): 151.78, 148.35, 145.06, 134.34, 132.46, 132.33, 128.89, 84.92, 69.06, 57.09, 47.65, 36.55, 36.19, 35.15, 33.45, 31.59, 29.62, 27.40, 22.74, 20.38, 18.44, 16.70, 12.62; ESI-MS: m/z calcd for C₂₄H₃₃NO: [M+H]+ 352.52. Found: 352.31.

Compound (5b): Ret time: 13.243 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.41 (s, 1H), 8.21 (s, 1H), 7.429 (s, 1H), 5.95 (s, 1H), 4.01 (m, 1H), 3.40 (m, 1H), 3.23 (s, 1H), 3.31 (s, 3H), 2.22 (m, 1H), 2.02 (m, 3H), 1.6-1.9 (m, 7H), 1.3-1.6 (m, 5H), 1.25 (m, 2H), 1.09 (m, 1H), 1.01 (s, 3H), 0.89 (s, 3H); ¹³C NMR (400 MHz, CDCl₃): 150.75, 147.24, 144.03, 133.44, 131.56, 131.32, 128.10, 81.54, 67.88, 59.38, 56.12, 46.59, 39.63, 34.55, 34.39, 33.38, 32.68, 30.69, 26.28, 24.83, 21.09, 20.36, 20.03, 19.63, 17.43, 15.64, 13.18; ESI-MS: m/z calcd for C₂₄H₃₃NO: [M+H]+ 352.52. Found: 352.3.

### Example 7

### Preparation of Compound (6a) and Compound (6b)

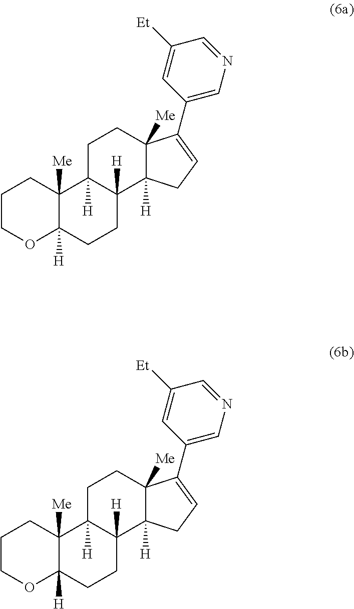

Using a synthetic procedure and conditions similar to Examples 2A -2G in the preparation of compound (1), replacing pyridin-3-yl boronic acid with 5-ethylpyridin-3-ylboronic acid in Example 2D, 5α-epimer compound (6a) and 53-epimer compound (6b) were made.

Compound (6a): Ret time: 13.350 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.43 (s, 1H), 8.31 (s, 1H), 7.45 (s, 1H), 5.95 (s, 1H), 3.98 (m, 1H), 3.45 (m, 1H), 2.97 (m, 1H), 2.61 (m, 1H), 2.21 (m, 1H), 2.01 (m, 2H), 1.67-1.9 (m, 4H), 1.35-1.65 (m, 10H), 1.26 (m, 3H), 1.10 (m, 3H), 1.01 (d, 6H); ¹³C NMR (400 MHz, CDCl₃): 151.87, 147.69, 145.34, 138.45, 133.11, 132.56, 128.83, 84.93, 69.06, 57.10, 52.08, 47.67, 36.56, 36.62, 35.17, 33.46, 31.60, 29.63, 27.40, 26.05, 22.75, 20.40, 16.72, 15.34, 12.63; ESI-MS: m/z calcd for C₂₅H₃₅NO: [M+H]+ 366.6. Found: 366.3.

Compound (6b): Ret time: 14.185 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.43 (s, 1H), 8.31 (s, 1H), 7.45 (s, 1H), 5.97 (s, 1H), 4.01 (m, 1H), 3.42 (m, 1H), 3.13 (s, 1H), 2.67 (m, 2H), 2.22 (m, 1H), 2.01 (m, 2H), 1.60-1.91 (m, 7H), 1.33-1.6 (m, 5H), 1.30 (m, 4H), 1.10 (m, 1H), 1.01 (s, 3H), 0.91 (s, 3H); ¹³C NMR (400 MHz, CDCl₃): 150.83, 146.58, 144.30, 137.44, 132.20, 131.64, 128.02, 81.53, 67.88, 56.13, 46.60, 39.63, 34.56, 34.39, 33.38, 32.69, 30.70, 26.29, 25.04, 24.83, 21.09, 20.37, 19.68, 15.65, 14.33; ESI-MS: m/z calcd for C₂₅H₃₅NO: [M+H]+ 366.6. Found: 366.3.

### Example 8

### Preparation of Compound (7)

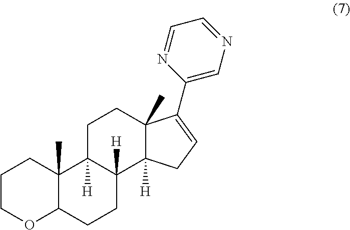

Using a synthetic procedure and conditions similar to Examples 2A -2G in the preparation of compound (1), replacing pyridin-3-yl boronic acid with 2-(tributylstannyl)pyrazine in Example 2D, 5α-epimer of compound (7) and 5β-epimer of compound (7) are made.

### Example 9

### Preparation of Compound (8)

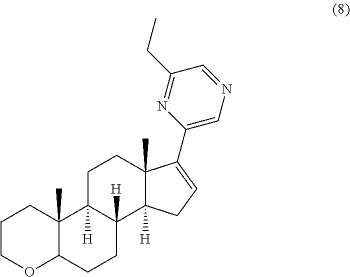

Using a synthetic procedure and conditions similar to Examples 2A -2G in the preparation of compound (1), replacing pyridin-3-yl boronic acid with 2-ethyl-6-(tributylstannyl)pyrazine in Example 2D, 5α-epimer of compound (8) and 5β-epimer of compound (8) are made.

### Example 10

### Preparation of Compound (9)

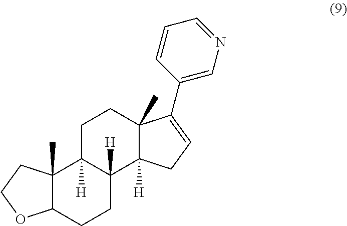

### Example 10A

### Preparation of Compound (9a)

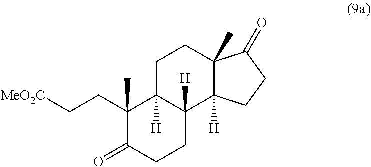

To a solution of compound (1a) (75 g) in MeOH (500 mL) was added dropwise with SOCl₂ (34.95 g, 21.4 ml) at 0° C. under nitrogen. After addition, the reaction mixture was stirred at reflux until all the starting material was consumed by TLC analysis (hexane: EtOAc=1:1). The solvent was removed in vacuo. The residue was diluted in EtOAc (300 ml), washed with water (200 ml), saturated aqueous NaHCO₃ (200 ml) and brine (200 ml), dried over anhydrous magnesium sulfate, filtered, and evaporated to afford 70.5 g of compound (9a).

### Example 10B

### Preparation of Compound (9b)

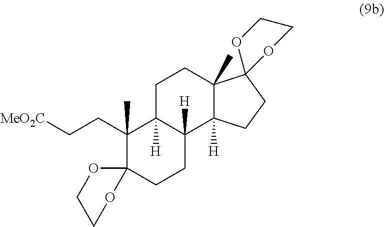

A solution of compound (9a) (12.7 g), ethylene glycol (13 ml), p-TsOH (0.1 g) in toluene (180 ml) was heated at reflux under a Dean-Stark trap for 5 hours (h). The reaction mixture was cooled to room temperature and evaporated to dryness. The residue was diluted with EtOAc (300 ml), washed with saturated aqueous NaHCO₃ (3×100 ml) and brine (3×100 ml), dried over sodium sulfate, filtered, and evaporated to dryness. The residue was purified by flash column chromatograph to afford 11.2 g of the compound (9b).

### Example 10C

### Preparation of Compound (9c)

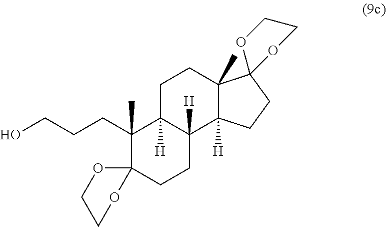

A solution of compound (9b) (30 g) in THF (250 ml) was treated with LiAlH₄ (11.15 g) at 0° C. The resulting mixture was warmed to room temperature and stirred until all the starting material was consumed as indicate by TLC analysis (hexane: EtOAc=3:1). The reaction was quenched with MeOH. The mixture was concentrated in vacuo. The residue was suspended in EtOAc (300 ml), washed with water (200 ml) and brine (200 ml), dried over magnesium sulfate, and evaporated to dryness. The residue was purified by column chromatograph (silica gel) to afford 27.2 g (97%) of compound (9c). MS calculated for (C₂₂H₃₆O₅) [M+H]+ 381.52 Found: 380.6

### Example 10D

### Preparation of Compound (9d)

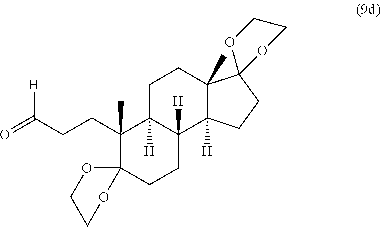

A catalytic amount of TEMPO (2,2,6,6-tetramethyl-1-piperidinyloxy) (90.78 mg) was added to a solution of compound (9c) (22 g) in dichloromethane (500 ml) at 0° C., followed by addition of trichloroisocyanuric acid (13.51 g, 58.1 mmol) at 0° C. After addition, the mixture was warmed to room temperature and stirred for 15 min. The reaction mixture was monitored by TLC analysis. The mixture was poured into saturated aqueous Na₂CO₃. The formed solid was removed away by filtration. The organic layer was separated, washed with 10% aqueous Na₂SO₃ (100 ml) and brine (150 ml), dried over anhydrous sodium sulfate, filtered, and evaporated to dryness. The residue was purified by flash column chromatograph (silica gel) to afford 17.2 g (80%) of compound (9d). ¹H NMR (CDCl₃): 9.75 (1H), 3.8-3.9 (8H), 2.05 (2H), 0.95 (3H), 0.85 (3H); ¹³C NMR (CDCl₃): 203.76, 128.49, 123.89, 119.27, 113.73, 68.38, 65.17, 64.55, 64.03, 30.41, 49.84

### Example 10E

### Preparation of Compound (9e)

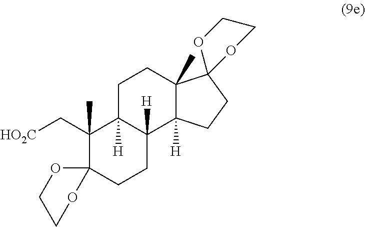

To a stirred solution of compound (9d) (17.2 g, 45.44 mmol) in acetone-water (750 ml, 10/1) was added solid NaIO₄ (39 g, 181.76 mmol), N-methylmorpholine (NMM) (5.00 mL), N-methylmorpholine N-oxide (NMO) (10.68 g, 90.88 mmol), and OsO₄ (2.27 mmol) at room temperature. The resulting mixture was stirred overnight and monitored by TLC analysis (EtOAc/Hexanes=1/6). The mixture was filtered though a pad of silica gel, the filtrate was washed with 1N aqueous Na₂SO₃ (twice, each at 300 ml) and brine (300 ml), dried over anhydrous sodium sulfate, filtered, and evaporated to give 10.4 g (65%) of one carbon short aldehyde. ¹H NMR (CDCl₃): 9.7 (1H), 3.8-3.9 (8H), 2.2 (2H), 1.12 (3H), 0.95 (3H). To a stirred solution of this compound (4.9 g) in CH₂Cl₂ (50 mL), m-chloroperbenzoic acid (MCPBA) (2.53 g) and NaHCO₃ (2.45 g) were added at room temperature. The mixture was heated at reflux and monitored by TLC analysis. The reaction mixture was quenched with 10% aqueous Na₂SO₃ (25 mL) and the mixture was stirred for an additional 30 min. the organic layers was separated, washed with saturated aqueous NaHCO₃ (3×50 mL) and brine, and dried over Na₂SO₄. After evaporating the solvent in vacuo, the residue was purified by column chromatograph to afford compound (9e) 5.4 g (98%). MS calculated for (C₂₁H₃₂O₆) [M+H]+ 381.48 Found: 381.3, [M−H]+ 379.48 Found: 379.6; ¹H NMR (CDCl₃): 3.8-4.0 (8H), 2.2-2.4 (2H), 1.1 (3H), 0.85 (3H); ¹³C NMR (CDCl₃): 177.75, 119.32, 112.63, 65.19, 64.60, 64.24, 63.95, 49.58

### Example 10F

### Preparation of Compound (9f)

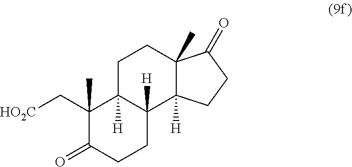

A solution of compound (9e) (3 g) in THF was treated with 3N aqueous HCl at room temperature. The mixture was stirred until all the starting material was consumed. The organic solvent was removed under reduce pressure. The aqueous phase was extracted with EtOAc (2×20 ml). The organic phase was washed with water and brine, dried over anhydrous sodium sulfate, evaporated to afford 2.87 g of compound (9f).

### Example 10G

### Preparation of Compound (9g)

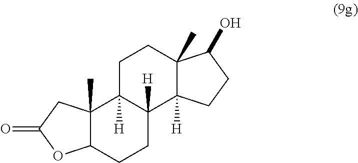

To a solution of compound (9f) (2.12 g, 7.25 mmol) in THF (15 ml) was added NaBH₄ (805 mg) at 0° C. under stirring, followed by dropwise addition of MeOH. The mixture was stirred until all the starting material was consumed by TLC analysis. The organic solvent was remove under reduce pressure, the residue was suspended in EtOAc and washed with water, the aqueous was reextracted with EtOAc (2×50 ml). The combined organic phases were washed with water and brine, dried over anhydrous sodium sulfate, and evaporated to afford 1.7 g of di-alcohol acid. ¹H NMR (CDCl₃): 3.75 (1H), 3.6 (1H), 2.2 (3H), 1.12 (3H), 0.96 (3H); ¹³C NMR (CDCl₃): 176.34, 86.86, 80.35, 49.66, 49.18. A solution of this dialcohol acid compound (50 mg) and p-TsOH (2 mg) in toluene was heated at reflux for 30 min, and all the starting material was consumed as indicate by TLC analysis. The solvent was removed under reduce pressure. The residue was diluted with dichloromethane (50 ml) and filtered though a pad of silica gel. The filtrate was concentrated in vacuo to provide compound (2g) which was used in the next step without further purification. MS calculated for compound (9g): (C₇H₂₆O₃) [M+H]+ 279.39 Found: 279.0; ¹H NMR (CDCl₃): 3.75-3.8 (1H), 3.55 (1H), 2.2 (2H).

### Example 10H

### Preparation of Compound (9h)

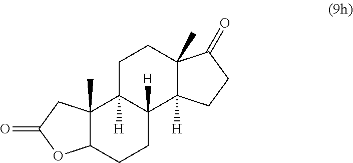

A catalytic amount of TEMPO (2,2,6,6-tetramethyl-1-piperidinyloxy) (5.63 mg) was added to a solution of compound (2g) (1 g) in dichloromethane (240 ml) at 0° C., followed by addition of trichloroisocyanuric acid (840 mg) at 0° C. After addition, the mixture was warmed to room temperature and stirred for 10 min, filtered though a pad of silica gel, the filtrate was washed with saturated aqueous Na₂CO₃, 10% aqueous Na₂SO₃ and brine, dried over anhydrous sodium sulfate, filtered, and evaporated to dryness. The residue was purified by flash column chromatograph (silica gel) to afford 830 mg (81.5%) of compound (9h) consisting of two 5-epimers.

### Example 101

### Preparation of Compound (91)

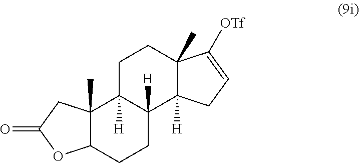

Using a synthetic procedure and conditions similar to Examples 2C in the preparation of compound (1c), replacing (4aR,4bS,6aS,9aS,9bR)-4a,6a-dimethyl-4,4a,4b,5, 6, 6a,9, 9a,9b,10-decahydroindeno[5,4-f]chromene-2, 7 (3H, 8H)-dione with compound (9h) a mixture of 5α-epimer of compound (91) and 5β-epimer of compound (91) were prepared. ¹H NMR (CDCl₃): 5.5 (1H), 3.8-3.9 (1H), 2.35 (2H), 1.0-1.1 (6H).

### Example 10J

### Prepared of Compound (2j)

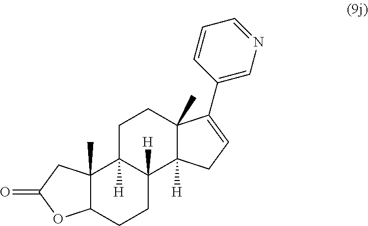

Following the procedure as described in the preparation of compound (1d) from compound (1c) as in Example 2D, reacting compound (91) with pyridin-3-yl boronic acid instead of reacting (4aR,4bS,6aS,9aS,9bR)-4a,6a-dimethyl-2-oxo-2,3,4,4a,4b,5, 6, 6a,9, 9a,9b,10-decahydroindeno[5,4-f]chromene-7-yl trifluoromethanesulfonate, compound (2j) was prepared. ¹H NMR (CDCl₃): 5.5 (1H), 3.8-3.9 (1H), 2.35 (2H), 1.0-1.1 (6H).

### Example 10K

### Preparation of Compound (9k)

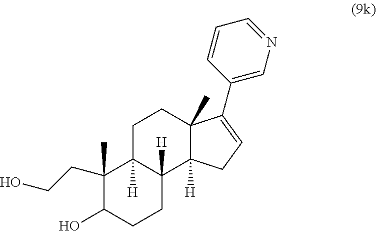

Following the procedure as described in the preparation of compound (9c) with the treatment with LiAlH₄ in Example 10C, replacing compound (9b) with compound (2j) the di-alcohol compound (9k) was prepared. ¹H NMR (CDCl₃): 8.4 (1H), 8.2 (1H), 7.45 (1H), 7.0 (1H), 5.8 (1), 4.15-4.31 (2H), 3.4-3.5 (2H), 3.2 (1H), 0.82 (3H), 0.75 (3H). MS calculated for (C₂₂H₃ᵢNO₂) [M+H]+ 342.49 Found: 342.4, [M+Na]+364.49 Found: 364.2 1.01 (s, 3H).

### Example 10L

### Preparation of Compound (9)

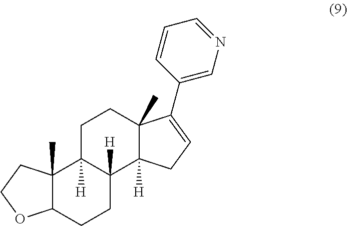

Using a synthetic procedure and conditions similar to Examples 2G in the preparation of compound (1), replacing compound (1f) with compound (9k) a mixture of, α-epimer of compound (9) and β-epimer of compound (9) were made. Retention time at HPLC: 10.441 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]; MS calculated for (C₂₂H₃₉NO) [M+H]+ 324.47 Found: 324.8, [M+Na]+346.47 Found: 346.7, [2M+Na]+669.94 Found: 670.4; ¹H NMR (CDCl₃): 8.6 (1H), 8.4 (1H), 7.6 (1H), 7.12 (1H), 5.9 (1H), 3.8-3.9 (2H), 3.02 (1H), 2.2 (1H), 0.98 (3H), 0.82 (3H); ¹³C NMR (CDCl₃): 150.77, 146.94, 146.88, 132.71, 131.95, 128.15, 122.0, 85.26, 64.76, 55.83, 50.76, 47.05, 42.41, 37.32

### Example 11

### Preparation of Compound (10)

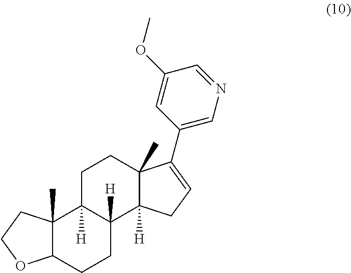

Using a synthetic procedure and conditions similar to Examples 10A -10L in the preparation of compound (9), and replacing pyridin-3-yl boronic acid with 5-methoxypyridin-3-ylboronic acid in Example 10J, a mixture of α- and β-epimers of compound (10) are made.

### Example 12

### Preparation of Compound (11a) and Compound (11b)

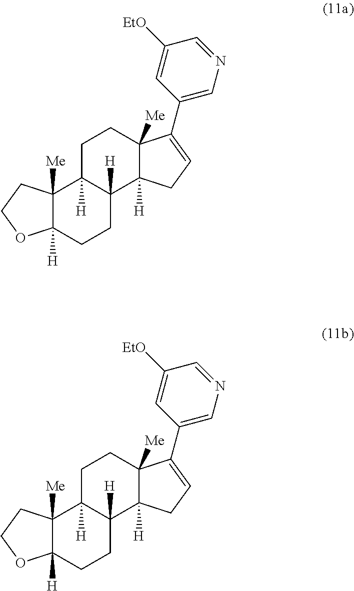

Using a synthetic procedure and conditions similar to Examples 10A -10L in the preparation of compound (9), and replacing pyridin-3-yl boronic acid with 5-ethoxypyridin-3-ylboronic acid in Example 10J, 5α-epimer compound (11a) and 5β-epimer compound (11b) were made.

Compound (11a): Ret time: 12.534 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.20 (s, 1H), 8.10 (s, 1H), 7.09 (s, 1H), 5.95 (s, 1H), 4.06 (m, 2H), 3.89 (m, 2H), 3.01 (m, 1H), 2.21 (m, 1H), 2.0 (m, 2H), 1.8-1.95 (m, 2H), 1.3-1.7 (m, 10H), 1.01 (s, 3H), 0.85 (s, 3H); ¹³C NMR (400 MHz, CDCl₃): 153.60, 150.56, 139.32, 134.51, 132.50, 128.30, 118.32, 85.21, 64.82, 62.80, 55.79, 50.73, 47.04, 42.38, 37.29, 34.01, 32.92, 30.83, 28.19, 23.94, 22.49, 15.84, 14.26, 13.76, 12.76; ESI-MS: m/z calcd for C₂₄H₃₃NO₂: [M+H]+ 368.52. Found: 368.3.

### Example 13

### Preparation of Compound (12)

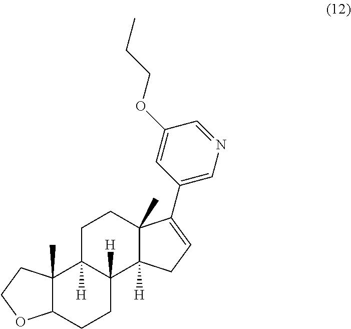

Using a synthetic procedure and conditions similar to Examples 10A -10L in the preparation of compound (9), and replacing pyridin-3-yl boronic acid with 5-propoxypyridin-3-ylboronic acid in Example 10J, a mixture of α- and β-epimers of compound (12) are made.

### Example 14

### Preparation of Compound (13)

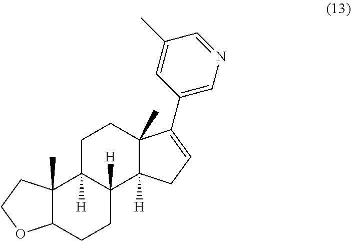

Using a synthetic procedure and conditions similar to Examples 10A -10L in the preparation of compound (9), and replacing pyridin-3-yl boronic acid with 5-methylpyridin-3-ylboronic acid in Example 10J, a mixture of α- and β-epimers of compound (13) are made.

### Example 15

### Preparation of Compound (14)

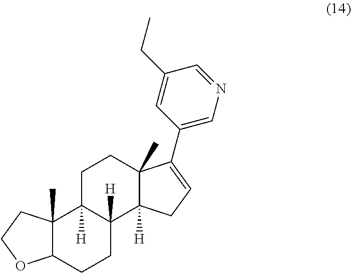

Using a synthetic procedure and conditions similar to Examples 10A -10L in the preparation of compound (9), and replacing pyridin-3-yl boronic acid with 5-ethylpyridin-3-ylboronic acid in Example 10J, a mixture of α- and β-epimers of compound (14) are made.

### Example 16

### Preparation of Compound (15)

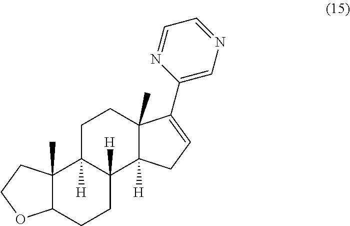

Using a synthetic procedure and conditions similar to Examples 10A -10L in the preparation of compound (9), and replacing pyridin-3-yl boronic acid with 2-(tributylstannyl)pyrazine in Example 10J, a mixture of α- and β-epimers compound (15) are made.

### Example 17

### Preparation of Compound (16)

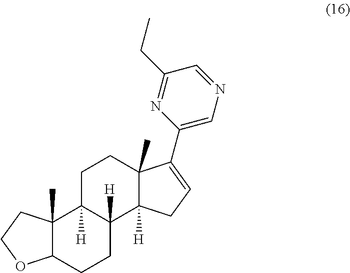

Using a synthetic procedure and conditions similar to Examples 10A -10L in the preparation of compound (9), and replacing pyridin-3-yl boronic acid with 2-ethyl-6-(tributylstannyl)pyrazine in Example 10J, a mixture of α- and β-epimers of compound (16) are made.

### Example 18

### Preparation of Compound (17)

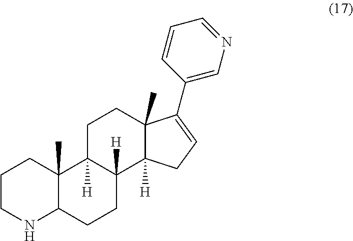

### Example 18A

### Preparation of Compound (17a)

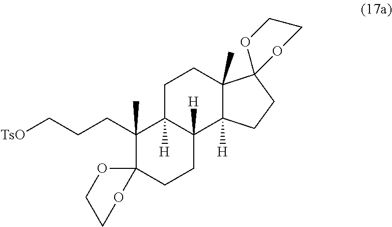

To a solution of compound (9c) (19 g) in dichloromethane (200 ml) at room temperature was added pyridine (11.8 g) and TsCl (14.28 g). The resulting mixture was stirred until all the starting material was consumed by TLC analysis (hexane: EtOAc=3:1). The mixture was concentrated in vacuo. The residue was suspended in EtOAc (300 ml), washed with saturated KHSO₄ (200 ml) and brine (200 ml), dried over magnesium sulfate, and evaporated to give 18.5 g (69.3%) of compound (17a). ¹H NMR (300 MHz, CDCl₃): 7.8 (2H), 7.3 (2H), 3.8-4.0 (10H), 2.5 (3H), 0.95 (3H), 0.85 (3H).

### Example 18B

### Preparation of Compound (17b)

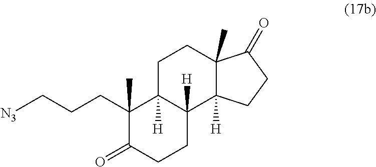

A solution of compound (17a) (14.4 g) in DMF (200 ml) was treated with NaN₃ (2.1 g) at 50° C. The reaction mixture was stirred for 5 h and monitored by TLC analysis (hexane: EtOAc=3:1). The mixture was poured into ice water (300 ml) and extracted with methyl tert-butyl ether (3×200 ml). The combined organic phases were washed with water (200 ml) and brine (200 ml), dried over magnesium sulfate, and evaporated to dryness. The residue (11 g) was dissolved in THF (150 mL) and treated with 5N aqueous HCl (30 ml) at room temperature. The reaction was monitored by TLC analysis (hexane: EtOAc=5:1), the solvent was removed in vacuo. The residue was dissolved in EtOAc (50 ml) and washed with water (50 ml) and brine (50 ml), dried over magnesium sulfate, and evaporated to give 8.45 g (99%) of compound (17b). ¹H NMR (CDCl₃): 3.15-3.25 (1H), 3.05-3.15 (1H), 2.3-2.5 (3H), 1.15-2.2 (1H), 1.0 (3H), 0.85 (3H). ¹³C NMR (CDCl₃): 213.80, 51.92, 50.72, 50.66, 47.50, 47.46, 37.67, 35.60, 34.25.

### Example 18C

### Preparation of Compound (17c)

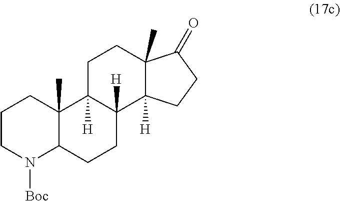

The compound (17b) (8.45 g) was dissolved in MeOH (300 ml) and hydrogenated under H₂ (1 atm) in the presence of 5% Pd—C (1.5 g) at room temperature overnight. The reaction mixture was filtered though a pad of silica gel and washed with MeOH. To the filtrate was added HOAc (0.08 g) and sodium cyanoborohydride (1.68 g) at 0° C. The resulting mixture was stirred for 8 h at room temperature and quenched with water (20 ml). The mixture was treated with (BOC)₂O (7 g) and NaHCO₃ (2.7 g) at room temperature. The reaction mixture was stirred overnight and monitored by TLC analysis. The solvent was removed under reduce pressure. The aqueous was extracted with EtOAc (400 ml). The organic phase was washed with brine (3×50 ml), dried over MgSO₄, and evaporated to dryness. The residue was purified by chromatograph to give 4.8 g of compound (17c).

### Example 18D

### Preparation of Compound (17d)

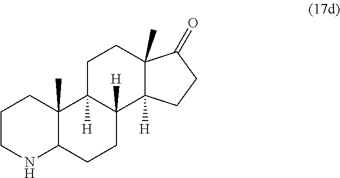

A solution of compound (17c) (2.8 g) in dichloromethane (30 ml) was treated with trifluoroacetic acid (5 ml) at room temperature. The reaction mixture was stirred for 2 h and all the starting material was consumed. The solution basified to PH 9 with saturated aqueous NaHCO₃. The organic layer was separated, and the aqueous layer was extracted with dichloromethane (3×25 ml). The combined organic layers were washed with water (50 ml) and brine (50 ml), dried over magnesium sulfate, and concentrated in vacuo giving crude compound (17d) (1.94 g) which was used for next synthetic step without purification. A small sample was purified to give pure compound (17d). ¹H NMR (CDCl₃): 4.5-4.7 (4H), 3.35 (1H), 2.8-2.9 (2H), 2.62 (1H), 2.5 (1H), 1.05 (3H), 0.95 (3H).

### Example 18E

### Preparation of Compound (17e)

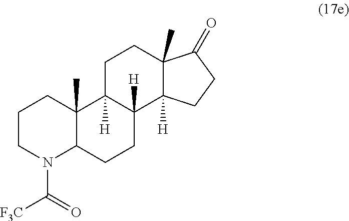

A solution of compound (17d) (1.94 g, 7.54 mmol) and triethylamine (2.1 ml, 15.08 mmol) in dichloromethane (30 ml) was added dropwise with trifluoroacetic anhydride (1.57 ml, 11.31 mmol) at −10° C., The resulting mixture was stirred for 15 min at room temperature. The reaction was quenched with water (20 ml). The organic layer was separated. The aqueous layer was extracted with dichloromethane (3×15 ml). The combined organic layers were washed with water (30 ml) and brine (30 ml), dried over magnesium sulfate, and evaporated to dryness. The residue was purified by column chromatograph (silica gel) to give 2.3 g (82%) of the compound (17e).

### Example 18F

### Preparation of Compound (RN

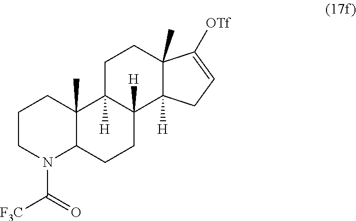

To a solution of compound (17e) (2.3 g) in DCM (20 mL) was added trifluoromethane sulfonic anhydride (1.9 mL, 11.31 mmol) at room temperature, the solution was stirred over 10 min and TEA (1.05 mL, 7.54 mmol) in DCM (10 mL) was added dropwise within 30 min. The mixture was stirred overnight and all the starting material was consumed by TLC analysis. Water (20 mL) was added and the layers were separated. The aqueous layer was extracted with DCM (3×20 mL). The combined organic layers were washed with water and brine, dried over MgSO₄ and concentrated in vacuo. The residue was purified by column chromatography on silica gel (hexanes/EA=25:1-15:1) to give 1.74 g (55.6%) of compound (17f). ¹H NMR (CDCl₃): 5.6 (1H), 4.6-4.8 (2H), 4.55 (1H), 2.7-2.8 (1H), 2.2-2.3 (1H), 1.15 (3H), 1.05 (3H). ¹³C NMR (CDCl₃): 158.96, 114.76, 67.30, 54.11, 44.83, 44.69, 42.34.

### Example 18G

### Preparation of Compound (17g)

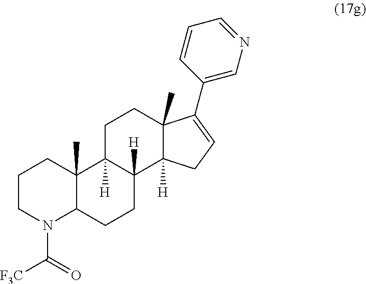

To a solution of compound (17f) (1.74 g) in THF (25 mL) was added diethyl (3-pyridyl)borane (1.26 g, 8.569 mmol), bis(triphenylphosphine)palladium(II) chloride (120 mg, 0.1714 mmol) and 2 N aqueous Na₂CO₃ (16 ml). The mixture was degassed and refilled with Argon gas three times. Then the mixture was heated at 80° C. overnight and monitored by TLC. The mixture was cooled to room temperature and extracted with dichloromethane (2×15 mL). The combined organic layers were washed with water (20 ml) and brine (2×20 mL), dried over Na₂SO₄. The solution was concentrated and purified by column chromatography to give 1.0 g (66.8%) of compound (17g).

### Example 18H

### Preparation of Compound (17)

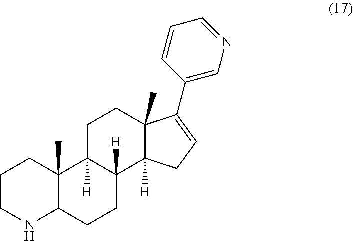

A solution of compound (17g) (1.0 g, 2.29 mmol) in MeOH (30 ml) was treated with 2N aqueous K₂CO₃ (12 ml). The reaction mixture was heated at 60° C. and monitored by TLC analysis. The mixture was cooled to room temperature and concentrated in vacuo. The aqueous layer was extracted with dichloromethane (3×15 ml). The combined organic layers were washed with water (20 ml) and brine (20 ml), dried over Na₂SO₄, and evaporated to dryness. The residue was purified by preparative HPLC to afford 570 mg of a mixture of α- and β-epimers of compound (17). Retention time at HPLC: 7.012 min [Mobile phase: B %=10-100 (gradient 20 min); B=MeCN, A=

H₂O (0.1% TFA); Flow rate: 0.8 mL/min; UV=266 nm column: zorbax Eclipse XDB-C8 5u, (150×4.6 mmID)] ¹H NMR (CDCl₃): 8.7 (1H), 8.45 (1H), 7.65 (1H), 7.19 (1H), 6.0 (1H), 3.12 (1H), 2.6-2.7 (1H), 2.5 (1H), 2.1-2.3 (1H), 0.9 (3H), 0.98 (3H. ¹³C NMR (CDCl₃): 151.73, 147.97, 147.79, 133.68, 133.04, 129.24, 122.95, 62.51, 57.19, 47.82, 47.66, 40.50; MS calculated for (C₂₃H₃₂N₂) [M+H]+ 336.51 Found: 337.2 .

### Example 19

### Preparation of Compound (18)

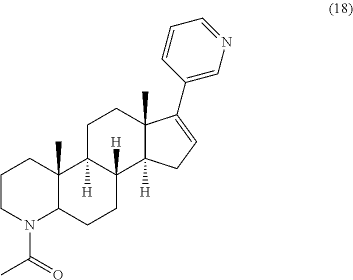

To a solution of compound (17) (110 mg, 0.327 mmol) and triethyl amine (0.136 mL, 0.98 mmol) in dichloromethane (20 mL) at 0° C. under nitrogen, was added acetic anhydride (0.05 mL, 0.49 mmol). The resulting mixture was stirred at room temperature until all the starting material was consumed by TLC analysis (EtOAc:hexane=1:1). The reaction was quenched with water (20 mL). The layers were separated, and the aqueous layer was extracted with dichloromethane (3×15 mL). The combined organic layers were washed with water (2×15 mL) and brine (2×15 mL), dried over Na₂SO₄, and evaporated to dryness. The crude product was purified by preparative HPLC to afford a mixture of α- and β-epimers of compound (18) (150 mg, 91%). MS calculated for (C₂₅H₃₄N₂O) [M+H]+ 379.55 Found: 379.4; [2M+H]+ 758.1 Found: 757.9; ¹H NMR (CDCl₃, 300 MHz): 8.59 (1H), 8.48 (1H), 7.62 (1H), 7.21 (1H), 5.97 (1H), 2.23-2.30 (1H), 2.05 (3H), 1.01 (3H), 0.98 (3H). ¹³C NMR (CDCl₃, 300 MHz): 169.65, 151.57, 147.92, 133.66, 132.94, 129.23, 123.03, 58.70, 49.07, 47.32, 35.01, 33.72, 31.78, 28.72, 27.48, 22.06, 21.86, 20.47, 16.51; Retention time at HPLC: 9.956 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)].

### Example 19A

### Preparation of Compound (18A)

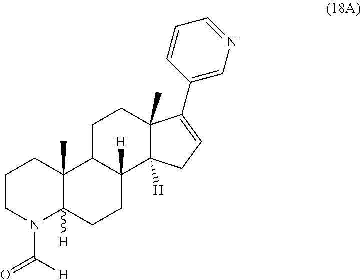

A solution compound (17) (60 mg) in ethyl formate (15 ml) in the presence of triethylamine (0.025 ml) was heated at reflux until all the starting material was consumed. The mixture was concentrated in vacuo. The residue was purified by preparative RP-HPLC to afford 20 mg of compound (18A) as a white powder. Ret time at HPLC: 9.833 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]; ¹H NMR (CDCl₃, 400 MHz): 8.61 (1H), 8.50 (1H), 8.34 (1H), 7.65 (1H), 7.20 (1H), 5.96 (1H), 4.0-4.4 (1H), 2.0-3.4 (2H), 1.0-1.1 (6H); ¹³C NMR (CDCl₃, 400 MHz): 161.55, 151.51, 147.9, 133.76, 129.09, 123.03, 62.68, 57.25; ESI-MS: m/z calcd for C₂₄H₃₂N₂O: [M+H]+ 365.52. Found: 366.2 .

### Example 19B

### Preparation of Compound (18B)

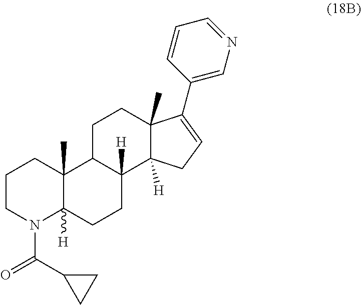

To a solution of compound (17) (70 mg, 0.208 mmol) and triethylamine (0.09 ml, 0.624 mmol) in dichloromethane (10 ml) was treated with cyclopropanecarbonyl chloride (20 μA 0.25 mmol) at 0° C. The mixture was stirred for 10 min and water (10 ml) was added. The layers were separated. The aqueous layer was extracted with dichloromethane (3*15 ml). The combined organic layers were washed with 1N aqueous K₂CO₃ (10 ml), water (15 ml) and brine (15 ml), dried over anhydrous Na₂SO₄, and evaporated to dryness. The residue was purified by preparative R^{P}-HPLC to afford 35 mg of compound (18B) as a white powder. ¹H NMR (CDCl₃, 400 MHz): 8.62 (IH), 8.46 (1H), 7.65 (1H), 7.19 (1H), 5.99 (1H), 4.33 (1H), 4.11 (1H), 2.88 (1H), 2.25 (1H); ¹³C NMR (CDCl₃, 400 MHz): 172.51, 151.56, 147.89, 133.66, 132.95, 129.22, 123.03, 58.67, 49.02, 47.32, 16.51, 14.13, 11.55, 7.18, 7.01. ESI-MS: m/z calcd for C₂₇H₃₆N₂O: [M+H]+ 405.59. Found: 405.5

### Example 19C

### Preparation of Compound (18C)

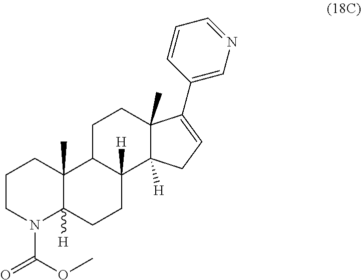

Using a synthetic procedure and conditions similar to Examples 19A in the preparation of compound (18A), and replacing ethyl formate with methyl chloroformate in Example 19A, a mixture of α- and β-epimers of compound (18C) was prepared. Ret. time at HPLC: 12.151 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]; ¹H NMR (CDCl₃, 300 MHz): 8.6 (1H), 8.45 (1H), 7.65 (1H), 7.18 (1H), 6.0 (1H), 4.0-4.1 (1H), 3.75 (1H), 3.65 (3H), 2.7-2.8 (1H), 2.2-2.3 (1H), 0.95-1.05 (6H); ¹³C NMR (CDCl₃, 300 MHz): 156.68, 151.58, 147.92, 147.86, 133.64, 132.97, 129.24, 123.00, 58.71, 56.63, 52.46, 48.86, 47.32, 39.83, 20.63, 16.52; ESI-MS: m/z calcd for C₂₅H₃₄N₂O₂: [M+H]+ 395.55. Found: 395.6.

### Example 19D

### Preparation of Compound (18D)

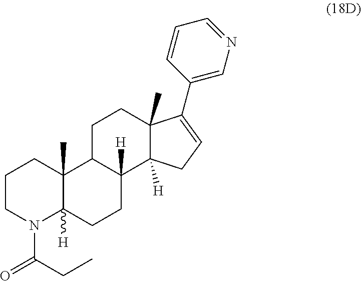

Using a synthetic procedure and conditions similar to Examples 19A in the preparation of compound (18A), and replacing ethyl formate with propionyl chloride in Example 19A, a mixture of α- and β-epimers of compound (18C) was prepared. Ret time at HPLC: 10.897 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]; ¹H NMR (CDCl₃,300 MHz): 8.59 (1H), 8.44 (1H), 7.62 (1H), 7.19 (1H), 5.97 (1H), 3.7-4.0 (2H), 2.6-2.9 (1H)<2.1-2.45 (3H), 1.21 (3H), 1.05 (3H), 0.95 (3H); ¹³C NMR (CDCl₃, 300 MHz): 171.92, 150.55, 146.89, 132.64, 131.93, 128.22, 122.01, 57.69, 48.08, 46.30, 33.99, 32.81, 15.49, 8.75; ESI-MS: m/z calcd for C₂₆H₃₆N₂O: [M+Na]+415.58 Found: 415.5; [2M+Na]+808.16. Found: 808.0.

### Example 20

### Preparation of Compound (19)

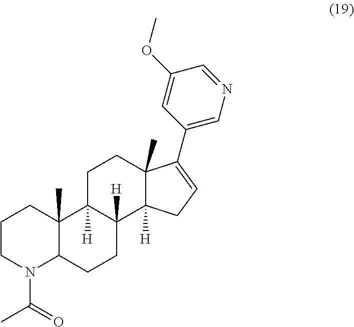

Using a synthetic procedure and conditions similar to Examples 18G in the preparation of compound (17g), and replacing diethyl (3-pyridyl)borane with 5-methoxypyridin-3-ylboronic acid and subjecting the product to reactions and conditions similar to Example 18H and Example 19, a mixture of α- and β-epimers of compound (19) were made. Ret time at HPLC: 12.692 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.20 (1H, s), 8.14 (1H, s), 7.11 (1H, s), 5.97 (1H, s), 3.83-4.0 (5H, m), 2.8 (1H, m), 2.25 (2H, m), 2.10 (3H, s), 1.0 (3H, s), 0.95 (3H, s); ¹³C NMR (CDCl₃, 400 MHz): 169.61, 155.23, 151.35, 140.46, 135.20, 133.49, 129.50, 118.63, 58.67, 55.50, 49.05, 47.33, 35.02, 33.69, 31.75, 28.68, 27.45, 22.06, 21.85, 20.45, 16.55; ESI-MS e/z calcd. For C₂₆H₃₆N₂O₂: [M+H]+ 409.58. Found: 409.8; [M+Na]+431.58. Found: 431.6.

### Example 21

### Preparation of Compound (20)

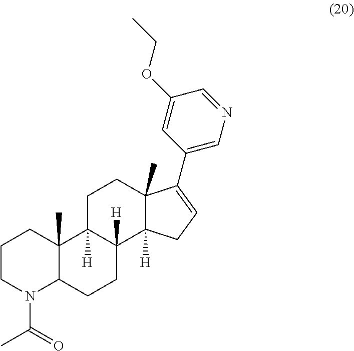

Using a synthetic procedure and conditions similar to Examples 18G in the preparation of compound (17g), and replacing diethyl (3-pyridyl)borane with 5-ethoxypyridin-3-ylboronic acid and subjecting the product to reactions and conditions similar to Example 18H and Example 19, a mixture of α- and β-epimers of compound (20) were made. Ret time at HPLC: 11.711 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.42 (1H, s), 8.20 (1H, s), 7.12 (1H, s), 5.98 (1H, s), 4.05 (2H, m), 3.3-4.0 (2H, m), 2.7 (1H, m), 2.25 (1H, m), 2.05 (3H, s), 1.01 (3H, s), 0.97 (3H, s); ¹³C NMR (CDCl₃, 400 MHz): 169.65, 154.64, 151.42, 140.39, 135.59, 133.52, 129.40, 119.31, 63.86, 58.70, 49.09, 47.36, 35.05, 33.73, 31.78, 28.72, 37.48, 22.10, 21.88, 20.49, 16.57, 14.80; ESI-MS e/z calcd. For C₂₇H₃₈N₂O₂: [2M+H]+ 846.2. Found: 846.3.

### Example 21A

### Preparation of Compound (20A)

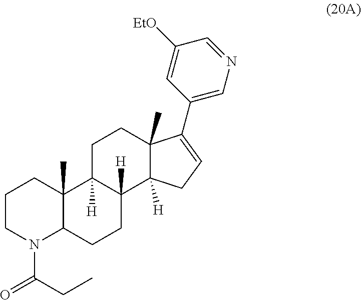

Using a synthetic procedure and conditions similar to Examples 18G in the preparation of compound (17g), and replacing diethyl (3-pyridyl)borane with 5-ethoxypyridin-3-ylboronic acid and subjecting the product to reactions and conditions similar to Example 18H and Example 19, and replacing acetic anhydride in Example 19 with propionic anhydride, a mixture of α- and β-epimers of compound (20A) were made. Ret time=12.673 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.207 (s, 1H), 8.153 (s, 1H), 7.135 (s, 1H), 5.987 (s, 1H), 4.10 (m, 4H), 2.78 (s, 1H), 2.2-2.4 (s, 4H), 2.07 (m, 4H), 1.85 (m, 3H), 1.12 (m, 3H), 1.03 (s, 3H), 0.98 (s, 3H); ¹³C NMR (400 MHz, CDCl₃): 172.98, 154.65, 151.41, 140.36, 135.55, 133.54, 129.42, 119.34, 63.86, 58.71, 49.11, 47.35, 35.05, 33.82, 31.76, 29.70, 28.71, 27.49, 26.95, 21.87, 20.51, 16.56, 14.78, 9.77; ESI-MS: m/z calcd for C₂₈H₄₀N₂O₂: [M+H]+ 437.63. Found: 437.6.

### Example 21B

### Preparation of Compound (20B)

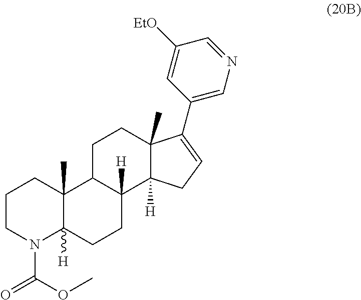

Using a synthetic procedure and conditions similar to Examples 18G in the preparation of compound (17g), and replacing diethyl (3-pyridyl)borane with 5-ethoxypyridin-3-ylboronic acid and subjecting the product to reactions and conditions similar to Example 18H and Example 19, and replacing acetic anhydride in Example 19 with methyl chloroformate, a mixture of α- and β-epimers of compound (20B) were made. Ret time at HPLC: 14.127 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.209 (s, 1H), 8.144 (s, 1H), 7.158 (s, 1H), 5.992 (s, 1H), 3.97-4.25 (m, 3H), 3.65-3.8 (m, 4H), 2.78 (m, 1H), 2.1-2.3 (m, 3H), 1.75-2.1 (m, 7H), 1.1-1.7 (m, 12H), 1.01 (d, 6H); ¹³C NMR (400 MHz, CDCl₃): 156.686, 154.677, 151.364, 140.170, 135.321, 133.632, 129.538, 119.465, 63.875, 58.700, 56.633, 52.462, 51.803, 48.860, 47.350, 39.821, 35.072, 34.684, 33.438, 31.767, 28.874, 27.409, 21.817, 20.619, 16.557, 14.757; ESI-MS: m/z calcd for C₂₇H₃₈N₂O₃: [M+H]+ 439.6. Found: 439.1.

### Example 21C

### Preparation of Compound (20C)

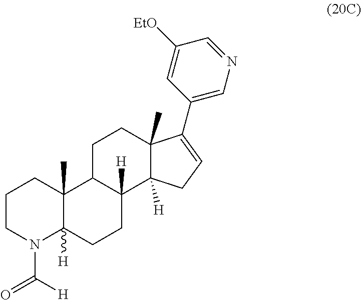

Using a synthetic procedure and conditions similar to Examples 18G in the preparation of compound (17g), and replacing diethyl (3-pyridyl)borane with 5-ethoxypyridin-3-ylboronic acid and subjecting the product to reactions and conditions similar to Example 18H and Example 19, and replacing acetic anhydride in Example 19 with ethyl formate, a mixture of α- and β-epimers of compound (20C) were made. ¹H NMR (400 MHz, CDCl₃): 8.32 (s, 0.7H), 8.19 (s, 1H), 8.14 (s, 1H), 8.05 (s, 0.3H), 5.95 (s, 1H), 3.9-4.61 (m, 3H), 2.9-3.5 (m, 1H), 2.3-2.7 (m, 1H), 1.95-2.3 (m, 4H), 1.3-1.9 (m, 10H), 1.01 (d, 6H); ¹³C NMR (400 MHz, CDCl₃): 161.485, 159.271, 154.615, 151.382, 140.135, 135.568, 133.353, 129.246, 119.308, 66.066, 63.812, 62.612, 57.210, 52.921, 47.485, 41.190, 40.650, 38.111, 36.40, 35.12, 32.870, 31.482, 26.20, 21.978, 20.771, 19.74, 16.56, 14.73, 11.99; ESI-MS: m/z calcd for C₂₆H₃₆N₂O₂: [M+H]+ 409.6. Found: 409.1.

### Example 22

### Preparation of Compound (21)

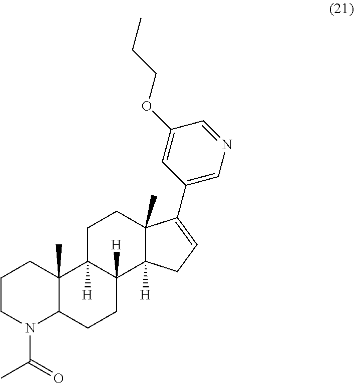

Using a synthetic procedure and conditions similar to Examples 18G in the preparation of compound (17g), and replacing diethyl (3-pyridyl)borane with 5-propoxypyridin-3-ylboronic acid and subjecting the product to reactions and conditions similar to Example 18H and Example 19, a mixture of α- and β-epimers of compound (21) were made. Ret time at HPLC: 12.692 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.16 (1H, s), 2.10 (1H, s), 7.08 (1H, s), 5.94 (1H, s), 3.89-3.93 (4H, m), 2.55-2.8 (1H, m), 2.21 (1H, m), 2.12 (3H, s), 0.8-1.2 (12H, m); ¹³C NMR (CDCl₃, 400 MHz): 168.60, 153.82, 150.42, 139.24, 134.67, 132.47, 128.34, 118.20, 68.78, 57.67, 48.06, 46.32, 34.02, 33.64, 32.69, 30.73, 30.56, 27.69, 26.45, 24.26, 21.63, 21.53, 21.03, 20.86, 19.44, 15.54, 13.11, 9.45; ESI-MS e/z calcd. For C₂₈H₄₀N₂O₂: [M+H]+ 436.63. Found: 437.5.

### Example 23

### Preparation of Compound (22)

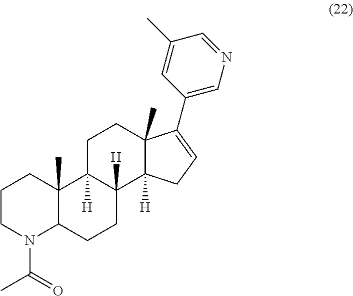

Using a synthetic procedure and conditions similar to Examples 18G in the preparation of compound (17g), and replacing diethyl (3-pyridyl)borane with 5-methylpyridin-3-ylboronic acid and subjecting the product to reactions and conditions similar to Example 18H and Example 19, a mixture of α- and β-epimers of compound (22) are made.

### Example 23A

### Preparation of Compound (22A)

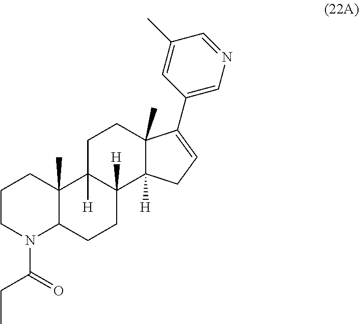

Using a synthetic procedure and conditions similar to Examples 18G in the preparation of compound (17g), and replacing diethyl (3-pyridyl)borane with 5-methylypyridin-3-ylboronic acid and subjecting the product to reactions and conditions similar to Example 18H and Example 19, replacing acetic anhydride in Example 19 with propionic anhydride, a mixture of α- and β-epimers of compound (22A) were made. Ret time: 11.309 min [Mobile phase: B %=10-100 (gradient 20 min); B=MeCN, A=H2O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.412 (s, 1H), 8.301 (s, 1H), 7.502 (s, 1H), 5.996 (s, 1H), 3.984 (m, 1H), 2.78 (m, 1H), 2.45 (m, 1H), 2.31 (s, 3H), 1.95-2.3 (m, 6H), 1.24-1.83 (m, 13H), 1.19 (m, 3H), 1.01 (s, 3H), 0.98 (s, 3H); ¹³C NMR (400 MHz, CDCl₃): 171.94, 150.57, 147.21, 143.89, 133.42, 131.41, 128.02, 57.69, 48.07, 46.28, 34.02, 32.79, 30.72, 27.68, 26.47, 25.93, 20.83, 19.47, 17.42, 15.51, 8.75; ESI-MS: m/z calcd for C₂₇H₃₈N₂O: [M+H]+ 407.6. Found: 407.6; [2M+H]+ 814.2. Found: 814.1.

### Example 23B

### Preparation of Compound (22B)

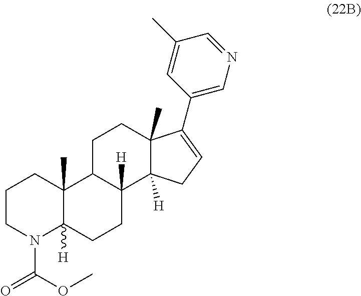

Using a synthetic procedure and conditions similar to Examples 18G in the preparation of compound (17g), and replacing diethyl (3-pyridyl)borane with 5-methylypyridin-3-ylboronic acid and subjecting the product to reactions and conditions similar to Example 18H and Example 19, replacing acetic anhydride in Example 19 with methyl chloroformate, a mixture of α- and β-epimers of compound (22B) were made. Ret time: 12.574 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.42 (s, 1H), 8.30 (s, 1H), 7.55 (s, 1H), 6.01 (s, 1H), 4.0-4.25 (m, 1H), 3.75 (m, 1H), 3.70 (s, 3H), 3.6-3.7 (m, 1H), 2.81 (m, 1H), 2.35 (s, 3H), 1.8-2.3 (m, 11H), 1.2-1.8 (m, 13H), 1.10 (m, 1H), 1.01 (d, 6H); ¹³C NMR (400 MHz, CDCl₃): 151.60, 148.24, 144.94, 134.36, 132.46, 129.01, 58.69, 56.60, 52.42, 48.83, 47.39, 39.79, 35.05, 34.65, 33.73, 33.73, 33.42, 31.72, 28.85, 27.39, 21.79, 21.02, 20.60, 18.41, 16.50, 12.63; ESI-MS: m/z calcd for C₂₆H₃₆N₂O₂: [M+H]+ 409.58. Found: 409.9.

### Example 24

### Preparation of Compound (23)

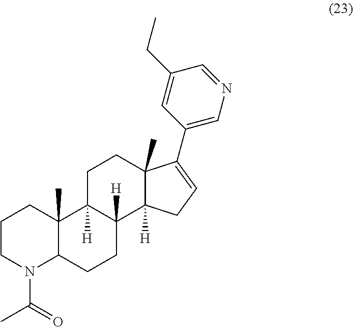

Using a synthetic procedure and conditions similar to Examples 18G in the preparation of compound (17g), and replacing diethyl (3-pyridyl)borane with 5-ethylpyridin-3-ylboronic acid and subjecting the product to reactions and conditions similar to Example 18H and Example 19, a mixture of α- and β-epimers of compound (23) were made. Ret time at HPLC: 11.229 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (CDCl₃, 400 MHz): 8.41 (1H, s), 8.30 (1H, s), 7.48 (1H, s), 5.97 (1H, s), 3.5-4.0 (2H, m), 2.5-2.9 (3H), 2.25 (1H, m), 2.10 (3H, s), 1.01 (3H, s), 0.98 (3H, s); ¹³C NMR (CDCl₃, 400 MHz): 169.63, 151.69, 147.65, 145.26, 138.49, 133.13, 132.53, 128.92, 58.68, 49.06, 47.29, 35.02, 33.69, 31.73, 28.70, 27.46, 26.03, 22.04, 21.86, 20.45, 16.53, 15.33; ESI-MS e/z calcd. For C₂₇H₃₈N₂O: [M+H]+ 407.6. Found: 407.5; [2M+H]+ 814. Found: 813.9.

### Example 24A

### Preparation of Compound (23A)

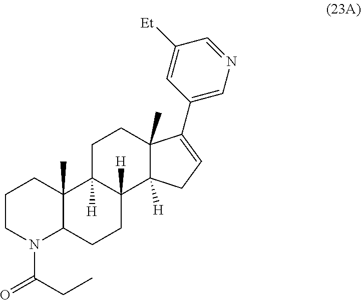

Using a synthetic procedure and conditions similar to Examples 18G in the preparation of compound (17g), and replacing diethyl (3-pyridyl)borane with 5-ethylpyridin-3-ylboronic acid and subjecting the product to reactions and conditions similar to Example 18H and Example 19, replacing acetic anhydride in Example 19 with propionic anhydride, a mixture of α- and β-epimers of compound (23A) were made. Ret time=11.821 min [Mobile phase: B %=10-100 (gradient 20 min); B=MeCN, A=H₂O (0.1% TFA); Flow rate: 0.8 mL/min; UV=266 nm; column: zorbax Eclipse XDB-C8 5u, (150*4.6 mm ID)]; ¹H NMR (400 MHz, CDCl₃): 8.414 (s, 1H), 8.281 (s, 1H), 7.614 (s, 1H), 5.941 (s, 1H), 4.08 (m, 1H), 2.75 (m, 2H), 2.61 (m, 2H), 2.15-2.4 (m, 4H), 1.95-2.10 (m, 4H), 1.85 (m, 3H), 1.25-1.80 (m, 8H), 1.22 (m, 3H), 1.12 (m, 3H), 1.01 (s, 3H), 0.97 (s, 3H); ¹³C NMR (400 MHz, CDCl₃): 172.92, 151.71, 147.66, 145.27, 138.48, 133.11, 132.53, 128.91, 58.70, 49.09, 47.03, 35.04, 34.97, 33.80, 31.73, 29.67, 28.70, 27.49, 26.93, 26.03, 21.87, 20.49, 16.52, 15.32, 9.75; ESI-MS: m/z calcd for C₂₈H₄₀N₂O: [2M+H]+ 842.26. Found: 842.3

### Example 24B

### Preparation of Compound (23B)

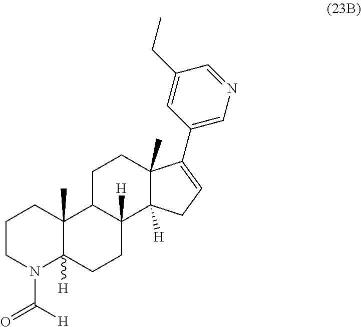

Using a synthetic procedure and conditions similar to Examples 18G in the preparation of compound (17g), and replacing diethyl (3-pyridyl)borane with 5-ethylpyridin-3-ylboronic acid and subjecting the product to reactions and conditions similar to Example 18H and Example 19, replacing acetic anhydride in Example 19 with methyl formate, a mixture of α- and β-epimers of compound (23B) were made. ¹H NMR (400 MHz, CDCl₃): 8.45 (s, 1H), 8.34 (s, 1.7H), 8.03 (s, 0.3H), 7.45 (s, 1H), 5.95 (s, 1H), 3.95-4.6 (m, 1H), 2.9-3.2 (m, 1H), 2.67 (m, 3H), 1.92-2.3 (m, 5H), 1.7-1.92 (m, 2H), 1.3-1.7 (m, 9H), 1.16-1.32 (m, 6H), 1.02 (d, 6H); ¹³C NMR (400 MHz, CDCl₃): 160.541, 150.711, 146.515, 144.028, 137.613, 132.438, 131.482, 127.890, 61.674, 56.235, 46.501, 40.194, 39.706, 35.451, 25.032, 21.029, 19.806, 18.767, 15.576, 14.334; ESI-MS: m/z calcd for C₂₆H₃₆N₂O: [M+H]+ 393.58. Found: 393.0.

### Example 24C

### Preparation of Compound (23C)

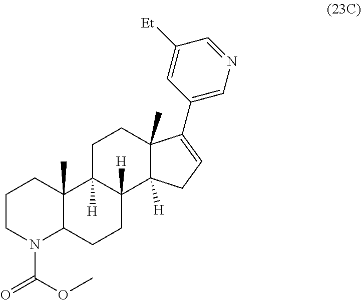

Using a synthetic procedure and conditions similar to Examples 18G in the preparation of compound (17g), and replacing diethyl (3-pyridyl)borane with 5-ethylpyridin-3-ylboronic acid and subjecting the product to reactions and conditions similar to Example 18H and Example 19, replacing acetic anhydride in Example 19 with methyl chloroformate, a mixture of α- and β-epimers of compound (23C) were made. ¹H NMR (400 MHz, CDCl₃): 8.45 (s, 1H), 8.32 (s, 1H), 7.47 (s, 1H), 5.959 (s, 1H), 4.25 (d, 0.2H), 4.02 (d, 0.8H), 3.80 (m, 1H), 3.73 (s, 2.4H), 3.62 (s, 0.6), 2.81 (m, 1H), 2.65 (m, 2H), 2.50 (m, 1H), 2.30 (m, 1H), 1.95-2.10 (m, 2H), 1.80-1.95 (m, 2H), 2.32-1.80 (m, 10H), 1.25 (m, 4H), 1.02 (s, 3H), 0.95 (s, 3H); ¹H NMR (400 MHz, CDCl₃): 156.683, 156.297, 151.766, 151.673, 147.472, 145.117, 138.565, 133.316, 132.643, 129.047, 58.711, 56.660, 52.443, 48.866, 47.432, 39.811, 38.797, 34.686, 33.437, 31.767, 28.883, 27.398, 26.045, 20.633, 16.553, 15.294; ESI-MS: m/z calcd for C₂₇H₃₈N₂O₂: [M+H]+ 423.6. Found: 423.5.

### Example 25

### Preparation of Compound (24)

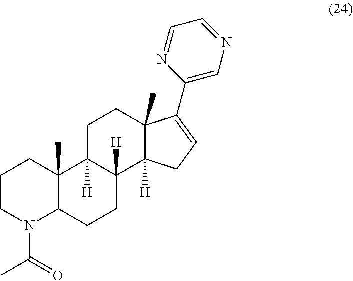

Using a synthetic procedure and conditions similar to Examples 18G in the preparation of compound (17g), and replacing diethyl (3-pyridyl)borane with 2-(tributylstannyl)pyrazine and subjecting the product to reactions and conditions similar to Example 18H and Example 19, a mixture of α- and β-epimers of compound (24) are made.

### Example 26

### Preparation of Compound (25)

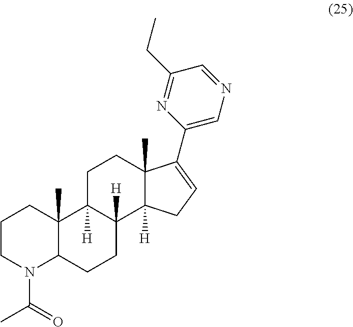

Using a synthetic procedure and conditions similar to Examples 18G in the preparation of compound (17g), and replacing diethyl (3-pyridyl)borane with 2-ethyl-6-(tributylstannyl)pyrazine and subjecting the product to reactions and conditions similar to Example 18H and Example 19, a mixture of α- and β-epimers of compound (25) are made.

### Example 27

### Preparation of Compound (26)

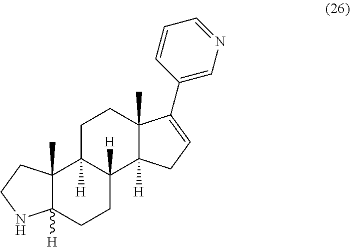

### Example 27A

### Preparation of Compound (26a)

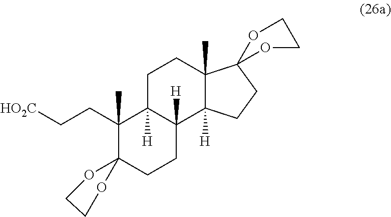

A solution of compound (9b) (7.8 g, 19.09 mmol) in MeOH (50 ml) was treated with 5N aqueous NaOH. The mixture was stirred overnight at room temperature and the starting material was completely consumed by TLC analysis. The volatile solvent was removed under reduce pressure. The aqueous solution was acidified with 1N aqueous HCl to PH 3 and extracted with EtOAc (3×60 ml). The combined organic layers were washed with water (100 ml) and brine (100 ml), dried over sodium sulfate, and evaporated to give 7.2 g (95%) of compound (26a).

### Example 27B

### Preparation of Compound (26b)

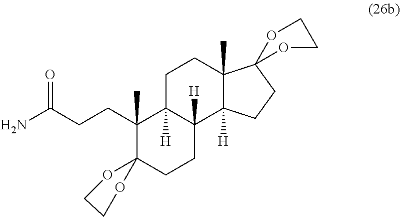

A solution of compound (26a) (1.0 g, 2.535 mmol) in acetonitrile (30 ml) was treated with (Boc)₂O (0.718 ml, 3.3 mmol) and pyridine (0.12 ml) at room temperature under nitrogen, followed by addition of NH₄HCO₃ (0.32 g, 4.0 mmol) in one portion. The resulting mixture was stirred at room temperature until all the starting material was consumed. The volatile solvent was removed under reduce pressure. The residue was diluted with EtOAc (45 ml) and washed with water (40 ml) and brine (40 ml), dried over Na₂SO₄, and concentrated in vacuo to give 0.92 g (92%) of compound (26b) which was used in the next step without further purification. Purification of a small sample yielded pure compound (26b), ¹H NMR (DMSO): 7.15 (1H), 6.55 (1H), 3.7-3.9 (8H), 0.98 (3H), 0.85 (3H). ¹³C NMR (DMSO): 175.46, 149.55, 123.84, 118.36, 112.80.

### Example 27C

### Preparation of Compound (26c)

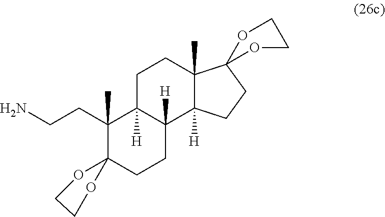

A solution of compound (26b) (3.5 g, 8.89 mmol) in MeCN—H₂O (30 ml, 4/1) was treated with (Diacetoxyiodo)benzene (3.44 g, 10.67 mmol) at room temperature. The mixture was stirred until all the starting material was consumed. The volatile solvent was removed under reduce pressure. The residue was diluted with water (10 ml) and washed with methyl tert-butyl ether (3×25 ml). The aqueous solution was basified with 2N aqueous Na₂CO₃ to PH 9 and extracted with EtOAc (3×25 ml). The combined organic layers were washed with water (40 ml) and brine (40 ml), dried over sodium sulfate, and evaporated to give 1.63 g (50%) of compound (26c). The crude product was used in the next step without further purification. Purification of a small sample yielded pure compound (26c), ¹H NMR (DMSO): 3.78-3.91 (8H), 2.7-2.8 (1H), 2.5-2.6 (1H), 1.8-1.9 (1H), 0.95 (3H), 0.85 (3H).

### Example 27D

### Preparation of Compound (26d)

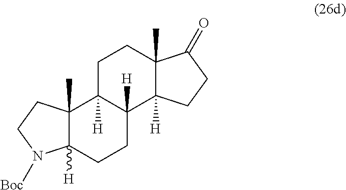

A solution of compound (26c) (1.1 g, 3 mmol) in THF (10 ml) was treated with 5N aqueous HCl (10 ml) at room temperature. The reaction mixture was stirred until all the starting material was consumed. The volatile solvent was removed under reduce pressure. The aqueous solution was basified with 2N aqueous NaOH to PH 10 and extracted with EtOAc (3×15 ml). The combined organic layers were washed with water (40 ml) and brine (40 ml), dried over Na₂SO₄, and evaporated to give 0.72 g (92.6%) of imine derivative. Following the experimental conditions as in Example 18C in the preparation of compound (17c), the imine was first reduced by the use of sodium cyanoborohydride in the presence of acetic acid and subsequently treated with (BOC)₂O and NaHCO₃ to give compound (26d). ¹H NMR (CDCl₃): 3.5 (1H), 3.2-3.4 (2H), 1.43 (9H), 1.02 (3H), 0.85 (3H).

### Example 27E

### Preparation of Compound (26e)

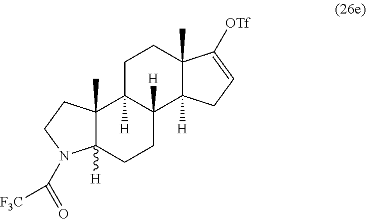

Using a synthetic procedure and conditions similar to Example 18D-Example 18F in the preparation of compound (17f), replacing compound (17c) with compound (26d) in Example 18D, compound (26e) was made.

### Example 27F

### Preparation of Compound (26f)

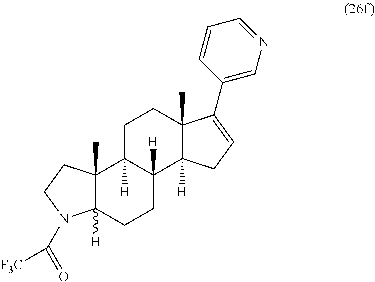

Using a synthetic procedure and condition similar to Examples 18G in the preparation of compound (17g), and replacing compound (17f) with compound (26e) to react with diethyl (3-pyridyl)borane a mixture of α- and β-epimers epimer of compound (26f) was made.

### Example 27G

### Preparation of Compound (26)

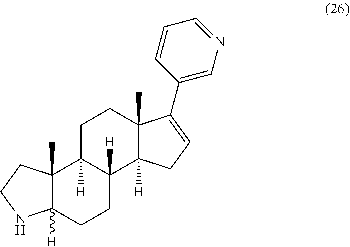

Using a synthetic procedure and condition similar to Examples 18H in the preparation of compound (17), and replacing compound (17g) with compound (26f) a mixture of α- and β-epimers of compound (26) were made. Retention time at HPLC: 6.833 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃): 8.65 (1H), 8.45 (1H), 7.15 (1H), 5.95 (1H), 3.2 (1H), 3.1 (1H), 2.95 (1H), 2.25 (1H), 1.1 (3H), 1.0 (3H). ¹H NMR (CDCl₃): 151.63, 147.85, 133.72, 132.90, 129.21, 123.02, 65.40, 56.84, 47.53; MS calculated for (C₂₂H₃₀N₂) [M+H]+ 322.49 Found: 323.1

### Example 28

### Preparation of Compound (27)

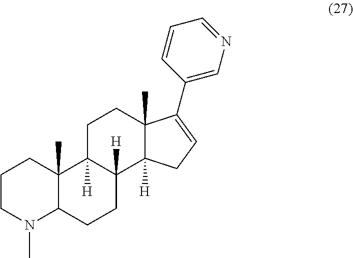

To a solution of compound (17) (50 mg, 0.1486 mmol) and formaldehyde (30 mg of 37 aqueous solution) in methanol (8 ml) was added sodium cyanoborohydride (93 mg, 1.486 mmol) at room temperature, followed by addition of acetic acid (0.03 ml). The reaction mixture was stirred overnight and monitored by analytical HPLC. The solution was concentrated in vacuo. The residue was basified with 2N aqueous K₂CO₃ (20 ml) to PH 9 and extracted with dichloromethane (3×15 ml). The combined organic layers were washed water (30 ml) and brine (30 ml), dried over sodium sulfate, evaporated to dryness. The crude product was purified by preparative HPLC to give a mixture of α- and β-epimers of compound (27). Retention time at HPLC: 7.120 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. MS calculated for (C₂₄H₃₄N₂) [M+H]+ 351.54 Found: 351.2; ¹H NMR (CDCl₃): 8.61 (1H), 8.4 (1H), 7.65 (1H), 7.15 (1H), 5.96 (1H), 2.85 (1H), 2.1-2.2 (5H), 0.953H), 0.9 (3H). ¹³C NMR (CDCl₃): 151.68, 147.93, 147.71, 133.67, 133.08, 129.26, 122.93, 71.17, 58.50, 56.90, 47.69, 43.14, 41.67.

### Example 29

### Preparation of Compound (28)

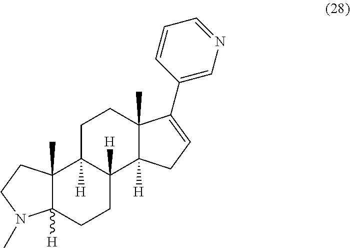

Using a synthetic procedure and condition similar to Examples 28 in the preparation of compound (27), and replacing compound (17) with compound (26) a mixture of α- and β-epimers of compound (28) were made. Retention time at HPLC: 6.794 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. MS calculated for (C₂₃H₃₂N₂) [M+H]+ 337.51 Found: 337.3 ¹H NMR (CDCl₃): 8.65 (1H), 8.4 (1H), 7.65 (1H), 7.15 (1H), 5.95 (1H), 3.15 (1H), 2.25-2.3 (4H), 2.15-2.2 (1H), 1.01 (6H). ¹³C NMR (CDCl₃): 151.80, 147.94, 147.76, 133.68, 133.03, 129.26, 122.96, 73.65, 57.02, 54.70, 47.75, 47.59, 43.03, 40.62

### Example 30

### Preparation of Compound (29)

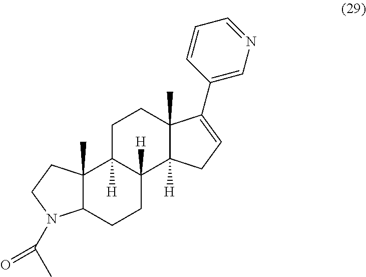

Using a synthetic procedure and conditions similar to Examples 19 in the preparation of compound (18), and replacing compound (17) with compound (26) and reacting it with acetic anhydride, a mixture of α- and β-epimers of compound (29) were made. MS calculated for (C₂₄H₃₂N₂O) [M+H]+365.52 Found: 365.0; ¹H NMR (CDCl₃, 300 MHz): 8.61 (1H), 8.45 (1H), 7.61-7.63 (1H), 7.19-7.24 (1H), 5.98 (1H), 3.3-3.5 (3H), 2.75 (1H), 2.26-2.69 (1H), 2.24 (1H), 2.12 (3H), 1.02 (3H), 0.99 (3H). ¹³C NMR (CDCl₃, 300 MHz): 170.86, 151.66, 147.88, 133.67, 132.93, 129.37, 123.04, 65.62, 57.59, 47.55, 46.82, 44.72, 42.99, 19.59, 16.71; Retention time at HPLC: 9.489 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)].

### Example 30A

### Preparation of Compound (29A)

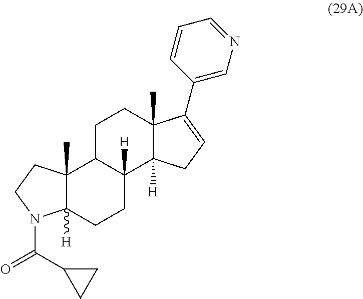

A solution of compound (26) (100 mg, 0.31 mmol) and triethylamine (0.15 ml, 1.0 mmol) in dichloromethane (10 ml) was treated with cyclopropanecarbonyl chloride (40 μl, 0.372 mmol) at 0° C. The mixture was stirred for 10 min and water (10 ml) was added. The layers were separated. The aqueous layer was extracted with dichloromethane (3*15 ml). The combined organic layers were washed with 1N aqueous K₂CO₃ (15 ml), water (15 ml) and brine (15 ml), dried over anhydrous Na₂SO₄, and evaporated to dryness. The residue will be purified by preparative RP-HPLC to afford 46 mg of compound (29A) as a white powder. Ret. time at HPLC: 10.566 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]; ¹H NMR: 8.60 (1H), 8.44 (1H), 7.64 (1H), 7.21 (1H), 6.0 (1H), 2.7-3.7 (3H), 2.28 (1H), 1.0-1.2 (11H), 0.7-0.9 ((4H); ¹³C NMR: 173.98, 151.68, 147.88, 133.72, 132.99, 129.42, 123.07, 65.81, 57.56; ESI-MS e/z calcd. For C₂₆H₃₄N₂O: [M+H]+ 391.56. Found: 391.8.

### Example 30B

### Preparation of Compound (29B)

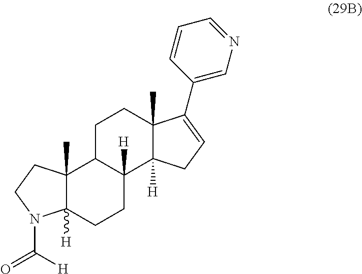

Compound (26) (50 mg) was treated with ethyl formate (8 ml) in the presence of triethylamine (0.5 ml). The reaction was heated at reflux until all the starting material was consumed. The mixture was concentrated in vacuo. The residue was purified by preparative RP-HPLC to afford 15 mg mixture of α- and β-epimers of compound (29B) as a white powder. Ret. time at HPLC: 9.045 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ESI-MS: m/z calcd for C₂₃H₃₀N₂O: [M+H]+ 351.5. Found: 351.2; ¹H NMR (CDCl₃, 300 MHz): 8.61 (1H), 8.45 (1H), 8.2 (1H), 8.65 (1H), 7.18 (1H), 5.95 (1H), 3.3-3.45 (3H), 2.15-2.25 (2H), 1.81-2.1 (5H), 0.95-1.12 (6H); ¹³C NMR (CDCl₃, 300 MHz): 160.53, 151.61, 147.95, 147.85, 133.74, 132.76, 129.09, 123.04, 64.13, 56.94, 47.56, 18.73, 16.69.

### Example 30C

### Preparation of Compound (29C)

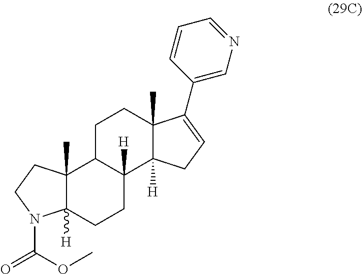

A solution of compound (26) (100 mg, 0.31 mmol) and triethylamine (0.15 ml, 1.0 mmol) in dichloromethane (10 ml) was treated with methyl chloroformate (30 μl, 0.39 mmol) at 0° C. The mixture was stirred for 10 min and water (10 ml) was added. The layers were separated. The aqueous layer was extracted with dichloromethane (3*15 ml). The combined organic layers were washed with 1N aqueous K2CO₃ (15 ml), water (15 ml) and brine (15 ml), dried over anhydrous Na2SO4, and evaporated to dryness. The resulting residue was solidified with hexane to yield 61 mg of a mixture of α- and β-epimers of compound (29C). Ret. time at HPLC: 11.351 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 300 MHz): 8.6 (1H), 8.45 (1H), 8.64 (1H), 7.18 (1H), 5.95 (1H), 3.7 (3H), 3.2-3.6 (3H), 2.2-2.3 (1H), 2.0-2.1 (2H), 1.85 (1H), 0.95-1.05 (6H). ¹³C NMR (CDCl₃, 300 MHz): 151.71, 147.89, 133.68, 132.93, 129.30, 123.02, 65.46, 57.44, 51.95, 47.57, 44.20, 19.47, 16.87. ESI-MS: m/z calcd for C₂₄H₃₂N₂O₂: [M+H]+ 381.52. Found: 381.7. [M+Na]+403.52. Found: 403.5; [2M+H]+ 762.04. Found: 761.7.

### Example 30D

### Preparation of Compound (29D)

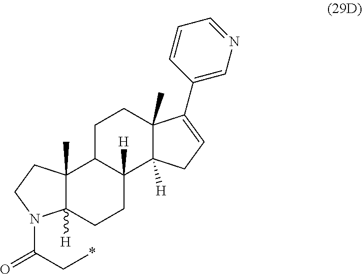

A solution of compound (26) (100 mg, 0.31 mmol) and triethylamine (0.15 ml, 1.0 mmol) in dichloromethane (10 ml) was treated with n-propyl chloride (40 μl, 0.39 mmol) at 0° C. The mixture was stirred for 10 min and water (10 ml) was added. The layers were separated. The aqueous layer was extracted with dichloromethane (3*15 ml). The combined organic layers were washed with 1N aqueous K₂CO₃ (15 ml), water (15 ml) and brine (15 ml), dried over anhydrous Na₂SO₄, and evaporated to dryness. The residue was purified by preparative RP-HPLC to afford 16 mg of a mixture of α- and β-epimers of compound (29D). Ret. time at HPLC: 10.166 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ESI-MS: m/z calcd for C₂₅H₃₄N₂O: [M+H]+ 379.55. Found: 379.5. ¹H NMR (CDCl₃,300 MHz): 8.573 (1H), 8.569 (1H), 8.428 (1H), 7.612 (1H), 7.18 (1H), 5.94 (1H), 3.2-3.6 (3H), 1.8-2.2 (6H), 0.91-1.2 (9H). ¹³C NMR (CDCl₃, 300 MHz): 174.04, 151.64, 147.82, 133.65, 132.92, 129.34, 123.02, 65.71, 57.59, 19.55, 16.70.

### Example 31

### Preparation of Compound (30)

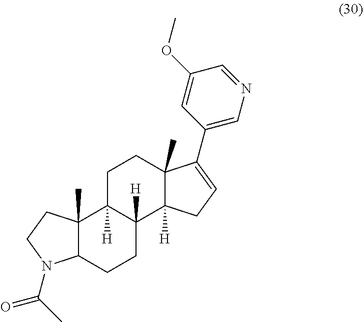

Using a synthetic procedure and conditions similar to Examples 18G in the preparation of compound (17g), replacing compound (17f) with compound (26e) and replacing diethyl (3-pyridyl)borane with 5-methoxypyridin-3-ylboronic acid and subjecting the product to reactions and conditions similar to Example 27G and Example 30, a mixture of α- and β-epimers of compound (30) were made. Ret time at HPLC: 10.395 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.22 (1H, s), 8.15 (1H, s), 7.15 (1H, s), 5.98 (1H, s), 3.85 (3H, s), 2.75-3.8 (4H, m), 2.25 (1H, m) 2.01-2.1 (5H, m), 0.95-1.1 (6H); ¹³C NMR (CDCl₃, 400 MHz): 170.89, 155.27, 151.48, 140.45, 135.16, 133.53, 129.68, 118.73, 65.63, 57.60, 55.54, 47.60, 46.84, 44.73, 43.00, 35.58, 35.51, 33.77, 31.69, 25.99, 23.84, 22.98, 22.57, 19.60, 16.77; ESI-MS e/z calcd. For C₂₅H₃₄N₂O₂: [M+H]+ 395.55. Found: 396.0; [2M+H]+ 790.1. Found: 789.9

### Example 32

### Preparation of Compound (31)

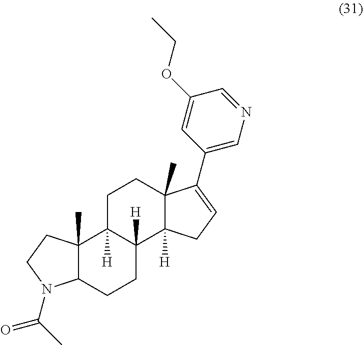

Using a synthetic procedure and conditions similar to Examples 18G in the preparation of compound (17g), replacing compound (17f) with compound (26e) and replacing diethyl (3-pyridyl)borane with 5-ethoxypyridin-3-ylboronic acid and subjecting the product to reactions and conditions similar to Example 27G and Example 30, a mixture of α- and β-epimers of compound (31) were made. Ret time at HPLC: 11.198 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.20 (1H, s), 8.14 (1H, s), 7.12 (1H, s), 4.02 (2H, m), 2.7-3.7 (4H, m), 2.25 (1H, m), 2.0-2.1 (7H, m), 0.9-1.1 (9H, m). ¹³C NMR (CDCl₃, 400 MHz): 170.88, 154.64, 151.50, 140.32, 135.53, 133.51, 129.54, 119.32, 65.62, 63.84, 57.58, 47.57, 46.83, 44.72, 42.98, 35.56, 35.50, 33.76, 31.67, 25.97, 23.83, 22.97, 22.56, 19.58, 16.75, 14.78; ESI-MS e/z calcd. For C₂₆H₃₆N₂O₂: [M+H]+ 409.58. Found: 409.9; [2M+H]+ 818.16. Found: 818.0.

### Example 32A

### Preparation of Compound (31A)

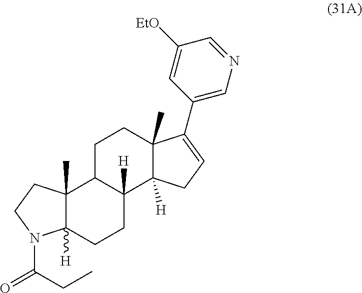

Using a synthetic procedure and conditions similar to Examples 18G in the preparation of compound (17g), replacing compound (17f) with compound (26e) and replacing diethyl (3-pyridyl)borane with 5-ethoxypyridin-3-ylboronic acid and subjecting the product to reactions and conditions similar to Example 27G and Example 30 and replacing acetic anhydride in Example 30 with propionic anhydride, a mixture of α- and β-epimers of compound (31A) were made. Ret. time at HPLC: 11.843 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.219 (s, 1H), 8.151 (s, 1H), 7.22 (s, 1H), 6.02 (s, 1H), 4.12 (m, 2H), 3.50 (m, 3H), 2.61 (m, 2H), 2.25 (m, 3H), 1.81-2.10 (4H), 1.41-1.70 (m, 8H), 1.03-1.19 (m, 12H). ¹³C NMR (400 MHz, CDCl₃): 174.10, 154.69, 151.44, 140.11, 135.30, 133.61, 129.67, 119.48, 65.73, 63.88, 57.59, 47.58, 45.90, 44.70, 42.80, 35.56, 33.80, 31.68, 28.84, 25.98, 23.03, 22.55, 19.57, 16.76, 14.78, 9.20. ESI-MS: m/z calcd for C₂₇H₃₈N₂O₂: [M+H]+ 423.6. Found: 423.7.

### Example 32B

### Preparation of Compound (31B)

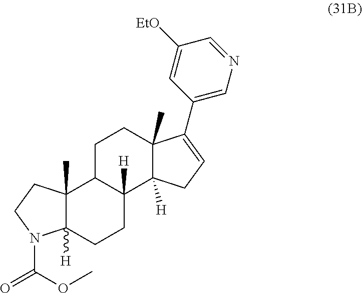

Using a synthetic procedure and conditions similar to Examples 18G in the preparation of compound (17g), replacing compound (17f) with compound (26e) and replacing diethyl (3-pyridyl)borane with 5-ethoxypyridin-3-ylboronic acid and subjecting the product to reactions and conditions similar to Example 27G and Example 30 and replacing acetic anhydride in Example 30 with methyl chloroformate, a mixture of α- and β-epimers of compound (31B) were made. Ret. time at HPLC: 13.044 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.208 (s, 1H), 8.14 (s, 1H), 7.162 (s, 1H), 5.98 (s, 1H), 4.106 (m, 2H), 3.67 (s, 3H), 3.2-3.7 (m, 3H), 2.15-2.41 (m, 3H), 1.98-2.10 (m, 2H), 1.84 (m, 1H), 1.36-1.60 (m, 11H), 1.05 (d , 6H). ¹³C NMR (400 MHz, CDCl₃): 153.62, 150.48, 139.20, 134.48, 134.48, 132.49, 128.48, 118.28, 64.38, 62.81, 56.39, 50.92, 46.56, 43.15, 42.23, 34.56, 33.93, 32.96, 30.63, 24.67, 21.44, 18.41, 15.73, 13.75. ESI-MS: m/z calcd for C₂₆H₃₆N₂O₃: [M+H]+ 425.58. Found: 425.5.

### Example 33

### Preparation of Compound (32)

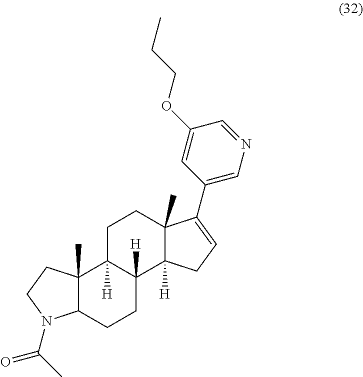

Using a synthetic procedure and conditions similar to Examples 18G in the preparation of compound (17g), replacing compound (17f) with compound (26e) and replacing diethyl (3-pyridyl)borane with 5-propoxypyridin-3-ylboronic acid and subjecting the product to reactions and conditions similar to Example 27G and Example 30, a mixture of α- and β-epimers of compound (32) were made. Ret. time at HPLC: 11.874 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.20 (1H, s), 8.14 (1H, s), 7.13 (1H, s), 5.97 (1H, s), 3.98 (2H, m), 2.7-3.7 (4H, m), 2.24 (1H, m), 1.98-2.1 (5H, m), 1.75-1.95 (5H, m), 1.3-1.7 (9H, m), 0.8-1.2 (14H, m). ¹³C NMR (CDCl₃, 400 MHz): 170.89, 154.85, 151.56, 140.27, 135.65, 133.51, 129.53, 119.32, 69.83, 65.63, 57.60, 47.60, 46.84, 44.74, 43.00, 35.59, 33.77, 31.68, 25.99, 23.83, 22.99, 22.56, 19.60, 16.77, 10.48. ESI-MS e/z calcd. for C₂₇H₃₈N₂O₂: [M+H]+ 423.6. Found: 423.7; [M+H]+ 846.2. Found: 846.0.

### Example 34

### Preparation of Compound (33)

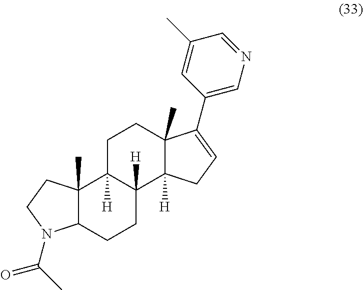

Using a synthetic procedure and conditions similar to Examples 18G in the preparation of compound (17g), replacing compound (17f) with compound (26e) and replacing diethyl (3-pyridyl)borane with 5-methylypyridin-3-ylboronic acid and subjecting the product to reactions and conditions similar to Example 27G and Example 30, a mixture of α- and β-epimers of compound (31) are made.

### Example 34A

### Preparation of Compound (33A)

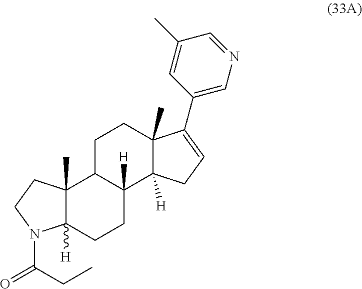

Using a synthetic procedure and conditions similar to Examples 18G in the preparation of compound (17g), replacing compound (17f) with compound (26e) and replacing diethyl (3-pyridyl)borane with 5-methylypyridin-3-ylboronic acid and subjecting the product to reactions and conditions similar to Example 27G and Example 30 and replacing acetic anhydride in Example 30 with propionic anhydride, a mixture of α- and β-epimers of compound (33A) were made. Ret time: 10.678 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.405 (s, 1H), 8.291 (s, 1H), 7.465 (s, 1H), 5.965 (s, 1H), 3.47 (m, 3H), 2.45 (s, 3H), 2.30 (m, 4H), 1.95-2.10 (m, 2H), 1.85 (m, 1H), 1.3-1.7 (m, 9H), 1.25 (m, 1H), 1.13 (m, 3H), 1.05 (d, 6H). ¹³C NMR (400 MHz, CDCl₃): 174.04, 151.68, 148.22, 144.87, 134.42, 132.45, 132.41, 129.15, 65.71, 57.58, 47.52, 45.86, 44.68, 42.77, 35.55, 33.78, 31.16, 28.80, 25.97, 23.01, 22.52, 19.55, 18.42, 16.71, 9.17. ESI-MS: m/z calcd for C₂₆H₃₆N₂O: [M+H]+ 393.58. Found: 393.5.

### Example 34B

### Preparation of Compound (33B)

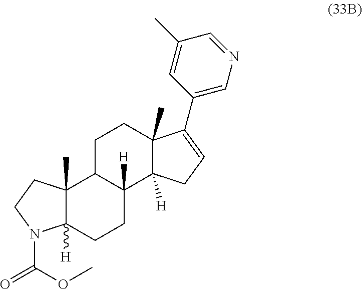

Using a synthetic procedure and conditions similar to Examples 18G in the preparation of compound (17g), replacing compound (17f) with compound (26e) and replacing diethyl (3-pyridyl)borane with 5-methylypyridin-3-ylboronic acid and subjecting the product to reactions and conditions similar to Example 27G and Example 30 and replacing acetic anhydride in Example 30 with methyl chloroformate, a mixture of α- and β-epimers of compound (33B) were made. Ret time: 11.994 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.400 (s, 1H), 8.285 (s, 1H), 7.450 (s, 1H), 5.948 (s, 1H), 3.668 (s, 3H), 3.2-3.6 (m, 3H), 2.37 (s, 3H), 2.20-2.31 (m, 2H), 1.97-2.10 (m, 2H), 1.82 (m, 1H), 1.31-1.65 (m, 9H), 1.25 (s, 1H), 1.03 (s, 3H), 1.01 (s, 3H). ¹³C NMR (400 MHz, CDCl₃): 150.72, 147.25, 143.90, 133.43, 131.44, 128.09, 64.41, 56.41, 50.93, 46.53, 43.17, 42.39, 34.58, 33.95, 32.98, 30.63, 28.67, 24.69, 21.44, 18.43, 17.43, 15.72; ESI-MS: m/z calcd for C₂₅H₃₄N₂O₂: [M+H]+ 395.55. Found: 396.4.

### Example 35

### Preparation of Compound (34)

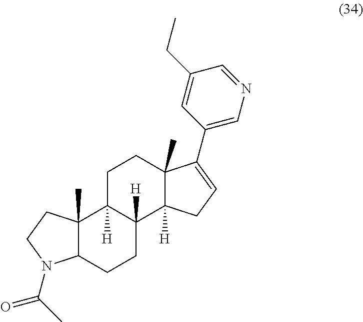

Using a synthetic procedure and conditions similar to Examples 18G in the preparation of compound (17g), replacing compound (17f) with compound (26e) and replacing diethyl (3-pyridyl)borane with 5-ethylpyridin-3-ylboronic acid and subjecting the product to reactions and conditions similar to Example 27G and Example 30, a mixture of α- and β-epimers epimer of compound (34) were made. Ret time at HPLC: 10.674 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.42 (1H, s), 8.30 (1H, s), 7.52 (1H, s), 5.95 (1H, s), 2.8-4.8 (4H, m), 2.65 (2H, m), 2.25 (1H, m), 1.8-2.15 (8H, m), 1.15-1.8 (14H, m), 0.95-1.1 (9H, m). ¹³C NMR (CDCl₃, 400 MHz): 170.91, 151.82, 147.66, 145.23, 138.55, 133.21, 132.57, 129.11, 65.64, 57.61, 47.57, 46.85, 44.75, 43.00, 35.59, 35.52, 33.78, 31.68, 26.06, 26.00, 23.83, 22.99, 22.58, 19.60, 16.75, 15.35; ESI-MS e/z calcd. For C₂₆H₃₆N₂O: [M+H]+ 393.58 Found: 423.7; [M+H]+ 786.16. Found: 785.8.

### Example 35A

### Preparation of Compound (34A)

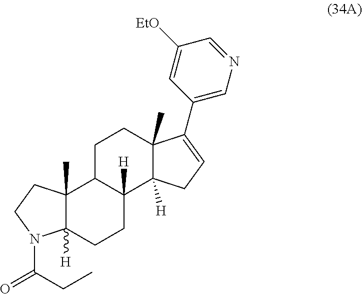

Using a synthetic procedure and conditions similar to Examples 18G in the preparation of compound (17g), replacing compound (17f) with compound (26e) and replacing diethyl (3-pyridyl)borane with 5-ethylpyridin-3-ylboronic acid and subjecting the product to reactions and conditions similar to Example 27G and Example 30, and replacing acetic anhydride in Example 30 with propionic anhydride, a mixture of α- and β-epimers of compound (34A) were made. Ret time: 11.341 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.420 (s, 1H), 8.310 (s, 1H), 7.442 (s, 1H), 5.957 (s, 1H), 3.517 (m, 3H), 2.65 (m, 2H), 2.2-2.4 (m, 3H), 1.98-2.09 (m, 2H), 1.88 (m, 1H), 1.76 (s, 1H), 1.41-1.71 (m, 9H), 1.25 (m, 4H), 1.12 (m, 3H), 1.09 (d, 6H). ¹³C NMR (400 MHz, CDCl₃): 174.08, 151.83, 147.70, 145.28, 138.50, 133.14, 132.54, 129.07, 65.74, 57.62, 47.56, 45.89, 44.72, 42.81, 35.59, 33.82, 31.67, 28.84, 26.05, 23.04, 22.57, 19.58, 16.75, 15.36, 9.20; ESI-MS: m/z calcd for C₂₇H₃₈N₂O: [M+H]+ 407.6. Found: 407.6 .

### Example 35B

### Preparation of Compound (34B)

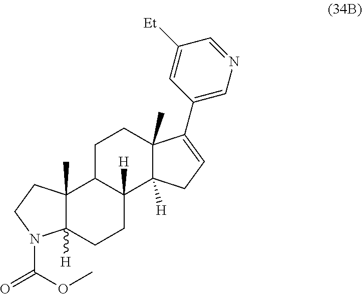

Using a synthetic procedure and conditions similar to Examples 18G in the preparation of compound (17g), replacing compound (17f) with compound (26e) and replacing diethyl (3-pyridyl)borane with 5-ethylpyridin-3-ylboronic acid and subjecting the product to reactions and conditions similar to Example 27G and Example 30, and replacing acetic anhydride in Example 30 with methyl chloroformate, a mixture of α- and β-epimers of compound (34B) were made. Ret. time: 12.628 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.422 (s, 1H), 8.311 (s, 1H), 7.446 (s, 1H), 5.954 (s, 1H), 3.912 (s, 3H), 3.2-3.6 (m, 3H), 2.67 (m, 2H), 2.22 (m, 1H), 2.01 (m, 2H), 1.85 (m, 1H), 1.32-1.75 (m, 9H), 1.25 (m, 3H), 1.0-1.14 (m, 8H). ¹³C NMR (400 MHz, CDCl₃): 151.84, 147.68, 145.26, 138.48, 133.15, 132.51, 128.99, 65.42, 57.43, 51.95, 47.56, 44.18, 43.13, 35.60, 34.96, 34.00, 31.64, 26.04, 25.70, 23.33, 22.47, 19.46, 16.47, 15.34; ESI-MS: m/z calcd for C₂₆H₃₆N₂O₂: [M+H]+ 409.58. Found: 409.5.

### Example 36

### Preparation of Compound (35)

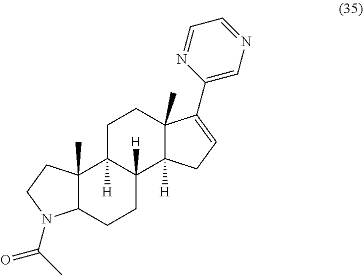

Using a synthetic procedure and conditions similar to Examples 18G in the preparation of compound (17g), replacing compound (17f) with compound (26e) and replacing diethyl (3-pyridyl)borane with 2-(tributylstannyl)pyrazine and subjecting the product to reactions and conditions similar to Example 27G and Example 30, a mixture of α- and β-epimers of compound (M) are made.

### Example 37

### Preparation of Compound (36)

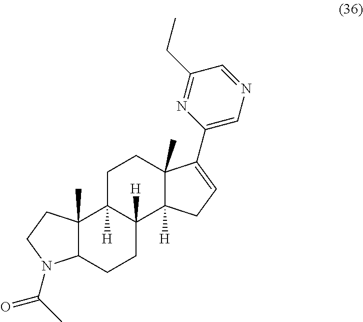

Using a synthetic procedure and conditions similar to Examples 18G in the preparation of compound (17g), replacing compound (17f) with compound (26e) and replacing diethyl (3-pyridyl)borane with 2-ethyl-6-(tributylstannyl)pyrazine and subjecting the product to reactions and conditions similar to Example 27G and Example 30, a mixture of α- and β-epimers of compound (36) are made.

### Example 38

### Preparation of Compound (37)

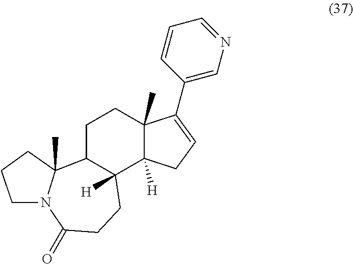

### Example 38A

### Preparation of Compound (37a)

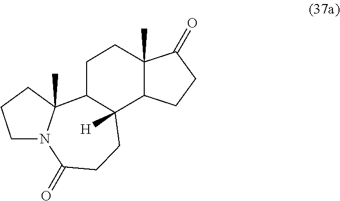

A solution of compound (17b) (67.3 mg) in dichloromethane (2 mL) was treated with TiCl₄ (30 mg) at 0° C. under argon. A precipitate immediately formed. The mixture was warmed to room temperature and stirred until all the starting material was consumed. The reaction was quenched with water (5 mL). The layers were separated. The aqueous layer was extracted with dichloromethane (2×5 mL). The combined layers were washed with water (5 mL) and brine (5 mL), dried over Na₂SO₄, and evaporated to give compound (37a). ¹H NMR (CDCl₃, 300 MHz): 3.95 (1H, m), 3.15 (1H, m), 2.45-2.55 (2H, m), 2.2-2.4 (2H, m), 1.25 (3H, s), 0.85 (3H, s). ¹³C NMR (CDCl₃, 300 MHz): 173.40, 65.15, 54.20, 53.46, 50.58, 48.66, 47.27, 44.10, 19.75, 13.72 .

### Example 38B

### Preparation of Compound (37b)

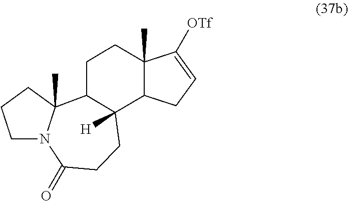

To a solution of compound (37a) (145 mg, 0.527 mmol) in dichloromethane (20 mL) was added trifluoromethane sulfonic anhydride (178.34 mg) at room temperature, the solution was stirred over 10 min and TEA (53.18 mg) in DCM (5 mL) was added dropwise within 15 min. The mixture was stirred for 18 h and all the starting material was consumed by TLC analysis. Water (10 mL) was added and the layers were separated. The aqueous layer was extracted with dichloromethane (3×18 mL). The combined organic layers were washed with water (15 mL) and brine (15 mL), dried over Na₂SO₄ and evaporated to dryness. The residue was purified by column chromatography on silica gel (hexanes/EA=1/1) to give 70 mg (34%) of compound (37b). MS calculated for (C₁₉H₂₆F₃NO₄S) [M+H]+ 422.47 Found: 422.6; [M+Na]+444.47 Found: 444.5; [2M+H]+ 844.4 Found: 843.6 .

### Example 38C

### Preparation of Compound (37)

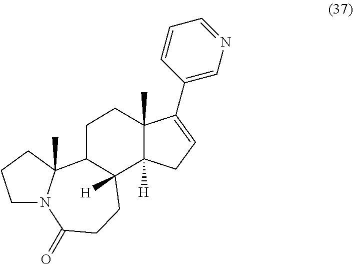

To a solution of compound (37b) (1 g) in THF (10 mL) was added diethyl (3-pyridyl)borane (1.2 g), bis(triphenylphosphine)palladium(II) chloride (26 mg) and 2N aqueous Na₂CO₃ (5.5 mL). The mixture was degassed and refilled with Argon gas three times. Then the mixture was heated at 80° C. overnight and monitored by TLC. The mixture was cooled to room temperature and extracted with dichloromethane (20 mL). The combined organic layers were washed with water (20 mL) and brine (2×20 mL), dried over Na₂SO₄. The solution was concentrated and purified by preparative HPLC to give 70 mg of compound (37). MS calculated for (C₂₃H₃₀N₂O) [M+H]+351.5 Found: 351.0; [M+Na]+373.5 Found: 372.9; ¹H NMR (CDCl₃, 300 MHz): 8.55 (1H), 8.36 (1H), 7.55 (1H), 7.11 (1H), 5.9 (1H), 3.92 (1H), 3.22 (1H), 2.4-2.6 (4H), 2.2 (1H), 1.25 (3H), 0.98 (23H). ¹³C NMR (CDCl₃, 300 MHz): 173.53, 151.37, 147.99, 147.74, 133.61, 132.57, 128.81, 123.07, 65.31, 56.66, 54.28, 48.70, 46.88, 43.75, 19.70, 16.56; Retention time at HPLC: 8.369 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)].

### Example 39

### Preparation of Compound (38)

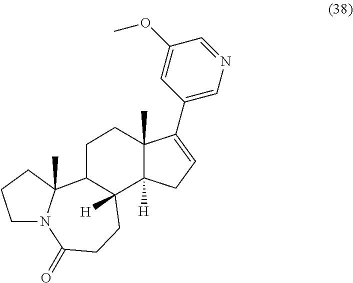

To a solution of (37b) (0.5 g, 1.2 mmol) in THF (20 mL) was added 5-methoxypyridin-3-ylboronic acid (450 mg, 2.966 mmol), bis(triphenylphosphine)palladium(II) chloride (42 mg, 5.9 mmol) and 20 wt % aqueous Na₂CO₃ solution (3 ml). The mixture was degassed and refilled with Ar gas three times. Then the mixture was heated at 60° C. under nitrogen overnight and monitored by TLC (hexane:EtOAc=1:2). The mixture was cooled to room temperature and extracted with dichloromethane (2×30 mL). The combined organic layers were washed with brine (2×30 mL) and dried over (Na₂SO₄). The solution was concentrated and purified by column chromatography (silica gel, hexane: EtOAc=1:2) to give 400 mg of compound (38). Ret. time at HPLC: 9.315 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR: 8.22 (1H, s), 8.17 (1H, s), 7.13 (1H, s), 6.0 (1H, s), 4.0 (1H, m), 3.9 (3H, s), 3.25 (1H, m), 2.67 (2H, m), 2.31 (1H, m), 2.0-2.1 (2H, m), 1.6-2.0 (9H, m), 2.5 (2H, m), 2.4 (3H, s), 2.3-2.37 (2H, s), 1.05 (3H, s). ¹³C NMR: 173.60, 155.29, 151.24, 140.36, 135.33, 133.19, 129.15, 118.69, 65.35, 56.70, 55.54, 54.32, 48.74, 46.95, 43.78, 37.42, 36.57, 35.45, 32.23, 28.77, 25.44, 20.94, 19.72,

16.63. ESI-MS e/z calcd. For C₂₄H₃₂N₂O₂: [M+H]+ 381.52. Found: 381.8.

### Example 40

### Preparation of Compound (39)

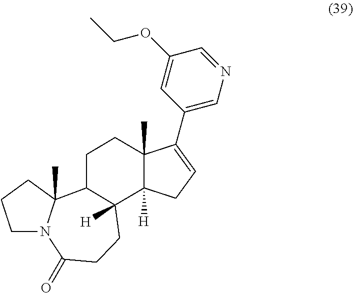

Using a synthetic procedure and condition similar to Example 39 in the preparation of compound (38), and replacing 5-methoxypyridin-3-ylboronic acid with 5-ethoxypyridin-3-ylboronic acid, compound (39) was made. Ret. time at HPLC: 10.142 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR: 8.26 (1H, s), 8.15 (1H, s), 7.12 (1H, s), 5.98 (1H), 4.0-4.1 (2H, m), 4.0 (1H, m), 3.25 (1H, m), 2.65 (2H, m), 2.3 (1H, m), 1.45 (3H, m), 1.4 (3H, s), 1.05 (3H, s). ¹³C NMR: 173.62, 154.69, 151.25, 140.17, 135.63, 133.21, 129.03, 119.36, 65.36, 63.87, 56.68, 54.31, 48.74, 46.93, 43.77, 37.42, 36.53, 35.45, 32.21, 28.75, 25.43, 20.92, 19.71, 16.61, 14.76; ESI-MS e/z calcd. For C₂₅H₃₄N₂O₂: [M+H]+ 395.55. Found: 395.5; [2M+H]+ 790.1. Found: 790.0.

### Example 41

### Preparation of Compound (40)

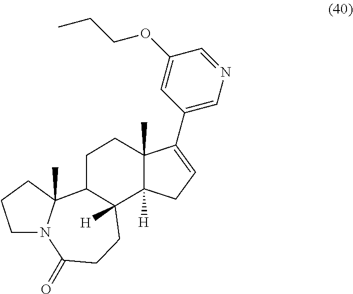

Using a synthetic procedure and condition similar to Example 39 in the preparation of compound (38), and replacing 5-methoxypyridin-3-ylboronic acid with 5-propoxypyridin-3-ylboronic acid, compound (40) was made. Ret. time at HPLC: 10.078 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.20 (1H, s), 8.16 (1H, s), 7.13 (1H, s), 5.98 (1H, s), 3.9-4.0 (3H, m), 3.29 (1H, m), 2.68 (2H, m), 2.31 (1H, m), 1.41 (3H, s), 1.05 (3H, s), 1.01 (3H, m). ¹³C NMR (CDCl₃, 400 MHz): 173.62, 154.89, 151.30, 140.10, 135.73, 133.20, 129.02, 119.33, 69.84, 65.36, 56.70, 54.32, 48.74, 46.94, 43.78, 37.43, 36.55, 35.46, 32.21, 28.76, 25.45, 22.54, 20.93, 19.71, 16.63, 10.46; ESI-MS e/z calcd. For C₂₆H₃₆N₂O₂: [2M+H]+ 818.16. Found: 818.0.

### Example 42

### Preparation of Compound (41)

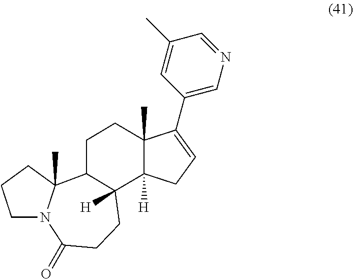

Using a synthetic procedure and condition similar to Example 39 in the preparation of compound (38), and replacing 5-methoxypyridin-3-ylboronic acid with 5-methylypyridin-3-ylboronic acid, compound (41) was made. Ret. time at HPLC: 8.968 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.40 (s, 1H), 8.29 (s, 1H), 7.44 (s, 1H), 5.96 (s, 1H), 3.95 (m, 1H), 3.25 (m, 1H), 2.69 (m, 2H), 2.37 (s, 3H), 2.32 (m, 2H), 1.98-2.10 (m, 2H), 1.68-1.98 (m, 8H), 1.5-1.65 (m, 2H), 1.32 (s, 3H), 1.32 (m, 2H), 1.03 (s, 3H). ¹³C NMR (400 MHz, CDCl₃): 173.577, 151.456, 148.424, 144.823, 134.415, 132.499, 132.146, 128.647, 65.342, 56.689, 54.308, 48.721, 46.889, 43.765, 37.419, 36.554, 35.450, 32.198, 28.762-25.426, 20.921, 19.713, 18.445, 16.589; ESI-MS: m/z calcd for C₂₄H₃₂N₂O: [M+H]+ 365.5. Found: 365.7.

### Example 43

### Preparation of Compound (42)

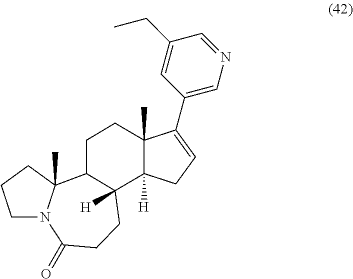

Using a synthetic procedure and condition similar to Example 39 in the preparation of compound (38), and replacing 5-methoxypyridin-3-ylboronic acid with 5-ethylpyridin-3-ylboronic acid, compound (42) was made. Ret. time at HPLC: 9.70 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.41 (1H, s), 8.31 (1H), 7.44 (1H , s), 5.96 (1H, s) 3.98 (1H, m), 3.30 (1H, m) 3.65 (4H), 2.26 (1H), 1.42 (3H, s), 1.25 (7H, m), 1.02 (3H, s). ¹³C NMR (CDCl₃, 400 MHz): 173.63, 151.57, 147.79, 145.11, 138.62, 133.18, 132.24, 128.57, 65.37, 56.70, 54.32, 48.74, 46.91, 43.78, 37.43, 36.55, 35.46, 32.21, 28.77, 26.04, 25.45, 20.93, 19.71, 16.60, 15.32; ESI-MS e/z calcd. For C₂₅H₃₄N₂O: [M+H]+ 379.55. Found: 379.5; [2M+H]+ 758.1. Found: 757.9.

### Example 43A

### Preparation of Compound (42A)

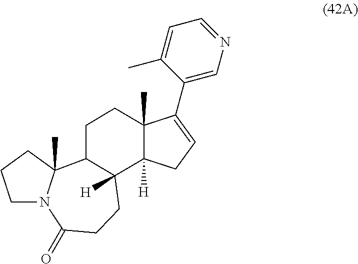

Using a synthetic procedure and condition similar to Example 39 in the preparation of compound (38), and replacing 5-methoxypyridin-3-ylboronic acid with 4-methylypyridin-3-ylboronic acid, compound (42A) was made. Ret. time at HPLC: 8.726 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.31 (1H), 8.24 (1H), 7.10 (1H), 5.66 (1H), 3.92-4.03 (m, 1H), 3.19-3.33 (m, 1H), 2.55-2.72 (m, 2H), 2.30-2.42 (m, 1H), 2.30 (s, 3H), 2.05-2.18 (m, 2H). ¹³C NMR 173.64, 150.28, 149.12, 147.69, 145.64, 133.23, 130.29, 125.15, 65.40, 56.32, 54.44, 49.02, 48.74, 43.77, 37.77, 36.58, 35.08, 32.64, 28.89, 25.33, 20.92, 20.23, 19.73, 16.33; ESI MS 365.1 [M+H]+.

### Example 43B

### Preparation of Compound (42B)

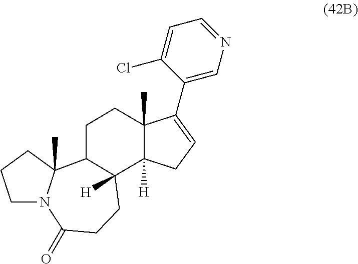

Using a synthetic procedure and condition similar to Example 39 in the preparation of compound (38), and replacing 5-methoxypyridin-3-ylboronic acid with 4-chloroypyridin-3-ylboronic acid, compound (42B) was made. Ret. time at HPLC: 11.540 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.37 (2H), 7.32 (1H), 5.83 (1H), 3.90-4.00 (m, 1H), 3.19-3.30 (m, 1H), 2.55-2.72 (m, 2H), 2.30-2.42 (m, 1H), 2.05-2.18 (m, 2H). ¹³C NMR 173.62, 150.53, 148.76, 148.11, 143.44, 132.70, 132.23, 124.71, 65.39, 56.08, 54.40, 49.27, 48.75, 43.79, 37.77, 36.59, 34.86, 32.77, 28.86, 25.33, 20.93, 19.74, 16.33; ESI MS 407.1 [M+Na]⁺.

### Example 44

### Preparation of Compound (43)

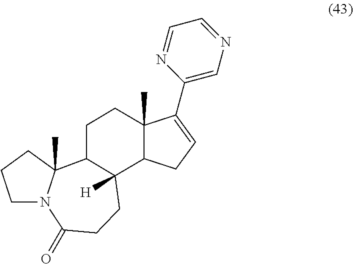

Using a synthetic procedure and condition similar to Examples 38C in the preparation of compound (37), and replacing diethyl (3-pyridyl)borane with 2-(tributylstannyl)pyrazine compound (43) is made.

### Example 45

### Preparation of Compound (44)

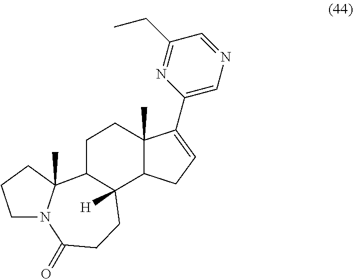

Using a synthetic procedure and condition similar to Examples 38C in the preparation of compound (37), and replacing diethyl (3-pyridyl)borane with 2-ethyl-6-(tributylstannyl)pyrazine compound (44) is made.

### Example 46

### Preparation of Compounds (45) and (46)

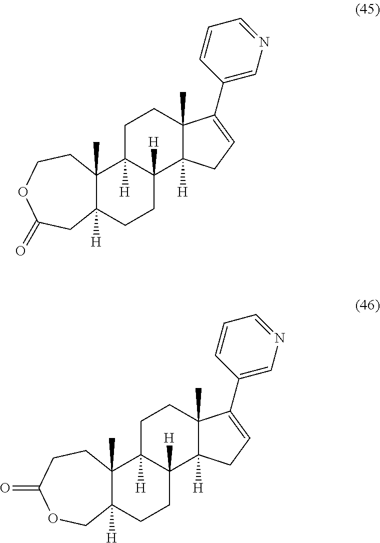

### Example 46A

### Preparation of Compound (45a)

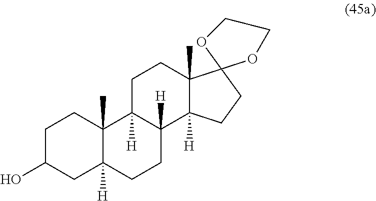

A solution of epiandrosterone (29 g), ethylene alcohol (16.8 mL) and p-toluenesulfonic acid (0.517 g) in toluene (200 ml) were refluxed under a Dean-Stark trap for 2 h. The mixture was cooled to room temperature, diluted with dichloromethane (100 mL), and washed with saturated NaHCO₃ (100 mL) and brine (100 mL). The organic phase was dried over anhydrous sodium sulfate, filtered, and evaporated to provide 35 g of compound (45a). ¹H NMR (CDCl₃, 300 MHz): 3.85-4.0 (4H), 3.75 (1H), 3.56 (1H), 0.9-0.95 (6H).

### Example 46B

### Preparation of Compound (45b)

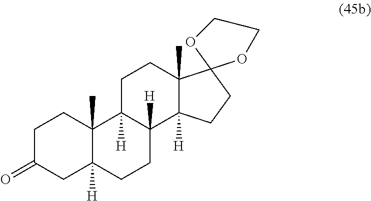

A solution of compound (45a) (5 g) in dichloromethane (300 mL) was treated with Dess-Martin Periodinane (9.5 g) at room temperature. The reaction was stirred until all the start material was consumed. The mixture was washed with sat.Na₂SO₃ (150 mL), saturated NaHCO₃ (2×150 mL) and brine (150 mL). The solvent was dried over anhydrous sodium sulfate, filtered, and evaporated to dryness. The residue was purified by flash column chromatograph (silica gel, hexane: EtOAc=95:5) to afford 3.5 g (69.9%) of compound (45b). ¹H NMR (CDCl₃, 300 MHz): 3.7-4.1 (4H), 2.2-2.5 (3H), 1.9-2.1 (3H), 1.05 (3H), 0.91 (3H). ¹³C NMR (CDCl₃, 300 MHz): 211.84, 119.27, 65.16, 64.49, 53.58, 50.10, 14.37, 11.43.

### Example 46C

### Preparation of Compounds (45c) and (46a)

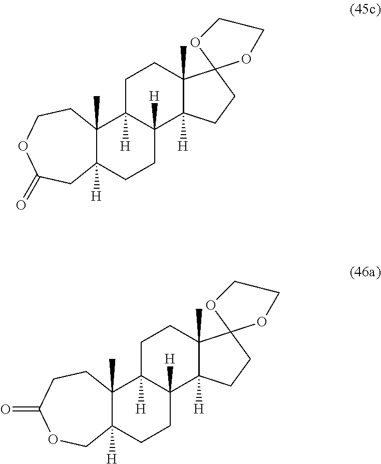

A solution of compound (45b) (3.2 g) in dichloromethane (250 mL) was treated with mCPBA (4.9 g). The mixture was stirred overnight at reflux. The reaction was monitored by TLC and the starting material was completely consumed. The mixture was washed with 10% aqueous Na₂CO₃ (150 mL), 10% aqueous NaHCO₃ (150 mL) and brine (150 mL). The organic phase was dried over anhydrous sodium sulfate, filtered, and evaporated to dryness. The residue was purified by flash column chromatograph to afford 3.1 g (92.4%) of the regioisomeric mixture of compound (45c) and compound (46a). ¹H NMR (CDCl₃, 300 MHz): 4.25-4.35 (1H), 4.1-4.2 (0.47H), 3.8-4.0 (4H), 3.65-3.7 (0.0.53H), 2.45-2.90 (2H), 0.95 (3H), 0.85 (3H). ¹³C NMR (CDCl₃, 300 MHz): 176.17, 119.19, 70.01, 65.19, 64.53, 53.59, 53.37, 14.33, 12.3.

### Example 46D

### Preparation of Compounds (45d) and (46b)

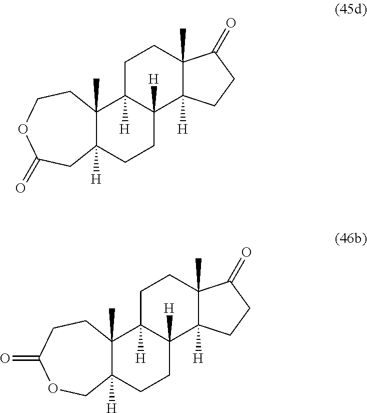

The regioisomeric mixture of compound (45c) and compound (46a) (2 g), p-TsOH (0.2 g) and water (150 mg) in acetone (60 mL) was stirred at room temperature for 4 h. The solvent was removed under reduce pressure, 20 mL of EtOAc was added, the organic phase was separated, washed with saturated NaHCO₃ (15 mL) and brine (20 mL). The solvent was dried over anhydrous sodium sulfate, filtered, and evaporated to give 1.5 g (85.8%) of the regioisomeric mixture of compound (45d) and compound (46b). ¹H NMR (CDCl₃, 300 MHz): 4.35-4.45 (1H), 4.15-4.2 (0.47H), 3.68-3.71 (0.52H), 2.6-2.9 (1H), 2.4-2.5 (1H), 2.0-2.1 (2H), 0.96 (3H), 0.85 (3H). ¹³C NMR (CDCl₃, 300 MHz): 175.94, 175.70, 69.83, 64.52, 53.83, 53.62, 51.14, 48.54, 47.46, 43.31, 41.63, 13.74, 12.31, 12.10.

### Example 46D

### Preparation of Compounds (45e) and (46c)

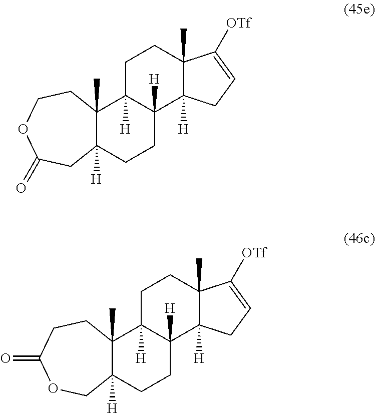

To a solution of compound (45d) and compound (46b) (1.45 g) in DCM (60 mL) was added trifluoromethane sulfonic anhydride (1.55 g, 1.5 equiv) at room temperature. The solution was stirred over 10 min and TEA (0.5 g, 1 eq.) in DCM (10 mL) was added dropwise within 30 min. The mixture was stirred overnight. The reaction mixture was poured into saturated NaHCO₃ (40 mL), and the layers were separated. The aqueous layer was extracted with DCM (3×50 mL). The organic layers were combined, washed with water (40 ml) and brine (40 ml), dried over sodium sulfate. The solvent was concentrated and purified by column chromatography on silica gel (hexanes/EA=95:5) to give 0.8 g (36.7%) of a mixture of compound (45e) and compound (46c). ¹H NMR (CDCl₃, 300 MHz): 5.6 (1H), 4.25-4.35 (1H), 4.1-4.15 (0.449H), 3.65-3.75 (0.646H), 2.5-2.9 (2H), 0.98-1.05 (6H). ¹³C NMR (CDCl₃, 300 MHz): 174.89, 174.66, 157.98, 119.14, 115.96, 113.55, 113.45, 68.82, 63.48, 53.06, 52.87, 47.70, 14.22, 11.24, 11.03.

### Example 46E

### Preparation of Compounds (45) and (46)

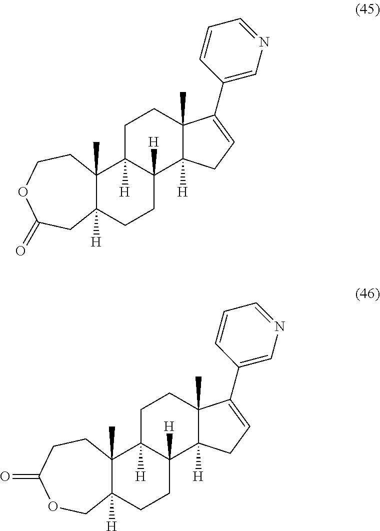

To a solution of compound (45e) and compound (46c) (0.6 g) in THF (30 mL) was added diethyl (3-pyridyl)borane (0.255 g), bis(triphenylphosphine)palladium(II) chloride (51 mg) and 2 N aqueous Na₂CO₃ (0.45 g). The mixture was degassed and refilled with Argon gas three times. The mixture was heated at 80° C. overnight. The reaction was monitored by TLC. The mixture was cool to room temperature and extracted with EtOAc (2×20 mL). The organic layers were combined, washed with brine (2×20 mL) and dried over Na₂SO₄. The solution was concentrated and purified by column chromatography on silica gel (EtOAc/Hexanes=1:5) to give the mixture of compound (45) and compound (46) (0.4 g, 79.6%). The regioisomeric mixture was purified by preparative HPLC to isolate compound (45) and compound (46). Compound (45): Retention time at HPLC: 10.426 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. MS calculated for (C₂₄H₃ᵢNO₂) [M+H]+366.51 Found: 366.5 ; [2M+Na]+754.02 Found: 753.6 ¹H NMR (CDCl₃, 300 MHz): 8.65 (1H), 8.45 (1H), 7.6-7.7 (1H), 7.19 (1H), 6.0 (1H), 4.26-4.31 (1H), 4.12 (1H), 2.8-2.9 (1H), 2.2-2.3 (1H), 2.1-2.13 (1H), 1.9-2.05 (2H). ¹³C NMR (CDCl₃, 300 MHz): 175.84, 151.51, 147.91, 147.82, 133.69, 132.82, 129.18, 123.05, 64.60, 57.11, 53.98. Compound (46): Retention time at HPLC: 10.56 min [Mobile phase: B %=10-100 (gradient 20 min); B=MeCN, A=H₂O 0.1% TFA); Flow rate: 0.8 mL/min; UV=266 nm column: zorbax Eclipse XDB-C8 5u, (150*4.6 mmID)]. MS calculated for (C₂₄H₃ᵢNO₂) [M+H]+366.51 Found: 366.4; [2M+Na]+754.02 Found: 753.5 . ¹H NMR (CDCl₃, 300 MHz): 8.65 (1H), 8.45 (1H), 7.65 (1H), 7.18 (1H), 6.0 (1H), 4.25 (1H), 3.68 (1H), 2.7 (1H), 2.5 (1H). ¹³C NMR (CDCl₃, 300 MHz): 176.08, 151.55, 147.92, 147.84, 133.66, 132.80, 129.09, 123.05, 69.98, 57.12, 53.78, 48.74, 47.25.

### Example 47

### Preparation of Compounds (47) and (48)

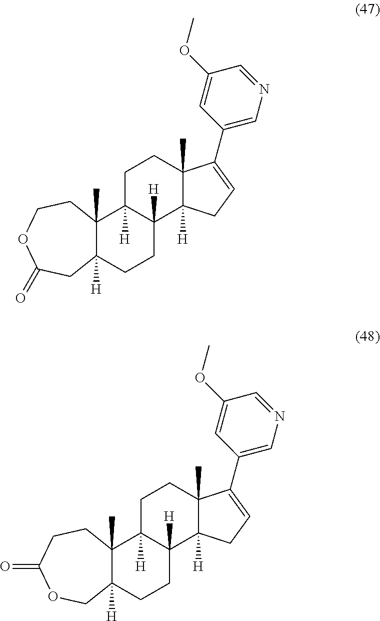

Using a synthetic procedure and condition similar to Examples 46E in the preparation of compound (45) and compound (46) replacing diethyl (3-pyridyl)borane with 5-methoxypyridin-3-ylboronic acid, compound (47) and compound (48) were made. Compound (48): Ret. time at HPLC: 11.414 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.22 (1H, s), 8.16 (1H, s), 7.14 (1H, s), 5.98 (1H, s), 4.30 (1H, m), 3.90 (3H, s), 3.70 (1H, d), 2.71 (1H, m), 2.51 (1H, m), 2.21 (1H, m), 2.01 (2H, m), 1.0 (6H, d). ¹³C NMR (CDCl₃, 400 MHz): 176.14, 155.29, 151.36, 140.40, 135.15, 133.43, 129.43, 118.81, 70.00, 57.13, 55.56, 53.80, 48.74, 47.31, 37.83, 35.34, 35.25, 33.45, 31.93, 31.60, 31.36, 29.71, 26.25, 21.04, 16.70, 12.32; ESI-MS: m/z calcd for C₂₅H₃₃NO₃: [M+H]+ 396.5. Found: 395.0.

### Example 48

### Preparation of Compounds (49) and (50)

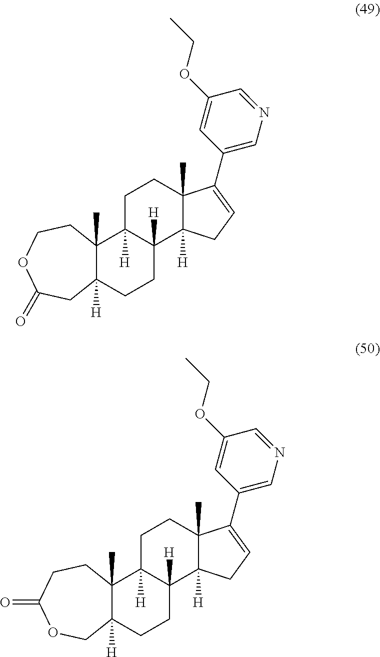

Using a synthetic procedure and condition similar to Examples 46E in the preparation of compound (45) and compound (46) replacing diethyl (3-pyridyl)borane with 5-ethoxypyridin-3-ylboronic acid, compound (49) and compound (50) were made. Compound (50): Ret. time at HPLC: 12.271 min [Mobile phase: B %=10-100 (gradient 20 min); B=MeCN, A=H20 (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.20 (1H, s), 8.15 (1H, s), 7.12 (1H, s), 5.97 (1H, s), 4.29 (1H, m), 4.10 (2H, m), 3.71 (1H, d), 2.75 (1H, m), 2.54 (1H, m), 2.21 (1H, m), 2.02 (2H, m), 1.45 (3H, m), 1.01 (6H, d). ¹³C NMR (CDCl₃,400 MHz): 175.10, 153.64, 150.42, 139.34, 134.62, 132.37, 128.25, 118.33, 68.98, 62.86, 56.11, 52.80, 47.73, 46.29, 36.82, 34.34, 34.25, 32.45, 30.57, 30.35, 28.69, 25.25, 20.03, 15.68, 13.78, 11.31; ESI-MS: m/z calcd for C₂₆H₃₅NO₃: [M+H]+ 410.56. Found: 410.3.

### Example 49

### Preparation of Compounds (51) and (52)

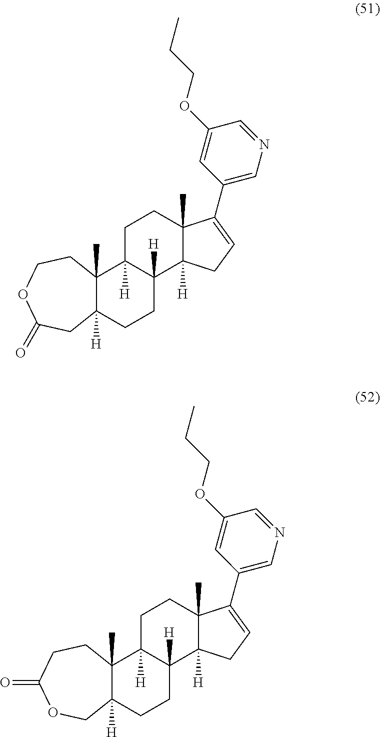

Using a synthetic procedure and condition similar to Examples 46E in the preparation of compound (45) and compound (46) replacing diethyl (3-pyridyl)borane with 5-propoxypyridin-3-ylboronic acid, compound (51) and compound (52) are made.

### Example 50

### Preparation of Compounds (53) and (54)

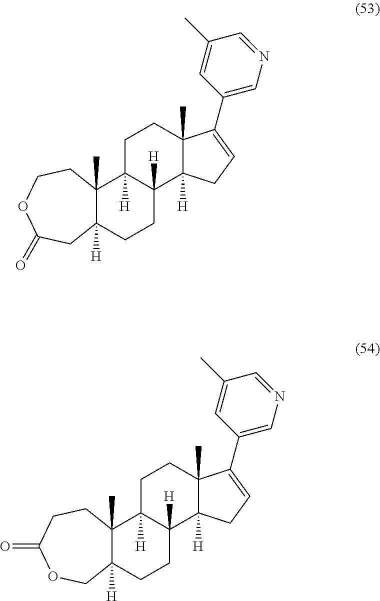

Using a synthetic procedure and condition similar to Examples 46E in the preparation of compound (45) and compound (46) replacing diethyl (3-pyridyl)borane with 5-methylypyridin-3-ylboronic acid, compound (53) and compound (54) were made. Ret. time at HPLC: 11.101 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.43 (s, 1H), 8.31 (s, 1H), 5.96 (s, 1H), 4.32 (m, 1H), 3.75 (m, 1H), 2.73 (m, 1H), 2.52 (m, 1H), 2.35 (s, 3H), 2.27 (m, 3H), 1.9-2.12 (m, 6H), 1.2-1.9 (m, 10H), 1.01 (d, 6H). ¹³C NMR (400 MHz, CDCl₃): 176.14, 151.62, 148.32, 144.90, 134.49, 132.49, 132.06, 128.93, 70.01, 57.14, 53.80, 48.74, 47.27, 37.83, 35.35, 35.26, 33.47, 31.58, 29.71, 26.27, 22.70, 21.04, 18.46, 16.67, 14.14, 12.33; ESI-MS: m/z calcd for C₂₅H₃₃NO₂ [M+H]+ 380.54. Found: 380.6.

### Example 51

### Preparation of Compounds (55) and (56)

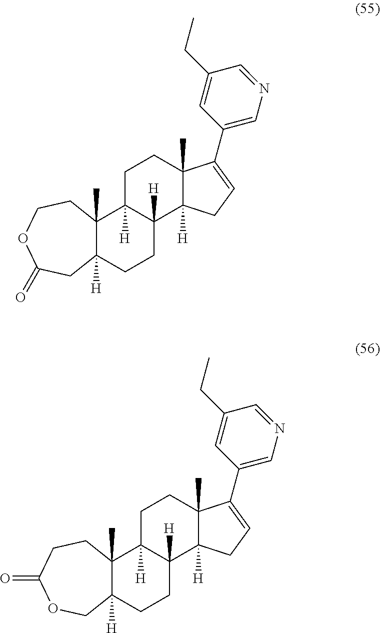

Using a synthetic procedure and condition similar to Examples 46E in the preparation of compound (45) and compound (46) replacing diethyl (3-pyridyl)borane with 5-ethylpyridin-3-ylboronic acid, compound (55) and compound (56) were made. The mixture of compound (55) and compound (56) (71:29 ratio) have the following analytical data: Ret. time at HPLC: 11.108 min (55)+11.352 min (56), [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.42 (1H, s), 8.31 (1H, s), 7.51 (1H, s), 5.95 (1H, s), 4.2-4.3 (1H), 3.9-4.1 (1H), 2.5-2.8 (3H), 2.19-2.31 (2H), 1.95-2.15 (2H), 1.35-2.95 (9H), 1.15-1.36 (4H), 1.02 (6H). ¹³C NMR (CDCl₃, 400 MHz): 170.47, 151.74, 147.85, 145.29, 138.56, 133.15, 132.33, 128.74, 80.81, 56.98, 49.99, 47.46, 47.34, 40.57, 34.93, 34.82, 33.83, 33.51, 33.36, 31.67, 30.85, 27.13, 26.06, 21.24, 16.68, 15.34, 12.62, 10.15; ESI-MS: m/z calcd for C₂₅H₃₃NO₂: [M+H]+ 380.54 Found: 380.5. Compound (56): Ret. time at HPLC: 11.846 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.40 (1H), 8.29 (1H), 7.41 (1H), 5.92 (1H, vinyl), 4.23-4.32 (m, 1H), 4.68-4.72 (d, 1H), 2.58-2.71 (m, 3H), 2.47-2.53 (m, 1H), 2.15-2.27 (m, 1H). ¹³C NMR 176.11, 151.69, 147.63, 145.13, 138.60, 133.25, 132.46, 128.86, 69.99, 57.13, 53.80 48.73, 47.27, 37.82, 35.34, 35.26, 33.46, 31.93, 31.58, 31.37, 29.70, 29.37, 26.26, 26.05, 22.67, 21.04, 16.67, 15.34, 14.13, 12.32; ESI MS 394.0 [M+H]+.

### Example 52

### Preparation of Compounds (57) and (58)

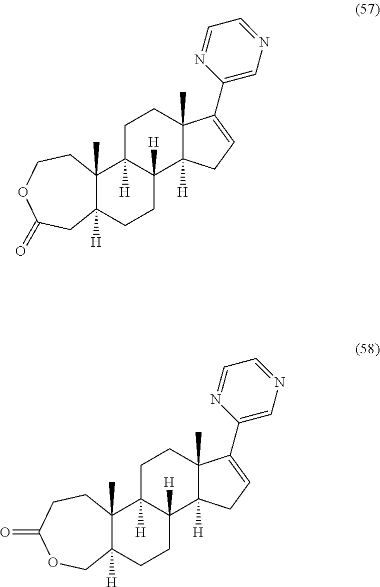

Using a synthetic procedure and condition similar to Examples 46E in the preparation of compound (45) and compound (46) replacing diethyl (3-pyridyl)borane with 2-(tributylstannyl)pyrazine, compound (57) and compound (58) are made.

### Example 53

### Preparation of Compounds (59) and (60)

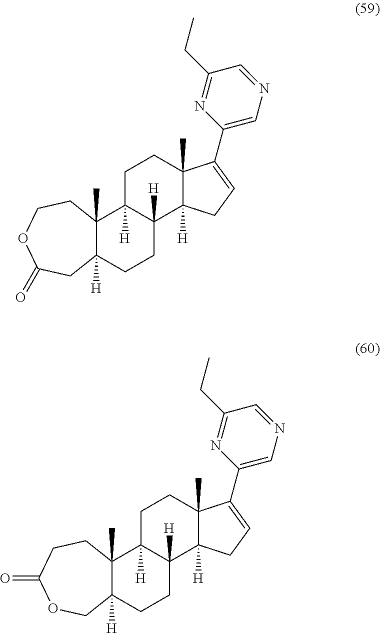

Using a synthetic procedure and condition similar to Examples 46E in the preparation of compound (45) and compound (46) replacing diethyl (3-pyridyl)borane with 2-ethyl-6-(tributylstannyl)pyrazine, compound (59) and compound (60) are made.

### Example 54

### Preparation of Compound (61)

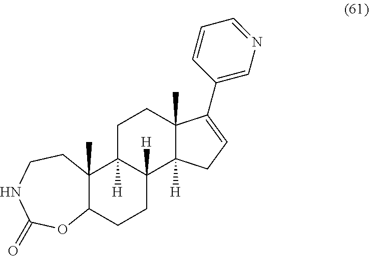

### Example 54A

### Preparation of Compound (61a)

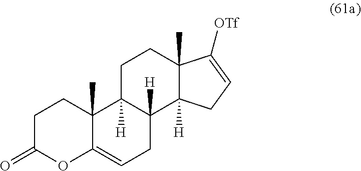

To a solution of (4aR,4bS,6aS,9aS,9bR)-4a,6a-dimethyl-4,4a,4b,5, 6, 6a,9, 9a,9b,10-decahydroindeno[5,4-f]chromene-2, 7 (3H, 8H)-dione (2.0 g, 6.9 mmol) in DCM (35 mL) was added trifluoromethane sulfonic anhydride (0.75 mL, 10.4 mmol, 1.5 equiv) at room temperature. The solution was stirred over 10 min and TEA (1 mL, 6.9 mmol, 1 eq.) in dichloromethane (DCM) (10 mL) was added dropwise within 30 min. The mixture was stirred for 5 h. The reaction was monitored by TLC (EtOAc:hexanes=1:3) and the starting material was completely consumed. Water (20 mL) was added and the layers were separated. The aqueous layer was extracted with DCM (3×50 mL). The organic layers were combined, washed with 2N HCl, brine, dried (MgSO₄). The solution was concentrated and purified by column chromatography on silica gel (hexanes/EA=1:1, 1% HOAc) to give a mixture of (4aR,4bS,6aS,9aS,9bR)-4a,6a-dimethyl-2-oxo-2,3,4,4a,4b,5, 6, 6a,9, 9a,9b,10-decahydroindeno[5,4-f]chromene-7-yl trifluoromethanesulfonate, compound (61a) [compound (1c)] and 3-((3aS,5aS,6R, 9aS,9bS)-3a,6-dimethyl-7-oxo-3-(trifluoromethylsulfonyloxy)-3a,4,5, 5a,6, 7, 8, 9, 9a,9b-decahydro-1H-cyclopenta[α]naphthalen-6-yl)propanoic acid (2 g, 66%), which was used in the next step without further purification. MS calcd for compound (61a) [compound (1c)] (C₁₉H₂₅F₃₀₆S), [M+H]+ 439.46 Found: 439.0; [2M−H]− 437.46 Found: 437.1.

### Example 54B

### Preparation of Compound (61b)

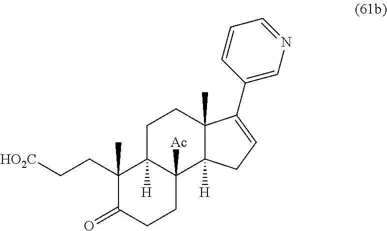

To a solution of (4aR,4bS,6aS,9aS,9bR)-4a,6a-dimethyl-2-oxo-2,3,4,4a,4b, 5, 6, 6a,9, 9a,9b,10-decahydroindeno[5,4-f]chromene-7-yl trifluoromethanesulfonate, compound (61a) [compound (1c)] and 3-((3aS,5aS,6R, 9aS,9bS)-3a,6-dimethyl-7-oxo-3-(trifluoromethylsulfonyloxy)-3a,4,5, 5a,6, 7, 8, 9, 9a,9b-decahydro-1H-cyclopenta[α]naphthalen-6-yl)propanoic acid (2.3 g) in THF (350 mL) was added pyridin-3-yl boronic acid (1.5 g, 2.5 equiv), (Ph₃P)₂PdCl₂ (160 mg, 0.05 equiv) and 2 N aqueous Na₂CO₃ (12 mL). The mixture was degassed and refilled with Argon three times. And, the mixture was heated at 80° C. overnight. The reaction was monitored by TLC. The mixture was cool to room temperature and extracted with DCM (2×30 mL). The organic layers were combined, washed with brine (2×20 mL) dried (Na₂SO₄). The solution was concentrated and purified by column chromatography on silica gel (EtOAc/Hexanes=1:1, 0.5% HOAc) to give 3-((3aS,5aS,6R, 9aS,9bS)-3a,6-dimethyl-7-oxo-3-(pyridin-3-yl)-3a,4,5, 5a,6, 7, 8, 9, 9a,9b-decahydro-1H-cyclopenta[α]naphthalen-6-yl)propanoic acid, compound (61b) [compound (1d)], (1.1 g, 65%) as a pale yellow solid. MS calcd for (C₂₃H₂₉NO₃) [2M+H]+ 735.96 Found: 735.5; [2M−H]− 733.96 Found: 733.6. ¹H NMR (CDCl₃, 300 MHz): δ 8.619 (s, 1H), 8.45 (brs, 1H), 7.68 (d, 1H), 7.29 (m, 1H), 6.00 (s, 1H), 1.163 (s, 3H), 1.062 (s, 3H).

### Example 54C

### Preparation of Compound (61c)

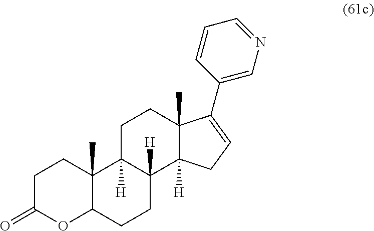

To a solution of 3-((3aS,5aS,6R, 9aS,9bS)-3a,6-dimethyl-7-oxo-3-(pyridin-3-yl)-3a,4,5, 5a,6, 7, 8, 9, 9a,9b-decahydro-1H-cyclopenta[α]naphthalen-6-yl)propanoic acid, compound (61b) [compound (1d)] (50 mg, 0.136 mmol) in Ac₂O (2 ml) was added solid NaOAc (22.2 mg, 0.163 mmol). The reaction mixture was refluxed overnight. The reaction was monitored by TLC and the starting material was completely consumed. The mixture was cooled to room temperature and concentrated. The residue was added DCM (10 ml) and saturated aqueous Na₂CO₃ (2 ml). The layers were separated and the aqueous was extracted with DCM (3×10 mL). The combined organic layers were washed with brine, dried over Na₂SO₄. The solution was concentrated under vacuum to afford (4aR,4bS,6aS,9aS,9bS)-4a,6a-dimethyl-7-(pyridin-3-yl)-4,4a,4b,5, 6, 6a,9, 9a,9b,10-decahydroindeno[5,4-f]chromen-2 (3H)-one, compound (61c) (25 mg). MS calcd for (C₂₃H₂₇NO₂) [2M+Na]+721.94. Found: 722.1. ¹H NMR (CDCl₃, 300 MHz): δ 8.61 (s, 1H), 8.46 (brs, 1H), 7.64 (d, 1H), 7.22 (m, 1H), 6.00 (s, 1H), 5.30 (s, 1H), 2.65 (m, 2H), 2.01-2.35 (m, 4H), 1.4-1.95 (m, 8H), 1.31 (m, 1H), 1.16 (s, 3H), 1.06 (s, 3H).

### Example 54D

### Preparation of Compound (61d)

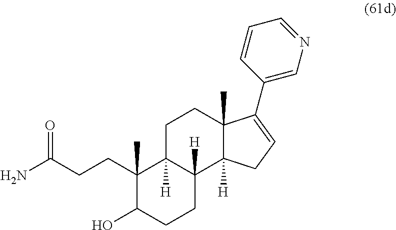

Dry NH₃ gas was bubbled into a solution of compound (61c) (6.34 g) in EtOH (100 ml) for 2 h at 0° C. The resulting solution was slowly warmed to room temperature and stirred under sealed condition until all the starting material was consumed by TLC analysis. The solution was concentrated in vacuo to provide 6.5 g (97.74%) of compound (61d). ¹H NMR (DMSO-d₆, 300 MHz): 8.65 (1H), 8.5 (1H), 7.8 (1H), 7.35 (1H), 7.25 (1H), 6.75 (1H), 6.15 (1H), 4.45 (1h), 3.2-3.3 (1H), 2.2 (1H), 1.95-2.1 (4H), 1.05 (3H), 0.85 (3H). ¹³C NMR (DMSO-d₆, 300 MHz): 175.94, 151.53, 148.25, 147.60, 133.78, 132.65, 129.39, 123.85, 71.60, 57.43, 56.51, 47.29, 45.95, 19.01, 15.53; MS calculated for (C₂₃H₃₂N₂O₂) [M+H]+ 369.51 Found: 369.4; [2M+H]+ 738.02 Found: 738.0.

### Example 54E

### Preparation of Compound (61e)

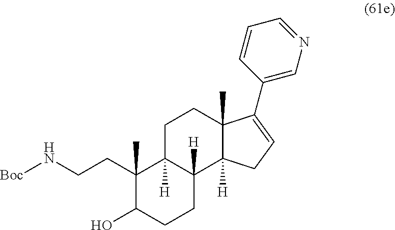

A solution of compound (61d) (7.5 g, 20.35 mmol) in MeCN-water (250 ml, 1:1) was treated with phenyliodine (III) diacetate (8.4 g) at 0° C. The reaction was stirred for 40 min and the starting material was completely consumed by TLC analysis. The solution was basified with NaHCO₃ (5.5 g, 61.05 mmol) and di-tert-butyl dicarbonate (4.7 g) was added. The resulting solution was heated at 50° C. for 4 h. The volatile solvent was removed in vacuo. The aqueous solution was extracted with ethyl acetate (3×70 ml). The combined organic layers were washed with water (150 ml) and brine (150 ml), dried over anhydrous Na₂SO₄, and evaporated to dryness to give crude compound (61e) (7.5 g, 83.7%). ¹H NMR (DMSO-d₆, 300 MHz): 8.6 (1H), 8.42 (1H), 7.6 (1H), 7.2 (1H), 5.95 (1H), 3.5-3.8 (1H), 2.95-3.0 (2H), 0.95 (3H), 0.85 (3H). ¹³C NMR (DMSO-d₆, 300 MHz): 156.39, 151.57, 147.80, 133.74, 132.96, 129.16, 123.05, 79.27.

### Example 54F

### Preparation of Compound (61f)

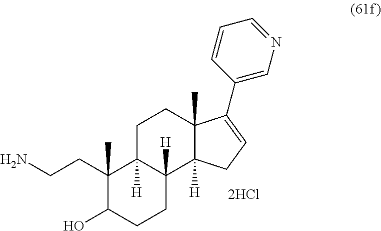

The crude compound (61e) (1.5 g, 3.4 mmol) was treated with a solution of HCl in methyl acetate [prepared by the slow addition of acetic chloride (7.065 g, 0.09 mol) to anhydrous MeOH (3.2 g, 0.1 mol) at 0° C.]. The solution was stirred for 10 min at room temperature and a precipitate was formed. The solid was collected by vacuum filtration, washed with EtOAc (10 ml) and dried under vacuum to provide 1.3 g (100%) of compound (61f) as di-hydrochloride salt. ¹H NMR (DMSO-d₆, 300 MHz): 8.8 (1H), 8.65 (1H), 8.55 (1H), 7.95 (1H), 6.35 (1H), 3.15-3.35 (1H), 2.65-2.75 (2H), 0.9 (3H), 0.75 (3H). MS calculated for (C₂₂H₃₂N₂O) [M+H]+ 341.50 Found: 340.7.

### Example 54G

### Preparation of Compound (61)

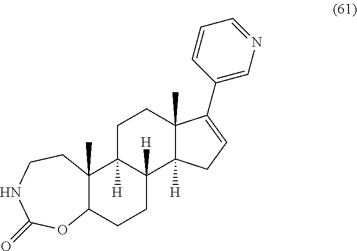

To a solution of compound (61f) di-hydrochloride salt (0.3 g, 0.8 mmol) and triethylamine (0.45 g, 4 mmol) in anhydrous tetrahydrofuran (40 ml) was added a solution of triphosgene (0.24 g, 0.8 mmol) in tetrahydrofuran (10 ml), followed by the treatment with 4-dimethylaminopyridine (20 mg). The resulting solution was stirred overnight at room temperature. The volatile solvent was removed in vacuo. Water (20 ml) and ethyl acetate (20 ml) were added and the layers were separated. The aqueous layer was extracted with ethyl acetate (20 ml). The combined layers were washed with brine (2×20 ml), dried over Na₂SO₄, and evaporated to dryness. The residue was purified by column chromatograph (silica gel) to afford a mixture of α- and β-epimers of compound (61) as a white solid (58 mg, 37%). Retention time at HPLC: 8.587 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)] ¹H NMR (DMSO-d₆, 300 MHz): 8.6 (1H), 8.45 (1H), 7.75 (1H), 7.32 (1H), 7.11 (1H), 6.10 (1H), 3.7-4.0 (1H), 3.1-3.15 (1H), 2.75-2.85 (1H), 2.1-2.2 (1H), 1.95-2.1 (2H), 0.9-1.1 (6H). MS calculated for (C₂₃H₃₀N₂O₂) [M+H]+ 367.50 Found: 367.7; [M+Na]+389.50 Found: 389.6; [2M+H]+ 734.0 Found: 734.6; [2M+Na]+756.0 Found: 755.8

### Example 55

### Preparation of Compound (62)

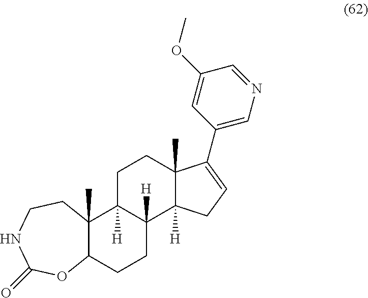

Using a synthetic procedure and condition similar to Examples 54A in the preparation of compound (61a), and replacing diethyl (3-pyridyl)borane with 5-methoxypyridin-3-ylboronic acid and subjecting the product to reactions and conditions similar to Examples 54B to Example 54G, a mixture of α- and β-epimers of compound (62) are made.

### Example 56

### Preparation of Compound (63)

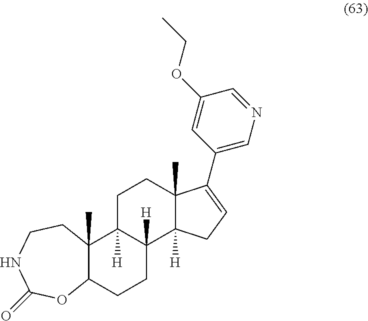

### Example 56A

### Preparation of Compound (63a)

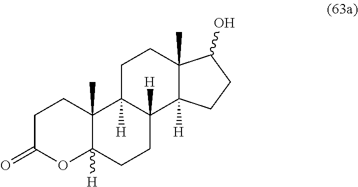

To a solution of compound (1a) (7.2 mmol) in THF (15 ml) was added NaBH₄ (805 mg) at 0° C. under stirring, followed by dropwise addition of MeOH. The mixture was stirred until all the starting material was consumed by TLC analysis. The organic solvent was remove under reduce pressure, the residue was suspended in EtOAc and washed with water, the aqueous was reextracted with EtOAc (2×50 ml). The combined organic phases were washed with water and brine, dried over anhydrous sodium sulfate, and evaporated to afford the di-alcohol acid. A solution of this dialcohol acid compound and p-TsOH in toluene was heated at reflux for 30 min, and all the starting material was consumed as indicate by TLC analysis. The solvent was removed under reduce pressure. The residue was diluted with dichloromethane (50 ml) and filtered though a pad of silica gel. The filtrate was concentrated in vacuo to provide compound (63a) which was used in the next step without further purification.

### Example 56B

### Preparation of Compound (63b)

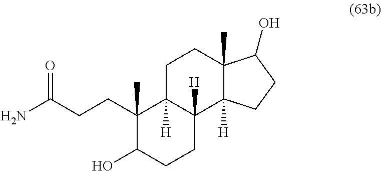

Compound (63a) (17.6 g) was treated with saturated methanolic NH₃ in sealed condition at 0° C. The reaction was stirred overnight at room temperature and the starting material was completely consumed as indicated by TLC analysis (EtOAc:Hexane=1:3). The solution was concentrated under reduced pressure to give 18g (97%) of compound (63b) which was used in next step without further purification.

### Example 56C

### Preparation of Compound (63c)

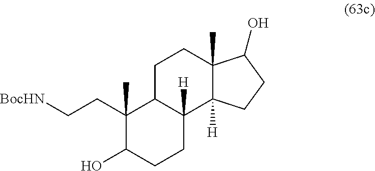

A solution of compound (63b) (18 g, 59.79 mmol) in MeCN/H₂O (700 mL, 1/1) was treated with phenyliodonium diacetate (20.92 g) in portions at 0° C. After addition, the reaction was stirred for additional 1.5 h at room temperature and all the starting material was consumed by TLC analysis (MeOH/EtOAc=1/10). The volatile solvents were removed under reduced pressure. The aqueous solution was washed with EtOAc (2*100 mL), followed by addition of THF (300 mL), NaHCO₃ (12.56 g), and treated with (Boc)₂O (15 g) in an ice-water bath. The reaction mixture was stirred overnight at room temperature and all the starting material was consumed. The volatile solvents were evaporated with a rotary evaporator. The aqueous solution was extracted with EtOAc (2*650 mL). The combined organic solution was washed with water (200 mL), brine (300 mL) and dried over Na₂SO₄. The solution was evaporated to dryness to afford 20 g (90%) of compound (63c) as a white solid. ¹H NMR (CDCl₃, 400 MHz): 4.6-4.9 (1H), 3.4-3.7 (2H), 2.9-3.2 (2H), 1.4 (9H), 0.85 (3H), 0.72 (3H).

### Example 56D

### Preparation of Compound (63d)

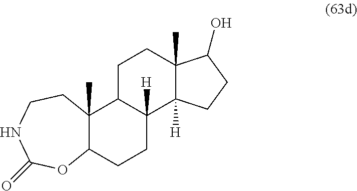

Compound (63c) (2.0 g) was treated with methanolic HCl (prepared from acetic chloride (4 g) and MeOH (2.5 mL)). The formed precipitate was collected and dried in vacuo to give 1 g of HCl salt. ¹H NMR (DMSO, 400 MHz): 3.7 (1H), 3.47 (1H), 3.0 (2H), 2.01 (1H), 0.91 (3H), 0.75 (3H). 319 mg of HCl salt was dissolved in THF/H₂O (14 mL, 5/2) and treated dropwise with a solution of triphosgene (0.52 g) in THF (5 mL) in the presence of DBU [1,8-Diazabicyclo[5.4.0]undec-7-ene] (240 mg). The volatile solvents were evaporated with a rotary evaporation. The aqueous solution was extracted with EtOAc (3*15 mL). The combined organic solution was washed with water (20 mL) and brine (20 mL), and evaporated to dryness. The residue was purified by flash column chromatograph (silica gel, eluent: EtOAc) to provide 150 mg (38.8%) of the compound (63d). ¹H NMR (DMSO, 400 MHz): 7.12 (1H, s), 4.47 (1H, s), 3.75 (1H, m), 3.09 (1H, m), 2.82 (1H, m), 1.82 (1H, m), 1.4-1.8 (7H, m), 1.1-1.4 (6H, m), 0.94 (1H, m), 0.7-1.0 (6H, m), 0.66 (3H, s).

### Example 56E

### Preparation of Compound (63e)

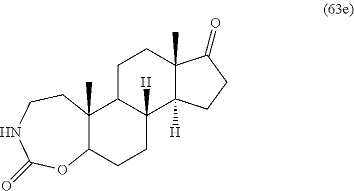

A solution of compound (63d) (900 mg, 2.93 mmol) in CH₂Cl₂ (100 mL) was treated with Dess-Martin Periodinate (2 g, 4.72 mmol) at room temperature. The reaction was stirred at room temperature until all the starting material was consumed as indicated by TLC analysis (EtOAc:Hexane=2:1). The reaction was quenched with 10% aqueous Na₂SO₃ solution (100 mL). The organic layer was separated and the aqueous layer was extracted with CH₂Cl₂ (50 mL). The combined organic layers were washed with 10% aqueous Na₂SO₃ solution (3*20 mL), brine (50 mL), dried over anhydrous sodium sulfate, and evaporated to dryness. The residue was purified by flash column chromatograph on silica gel (eluent: EtOAc/Hexane=2/1) to afford 675 mg (75%) of the compound (63e). ¹H NMR (CDCl₃, 400 MHz): 5.65 (1H, br), 3.86 (1H, m), 3.32 (1H, m), 2.98 (1H, m), 2.49 (1H, m), 1.0 (3H, s), 0.88 (3H, s). ¹³C NMR (CDCl₃, 400 MHz): 161.40, 86.09, 51.39, 50.81, 47.57, 13.72, 12.00.

### Example 56F

### Preparation of Compound (63f)

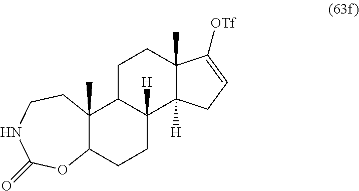

A solution of compound (63e) (508 mg, 1.66 mmol) in dichloromethane (5.5 mL) was treated with Tf₂O (784.5 mg, 2.5 mmol) at 0° C. After 10 min stirring, a solution of triethylamine (168.5 mg, 1.67 mmol) in CH₂Cl₂ (5.5 mL) was added dropwise. The reaction was monitored by TLC (EtOAc/Hexane=1/1). Silica gel (1 g) was added to the solution and evaporated to dryness. Compound (630 (257 mg, 35.4%) was isolated by column chromatograph on silica gel (eluent: EtOAc/hexane=1/1). ¹H NMR (CDCl₃, 400 MHz): 5.5-5.6 (2H), 3.8 5 (1H, m), 3.34 (1H, m), 2.98 (1H, m), 2.25 (2H, m), 0.95-1.02 (6H).

### Example 56G

### Preparation of Compound (63)

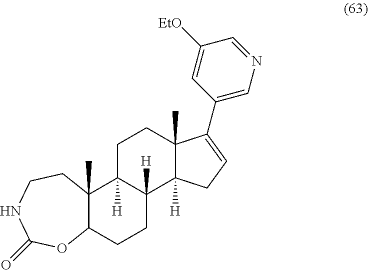

To a solution of compound (63f) (83 mg) in THF (10 mL) were added 2N aqueous Na₂CO₃ solution (1.0 mL), 5-ethyloxypyridin-3-ylboronic acid (110 mg, 0.606 mmol) and bis(triphenylphosphine)palladium (II) chloride (2.0 mg, 2.28*10⁻³ mmol). The mixture was degassed by Ar gas flushing for 50 min. The resulted mixture was heated at 80-90° C. under Ar gas atmosphere overnight and all the starting material was consumed by TLC analysis (hexane: EtOAc=1:1). The volatile solvents were removed in vacuo and the residue was partitioned between water (10 mL) and EtOAc (25 mL). The aqueous layer was extracted with EtOAc (25 mL). The combined organic layers were washed with brine (25 mL), dried over anhydrous sulfate sodium, and evaporated to dryness. The residue was purified by preparative HPLC to give 10.5 mg of compound (63). Ret. time at HPLC: 10.204 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 MHz): 8.37 (1H, s), 8.16 (1H, s), 7.10 (1H, s), 6.0 (1H, s), 5.2 (1H, d), 4.10 (2H, m), 3.87 (1H, m), 3.35 (1H, m), 2.95 (1H, m), 2.25 (1H, m) 1.05 (3H, s), 0.99 (3H, s). ¹³C NMR (CDCl₃, 400 MHz): 160.31, 150.33, 139.31, 134.59, 128.34, 118.41, 85.47, 62.89, 55.81, 50.57, 46.38, 39.52, 39.21, 37.74, 34.16, 31.90, 30.64, 28.50, 27.59, 20.11, 15.66, 13.79, 13.31, 10.95 ESI-MS: m/z calcd for C₂₅H₃₄N₂O₃: [M+H]+ 411.55. Found: 411.3.

### Example 57

### Preparation of Compound (64)

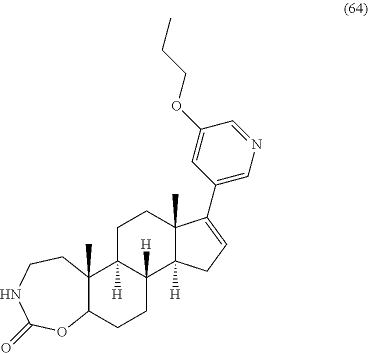

Using a synthetic procedure and condition similar to Examples 56G in the preparation of compound (63), and replacing 5-ethyloxypyridin-3-ylboronic acid with 5-propoxypyridin-3-ylboronic acid, a mixture of α- and β-epimers of compound (64) are made.

### Example 58

### Preparation of Compound (65)

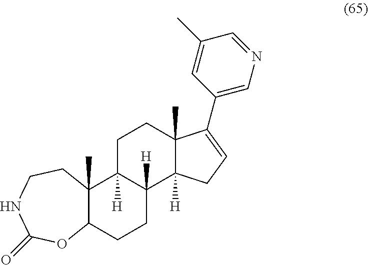

Using a synthetic procedure and condition similar to Examples 56G in the preparation of compound (63), and replacing 5-ethyloxypyridin-3-ylboronic acid with 5-methylypyridin-3-ylboronic acid, a mixture of α- and β-epimers of compound (65) were made. Ret. time at HPLC: 9.060 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.42 (s, 1H), 8.25 (s, 1H), 7.48 (s, 1H), 5.95 (s, 1H), 5.55 (s, 1H), 3.90 (m, 1H), 3.31 (m, 1H), 2.98 (m, 1H), 2.70 (br, 1H), 2.35 (s, 3H), 2.24 (m, 1H), 1.9-2.1 (m, 3H), 1.7-1.85 (m, 3H), 1.3-1.7 (m, 7H), 1.25 (s, 1H), 1.01 (s, 3H), 0.98 (s, 3H). ¹³C NMR (400 MHz, CDCl₃): 160.50, 150.30, 146.76, 143.31, 133.94, 131.81, 128.34, 85.45, 55.80, 50.51, 46.33, 39.45, 37.64, 34.12, 31.81, 30.63, 28.69, 28.48, 27.57, 20.07, 17.47, 15.62, 10.95. ESI-MS: m/z calcd for C₂₄H₃₂N₂O₂: [M+H]+ 381.52. Found: 381.6.

### Example 59

### Preparation of Compound (66)

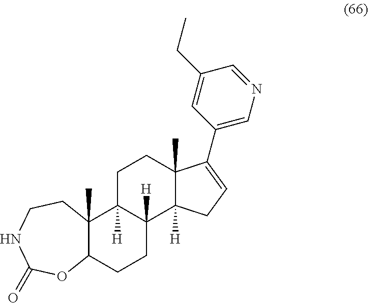

Using a synthetic procedure and condition similar to Examples 56G in the preparation of compound (63), and replacing 5-ethyloxypyridin-3-ylboronic acid with 5-ethylpyridin-3-ylboronic acid, a mixture of α- and β-epimers of compound (66) are made.

### Example 60

### Preparation of Compound (67)

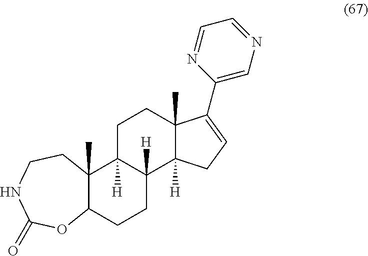

Using a synthetic procedure and condition similar to Examples 56G in the preparation of compound (63), and replacing 5-ethyloxypyridin-3-ylboronic acid with 2-(tributylstannyl)pyrazine, a mixture of α- and β-epimers of compound (67) are made.

### Example 61

### Preparation of Compound (68)

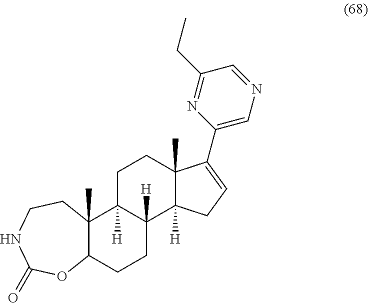

Using a synthetic procedure and condition similar to Examples 56G in the preparation of compound (63), and replacing 5-ethyloxypyridin-3-ylboronic acid with 2-(tributylstannyl)-6-ethylpyrazine, a mixture of α- and β-epimers of compound (68) are made.

### Example 62

### Preparation of Compound (69)

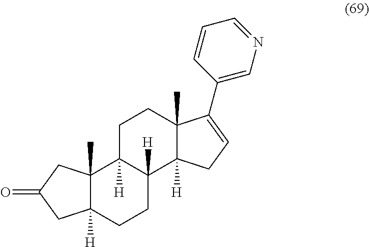

### Example 62A

### Preparation of Compound (69a)

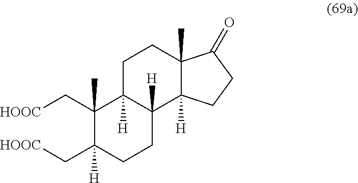

Epiandrosterone (50 g, 0.1722 mol) was dissolved in glacial acetic acid (250 mL) and heated to 75° C. To the resulting solution was added dropwise with a solution of CrO₃ (23 g) in H₂O (160 mL) and sulfuric acid (34 mL) at this temperature. After addition, the mixture was heated to 90-100° C. and stirred for additional 2 h. The reaction was monitored by TLC analysis (Hexane/EtOAc=3/1, 5% HOAc). The mixture was cooled to room temperature with ice water bath and extracted with EtOAc (2×500 mL). The combined organic solution was concentrated to dryness in vacuo. The residue was then dissolved in 5N aqueous NaOH (1.5 L) and washed with t-butyl methyl ether (2×500 mL). The aqueous solution was acidified to PH 2 with 10N aqueous HCl. The product was extracted with t-butyl methyl ether (2×500 mL). The combined organic layers were washed with water and brine, dried over anhydrous Na₂SO₄, filtered, and evaporated in vacuo. The residue was purified by column chromatograph (silica gel) to afford 33.6 g (58%) of compound (69a) as a white powder. ¹H NMR (DMSO, 300 MHz): 11.6-11.9 (2H), 2.2-2.3 (2H), 1.9-2.2 (3H), 1.75-1.85 (2H), 0.85-1.0 (6H). ¹³C NMR (DMSO, 300 MHz): 174.31, 172.35, 50.70, 47.93, 46.75, 35.36, 34.32, 15.35, 13.30 .

### Example 62B

### Preparation of Compound (69b)

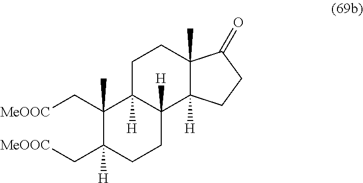

Compound (69a) (5 g) was dissolved in methanol (75 mL) and treated with SOCl₂ (8.8 g) at 5° C. After addition, the mixture was heated at reflux for 2 h and all the starting material was consumed by TLC analysis (Hexane/EtOAc=3/1, 5% HOAc). The volatile solvent was removed under reduce pressure. The residue was then dissolved in t-butyl methyl ether (50 mL), washed with diluted NaHCO₃ (2×25 mL) and brine (25 mL), dried over anhydrous Na₂SO₄, and evaporated to afforded 4.8 g (88.6%) of compound (69b) as a white powder. ¹H NMR (CDCl₃, 300 MHz): 3.3.6-3.7 (6H), 3.6-3.65 (1H), 2.3-2.45 (2H), 2.2-2.25 (1H), 0.75-0.85 (6H).

### Example 62C

### Preparation of Compound (69c)

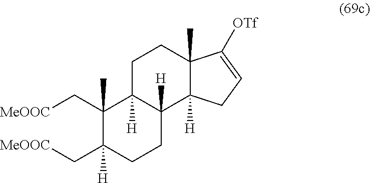

To a solution of compound (69b) (3.2 g) in dichloromethane (30 mL) was added dropwise with Tf₂O (3.7 g) at 5° C., followed by addition of Et₃N (0.8 g). The mixture was warmed to room temperature and stirred overnight. The reaction was monitored by TLC analysis (hexane: EtOAc=3:1) and all the starting material was consumed. The reaction was quenched with water (20 mL). The mixture was extracted with dichloromethane (2×20 mL). The combined organic layers were washed brine (20 mL), dried over anhydrous Na₂SO₄, and evaporated to dryness. The residue was purified by column chromatograph (silica gel) to give 4.1 g (94%) of compound (69c). ¹H NMR (CDCl₃, 300 MHz): 5.55 (1H), 3.3.6-3.7 (6H), 3.6-3.65 (1H), 3.3-3.4 (1H), 2.1-2.3 (2H), 0.95 (3H), 0.85 (3H). ¹³C NMR (CDCl₃, 300 MHz): 173.74, 171.36, 159.10, 120.13, 116.94, 114.38, 53.93, 14.03, 13.90.

### Example 62D

### Preparation of Compound (69d)

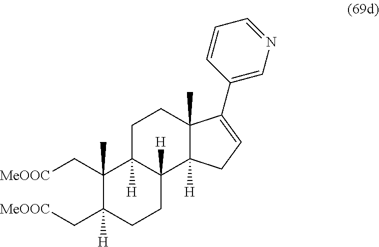

To a solution of compound (69c) (4.56 g) in THF/H₂O (30 mL, 1/1) were added solid Na₂CO₃ (2 g), pyridin-3-ylboronic acid (2.3 g) and bis(triphenylphosphine)palladium (II) chloride (0.3 g). The mixture was degassed by Argon gas flushing for 2 h. The resulted mixture was heated at reflux overnight under Argon and all the starting material was consumed by TLC analysis (hexane: EtOAc=4:1). The volatile solvent was removed in vacuo and the product was extracted with EtOAc (2×50 mL). The combined organic layers were washed with brine (25 mL), dried over anhydrous sulfate sodium, and evaporated to dryness. The residue was purified by column chromatograph on silica gel to give 3.3 g (84.4%) of compound (69d). ¹H NMR (CDCl₃, 300 MHz): 8.6 (1H), 8.4 (1H), 7.6 (1H), 7.12 (1H), 5.95 (1H), 2.6-2.7 (1H), 2.4-2.5 (1H), 2.1-2.2 (2H), 1.95-2.05 (4H), 1.89 (1H), 0.98 (3H), 0.81 (3H).

### Example 62E

### Preparation of Compound (69e)

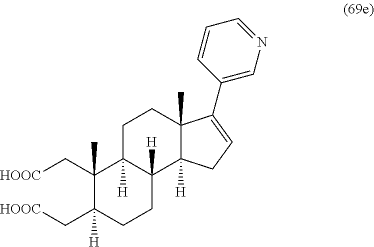

A solution of compound (69d) (600 mg) in THF (10 mL) was treated with a solution of NaOH (0.28 g) in water (10 mL). The mixture was stirred over overnight at room temperature and all the starting material was consumed. The volatile solvent was removed in vavuo. The PH of the aqueous solution was adjusted to 6-7 and the product was precipitated out. The resulting solid was filtered and dried under vacuo to provide 600 mg of compound (69e).

### Example 62F

### Preparation of Compound (69)

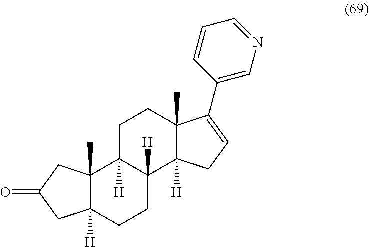

A solution of compound (69e) (340 mg) in acetic anhydride (5 mL) was treated with sodium acetate (70 mL). The resulting solution was heated at reflux for 3.5 h and all the starting material was consumed by TLC analysis (hexane: EtOAc=3:1). The mixture was distilled under reduce pressure at 60-80° C. to removed the acetic anhydride. The residue was then dissolved in EtOAc (20 mL), washed with saturated aqueous NaHCO₃ (20 mL) and brine (20 mL), dried over magnesium sulfate, and evaporated to dryness. The residue slurry (240 mg) was purified by preparative RP-HPLC to afford 35 mg of compound (69) as a white powder. Retention time at HPLC: 11.279 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ESI-MS: m/z calcd for C₂₃H₂₉NO [M+H]+ 336.48 Found: 336.2; ¹H NMR (CDCl₃, 300 MHz): 8.62 (1H), 8.55 (1H), 7.7 (1H), 7.18 (1H), 6.0 (1H), 1.05 (3H), 0.95 (3H). ¹³C NMR (CDCl₃, 300 MHz): 151.79, 147.97, 133.71, 129.14, 123.06, 57.20, 54.40, 53.81, 47.88, 16.82, 13.73.

### Example 63

### Preparation of Compound (70)

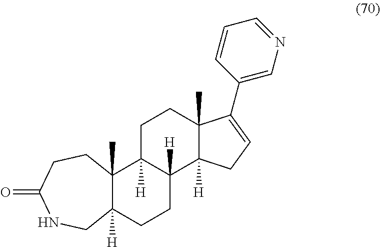

### Example 63A

### Preparation of Compound (70a) and Compound (71a)

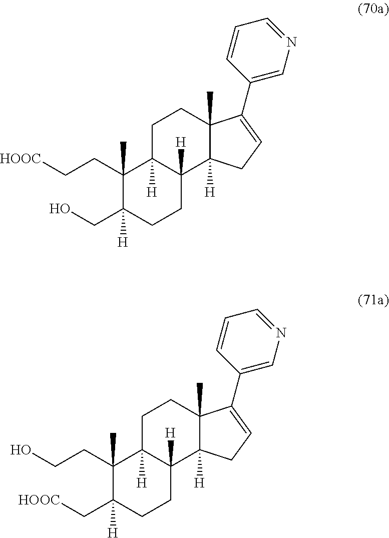

A solution of the mixture of compound (45) and compound (46) (3 g, 8.2 mmol) was treated with 8N aqueous NaOH (10 ml) at room temperature. The reaction was stirred for 1 h and all the starting material was consumed. The volatile solvent was removed in vacuo. The aqueous solution was neutralized with 5N aqueous HCl to PH7 and a precipitate was formed. The solid was collected by filtration and dried under vacuo, yielding 2.79 g of the mixture of compound (70a) and compound (71a). ¹H NMR (MeOD, 300 MHz): 8.47 (1H), 8.3 (1H), 8.71 (1H), 5.98 (1H), 3.71 (0.636H), 3.60 (0.354H), 3.50 (0.352H), 3.15 (0.620H), 1.95-2.24 (5H), 0.98 (3H), 0.81 (3H). ESI-MS: m/z calcd for C₂₄H₃₃NO₃ [M+H]+ 384.52 Found: 385.2; [2M+H]+ 768.04 Found: 767.9

### Example 63B

### Preparation of Compound (70b) and Compound (71b)

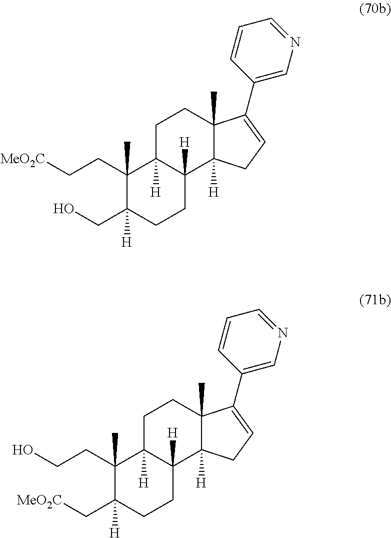

A solution of the mixture of compound (70a) and compound (71a) (2.79 g, 7.27 mmol) in anhydrous methanol (20 ml) was treated with SOCl₂ (0.63 ml, 8.72 mmol) at 0° C. The resulting mixture was stirred overnight and all the starting material was consumed. The volatile solvent was removed in vacuo. The residue was dissolved in dichloromethane (20 ml) and basified with saturated aqueous NaHCO₃ (15 ml). The organic layer was separated and the aqueous solution was extracted with dichloromethane (3×10 ml). The combined organic layers were washed with brine (20 ml), dried over Na₂SO₄, and evaporated to dryness. The resulting residue was purified by column chromatograph on silica gel (hexane: EtOAc=2:1-1:1) to isolate 1.21 g of compound (70b) and 0.60 g of compound (71b). Compound (70b): ¹H NMR (CDCl₃, 300 MHz): 8.61 (1H), 8.45 (1H), 7.61 (1H), 7.21 (1H), 5.99 (1H), 3.82 (1H), 3.65 (3H), 3.31 (1H), 0.98 (3H), 0.86 (3H). Compound (71b): ¹H NMR (CDCl₃, 300 MHz): 8.61 (1H), 8.45 (1H), 7.62 (1H), 7.21 (1H), 6.0 (1H), 3.65 (3H), 3.6-3.7 (2H), 1.0 (3H), 0.89 (3H).

### Example 63C

### Preparation of Compound (70c)

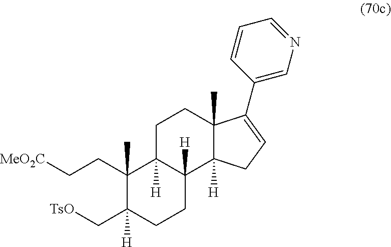

A solution of compound (70b) (1.10 g, 2.77 mmol) in anhydrous pyridine (20 ml) were treated with tosyl chloride (1.06 g, 5.53 mmol) and 4-dimethylaminopyridine (70 mg, 0.55 mmol) at 0° C. After stirring for additional 30 min at 0° C., the mixture was warmed to room temperature and stirred overnight. The reaction was monitored by TLC analysis (hexane: EtOAc=4:1). The solvent was removed in vacuo. The residue was suspended in water (20 ml) and extracted with EtOAc (3×10 ml). The combined organic layers were washed with saturated aqueous NaHCO₃, dried (Na₂SO₄), and evaporated to dryness. The residue was purified by column chromatograph on silica gel to afford 978 mg (64%) of the pure compound (70c). ¹H NMR (CDCl₃, 300 MHz): 8.61 (1H), 8.45 (1H), 7.8 (2H), 7.65 (1H), 7.45 (2H), 7.21 (1H), 6.0 (1H), 3.8-4.1 (2H), 3.65 (3H), 2.5 (2H), 0.98 (3H), 0.81 (3H).

### Example 63D

### Preparation of Compound (MD)

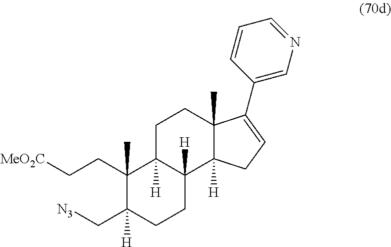

Compound (70c) (680 mg, 1.23 mmol) was dissolved in anhydrous dimethylformamide (15 ml) and treated with sodium azide (640 mg, 9.84 mmol). The reaction was stirred at 50° C. overnight and all the starting material was consumed by TLC analysis. The mixture was diluted with H₂O (10 ml) and extracted with EtOAc (3×10 ml). The combined organic layers were washed with saturated aqueous NH₄Cl (2×10 ml) and brine (10 ml), dried (Na₂SO₄), and evaporated to dryness. The residue was purified by column chromatograph (silica gel) to give 484 mg (93%) of compound (70d). ¹H NMR (CDCl₃, 300 MHz): 8.61 (1H), 8.45 (1H), 7.65 (1H), 7.21 (1H), 6.0 (1H), 3.72 (3H), 3.61 (1H), 3.0 (1H), 1.91-2.31 (7H), 1.02 (3H), 0.95 (3H).

### Example 63E

### Preparation of Compound (70)

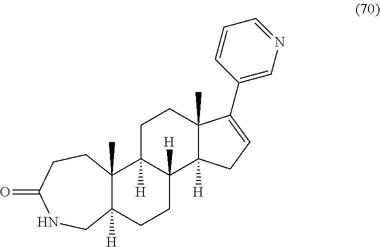

To a degassed solution of compound (70d) (220 mg, 0.52 mmol) in anhydrous THF (5 ml) was added tributylphosphine (211 mg, 1.04 mmol) under Argon gas. After heating at reflux for 4 h, water (0.2 ml) was added and the resulting mixture was continued to reflux overnight. The mixture was cooled to room temperature and acidified to PH 1 with 5% aqueous HCl. The resulting mixture was stirred for additional 30 min, and then the solution was basified to PH 8 with saturated aqueous NaHCO₃. The product was extracted with EtOAc (3×10 ml). The combined organic layers were washed brine (15 ml), dried over Na₂SO₄, and evaporated to dryness. The residue (180 mg) was purified by preparative R^{P}HPLC to afford the pure compound (70) as a white solid. Retention time at HPLC: 9.291 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃, 300 MHz): 8.61 (1H), 8.45 (1H), 7.67 (1H), 7.21 (1H), 6.25 (1H), 5.95 (1H), 3.41 (1H), 2.56 (1H), 0.98-1.05 (6H). ¹³C NMR (CDCl₃, 300 MHz): 177.57, 150.56, 146.84, 132.65, 131.84, 128.09, 122.02, 56.25, 52.99, 48.77, 46.22, 43.46, 15.62, 11.02

### Example 64

### Preparation of Compound (71)

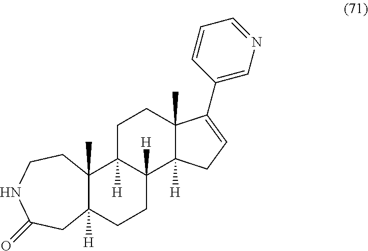

### Example 64A

### Preparation of Compound (71c)

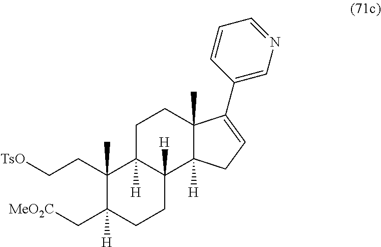

Using a synthetic procedure and condition similar to Examples 63C in the preparation of compound (70c), and replacing compound (70b), with compound (71b), compound (71c) is made.

### Example 64B

### Preparation of Compound (71d)

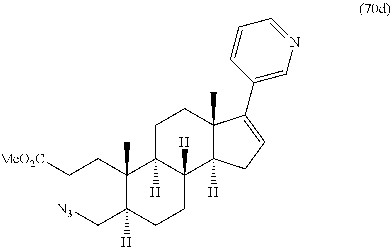

Using a synthetic procedure and condition similar to Examples 63D in the preparation of compound (70d), and replacing compound (70c), with compound (71c), compound (71d) is made. Compound (71d): ¹H NMR (CDCl₃, 300 MHz): 8.61 (1H), 8.45 (1H), 7.62 (1H), 7.21 (1H), 5.98 (1H), 3.71 (3H), 3.1-3.3 (2H), 1.97-2.1 (4H), 1.02 (3H), 0.91 (3H).

### Example 64C

### Preparation of Compound (71)

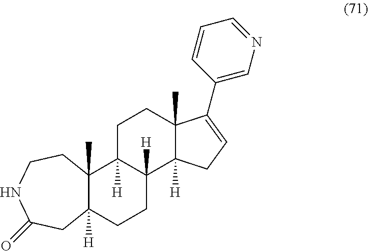

Using a synthetic procedure and condition similar to Examples 63E in the preparation of compound (70), and replacing compound (70d), with compound (71d), compound (71) is made. Compound (71): Retention time at HPLC: 9.147 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃, 300 MHz): 8.58 (1H), 8.44 (1H), 7.62 (1H), 7.20 (1H), 6.34 (1H), 5.95 (1H), 3.3-3.4 (1H), 2.9-3.1 (1H), 2.7-2.8 (1H), 1.9-2.3 (5H), 0.95-1.1 (6H). ¹³C NMR (CDCl₃, 300 MHz): 171.56, 150.51, 146.85, 146.81, 132.66, 131.87, 128.21, 122.02, 56.25, 53.28, 46.17, 42.41, 40.79, 15.62, 10.91. ESI-MS: m/z calcd for C₂₄H₃₂N₂O [M+H]+ 365.52 Found: 365.5

### Example 65

### Preparation of Compound (72A) and Compound (72B)

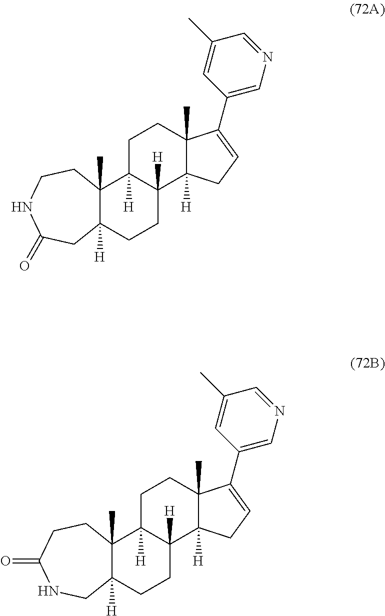

### Example 65A

### Preparation of Compound (72a)

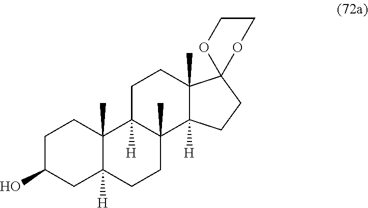

A solution of epiandrosterone (50 g, 172.2 mmol), ethylene alcohol (32 g, 516.4 mmol) and p-toluenesulfonic acid (0.88 g) in toluene (400 mL) were refluxed under a Dean-Stark trap over night. The mixture was cooled to room temperature and concentrated to dryness. The residue was diluted with EtOAc (1000 mL), washed with sat. NaHCO₃ (2*500 mL) and brine (500 mL). The organic phase was dried over anhydrous sodium sulfate, filtered, and evaporated to afford 54.6 g (94.8%) of compound (72a). ¹H NMR (400 MHz, CDCl₃): 3.85-4.0 (4H), 3.75 (1H), 3.56 (1H), 0.9-0.95 (6H).

### Example 65B

### Preparation of Compound (72b)

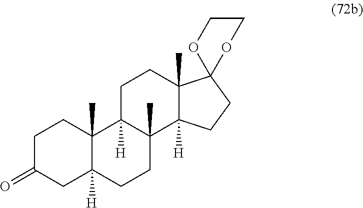

A solution of compound (72a) (30 g, 89.7 mmol) in dichloromethane (400 mL) was treated with Dess-Martin Periodinane (54.7 g, 129.1 mmol) at room temperature. The reaction was stirred until all the start material was consumed. The mixture was washed with sat.NaHCO₃ solution (2*200 mL), sat.Na₂SO₃ solution (200 mL) and brine (200 mL). The solution was dried over anhydrous sodium sulfate, filtered, and evaporated to dryness to afford 30 g (100%) of compound (72b). NMR (400 MHz, CDCl₃): 3.7-4.1 (4H), 2.2-2.5 (3H), 1.9-2.1 (3H), 1.05 (3H), 0.91 (3H). ¹³C NMR (400 MHz, CDCl₃): 211.84, 119.27, 65.16, 64.49, 53.58, 50.10, 14.37, 11.43.

### Example 65C

### Preparation of Compound (72c)

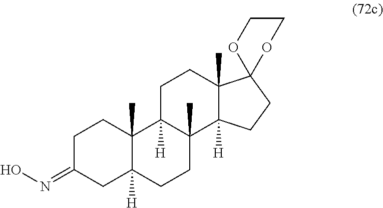

A solution of compound (72b) (30 g, 90.2 mmol) in EtOH (400 mL) was treated with NH₂OH.HCl (13 g, 180.5 mmol) and pyridine (16 mL, 180.5 mmol). The resulting mixture was stirred for 1 h at room temperature. The volatile solvents were removed under reduced pressure. The residue was dissolved in 500 mL of dichloromethane and washed with water (300 mL*2). The organic solution was evaporated to dryness to afford 27 g (86%) of compound (72c), which was used in the next step without further purification.

### Example 65D

### Preparation of Compound (72d)

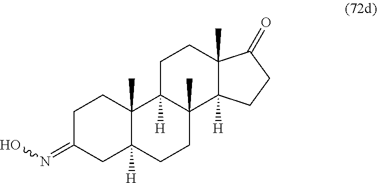

A solution of compound (72c) (22.7 g, 63.5 mmol) in THF (200 mL) was treated with 2N aqueous HCl (10 mL). The resulting mixture was stirred at room temperature until all the starting material was consumed (Hexane/EtOAc=4/1). The volatile solvents were removed under reduced pressure. The residue was dissolved in dichloromethane (500 mL) and washed with water (2*250 mL). The organic solution was evaporated to dryness and purified by column chromatograph (silica gel, hexhane/EtOAc=6/1-MeOH) to afford 16.7 g (84%) of compound (72d). ESI-MS: m/z calcd for C₁₉H₂₉NO₂ [M+Na]+326.4. Found: 326.6; [2M+Na]+629.8. Found: 629.7.

### Example 65E

### Preparation of Compound (72e) and Compound (72f)

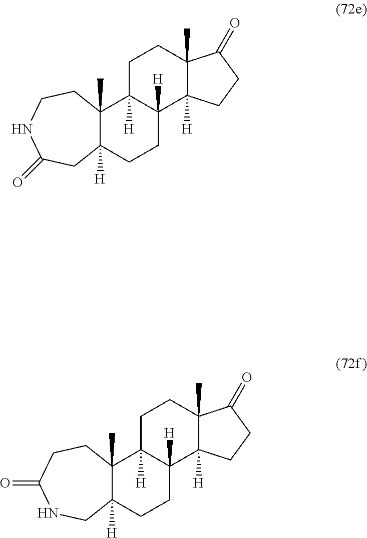

A solution of compound (72d) (16.7 g, 55 mmol) in dioxane (200 mL) was added dropwise with SOCl₂ (26.73 mL) at 0° C. The resulting mixture was stirred for 2 hour and the starting material was completely consumed (hexane/EtOAc=4/3). The solvent was removed under reduced pressure. The residue was dissolved in dichloromethane (500 mL), washed with water (2*250 mL) and diluted NaHCO₃ (250 mL), and evaporated to dryness. The residue was purified by column chromatograph on silica gel (dichloromethane/methanol=12/1) to afford 15 g of regioisomer mixture of compound (72e) and compound (72f). ESI-MS: m/z calcd for C₁₉H₂₉NO_{2 [}2M+Na]+629.8 Found: 630.1.

### Example 65F

### Preparation of Compound (72g) and Compound (72h)

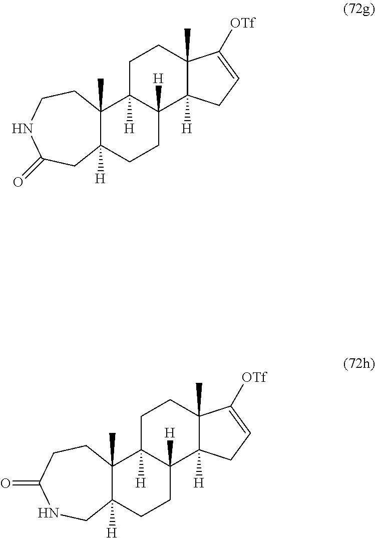

A solution of a regioisomer mixture of compound (72e) and compound (72f) (15 g, 49.4 mmol) in dichloromethane (250 mL) was treated dropwise with Tf₂O at 0° C., followed by addition of Et₃N (6.9 mL, 49.4 mmol). The resulting mixture was stirred overnight at room temperature. The reaction was diluted with dichloromethane (100 mL) and quenched with water (100 mL). The organic solution was separated and washed with water (200 mL), then evaporated to dryness. The residue was purified by flash column chromatograph on silica gel to afford 3.2 g of the regioisomer mixture of compound (72g) and compound (72h).

### Example 65G

### Preparation of Compound (72A) and Compound (72B)

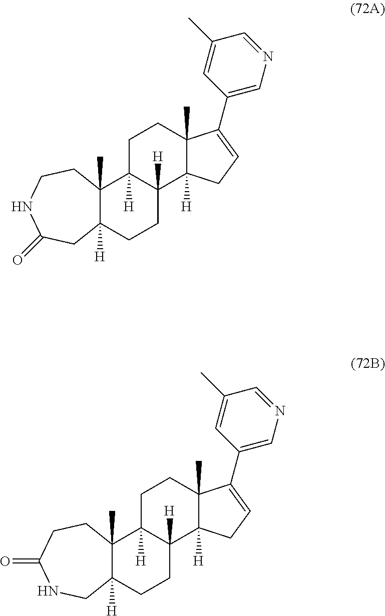

To a solution of regioisomer mixture of compound (72g) and compound (72h) (0.15 g, 0.34 mmol) in THF (8 mL) were added 2N aqueous Na₂CO₃ (8 mL), (5-methylpyridin-3-yl) boronic acid (94 mg, 0.69 mmol) and bis(triphenylphosphine)-palladium (II) chloride (12 mg). The mixture was degassed by Argon gas flushing for 1.5 h. The resulted mixture was heated at reflux overnight under Ar and all the starting material was consumed by TLC analysis (hexane/EtOAc/HOAc=1/1/3%). The volatile solvents were removed in vacuo and the residual aqueous solution was extracted with dichloromethane (3*10 mL). The combined organic layers were washed with brine (20 mL), dried over anhydrous sulfate sodium, and evaporated to dryness. The residue was purified by preparative HPLC to give 15.23 mg of a mixture of compound (72A) and compound (72B) which were separated by chromatography. Compound (72A): Ret. time at HPLC: 9.735 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.35 (s, 1H), 8.21 (s, 1H), 7.35 (s, 1H), 6.34 (s, 1H), 5.87 (s, 1H), 3.31 (m, 1H), 2.95 (m, 1H), 2.71 (m, 1H), 2.25 (s, 3H), 2.16 (m, 1H), 1.95 (m, 2H), 1.26-1.87 (m, 13H), 1.19 (m, 1H), 0.92 (d, 6H). ¹³C NMR (400 MHz, CDCl₃): 178.64, 151.62, 148.38, 144.98, 134.38, 132.39, 128.99, 57.28, 54.31, 47.19, 43.44, 41.80, 39.59, 39.03, 37.84, 35.42, 33.19, 31.82, 31.61, 30.90, 21.23, 18.45, 16.66, 11.94; ESI-MS: m/z calcd for C₂₅H₃₄N₂O: [M+H]+ 379.55. Found: 379.1. Compound (72B): ¹H NMR (400 MHz, CDCl₃) 8.22 (1H), 8.16 (1H), 7.13 (1H), 6.35 (b, 1H), 5.95 (1H, vinyl), 3.88 (s, 3H), 3.95 (2H), 3.67-3.77 (m, 1H), 2.51-2.69 (m, 2H), 2.36 (s, 3H), 2.15-2.25 (m, 2H), 0.95 (s, 3H), 0.90 (s, 3H). ESI MS: 379.1 [M+H]+.

### Example 66A

### Preparation of Compound (73A) and Compound (73B)

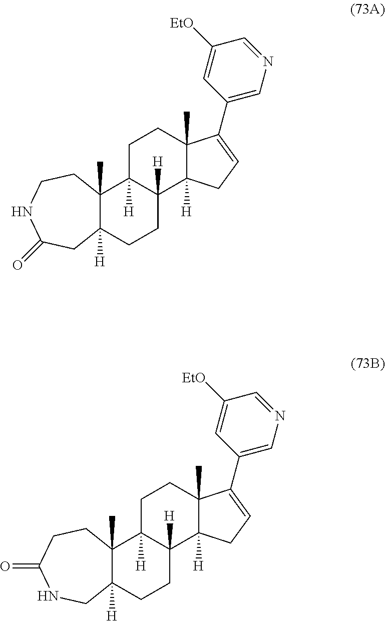

Using a synthetic procedure and conditions similar to Examples 65G in the preparation of compound (72A) and compound (72B) and replacing (5-methylpyridin-3-yl) boronic acid with (5-ethoxypyridin-3-yl) boronic acid, compound (73A), compound (73B) were made. Compound (73A): Ret. time at HPLC: 10.858 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.21 (s, 1H), 8.15 (s, 1H), 7.11 (s, 1H), 6.72 (s, 1H), 5.95 (s, 1H), 4.05 (m, 2H), 3.32 (m, 1H), 2.98 (m, 1H), 2.75 (m, 1H), 1.9-2.2 (m, 4H), 1.3-1.9 (m, 18H), 0.98 (d, 6H). ¹³C NMR (400 MHz, CDCl₃): 177.75, 153.62, 150.37, 139.28, 134.56, 132.44, 128.36, 118.27, 62.83, 56.26, 53.31, 46.21, 42.43, 40.77, 38.62, 38.01, 36.77, 34.41, 32.17, 30.80, 30.61, 29.88, 20.22, 15.66, 13.77, 10.92. ESI-MS: m/z calcd for C₂₆H₃₆N₂O₂: [M+H]+ 409.58. Found: 409.6. Compound (73B): ¹H NMR (400 MHz, CDCl₃): 8.22 (1H), 8.16 (1H), 7.13 (1H), 6.15 (b, 1H), 5.95 (1H, vinyl), 3.67-3.77 (m, 1H), 2.51-2.69 (m, 2H), 2.15-2.25 (m, 2H), 0.95 (s, 3H), 0.90 (s, 3H). ESI MS: 409.2 [M+H]+.

### Example 66B

### Preparation of Compound (73C) and Compound (73D)

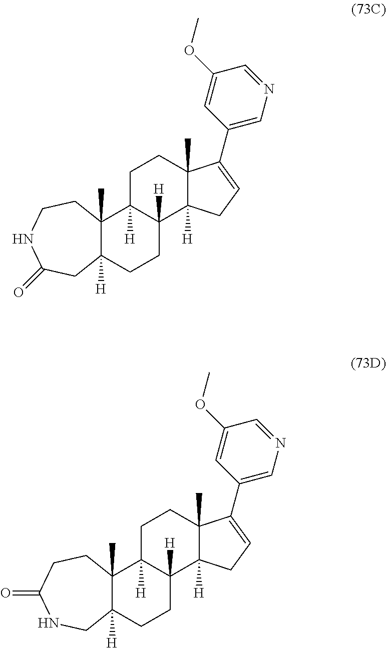

Using a synthetic procedure and conditions similar to Examples 65G in the preparation of compound (72A) and compound (72B) and replacing (5-methylpyridin-3-yl) boronic acid with (5-methoxypyridin-3-yl) boronic acid, compound (73C), compound (73D) were made. Compound (73C): Ret time: 10.017 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.22 (s, 1H), 8.14 (s, 1H), 7.12 (s, 1H), 5.95 (s, 1H), 3.85 (s, 3H), 3.36 (m, 1H), 3.01 (m, 1H), 2.75 (m, 1H), 2.21 (m, 1H), 2.01 (m, 3H), 1.7-1.9 (m, 3H), 1.3-1.7 (m, 9H), 1.21 (m, 2H), 0.99 (d, 6H). ¹³C NMR (400 MHz, CDCl₃): 178.51, 155.24, 151.36, 140.43, 135.19, 133.46, 129.51, 118.68, 57.26, 55.52, 54.29, 47.23, 43.42, 41.81, 39.58, 39.01, 37.84, 35.41, 33.17, 31.78, 31.62, 30.88, 21.24, 16.68, 11.93; ESI-MS: m/z calcd for C₂₅H₃₄N₂O₂: [M+H]+ 395.55. Found: 396.1; Compound (73D): ¹H NMR (400 MHz, CDCl₃) 8.22 (1H), 8.16 (1H), 7.13 (IH), 6.39 (b, 1H), 5.95 (1H, vinyl), 3.88 (s, 3H), 3.67-3.77 (m, 1H), 2.51-2.69 (m, 2H), 2.15-2.25 (m, 2H), 0.95 (s, 3H), 0.90 (s, 3H). ESI MS: 395.1 [M+H]+.

### Example 66C

### Preparation of Compound (73E) and Compound (73F)

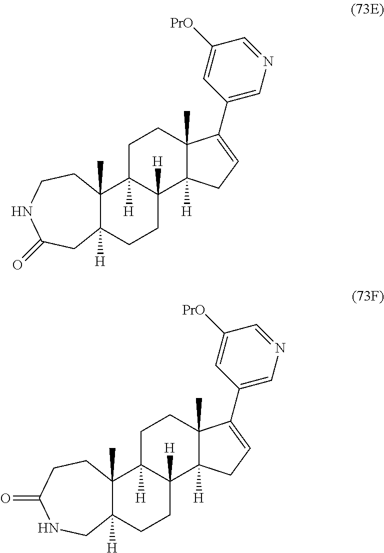

Using a synthetic procedure and conditions similar to Examples 65G in the preparation of compound (72A) and compound (72B) and replacing (5-methylpyridin-3-yl) boronic acid with (5-propoxypyridin-3-yl) boronic acid, compound (73E), compound (73F) were made. Compound (73E): Ret. time at HPLC: 13.599 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.15 (s, 1H), 8.09 (s, 1H), 7.05 (s, 1H), 6.55 (s, 1H), 5.89 (s, 1H), 3.91 (m, 1H), 3.31 (m, 1H), 2.95 (m, 1H), 2.70 (m, 1H), 2.10-2.25 (m, 2H), 1.95 (m, 2H), 1.64-1.87 (m, 6H), 1.21-1.64 (m, 9H), 1.15 (m, 1H), 1.01 (m, 5H), 0.91 (d, 6H). ¹³C NMR (400 MHz, CDCl₃): 177.65, 153.82, 150.41, 139.20, 134.63, 132.43, 128.35, 118.27, 68.80, 56.26, 53.30, 46.21, 42.42, 40.78, 38.59, 38.01, 36.80, 34.40, 32.17, 30.79, 30.61, 29.88, 21.53, 20.22, 15.67, 10.92, 9.46; ESI-MS: m/z calcd for C₂₇H₃₈N₂O₂: [M+H]+ 423.6. Found: 423.1; Compound (73F): ¹H NMR (400 MHz, CDCl₃) 8.22 (1H), 8.16 (1H), 7.13 (1H), 6.35 (b, 1H), 5.95 (1H, vinyl), 3.88 (s, 3H), 3.95 (2H), 3.67-3.77 (m, 1H), 2.51-2.69 (m, 2H), 2.15-2.25 (m, 2H), 0.95 (s, 3H), 0.90 (s, 3H); ESI MS: 423.2 [M+H]+.

### Example 66D

### Preparation of Compound (73G) and Compound (73H)

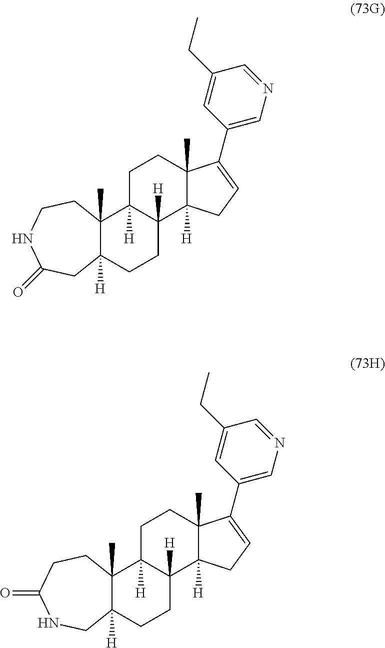

Using a synthetic procedure and conditions similar to Examples 65G in the preparation of compound (72A) and compound (72B) and replacing (5-methylpyridin-3-yl) boronic acid with (5-ethylpyridin-3-yl) boronic acid, compound (73G), compound (73H) were made. Compound (73G): Ret time at HPLC: 10.385 min. [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.42 (s, 1H), 8.31 (s, 1H), 7.42 (s, 1H), 6.61 (s, 1H), 5.96 (s, 1H), 3.41 (m, 1H), 3.01 (m, 1H), 2.72 (m, 1H), 2.62 (m, 2H), 2.35 (m, 1H), 1.9-2.19 (m, 4H), 1.3-1.9 (m, 9H), 1.05-1.21 (m, 5H), 0.98 (d, 6H). ¹³C NMR (400 MHz, CDCl₃): 178.71, 151.68, 147.67, 145.21, 138.50, 133.15, 132.48, 128.91, 57.27, 54.31, 47.18, 43.42, 41.77, 39.60, 39.01, 37.80, 35.41, 33.18, 31.80, 31.60, 30.88, 26.03, 21.23, 16.65, 15.34, 11.93; ESI-MS: m/z calcd for C₂₆H₃₆N₂O: [M+H]+ 393.58. Found: 394.0; Compound (73H): ¹H NMR (400 MHz, CDCl₃) 8.22 (IH), 8.16 (1H), 7.13 (1H), 6.01 (b, 1H), 5.95 (1H, vinyl), 3.67-3.77 (m, 1H), 2.51-2.69 (m, 4H), 2.15-2.25 (m, 2H), 0.95 (s, 3H), 0.90 (s, 3H). ESI MS: 393.1 [M+H]+.

### Example 67

### Preparation of Compound (74A) and Compound (74B)

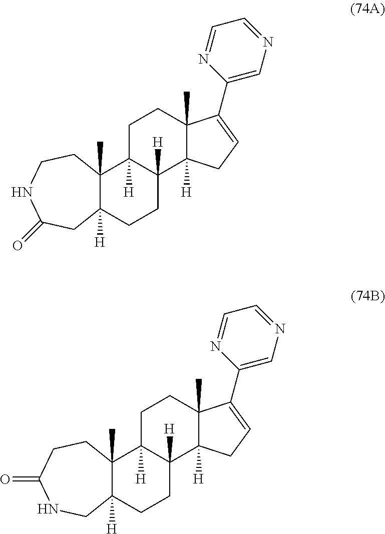

Using a synthetic procedure and conditions similar to Examples 65G in the preparation of compound (72A) and compound (72B) and replacing (5-methylpyridin-3-yl) boronic acid with 2-bromopyrazine, Na₂CO₃ with sodium acetate, and addition of bis(pinacolato)diboron, compound (74A), compound (74B) were made. Compound (74A): Ret. time at HPLC: 15.364 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 M) 8.68 (1H, pyrazine), 8.45 (1H, pyrazine), 8.30 (1H, pyrazine), 6.45 (1H, vinyl), 6.13 (1H, NHCO), 3.33-3.48 (1H), 2.52-5.68 (2H), 2.44-2.48 (1H), 2.22-2.34 (2H), 2.02-2.15 (1H), 1.00 (3H), 0.88 (3H). ¹³C NMR (CDCl₃, 400 M): 178.5556, 151.6793, 143.5478, 142.6192, 141.7857, 133.3341, 56.9562, 54.1182, 49.8236, 47.0490, 44.5301, 38.8910, 35.0950, 34.9959, 33.4478, 31.9964, 31.5656, 27.6765, 20.9400, 16.1692, 12.0429. ESI MS 388.1 [M+Na]⁺. Compound (74B): Ret. time at HPLC: 15.115 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (CDCl₃, 400 M) 8.68 (1H, pyrazine), 8.45 (1H, pyrazine), 8.30 (1H, pyrazine), 6.45 (1H, vinyl), 6.13 (1H, NHCO), 3.36-3.48 (m, 1H), 2.93-3.08 (m, 1H), 2.72-2.81 (dd, 1H), 2.37-2.42 (1H), 2.01-2.19 (1H), 1.00 (3H), 0.88 (3H). ¹³C NMR (CDCl₃, 400 M) 178.5141, 151.6114, 151.5739, 143.4917, 142.6230, 141.7454, 133.4873, 56.9215, 54.3930, 46.9994, 43.4464, 41.8053, 39.5908, 39.0513, 37.8904, 35.0279, 33.0741, 32.0052, 31.8541, 30.9020, 29.6991, 21.1895, 16.1432, 11.9354; ESI MS 388.3 [M+Na]⁺.

### Example 68

### Preparation of Compound (75A) and Compound (75B)

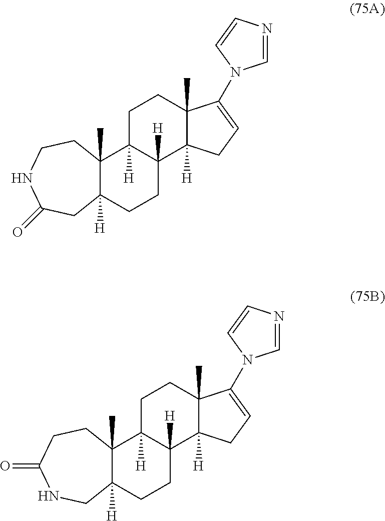

### Example 68A

### Preparation of Compound (75a)

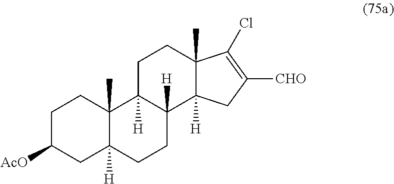

Epiandrosterone (50.0 g, 172 mmol) was dissolved to 300 mL of pyridine, followed by the addition of 25.5 g of Ac₂O and 430 mg of DMAP with stirring. The resulting mixture was heated at 85° C. overnight. After being cooled to room temperature the mixture was admixed with 700 mL of ice-water. 500 mL of EtOAc was added. The layers were partitioned. The water phase was extracted with 500 mL of EtOAc once again. The combined organic layers were washed with 5% citric acid, 5% NaHCO₃ and brine in turn. After being dried over MgSO₄, EtOAc was evaporated under reduced pressure until 80% of EtOAc was removed. The left organic solution was washed with 1 M HCl to get rid of pyridine, followed by washing 10% NaHCO₃ and brine. The organic phase was dried on MgSO₄ and condensed under reduced pressure to give 48.3 g of a product. Within an ice-water bath, 5.00 mL of DMF was pre-mixed with 5.00 mL of POCl₃ in 5 mL of CHCl₃ under argon protection. The resulting reagent was added dropwise to a solution of 1.00 g of the above product in 15.0 mL of CHCl₃ within an ice bath. The reaction mixture was allowed to warm to room temperature and heated at reflux for 5 hr until the starting material was completely consumed. After being cooled to room temperature, the mixture was poured slowly into 20 g of ice-water mixture. Extracted with ether/EtOAc (1/4), dried on MgSO₄, and purified through FCC to give 450 mg of compound (75a) as yellowish solid.

### Example 68B

### Preparation of Compound (75b)

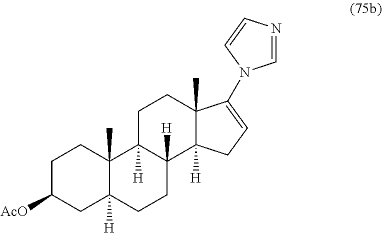

The mixture of compound (75a) (18.0 g, 47.5 mmol), imidazole (10.3 g, 148 mmol), and K₂CO₃ (24.6 g, 178 mmol) in 300 mL of DMF was heated at 85° C. with stirring for 1 hr. After being cooled to room temperature, the mixture was treated with 600 mL of ice-water mixture. The resulting mixture was extracted with EtOAc three times (each 400 mL). The combined organic layers were washed with 5% citric acid three times (each 200 mL) and brine once (200 mL). The solution was dried on MgSO₄ and condensed under reduced pressure to get the crude. Purification through FCC on silica gel (eluent: EtOAc/Hexanes=1/8→1/3) gave a product. A wet Pd/C was pre-dehydrated through washing with MeOH and PhCN. Then, 10 g of dry Pd/C (10%) was added to a solution of the above product (5.00 g). After being degas sed within an ultrasonic cleaner for 30 min, the mixture was heated at reflux overnight. The palladium on charcoal was removed by filtration. The filtrate was purified through FCC on silica gel (eluent: EtOAc/Hexanes=1/1) to give compound (75b).

### Example 68C

### Preparation of Compound (75c)

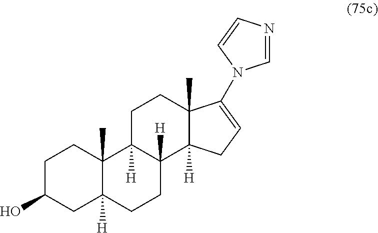

A solution of compound (75b) (1.50 g, 3.92 mmol) in 40 mL of methanol was treated with potassium hydroxide (0.660 g, 11.8 mmol) at rt for 3 hr. TLC indicated that the reaction came to the end. The solvent was removed under reduced pressure. The residue was treated with 30 mL of water and extracted with EtOAc (2×30 mL). The combined organic layers were washed with brine, dried on magnesium sulfate, and condensed by rotary evaporator to give compound (75c) (1.30 g, 97.4%).

### Example 68D

### Preparation of Compound (75d)

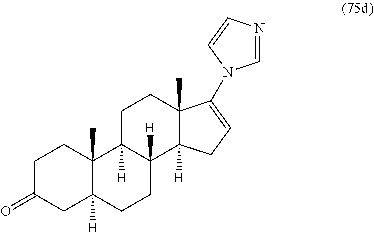

To a solution of compound (75c) (1.30 g, 3.82 mmol) in 50 mL of dichloromethane was added Dess-Martin periodinane (DMP) (3.30 g, 7.64 mmol) at room temperature. The mixture was heated to reflux for 4 hr. After being cooled to rt, the undissolved solid was removed by filtration. The filtrate was condensed under reduced pressure and purified through FCC on silica gel (eluent: dichloromethane/methanol=50/1) to give compound (75d) (1.23 g, 95.3%).

### Example 68E

### Preparation of Compound (75e)

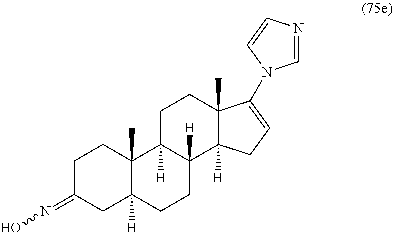

To a solution of compound (75d) (1.23 g, 3.63 mmol) in 50 mL of ethanol was added hydroxylamine hydrochloride (0.510 g, 7.26 mmol) and pyridine (0.580 g, 7.26 mmol) at rt. The reaction mixture was stirred at rt for 5 hr. The solvents were removed under reduced pressure. The residue was dissolved into 50 mL of EtOAc, washed with brine (2×20 mL), dried on sodium sulfate and condensed by rotary evaporator to give 1.10 g of compound (75e) in 85.9% yield.

### Example 68F

### Preparation of Compound (75A) and Compound (75B)

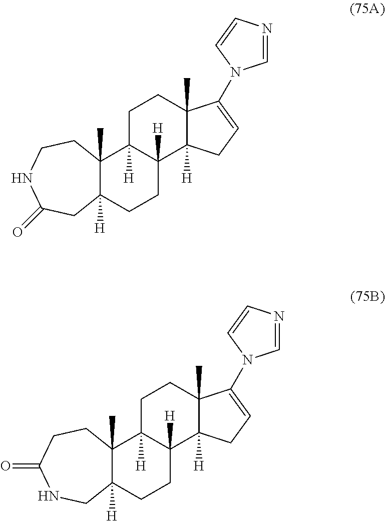

To a solution of compound (75e) (1.00 g, 2.83 mmol) in 100 mL of dichloromethane at 0° C., was added dropwise 3 mL of thionyl chloride keeping the temperature below 0° C. At that temperature, the reaction mixture was stirred for 20 min. The reaction was slowly poured into 100 mL of icy saturated sodium biscarbonate. The layers were portioned. The organic phase was washed with water (2×20 mL), dried on magnesium, and condensed under reduced pressure to give 0.840 g of the mixture of compound (75A) and compound (75B), which was subjected to the isolation by preparative HPLC to give 21 mg of compound (75A) and 23 mg of compound (75B). Compound (75A): ¹H NMR 7.61 (s, 1H, imidazole), 7.02-7.09 (dd, 2H, imidazole), 5.68 (1H, vinyl), 3.35 (m, 1H), 3.00 (m, 1H), 2.77 (m, 1H), 2.25 (1H); ¹³C NMR 177.5, 147.3, 134.8, 128.0, 118.1, 117.3, 54.9, 53.4, 45.1, 42.4, 40.8, 38.5, 38.0, 36.8, 33.9, 31.9, 30.3, 29.7, 28.6, 20.0, 14.9, 10.9. Compound (75B): ¹H NMR 7.61 (s, 1H, imidazole), 7.02-7.09 (dd, 2H, imidazole), 5.68 (1H, vinyl), 3.35 (m, 1H), 2.70 (m, 2H), 2.22 (m, 2H); ¹³C NMR 177.6, 147.4, 134.9, 128.2, 117.9, 117.2, 54.9, 53.1, 48.8, 45.2, 43.4, 37.9, 34.1, 33.9, 32.3, 30.3, 29.9, 28.6, 26.5, 19.8, 14.9, 11.0.

### Example 69

### Preparation of Compound (76)

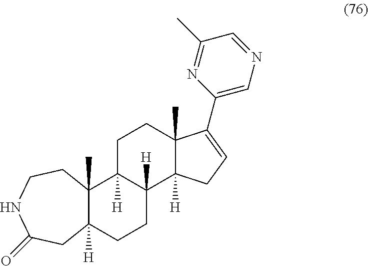

Using a synthetic procedure and condition similar to Examples 67 in the preparation of compound (74A), and replacing 2-bromopyrazine with 2-bromo-6-methylpyrazine compound (76) was made.

### Example 70

### Preparation of Compound (77)

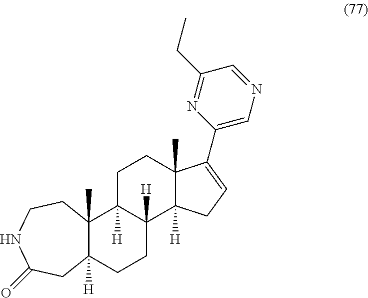

Using a synthetic procedure and condition similar to Examples 67 in the preparation of compound (74A), and replacing 2-bromopyrazine with 2-bromo-6-ethylpyrazine compound (77) is prepared.

### Example 71

### Preparation of Compound (78A) and Compound (78B)

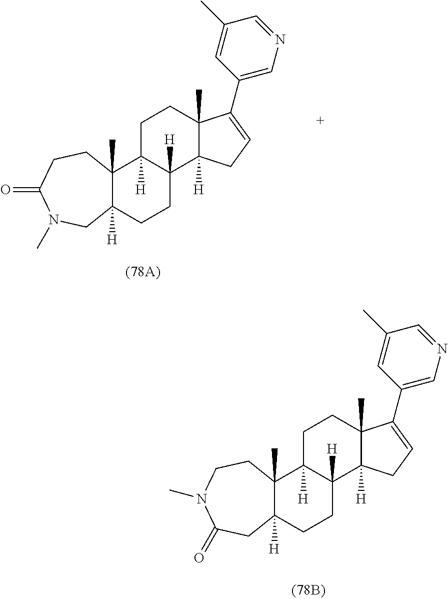

### Example 71A

### Preparation of Compound (78a)

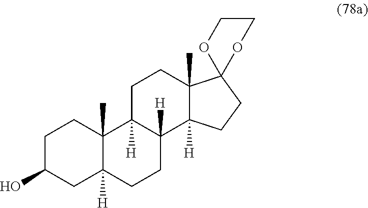

A solution of epiandrosterone (50 g, 172.2 mmol), ethylene alcohol (32 g, 516.4 mmol) and p-toluenesulfonic acid (0.88 g) in toluene (400 mL) were refluxed under a Dean-Stark trap over night. The mixture was cooled to room temperature and concentrated to dryness. The residue was diluted with EtOAc (1000 mL), washed with saturated NaHCO₃ (2*500 mL) and brine (500 mL). The organic phase was dried over anhydrous sodium sulfate, filtered, and evaporated to afford 54.6 g (94.8%) of compound (78a). ¹H NMR (400 MHz, CDCl₃): 3.85-4.0 (4H), 3.75 (1H), 3.56 (1H), 0.9-0.95 (6H).

### Example 71B

### Preparation of Compound (78b)

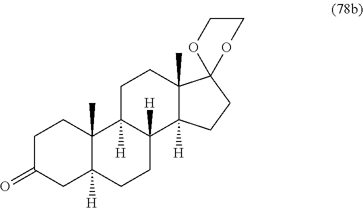

A solution of compound (78a) (30 g, 89.7 mmol) in dichloromethane (400 mL) was treated with Dess-Martin Periodinane (54.7 g, 129.1 mmol) at room temperature. The reaction was stirred until all the start material was consumed. The mixture was washed with sat.NaHCO₃ solution (2*200 mL), saturated Na₂SO₃ solution (200 mL) and brine (200 mL). The solution was dried over anhydrous sodium sulfate, filtered, and evaporated to dryness to afford 30 g of compound (78b). ¹H NMR (400 MHz, CDCl₃): 3.7-4.1 (4H), 2.2-2.5 (3H), 1.9-2.1 (3H), 1.05 (3H), 0.91 (3H). ¹³C NMR (400 MHz, CDCl₃): 211.84, 119.27, 65.16, 64.49, 53.58, 50.10, 14.37, 11.43.

### Example 71C

### Preparation of Compound (78c)

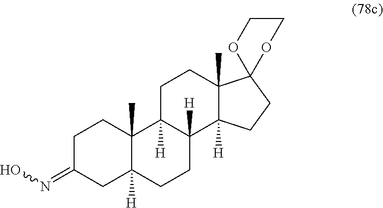

A solution of compound (78b) (30 g, 90.2 mmol) in EtOH (400 mL) was treated with NH₂OH.HCl (13 g, 180.5 mmol) and pyridine (16 mL, 180.5 mmol). The resulting mixture was stirred for 1 h at room temperature. The volatile solvents were removed under reduced pressure. The residue was dissolved in 500 mL of dichloromethane and washed with water (300 mL*2). The organic solution was evaporated to dryness to afford 27 g (86%) of compound (78c), which was used in the next step without further purification.

### Example 71D

### Preparation of Compound (78d) and Compound (78e)

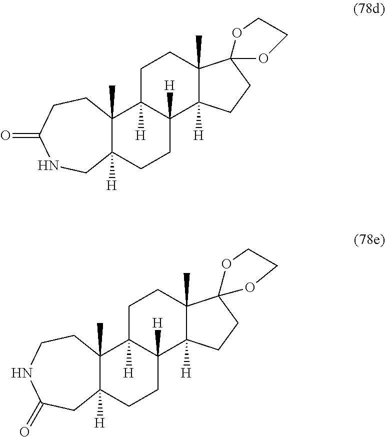

A solution of compound (78c) (12 g, 34.5 mmol) in pyridine (450 mL) was added dropwise with SOCl₂ (39.6 mL, 557.9 mmol) at 0°. The resulting mixture was stirred for 10 min and the starting material was completely consumed (dichloromethane/MeOH=30/1). The reaction was quenched with saturated K₂CO₃ solution. The mixture was extracted with dichloromethane (1000 mL), washed with water (2*300 mL) and diluted NaHCO₃ (300 mL), and evaporated to dryness. The residue was purified by column chromatograph on silica gel (dichloromethane/methanol=30/1) to afford 5 g (43%) of regioisomer mixture compound (78d) and compound (78e). ¹H NMR (400 MHz, CDCl₃): 6.12-6.35 (d, 1H), 3.81-3.97 (m, 4H), 3.38 (m, 1H), 2.2-3.0 (m, 3H), 0.93 (s, 3H), 0.85 (s, 3H). ESI-MS: m/z calcd for C₂₁H₃₃NO₃ [M+Na]+370.49 Found: 370.1; [2M+Na]+717.98. Found: 717.5.

### Example 71E

### Preparation of Compound (78f) and Compound (78g)

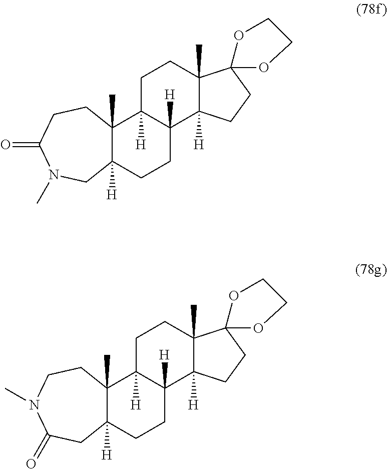

A solution of regioisomer mixture compound (78d) and compound (78e) (2 g, 5.8 mmol) in anhydrous THF (30 mL) was treated with NaH (0.7 g, 28.8 mmol, 60 wt % dispersion in mineral oil) at 0° C. After stirring for 1.5 h at room temperature, MeI (4.08 g, 1.5 mL, 28.8 mmol) was added. The resulting mixture was stirred overnight and the starting material was completely consumed (dichloromethane/MeOH=40/1). The reaction was quenched with 20 mL of saturated NH₄Cl solution and extracted with EtOAc (3*25 mL). The combined organic solution was washed with brine (50 mL), dried (Na₂SO₄), and evaporated to dryness to afford 2.4 g of regioisomer mixture compound (78f) and compound (78g). ¹H NMR (400 MHz, CDCl₃): 3.85 (m, 4H), 3.64 (m, 1H), 2.89 (d, 3H), 2.2-2.7 (m, 3H), 0.83 (s, 3H), 0.75 (s, 3H). ¹³C NMR (400 MHz, CDCl₃): 174.51, 174.43, 118.26, 64.17, 64.14, 63.54, 63.48, 52.93, 52.70, 52.31, 49.20, 46.86, 45.59, 44.68, 44.61, 42.61, 13.41, 11.32; ESI-MS: m/z calcd for C₂₂H₃₅NO₃ [M+Na]+384.52 Found: 384.0.

### Example 71F

### Preparation of Compound (78h) and Compound (78i)

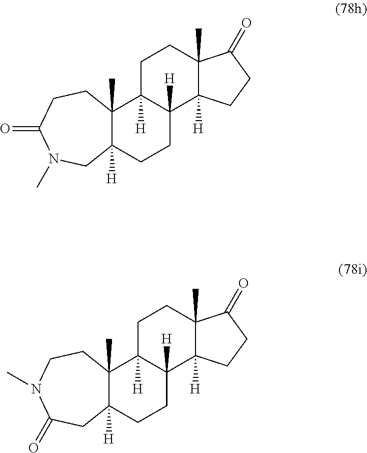

The regioisomer mixture compound (78f) and compound (78g) (2.4 g, 6.64 mmol), p-TsOH (1.7 g) and water (20 mL) in acetone (30 mL) was stirred at room temperature for 4 h. The volatile solvents were removed under reduced pressure. The aqueous solution was extracted with dichloromethane (2*25 mL). the combined organic solution was washed with saturated NaHCO₃ (20 mL) and brine (20 mL), dried over anhydrous sodium sulfate, filtered, and evaporated to afford 2 g (95%) of the regioisomer mixture compound (78h) and compound (78i). ¹H NMR (400 MHz, CDCl₃): 3.69 (m, 1H), 2.92 (d, 3H), 2.24-2.80 (3H), 2.01 (m, 1H), 0.92 (s, 3H), 0.85 (s, 3H). ¹³C NMR (400 MHz, CDCl₃): 175.34, 175.28, 54.19, 53.95, 53.18, 51.25, 47.84, 47.45, 46.50, 43.58, 13.52, 12.01.

### Example 71G

### Preparation of Compound (78j) and Compound (78k)

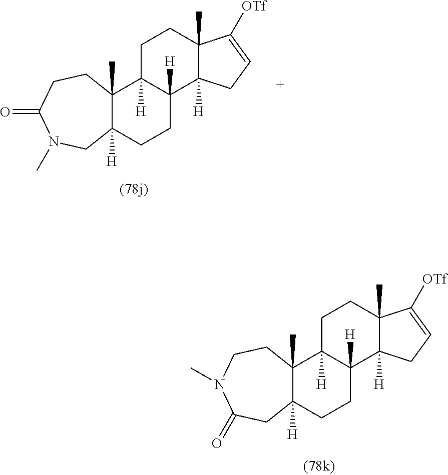

A solution of the regioisomer mixture compound (78h) and compound (78i) (1 g, 3.1 mmol) in dichloromethane (25 mL) was treated dropwise with Tf₂O at 0° C., followed addition of Et₃N (0.44 mL, 3.1 mmol). The resulting mixture was stirred overnight at room temperature. The reaction was diluted with dichloromethane (30 mL) and quenched with water (30 mL). The organic solution was separated, washed with water (50 mL), dried over Na₂SO₄, and evaporated to dryness. The residue was purified by flash column chromatograph on silica gel (hexane/EtOAc=1/1) to afford 0.84 g (59.3%) of the regioisomer mixture compound (78j) and compound (78k). ¹H NMR (400 MHz, CDCl₃): 5.54 (s, 1H), 3.65 (m, 1H), 2.95 (d, 3H), 2.1-2.8 (m, 4H), 0.95 (s, 3H), 0.88 (s, 3H).

### Example 71H

### Preparation of Compound (78A) and Compound (78B)

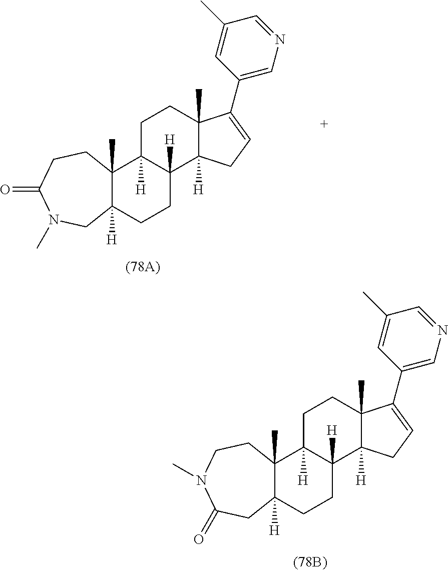

To a solution of the regioisomer mixture compound (78j) and compound (78k) (0.2 g, 0.56 mmol) in THF (8 mL) was added 2N aqueous Na₂CO₃ (8 mL), (5-methylpyridin-3-yl) boronic acid (150 mg, 1.11 mmol) and bis(triphenylphosphine)-palladium (II) chloride (20 mg). The mixture was degassed for 1.5 h. The resulted mixture was heated for 1.5 h at 80° C. under Argon and all the starting material was consumed by TLC analysis (hexane/EtOAc=1/1). The volatile solvents were removed under reduced pressure and the residual aqueous solution was extracted with dichloromethane (3*10 mL). The combined organic layers were washed with brine (20 mL), dried over anhydrous sulfate sodium, and evaporated to dryness. The residue was purified by preparative HPLC to give 14.55 mg of the regioisomer mixture compound (78A) and compound (78B) which were separated by HPLC. Compound (78B): Ret. time at HPLC: 10.035 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.49 (s, 1H), 8.40 (s, 1H), 7.52 (s, 1H), 5.94 (s, 1H), 3.68 (m, 1H), 3.01 (m, 1H), 2.94 (s, 3H), 2.77 (m, 1H), 2.30 (s, 1H), 2.23 (m, 1H), 1.9-2.1 (m, 4H), 1.4-1.9 (m, 11H), 1.25 (m, 1H), 1.09 (m, 1H), 1.01 (s, 3H), 0.98 (s, 3H), 0.88 (m, 1H). ¹³C NMR (400 MHz, CDCl₃): 175.54, 151.65, 148.39, 145.00, 134.37, 132.39, 129.02, 57.30, 54.37, 47.22, 46.60, 43.80, 40.06, 40.02, 38.49, 35.52, 35.39, 33.32, 31.77, 31.61, 30.77, 18.46, 16.68, 12.03. ESI-MS: m/z calcd for C₂₆H₃₆N₂O [M+H]+ 393.58. Found: 393.0.

### Example 72

### Preparation of Compound (79)

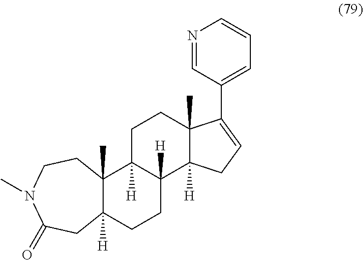

Using a synthetic procedure and conditions similar to Examples 71H in the preparation of compound (78B) and replacing (5-methylpyridin-3-yl) boronic acid with pyridin-3-ylboronic acid, compound (79) was made. Retention time at HPLC: 9.457 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.62 (s, 1H), 8.45 (d, 1H), 7.64 (d, 1H), 7.20 (m, 1H), 5.95 (s, 1H), 3.70 (m, 1H), 2.98 (m, 4H), 2.77 (m, 1H), 2.25 (m, 1H), 1.9-2.1 (m, 4H), 1.3-1.85 (m, 10H), 1.21 (m, 1H), 1.09 (m, 1H), 1.05 (s, 3H), 0.98 (s, 3H), 0.85 (m, 1H). ¹³C NMR (400 MHz, CDCl₃): 174.52, 150.52, 146.82, 146.79, 132.67, 131.89, 128.25, 122.03, 56.27, 53.33, 46.21, 45.57, 42.78, 39.01, 37.47, 34.50, 34.33, 32.28, 30.75, 30.62, 29.74, 20.23, 15.64, 11.00; ESI-MS: m/z calcd for C₂₅H₃₄N₂O [M+H]+ 379.55. Found: 379.0.

### Example 73

### Preparation of Compound (80)

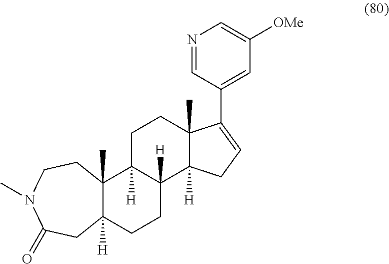

Using a synthetic procedure and conditions similar to Examples 71H in the preparation of compound (78B) and replacing (5-methylpyridin-3-yl) boronic acid with (5-methoxypyridin-3-yl)boronic acid, compound (80) was made. Retention time at HPLC: 10.659 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.23 (s, 1H), 8.15 (s, 1H), 7.13 (s, 1H), 5.96 (s, 1H), 3.88 (s, 3H), 3.70 (m, 1H), 2.97 (m, 4H), 2.78 (m, 1H), 2.23 (m, 1H), 1.9-2.07 (m, 4H), 1.74 (m, 2H), 1.3-1.7 (m, 9H), 1.25 (m, 1H), 1.07 (m, 1H), 1.01 (s, 3H), 0.95 (S,3H), 0.85 (m, 1H). ¹³C NMR (400 MHz, CDCl₃): 174.50, 154.26, 150.39, 139.43, 134.17, 132.48, 128.53, 117.71, 56.29, 54.52, 53.36, 46.26, 45.57, 42.79, 39.05, 37.48, 34.50, 34.38, 32.30, 30.75, 30.75, 30.61, 29.75, 20.25, 15.70, 11.01. ESI-MS: m/z calcd for C₂₆H₃₆N₂O₂ [M+Na]+431.58. Found: 431.1.

### Example 74

### Preparation of Compound (81)

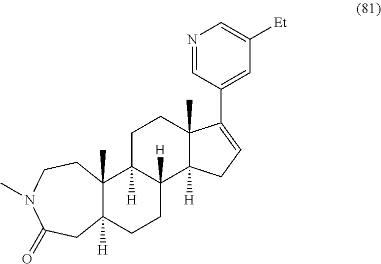

Using a synthetic procedure and conditions similar to Examples 71H in the preparation of compound (78B) and replacing (5-methylpyridin-3-yl) boronic acid with (5-ethylpyridin-3-yl)boronic acid, compound (81) was made. Retention time at HPLC: 10.752 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.42 (s, 1H), 8.29 (s, 1H), 7.42 (s, 1H), 5.95 (s, 1H), 3.70 (m, 1H), 2.95 (m, 4H), 2.77 (m, 1H), 2.63 (m, 2H), 2.22 (m, 1H), 1.9-2.1 (m, 4H), 1.75 (m, 2H), 1.3-1.7 (m, 9H), 1.24 (m, 4H), 1.09 (m, 1H), 1.01 (s, 3H), 0.95 (s, 3H), 0.85 (m, 1H). ¹³C NMR (400 MHz, CDCl₃): 174.52, 150.72, 146.68, 144.20, 137.51, 132.15, 131.50, 127.95, 56.29, 53.36, 46.22, 45.58, 42.79, 39.05, 37.48, 34.50, 34.38, 32.30, 30.76, 30.59, 29.76, 25.04, 20.25, 15.68, 14.32, 11.01; ESI-MS: m/z calcd for C₂₇H₃₈N₂O [M+H]+ 407.6. Found: 407.1.

### Example 75

### Preparation of Compound (82)

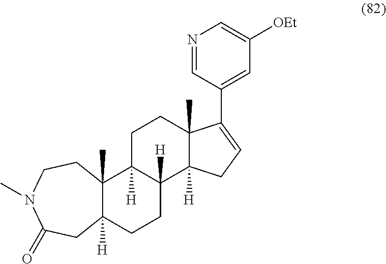

Using a synthetic procedure and conditions similar to Examples 71H in the preparation of compound (78B) and replacing (5-methylpyridin-3-yl) boronic acid with (5-ethoxylpyridin-3-yl)boronic acid, compound (82) was made. Retention time at HPLC: 11.442 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.23 (s, 1H), 7.15 (s, 1H), 7.15 (s, 1H), 5.98 (s, 1H), 4.11 (m, 2H), 3.70 (m, 1H), 3.01 (m, 4H), 2.77 (m, 1H), 2.25 (m, 1H), 1.9-2.15 (m, 4H), 1.3-1.9 (m, 14H), 1.21 (m, 1H), 1.08 (m, 1H), 1.02 (s, 3H), 0.98 (s, 3H), 0.85 (m, 1H). ¹³C NMR (400 MHz, CDCl₃): 175.51, 154.63, 151.40, 140.28, 135.53, 133.47, 129.41, 119.32, 63.84, 57.27, 54.35, 47.24, 46.57, 43.78, 40.04, 38.47, 35.50, 35.37, 33.28, 31.74, 31.60, 21.24, 16.68, 14.76, 12.00; ESI-MS: m/z calcd for C₂₇H₃₈N₂O₂ [M+H]+ 423.6. Found: 423.1.

### Example 76

### Preparation of Compounds (83)

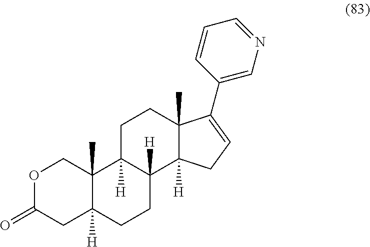

### Example 76A

### Preparation of Compounds (83a)

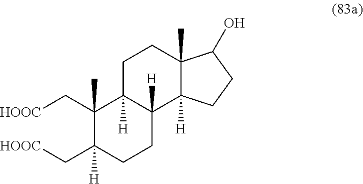

A solution of compound (69a) (20 g) in MeOH (200 ml) was basified with solid NaOH (6 g) at room temperature. After stirring for 20 min, water (20 ml) was added and the system was turned clear, followed by addition of solid NaBH₄ in portions. The solution was heated at 60° C. for 2 h. The reaction was quenched with 2N HCl until the PH<7 and water (50 ml) was added. The precipitate was collected by filtration and dried under vacuum to give 12 g of compound (83a).

### Example 76B

### Preparation of Compound (83b)

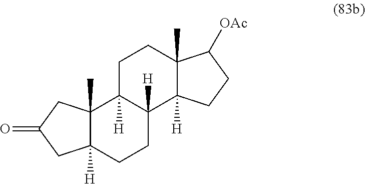

A solution of compound (83a) (17g) in acetic anhydride (150 ml) was heated with sodium acetate (4 g) at 80° C. for 2 h. The starting material was consumed by TLC analysis. The mixture was cooled to room temperature and the solid was removed away by filtration. The filtrate was concentrated under reduce pressure. The residue was dissolved in dichloromethane (100 ml), filtered though a pad of silica gel and rinsed with dichloromethane (20 ml). The filtrate was concentrated under reduce pressure. The residue was heated with a hot air blow dryer in vacuo to provide 14.8 g of compound (83b). ¹H NMR (CDCl₃, 300 MHz): 4.5-4.69 (m, 1H), 2.1-2.3 (m, 6H), 2.02 (s, 3H), 2.55-1.95 (m, 8H), 0.95 (s, 3H), 0.90 (s, 3H).

### Example 76C

### Preparation of Compound (83c)

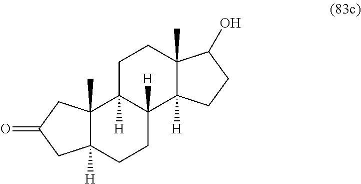

A solution of compound (83b) (9.5 g) in methanol (50 ml) was treated with NaOH (2 g). The reaction was heated at reflux for 1.5 h and the starting material was consumed. The mixture was concentrated under reduce pressure. The residue was suspended in EtOAc (50 ml) and water (50 ml). The organic layer was separated, washed with water (30 ml) and brine (30 ml), dried (Na₂SO₄), and evaporated to dryness. The residue was purified by column chromatograph on silica gel (EtOAc:Hexane=1:4) to afford 6 g of compound (83c).

### Example 76D

### Preparation of Compound (83d) and Compound (84a)

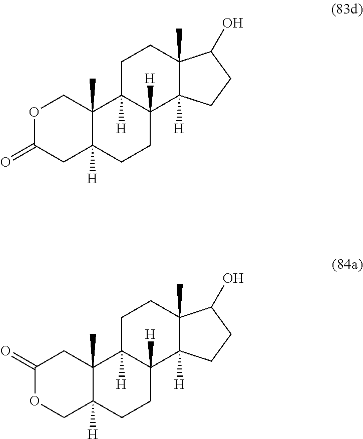

A solution of compound (83c) (4.5 g) in dichloromethane (200 ml) was treated with 3-chloroperoxybenzoic acid (8.5 g). The solution was stirred overnight at room temperature. TLC analysis indicated a trace of unreacted starting material was detected and additional 3 g of 3-chloroperoxybenzoic acid was added. The resulting mixture was stirred for additional 24 h. The mixture was washed with saturated aqueous Na₂SO₃, saturated aqueous NaHCO₃ and brine, dried over Na₂SO₄, and evaporated to dryness giving a mixture of compound (83d) and compound (84a) in 5:1 ratio. The residue was purified by column chromatograph on silica gel (EtOAc:Hexane=1:3) to afford 3.3 g of the pure compound (83d). ¹H NMR (CDCl₃, 300 MHz): 4.2 (1H), 3.9-4.1 (1H), 3.6-3.7 (1H), 2.5-2.7 (1H), 1.95-2.3 (2H), 1.02 (3H), 0.82 (3H). ¹³C NMR (CDCl₃, 300 MHz): 170.96, 170.61, 81.68, 81.02, 70.94, 53.46, 52.91, 50.61, 50.54, 11.07, 10.17

### Example 76E

### Preparation of Compound (83e)

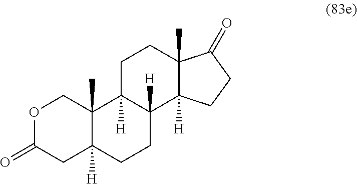

A solution of compound (83d) in dichloromethane (60 ml) was treated with Dess-Martin Periodinane (4.5 g) at room temperature. The mixture was stirred overnight at room temperature and the starting material was completely consumed. The solution was washed with saturated aqueous Na₂SO₃ (50 ml), saturated NaHCO₃ (50 ml) and brine (50 ml), dried over anhydrous Na₂SO₄, and evaporated to dryness. The residue was purified by column chromatograph on silica gel to afford 1.5 g of the pure compound (83e) as a white powder. ¹H NMR (CDCl₃, 300 MHz): 4.2 (1H), 3.9-4.1 (1H), 2.3-2.7 (3H), 2.0-2.3 (3H), 1.02 (3H), 0.95 (3H). ¹³C NMR (CDCl₃, 300 MHz): 170.54, 170.28, 80.70, 70.76, 52.83, 50.93, 49.70, 13.69, 10.12

### Example 76F

### Preparation of Compound (83f)

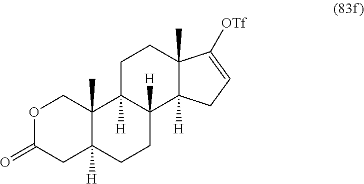

A solution of compound (83e) (1.5 g) in dichloromethane (50 ml) was treated with trifluorosulfonate anhydride (1.3 ml) at room temperature. After stirring for 15 min. a solution of triethyl amine (0.72 ml) in dichloromethane (8 ml) was added. The resulting mixture was stirred overnight and all the starting material was consumed. The reaction was quenched with water (50 ml) and the organic layer was separated. The aqueous layer was back-extracted with dichloromethane (30 ml). The combined organic layers were washed with saturated aqueous NaHCO₃ (2×30 ml) and brine (50 ml), dried over anhydrous Na₂SO₄, and evaporated to dryness. The residue was purified by column chromatograph on silica gel (hexane: EtOAc=4:1) to afford 1 g of compound (83f). ¹H NMR (CDCl₃, 300 MHz): 5.6 (1H), 4.2 (1H), 3.9-4.1 (1H), 2.5-2.65 (1H), 2.1-2.2 (2H), 1.9-2.1 (2H), 0.95-1.05 (6H). ¹³C NMR (CDCl₃, 300 MHz): 170.21, 158.83, 120.14, 114.45, 80.50, 70.78, 53.73, 50.06, 44.75, 40.52, 15.22, 10.07

### Example 76G

### Preparation of Compounds (83)

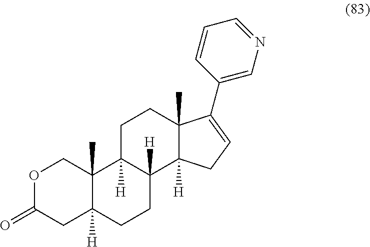

To a solution of compound (83f) (1 g) in THF (20 mL) were added 2N aqueous Na₂CO₃ (1.5 ml), pyridin-3-ylboronic acid (0.7 g) and bis(triphenylphosphine)palladium (II) chloride (83 mg). The mixture was degassed by Argon gas flushing for 15 min. The resulted mixture was heated at reflux overnight under Argon and all the starting material was consumed by TLC analysis (hexane: EtOAc=1:1). The volatile solvent was removed in vacuo and the residual aqueous solution was acidified with 2N aqueous HCl to pH 3. The mixture was stirred for 30 min and neutralized with NaHCO₃. The product was extracted with EtOAc (2×30 mL). The combined organic layers were washed with brine (30 mL), dried over anhydrous sulfate sodium, and evaporated to dryness. The residue was purified by preparative HPLC to give 34.37 mg of compound (83). Retention time at HPLC: 9.885 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ESI-MS: m/z calcd for C₂₃H₂₉NO₂ [M+H]+ 352.48 Found: 352.3; [2M+H]+ 703.96 Found: 703.5; ¹H NMR (CDCl₃, 300 MHz): 8.6 (1H), 8.45 (1H), 7.65 (1H), 7.19 (1H), 5.97 (1H), 4.25 (1H), 3.98 (1H), 3.5-3.6 (1H), 2.2-2.3 (2H), 1.95-2.05 (2H), 2.4-2.9 (8H), 2.15-2.3 (1H), 1.05 (6H), 0.95-1.02 (2H). ¹³C NMR (CDCl₃, 300 MHz): 169.44, 150.51, 146.98, 146.86, 132.64, 131.69, 128.02, 122.05, 79.76, 55.92, 48.93, 46.30, 39.53, 33.84, 33.79, 32.80, 32.31, 30.67, 29.81, 26.08, 20.19, 15.62, 9.12

### Example 77

### Preparation of Compounds (84)

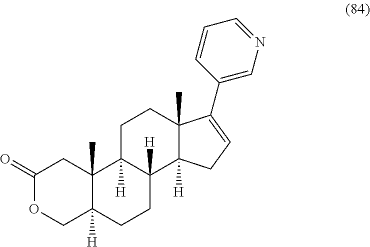

Using a synthetic procedure and conditions similar to Examples 76E to 76G in the preparation of compound (83) and replacing compound (83d) with compound (84a), compound (84) is made.

### Example 78

### Preparation of Compound (85)

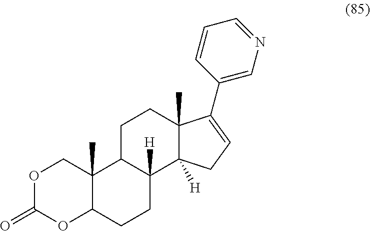

### Example 78A

### Preparation of Compound (85b)

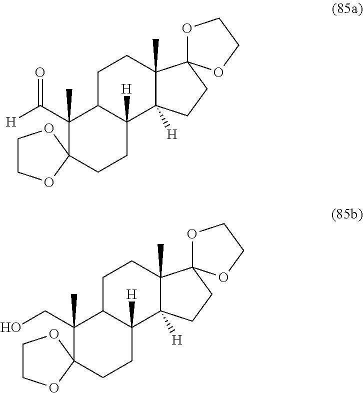

Compound (85a) (7.82 g, 22.31 mmol) in MeOH (150 mL) was treated with NaBH₄ (1.02 g, 26.77 mmol) at room temperature. The mixture was stirred for 3 h and quenched with water (30 mL). The volatile solvents were removed under reduced pressure. The aqueous solution was extracted with dichloromethane (2*30 mL). The combined organic solution was washed with brine, dried over Na₂SO₄, and evaporated to dryness to afford 7.3 g (93%) of compound (85b).

### Example 78B

### Preparation of Compound (85c)

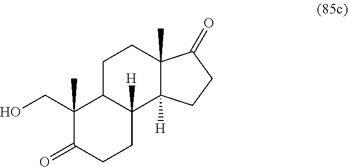

A solution of compound (85b) (7.3 g, 20.71 mmol) in THF (20 mL) was treated with 5N HCl and stirred for 2 h at room temperature. The volatile solvents were removed under reduced pressure and extracted with dichloromethane (3*20 mL). The combined organic solution was washed with brine, dried over Na₂SO₄, and evaporated to dryness to afford 5 g (92%) of compound (85c).

### Example 78C

### Preparation of Compound (85d)

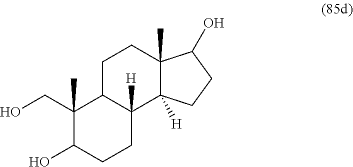

A solution of compound (85c) (6.8 g, 25.72 mmol) in MeOH (150 mL) was treated with NaBH₄ and stirred for 3 h at room temperature. The reaction was quenched with water (30 mL). The volatile solvents were removed under reduced pressure. The aqueous solution was extracted with dichloromethane (2*30 mL). The combined organic solution washed with brine, dried over Na₂SO₄, and evaporated to dryness to afford 5.8 g (85%) of compound (85d). ¹H NMR (400 MHz, DMSO): 4.41 (d, 1H), 4.31 (m, 1H), 4.29 (d, 1H), 3.44 (m, 3H), 3.12 (m, 1H), 1.84 (m, 1H), 1.76 (m, 1H), 1.45-1.66 (m, 4H), 1.1-1.4 (m, 6H), 0.7-1.05 (m, 5H0, 0.55 (d, 6H).

### Example 78D

### Preparation of Compound (85e)

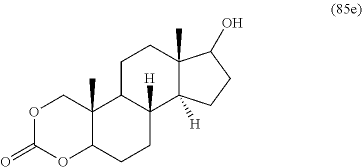

A solution of compound (85d) (1.8 g, 6.7 mmol) in THF (200 mL) was treated with triphosgene (3 g, 10.1 mmol) at 0° C., followed by addition of triethylamine (2 mL). The mixture was stirred over night at room temperature. TLC indicated some starting material was still present. 1 g of triphosgene was added, followed by treatment with triethylamine (2 mL) and DMAP (4 g, 32.7 mmol). The insoluble solid was removed by filtration. The resulting filtrate was stirred continued for 1 h. The volatile solvent was removed under reduced pressure. The residue was partitioned with water (50 mL) and EtOAc (50 mL). The organic solution was evaporated to dryness in vacuo. The residue was purified by column chromatograph on silica gel to afford 750 mg (38%) of compound (85e). ¹H NMR (400 MHz, CDCl₃): 3.8-4.3 (m, 3H), 3.57 (m, 1H), 1.04 (s, 3H), 0.68 (s, 3H). ¹³C NMR (400 MHz, CDCl₃): 148.78, 83.38, 81.39, 80.35, 49.95, 47.83, 43.00, 35.89, 34.58, 34.15, 30.08, 29.14, 28.20, 25.64, 23.22, 20.99, 11.06, 10.64.

### Example 78E

### Preparation of Compound (85f)

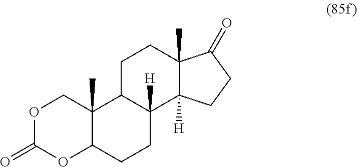

A solution of compound (85e) (750 mg, 2.55 mmol) in dichloromethane (30 mL) was treated with Dess-Martin periodinane (3 g, 7.07 mmol) and refluxed for 4 h. The reaction mixture was washed with saturated aqueous NaHCO₃ solution (20 mL), saturated aqueous NaHSO₃ solution (20 mL) and brine (20 mL). The organic solution was dried and evaporated. The residue was purified by flash column chromatograph to afford 600 mg (80%) of compound (85f). ¹H NMR (400 MHz, CDCl₃): 4.27 (m, 1H), 4.14 (m, 1H0, 2.52 (m, 1H), 1.12 (s, 3H), 0.89 (s, 3H). ¹³C NMR (400 MHz, CDCl₃): 148.46, 83.05, 80.09, 50.34, 47.81, 47.57, 35.43, 34.64, 33.72, 30.84, 27.49, 25.50, 21.64, 20.67, 13.72, 10.63.

### Example 78F

### Preparation of Compound (85g)

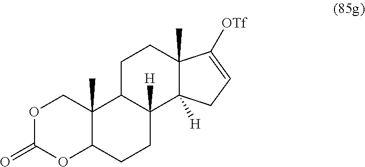

A solution of compound (85f) (600 mg, 2 mmol) in dichloromethane (10 mL) was treated dropwise with a solution of Tf₂O (900 mg, 3.2 mmol) in dichloromethane (2 mL). After stirring for 15 min, a solution of Et₃N (210 mg, 2 mmol) in dichloromethane (2 mL) was added. The resulting mixture was stirred for overnight at room temperature. The reaction was quenched with water (15 mL) and 20 mL of dichloromethane was added. The organic layer was separated and washed with saturated aqueous NaHCO₃ solution (15 mL) and brine (15 mL), dried over Na₂SO₄, and evaporated to dryness. The residue was purified by column chromatograph on silica gel (EtOAc/Hexane=1/4) to afford 250 mg (28.7%) of compound (85g).

### Example 78G

### Preparation of Compound (85)

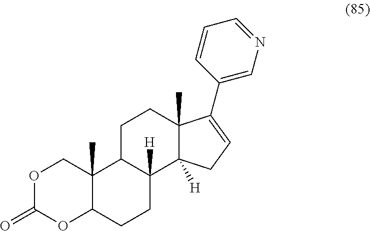

To a solution of compound (85g) (170 mg) in THF (10 mL) was added 3-(diethylboryl)pyridine (116 mg), Pd(PPh₃)₂Cl₂ (14 mg) and 2N aqueous Na₂CO₃ (5 mL). The mixture was degassed for 30 min and heated at 80° C. for 40 min under Ar. The volatile solvents were removed under reduced pressure. The residue partitioned between EtOAc (10 mL) and water (10 mL). The aqueous solution was back-extracted with EtOAc (10 mL). The combined organic solution was washed with brine (15 mL), dried over sodium sulfate, and evaporated to dryness. The residue was purified by RP-HPLC to afford 35 mg (24.7%) of compound (85). Ret. time at HPLC: 10.047 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150*4.6 mmID)]. ¹H NMR (400 MHz, CDCl₃): 8.80 (s, 1H), 8.68 (d, 1H), 7.55 (d, 1H), 7.20 (m, 1H), 5.96 (s, 1H), 4.20 (m, 1H), 4.07 (m, 2H), 2.48 (m, 1H), 1.9-2.1 (m, 4H), 1.3-1.8 (m, 6H), 1.12 (s, 3H), 1.05 (m, 1H), 1.01 (s, 3H), 0.98 (m, 1H). ¹³C NMR (400 MHz, CDCl₃): 151.33, 148.62, 148.12, 147.85, 133.71, 132.55, 128.98, 128.93, 123.07, 83.37, 80.17, 56.30, 48.04, 47.48, 34.71, 34.50, 32.76, 31.56, 28.44, 25.68, 16.64, 10.69; ESI-MS: m/z calcd for C₂₂H₂₇NO₃ [M+H]+ 354.45. Found: 354.0.

### Example 79

### Preparation of Compound (86)

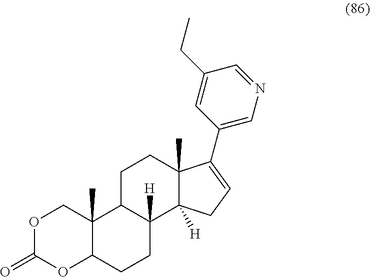

Using a synthetic procedure and conditions similar to Examples 78g in the preparation of compound (85) and replacing 3-(diethylboryl)pyridine with 3-(diethylboryl)-5-ethylpyridine, compound (86) was made. Retention time at HPLC: 10.242 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.419 (s, 1H), 8.33 (s, 1H), 7.43 (s, 1H0, 5.96 (s, 1H), 4.20 (m, 1H), 4.11 (m, 2H), 2.67 (m, 2H), 2.31 (m, 1H), 1.85-2.15 (m, 5H), 1.18 (s, 3H), 1.07 (s, 3H). ¹³C NMR (400 MHz, CDCl₃): 150.55, 147.62, 146.96, 144.23, 137.65, 132.21, 131.18, 127.67, 82.38, 79.20, 55.37, 52.44, 47.13, 46.52, 33.74, 33.58, 31.80, 30.55, 28.71, 27.48, 25.05, 24.72, 20.30, 15.68, 14.33, 9.70; ESI-MS: m/z calcd for C₂₄H₃₁NO₃ [M+H]+ 382.51. Found: 382.0.

### Example 80

### Preparation of Compound (87)

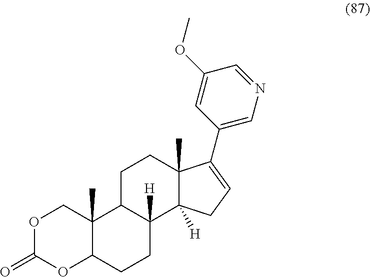

Using a synthetic procedure and conditions similar to Examples 78g in the preparation of compound (85) and replacing 3-(diethylboryl)pyridine with 3-(diethylboryl)-5-methoxypyridine, compound (87) was made. Retention time at HPLC: 9.874 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.31 (s, 1H), 8.24 (s, 1H), 7.26 (s, 1H), 6.01 (s, 1H), 4.24 (m, 1H0, 4.17 (m, 1H), 3.92 (s, 3H), 2.36 (m, 1H), 1.9-2.2 (m, 4H), 1.3-1.8 (m, 6H), 1.29 (m, 1H), 1.15 (s, 3H), 1.03 (s, 3H). ¹³C NMR (400 MHz, CDCl₃): 154.76, 149.60, 147.58, 137.88, 132.88, 132.50, 129.21, 119.40, 82.29, 79.13, 55.32, 54.85, 47.03, 46.55, 33.70, 33.48, 31.73, 30.59, 27.41, 24.66, 20.24, 15.68, 9.65; ESI-MS: m/z calcd for C₂₃H₂₉NO₃: [M+H]+ 384.48. Found: 383.9.

### Example 81

### Preparation of Compound (88)

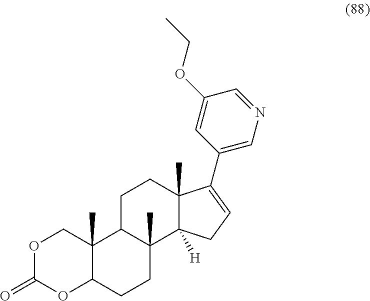

Using a synthetic procedure and conditions similar to Examples 78g in the preparation of compound (85) and replacing 3-(diethylboryl)pyridine with 3-(diethylboryl)-5-ethoxypyridine, compound (88) was made. Retention time at HPLC: 10.987 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (400 MHz, CDCl₃): 8.26 (s, 1H), 8.22 (s, 1H), 7.30 (s, 1H), 6.06 (s, 1H), 4.0-4.3 (m, 5H), 2.26 (m, 1H), 1.82-2.15 (m, 4H), 1.2-1.8 (m, 11H), 1.18 (s, 3H), 1.03 (s, 3H). ¹³C NMR (400 MHz, CDCl₃): 154.54, 149.16, 147.54, 136.46, 133.49, 131.30, 129.92, 121.22, 82.26, 79.33, 63.52, 55.31, 47.00, 46.56, 33.70, 33.43, 31.71, 30.64, 28.68, 27.39, 24.65, 21.67, 20.22, 15.68, 13.62; ESI-MS: m/z calcd for C₂₄H₃₁NO₄: [M+H]+ 398.51. Found: 398.0.

### Example 82

### Preparation of Compound (89)

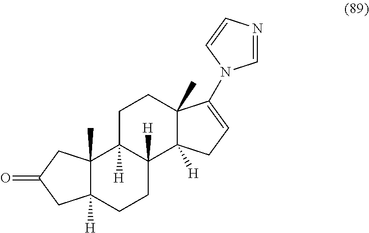

### Example 82A

### Preparation of Compound (89a)

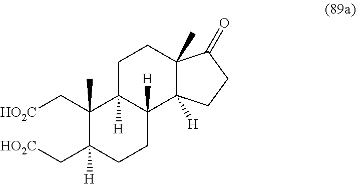

172 g of CrO₃, 1200 mL of H₂O and 250 mL of H₂SO₄ were pre-mixed. This mixed acid was added dropwise into a solution of 150 g of epiandrosterone in 700 mL of HOAc with stirring. The mixture was heated to 90-100° C. and kept for 2 hr. The reaction mixture was cooled within an ice-water bath, and then was extracted with EtOAc (2×1500 mL). The combined organic layers were condensed to form solid under reduced pressure. The solid was dissolved into 2 L of 5 mol/L of aqueous sodium hydroxide. The solution was washed with EtOAc (2×1500 mL) to remove the unreacted starting material and liposoluble by-products. The combined EtOAc phase was back-extracted with H₂O (500 mL). The aqueous phases were combined totally and adjusted to pH2 by conc. HCl. The resulting mixture was re-extracted with EtOAc (2×1500 mL). The combined organic layers were washed with brine, dried on Na₂SO₄, and condensed under reduced pressure to a point that a large volume of solid was formed. The precipitate was collected by filtration. The filtrate was purified through FCC. Totally, 146 g compound (89a) was obtained in 83.7% yield.

### Example 82B

### Preparation of Compound (89b)

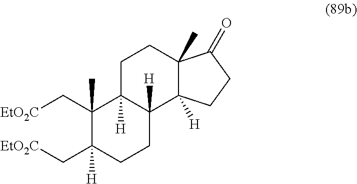

To a solution of compound (89a) (20 g) in absolute ethanol (100 mL) was added dropwise SOCl₂ (17 mL) with stirring within an ice bath. The resulting mixture was refluxed overnight. The volatiles were removed under reduced pressure. 300 mL of EtOAc and 300 mL of H₂O was added. The organic phase was partitioned. The water phase was re-extracted with EtOAc. The combined EtOAc layers were washed with NaHCO₃, dried on MgSO₄ and condensed under reduced pressure to give 21 g compound (89b).

### Example 82C

### Preparation of Compound (89c)

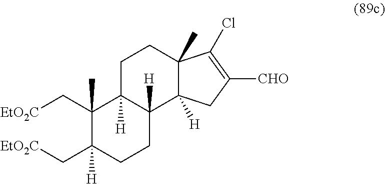

A solution of compound (89b) (785 mg, 2.0 mmol) in 10 mL of chloroform was treated with a mixture of DMF-POCl₃-CHCl₃ (volume: 3.35 mL-3.35 mL-3.35 mL) within an ice-bath. The reaction was heated at reflux overnight. After being cooled to room temperature, the mixture was poured into 15 mL of ice-water. It was extracted with chloroform, washed with 10% citric acid, 10% sodium biscarbonte and saturated sodium chloride, dried on magnesium sulfate, and condensed by rotary evaporator to give the compound (89c). Purification by FCC on silica gel provided 560 mg of compound (89c) in 64% yield.

### Example 82D

### Preparation of Compound (80d)

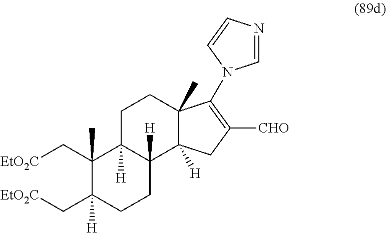

Compound (89c) (560 mg, 1.28 mmol) was treated with imidazole (275 mg, 4.0 mmol) and potassium carbonate (663 mg, 4.8 mmol) in 10 mL of DMF at 80° C. for 1 hr. The reaction was poured into icy water, and was extracted with dichloromethane (2×50 mL). The combined organic phases were washed with 10% citric acid and saturated brine, dried on magnesium sulfate and condensed by rotary evaporator to give 420 mg compound (89d) in 70% yield.

### Example 82E

### Preparation of Compound (89e)

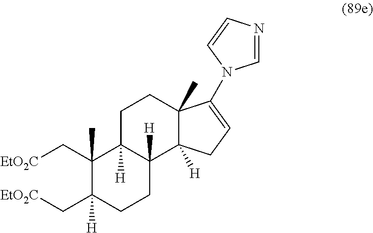

A mixture of compound (89d) (430 mg) and Pd/C (850 mg, the catalyst was pre-treated with absolute methanol) in benzonitrile (10 mL) was heated at 200° C. for 4 hr. The solvent was removed under reduced pressure. The residue was treated with a mixture of dichloromethane (20 mL) and water (20 mL). The organic phase was partitioned, washed with brine and purified through FCC on silica gel to give 230 mg compound (89e) in 57% yield. ¹H NMR: 7.56 (singlet, 1H, imidazole), 6.97-7.05 (dd, 2H, imidazole), 5.61 (1H, vinyl), 4.00-4.10 (4H, 2CH₃CH₂O—), 2.65 (d, 1H), 2.48 (d, 1H), 2.20 (2H), 1.80-2.20 (5H), 0.90 (3H), 0.80 (3H). ¹³C NMR 173.6, 171.3, 148.8, 136.2, 129.6, 119.3, 118.6, 60.5, 60.4, 56.3, 48.9, 46.4, 41.3, 40.8, 40.1, 36.3, 35.1, 33.9, 30.9, 29.9, 27.7, 21.8, 16.3, 15.9, 14.6, 14.5.

### Example 82F

### Preparation of Compound (89f)

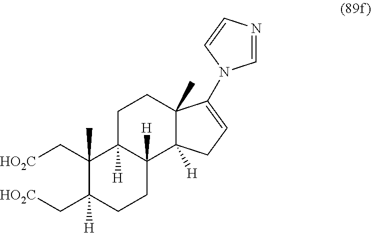

A solution of compound (89e) (13.0 g, 29.3 mmol) in 100 mL of methanol was treated with LiOH.H₂O (15.0 g, 176 mmol, 6 equiv.) in 30 mL of water at reflux overnight. Methanol was removed under reduced pressure. The aqueous phase was washed with cool EtOAc (3×40 mL), and then adjusted with 5 M HCl to pH→2. A mass of white solid precipitated and was collected by filtration. After dried under reduced pressure, 7.4 g compound (89f) was obtained.

### Example 82G

### Preparation of Compound (89)

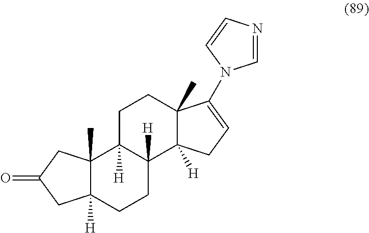

A mixture of C0748 compound (89f) (1.00 g), acetic anhydride (80 mL) and sodium acetate (210 mg) was heated at reflux overnight. The volatile solvents were removed under reduced pressure. The residue was added into 100 mL of water, and was extracted with dichloromethane (3×150 mL). The combined organic layers were washed with 10% sodium biscarbonate and brine, dried on magnesium sulfate. Purification through prep HPLC provided 80 mg of compound (89).

### Example 83

### Preparation of Compound (90)

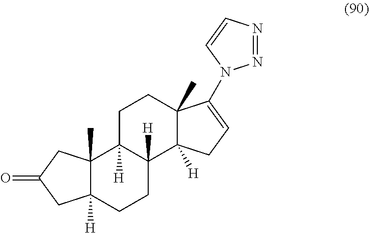

Using a synthetic procedure and conditions similar to Examples 82D to 82G in the preparation of compound (90) and replacing imidazole with 1H-1,2,3-triazole in Example 82D, compound (90) was made.

### Example 84

### Preparation of Compound (91)

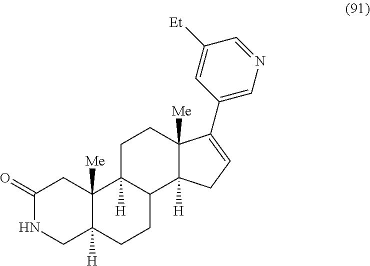

### Example 84A

### Preparation of Compound (91a)

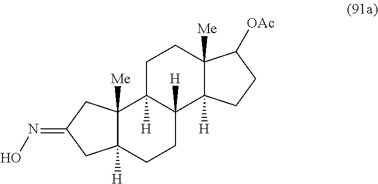

A solution of compound (83b) (3.7 g, 11.62 mmol) in EtOH (50 mL) was treated with hydroxylamine hydrochloride (1.62 g, 23.24 mmol) at room temperature, followed by addition of pyridine (1.84 g, 23.24 mmol). The reaction was stirred until complete conversion of the starting material as indicated by TLC (hexane: EtOAc=3:1). The volatile solvents were removed away under reduced pressure. The residue was partitioned with between H₂O (20 mL) and EtOAc (50 mL). The organic layer was washed with water (2*20 mL), dried over Na₂SO₄, filtered, and evaporated to dryness to give 3.6 g (93.0%) of compound (91a), which was used directly in the next step without further purification.

### Example 84B

### Preparation of Compound (91b)

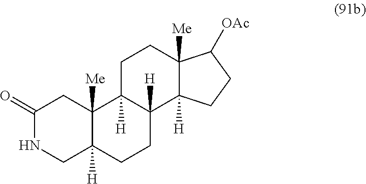

A stirring solution of compound (91a) (5.10 g, 15.29 mmol) in dry dioxane (85 mL) was treated dropwise with freshly distilled thionyl chloride (15 mL) at room temperature. After the addition, the mixture was stirred for a further 30 min. The volatile solvents were removed away under reduced pressure. The residue was partitioned between H₂O (20 mL) and dichloromethane (50 mL) and neutralized with solid Na₂CO₃. The aqueous layer was extracted with dichloromethane (2*20 mL). The combined organic layers were concentrated to dryness. The residue was purified by column chromatograph (silica gel, hexane:EtOAc:HOAc=2:1:0.07) to afford 4.24 g (83.14%) of compound (91b). ¹H NMR (CDCl₃, 400 MHz): 6.61 (1H, br), 4.52 (1H, m), 3.12 (1H , m), 2.95 (1H, d), 2.2 (2H, m), 2.12 (1H, m), 2.01 (3H, s), 0.92 (3H, s), 0.80 (3H, s). ¹³C NMR (CDCl₃, 400 MHz): 172.16, 171.19, 82.59, 54.56, 51.26, 50.41, 42.46, 40.38, 36.56, 35.05, 34.75, 34.64, 30.79, 27.59, 27.37, 23.47, 21.17, 20.69, 12.01, 10.75.

### Example 84C

### Preparation of Compound (91c)

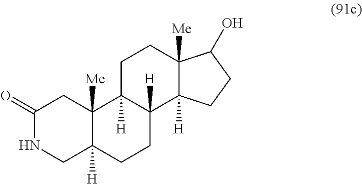

A stirring solution of compound (91b) (4.24 g, 12.71 mmol) in methanol (100 mL) and treated with solid KOH (2.14 g, 38.14 mmol) at room temperature. The reaction mixture was stirred overnight and the starting material was completely consumed. The mixture was concentrated under reduced pressure. The residue was partitioned between dichloromethane (100 mL) and H₂O (20 mL). The aqueous layer was extracted with dichloromethane (2*20 mL). The combined organic solution was dried over anhydrous sodium sulfate, and evaporated to give 3.7 g (100%) of compound (91c). ¹H NMR (CDCl₃, 400 MHz): 3.62 (1H, m), 3.12 (1H, d), 2.93 (1H, d), 2.2-2.3 (1H, m), 2.02 (2H, m), 0.95 (3H, s), 0.76 (3H, s). ¹³C NMR (CDCl₃, 400 MHz): 172.11, 81.72, 54.68, 51.49, 50.69, 42.84, 40.49, 36.41, 35.09, 35.03, 34.70, 30.37, 29.70, 27.64, 23.36, 11.07, 10.79.

### Example 84D

### Preparation of Compound (91d)

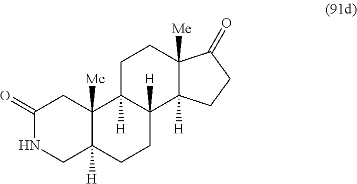

A solution of compound (91c) (3.70 g, 12.71 mmol) in chloromethane (150 mL) was treated with Dess-Martin periodinane (8.10 g, 19.04 mmol) at room temperature. The mixture was stirred overnight until the starting material was completely consumed. The reaction was quenched with saturated aqueous Na₂SO₃ solution. The organic layer was separated and concentrated to dryness. The residue was purified by column chromatograph (silica gel, dichloromethane/methanol=40/1) to afford 3.2 g (87%) of the compound (91d). ¹H NMR (CDCl₃, 400 MHz): 6.98 (1H, d), 3.12 (1H, m), 2.89 (1H, d), 2.35-2.45 (1H, m), 2.2 (1H, m), 1.9-2.1 (3H, m), 1.7-1.85 (2H, m), 0.85 (3H, s), 0.80 (3H, s). ¹³C NMR (CDCl₃, 400 MHz): 172.16, 54.35, 53.48, 51.41, 51.05, 47.51, 40.39, 35.67, 34.99, 34.66, 34.51, 31.20, 30.08, 27.42, 21.71, 20.47, 13.67, 12.18, 10.71.

### Example 84E

### Preparation of Compound (91e)

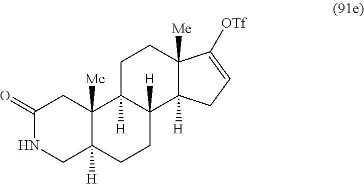

To a solution of compound (91d) (3.20 g, 11.06 mmol) in dichloromethane (50 mL) was added dropwise with trifluoromethane sulfonic anhydride (4.6 8 g, 16.58 mmol) at 0° C. After the addition, the solution was stirred for a further 15 min and triethylamine (0.96 g, 9.5 mmol) was added. The mixture was stirred overnight at room temperature and the starting material was completely consumed by TLC analysis. The reaction was quenched with water (10 mL). The organic layer was separated and the aqueous layer was back-extracted with DCM (2 *20 mL). The combined organic layers were washed with water and brine, dried over (Na₂SO₄) and concentrated in vacuo. The residue was purified by column chromatography on silica gel (hexanes/EA/HOAc=4/1/0.07) to give 2.84 g (61%) of the compound (91e). ¹H NMR (CDCl₃, 400 MHz): 6.35 (1H, d), 5.57 (1H, s), 3.12 (1H, m), 2.95 (1H, d), 2.2-2.3 (3H, m), 0.98 (6H). ESI-MS: m/z calcd for C₁₉H₂₆F₃NO₄S: [M+H]+ 422.47. Found: 422.9; [2M+Na]+865.94. Found: 865.6.

### Example 84F

### Preparation of Compound (91)

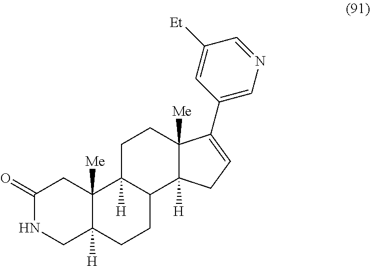

To a mixture solution of compound (91e) (214 mg, 0.51 mmol) in THF (10 mL) and water (10 mL), was added 5-ethylpyridin-3-ylboronic acid (154 mg, 1.02 mmol), bis(triphenylphosphine)palladium(II) chloride (18 mg, 0.02 mmol) and solid Na₂CO₃ (108 mg, 1.02 mmol). The mixture was degassed and refilled with Ar gas for 30 min. Then the mixture was heated at 80° C. under nitrogen overnight. The mixture was cooled to room temperature and the volatile solvents were removed away under reduced pressure. The aqueous solution was extracted with dichloromethane (3×10 mL). The combined organic layers were dried over (Na₂SO₄) and evaporated to dryness. The residue was purified by RP-HPLC to give 42 mg of compound (91). Retention time at HPLC: 9.798 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃, 400 MHz): 8.42 (1H, s), 8.31 (1H, s), 7.44 (1H, s), 6.02 (1H, s), 5.96 (1H, s), 3.14 (1H, m), 2.99 (1H, d), 2.64 (2H, m), 2.32 (2H, m), 1.95-2.15 (3H m), 1.75-1.95 (3H, m), 1.32-1.71 (8H, m), 1.2-1.3 (5H, m), 1.01 (6H, d). ¹³C NMR (CDCl₃, 400 MHz): 172.34, 151.72, 147.71, 145.26, 138.46, 133.07, 132.38, 128.78, 57.08, 54.27, 53.47, 51.62, 47.32, 40.57, 35.09, 35.05, 34.66, 33.49, 31.64, 31.06, 29.66, 27.63, 26.01, 21.08, 16.64, 15.34, 10.70; ESI-MS: m/z calcd for C₂₅H₃₄N₂O: [M+H]+ 379.55. Found: 379.5.

### Example 85

### Preparation of Compound (92)

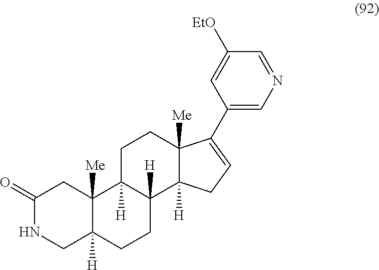

Using a synthetic procedure and conditions similar to Examples 84F in the preparation of compound (91) and replacing 5-ethylpyridin-3-ylboronic acid with 5-ethoxypyridin-3-ylboronic acid, compound (92) was made. Compound (92): Retention time at HPLC: 10.090 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃, 400 MHz): 8.20 (1H, s), 8.14 (1H, s), 7.12 (1H, s), 6.17 (1H, s), 5.97 (1H, s), 4.10 (2H, m), 3.13 (1H, m), 2.98 (1H, d), 2.26 (2H, m), 1.91-2.11 (5H, m), 1.79 (1H, m), 1.38-1.76 (11H, m), 1.22 (2H, m), 1.01 (6H, d). ¹³C NMR (CDCl₃, 400 MHz): 171.96, 154.63, 151.42, 140.36, 135.55, 133.39, 129.29, 119.38, 63.87, 57.09, 54.49, 51.64, 47.38, 40.64, 35.12, 35.06, 34.79, 33.51, 31.67, 31.07, 27.65, 21.13, 16.67, 14.77, 10.74; ESI-MS: m/z calcd for C₂₅H₃₄N₂O₂: [M+H]+ 395.55. Found: 395.4.

### Example 86

### Preparation of Compound (93)

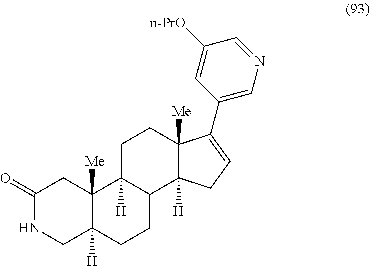

Using a synthetic procedure and conditions similar to Examples 84F in the preparation of compound (91) and replacing 5-ethylpyridin-3-ylboronic acid with 5-propoxypyridin-3-ylboronic acid, compound (93) was made. Compound (93): Retention time at HPLC: 11.071 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃, 400 MHz): 8.19 (1H, s), 8.14 (1H, s), 7.12 (1H, s), 6.37 (1H, s), 5.96 (1H, s), 3.96 (2H, m), 3.14 (1H, m), 2.97 (1H, d), 2.28 (2H, m), 1.95-2.14 (4H, m), 1.73-1.92 (4H, m), 1.3-1.7 (8H, m), 1.22 (3H, m), 1.08 (3H, m), 0.98 (6H, d). ¹³C NMR (CDCl₃, 400 MHz): 172.07, 154.84, 151.46, 140.27, 135.64, 133.39, 129.29, 119.34, 69.83, 57.10, 54.45, 51.65, 47.39, 40.63, 35.12, 35.06, 34.78, 33.52, 31.68, 31.08, 29.70, 27.66, 22.55, 21.13, 16.69, 10.75, 10.48; ESI-MS: m/z calcd for C₂₆H₃₆N₂O₂ [M+H]+ 409.58. Found: 409.4.

### Example 87

### Preparation of Compound (94)

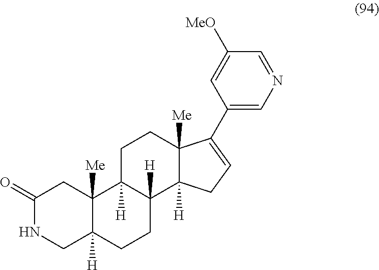

Using a synthetic procedure and conditions similar to Examples 84F in the preparation of compound (91) and replacing 5-ethylpyridin-3-ylboronic acid with 5-methoxypyridin-3-ylboronic acid, compound (94) was made. Compound (94): Retention time at HPLC: 9.271 min [Mobile phase: B %=10-100 (gradient 20 min); B=MeCN, A=H₂O (0.1% TFA); Flow rate: 0.8 mL/min; UV=266 nm column: zorbax Eclipse XDB-C8 5u, (150×4.6 mmID)]. ¹H NMR (CDCl₃, 400 MHz): 8.216 (1H, s), 8.16 (1H, s), 7.13 (1H, s), 6.17 (1H, s), 5.98 (1H, s), 3.85 (3H, s), 3.15 (1H, m), 2.98 (1H, d), 2.29 (2H, m), 1.85-2.17 (5H, m), 0.98 (6H, d). ¹³C NMR (CDCl₃, 400 MHz): 171.99, 155.27, 151.40, 140.48, 135.19, 133.41, 129.45, 118.78, 57.11, 55.55, 54.49, 51.65, 47.41, 40.64, 35.13, 35.07, 34.80, 33.52, 31.69, 31.08, 27.66, 27.66, 21.13, 16.69, 10.75; ESI-MS: m/z calcd for C₂₄H₃₂N₂O₂: [M+H]+ 381.52. Found: 381.4.

### Example 88

### Preparation of Compound (95)

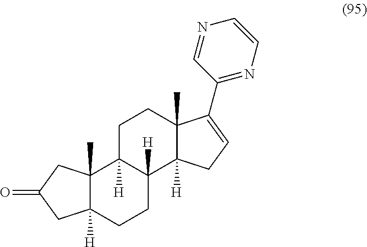

### Example 88A

### Preparation of Compound (95a)

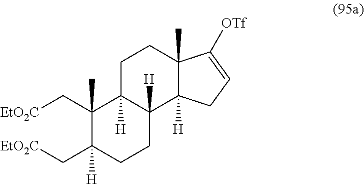

To a solution of compound (89a) (22.4 g, 0.0571 mol) in CH₂Cl₂ (100 mL) was added dropwise Tf₂O (24.2 g, 0.0856 mol, 1.5 equiv.) at 0° C. with stirring, followed by TEA (5.80 g, 0.0571 mol, 1 equiv.). The reaction mixture was allowed to warm to room temperature and stirred overnight. 30 mL of water was added. The organic layer was partitioned, washed with brine, dried over MgSO₄ and purified through FCC to give 21.4 g of compound (95a) in 71.5% yield.

### Example 88B

### Preparation of Compound (95b)

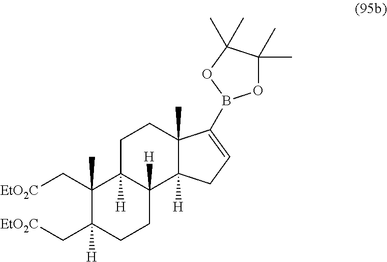

A mixture of compound (95a) (2.00 g), Pin₂B₂ (1.00 g, 1.1 equiv.), K₂CO₃ (0.560 g, 1.5 equiv.), Pd(Ph₃P)₂Cl₂ (80.0 mg, 3%), Ph₃P (150 mg, 12%) in dioxane (50 mL) was degassed within an ultrasonic cleaner for 30 min under argon protection. The mixture was heated at reflux with stirring for 4 hr. The solvent was removed by rotovapor. The residue was partitioned with EtOAc (80 mL) and H₂O (30 mL). The EtOAc layer was washed with brine, dried on MgSO₄ and purified through FCC(CH₂Cl₂/Hexanes=1/1) to give 1.8 g of compound (95b).

### Example 88C

### Preparation of Compound (95c)

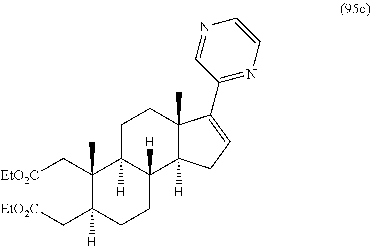

A mixture of compound (95b) (1.70 g), K₂CO₃ (1.87 g, 4 equiv.), Pd(Ph₃P)₂Cl₂ (72.0 mg, 3%), and 2-bromopyrazine (1.07 g, 2 equiv.) in dioxane (18 mL) and H₂O (6 mL) was degassed within an ultrasonic cleaner for 1 hr under argon protection. The mixture was heated at reflux with stirring for 3 hr. The residue was partitioned with EtOAc (80 mL) and H₂O (30 mL). The EtOAc layer was washed with brine, dried on MgSO₄ and purified through FCC (EtOAc/Hexanes=1/1) to give 1.5 g of compound (95c).

### Example 88D

### Preparation of Compound (95d)

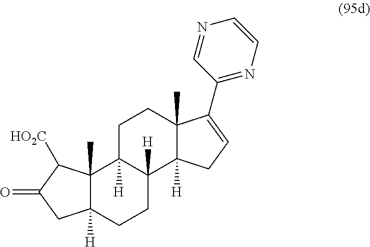

Sodium ethoxide (120 mg) was added to a solution of compound (95c) (400 mg) in toluene (15 mL), followed with heating at reflux with stirring overnight. TLC eluenting with EtOAc/Hexanes (1/2) showed the reaction came to the end. Toluene was evaporated under reduced pressure. To the residue was added 100 mg of LiOH.H₂O, 15 mL of H₂O and 20 mL of THF. The resulting mixture was refluxed overnight. TLC(CH₂Cl₂/MeOH=15/1) indicated the reaction was completed. The solvents were removed by rotovapor. The residue was adjusted to pH=4 with 2 mol/L HCl. The formed mixture was extracted with CH₂Cl₂ (2×30 mL). The combined organic layers were washed with brine, dried over Na₂SO₄ and condensed under reduced pressure to give 350 mg of C0847 compound (95d).

### Example 88E

### Preparation of Compound (95)

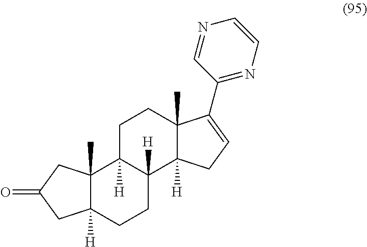

Under agon protection, 1.1 g of compound (95d) was heated till CO₂ was released. At that point, the heating was ceased. After being cooled to room temperature, the residue was purified through FCC (EtOAc/Hexanes=1/1) to give 710 mg of compound (95).

### Example 89

### Preparation of Compound (96A) and Compound (96B)

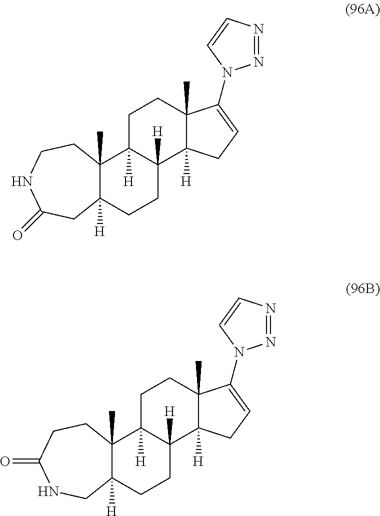

Using a synthetic procedure and conditions similar to Example 68 in the preparation of compound (75A) and compound (75B), and replacing imidazole with triazole in Example 68B and carrying on subsequent experiments with procedure similar to Example 68C to Example 68F, compound (96A) and Compound (96B) were made.

The examples and embodiments described herein are for illustrative purposes only and various modifications or changes suggested to persons skilled in the art are to be included within the spirit and purview of this application and scope of the appended claims.

1. A compound having the structure of Formula (I): 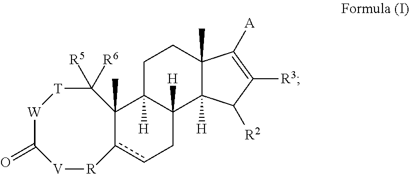
wherein:
R is (CH₂)ₚ wherein p is an integer from 0 to 1;
T is (CH₂)_{q} wherein q is an integer from 0 to 1;
W is O, NR¹, NCOR¹, N—COOR¹or null;
V is CR⁷, R⁸, O, NR¹, N—COR¹or N—COOR¹;
with the proviso that when W is O, NR¹, NCOR¹ or N—COOR¹ and p and q are each 0, or when W is null and one of p and q is 0, V cannot be CR⁷R⁸;
A is a heteroaryl optionally substituted with 1, 2, 3, or 4 R⁴; [symbol]
is a single bond or double bond;
R¹ is selected from the group consisting of hydrogen, alkyl, cycloalkyl, alkenyl, alkynyl, alkoxyalkyl, hydroxyl, and haloalkoxyalkyl; wherein the alkyl, cycloalkyl, alkenyl, alkynyl, alkoxyalkyl, and haloalkoxyalkyl groups are optionally substituted with 1, 2, or 3 substituents independently selected from the group consisting of halogen, alkyl, alkenyl, aryl, heteroaryl, alkoxy, alkoxycarbonyl, hydroxyl, hydroxyalkyl, alkynyl, cyano, haloalkoxy, haloalkyl, nitro, NR_{A}R_{B}, and (NR_{A}R_{B})carbonyl;
R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and optionally substituted heteroarylalkyl; or
R_{A} and R_{B} taken together with the nitrogen atom form an optionally substituted 4 to 7 membered heterocyclic ring having one or two heteroatoms;
R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;
R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and
R⁵ and R⁶ are each independently selected from the group consisting of hydrogen, halogen, optionally substituted alkyl, and optionally substituted alkoxyalkyl;
R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and/Elf NR_{A}R_{B}; and
R⁷ and R⁸ are each independently selected from the group consisting of hydrogen, halogen, optionally substituted alkyl, and optionally substituted alkoxyalkyl;
or a single stereoisomer or single tautomer or mixture thereof, or a pharmaceutically acceptable salt or solvate thereof.

2.-5. (canceled)

6. A compound having the structure of Formula (II): 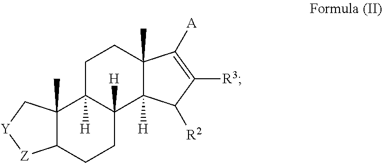
wherein:
Y is (CH₂)ₘ wherein m is an integer from 1 to 3;
Z is O, S(O)ᵤ, NR¹, N—COR¹or N—COOR¹; wherein u is an integer from 0 to 2;
A is a heteroaryl optionally substituted with 1, 2, 3, or 4 R⁴;
R¹ is selected from the group consisting of hydrogen, alkyl, cycloalkyl, alkenyl, alkynyl, alkoxyalkyl, hydroxyl, and haloalkoxyalkyl; wherein the alkyl, cycloalkyl, alkenyl, alkynyl, alkoxyalkyl, and haloalkoxyalkyl groups are optionally substituted with 1, 2, or 3 substituents independently selected from the group consisting of halogen, alkyl, alkenyl, aryl, heteroaryl, alkoxy, alkoxycarbonyl, hydroxyl, hydroxyalkyl, alkynyl, cyano, haloalkoxy, haloalkyl, nitro, NR_{A}R_{B}, and (NR_{A}R_{B})carbonyl;
R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and optionally substituted heteroarylalkyl; or
R_{A} and R_{B} taken together with the nitrogen atom form an optionally substituted 4 to 7 membered heterocyclic ring having one or two heteroatoms;
R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;
R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and
R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and NR_{A}R_{B};
or a single stereoisomer or single tautomer or mixture thereof, or a pharmaceutically acceptable salt or solvate thereof.

7. A compound having the structure of Formula (III): 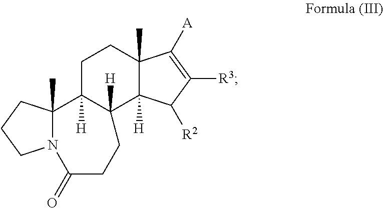
wherein:
A is a heteroaryl optionally substituted with 1, 2, 3, or 4 R⁴;
R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, and optionally substituted heteroarylalkyl; or
R_{A} and R_{B} taken together with the nitrogen atom form an optionally substituted 4 to 7 membered heterocyclic ring having one or two heteroatoms;
R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;
R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and
R⁴ is independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, and NR_{A}R_{B};
or a single stereoisomer or single tautomer or mixture thereof, or a pharmaceutically acceptable salt or solvate thereof.

8. The compound of claim 1 having the structure of Formula (IA): 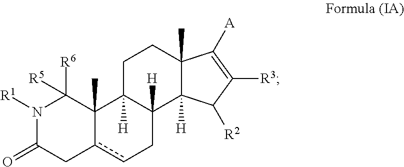
or a single stereoisomer or single tautomer or mixture thereof, or a pharmaceutically acceptable salt or solvate thereof.

9. The compound of claim 1 having the structure of Formula (IB): 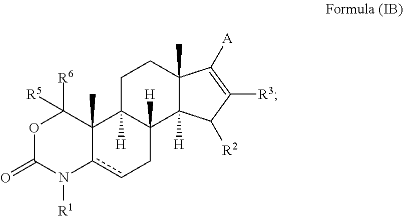
or a single stereoisomer or single tautomer or mixture thereof, or a pharmaceutically acceptable salt or solvate thereof.

10. The compound of claim 1 having the structure of Formula (IC): 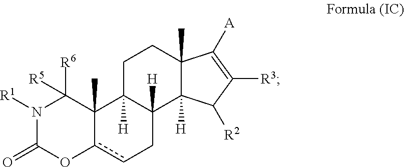
or a single stereoisomer or single tautomer or mixture thereof, or a pharmaceutically acceptable salt or solvate thereof.

11. The compound of claim 1 having the structure of Formula (ID): 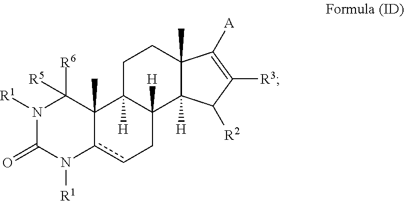
or a single stereoisomer or single tautomer or mixture thereof, or a pharmaceutically acceptable salt or solvate thereof.

12. The compound of claim 1 having the structure of Formula (IE): 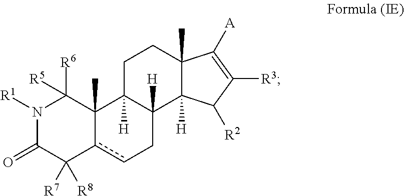
or a single stereoisomer or single tautomer or mixture thereof, or a pharmaceutically acceptable salt or solvate thereof.

13. The compound of claim 1 having the structure of Formula (IF): 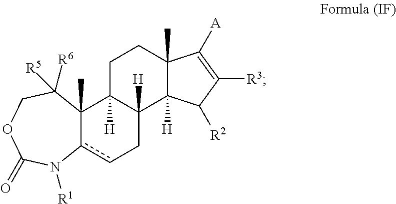
or a single stereoisomer or single tautomer or mixture thereof, or a pharmaceutically acceptable salt or solvate thereof.

14. The compound of claim 1 having the structure of Formula (IG): 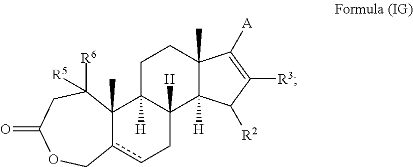
or a single stereoisomer or single tautomer or mixture thereof, or a pharmaceutically acceptable salt or solvate thereof.

15. The compound of claim 1 having the structure of Formula (IH): 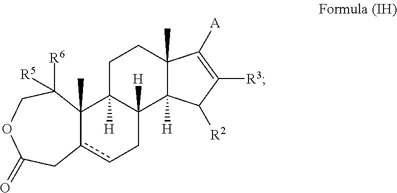
or a single stereoisomer or single tautomer or mixture thereof, or a pharmaceutically acceptable salt or solvate thereof.

16. The compound of claim 1 having the structure of Formula (IJ): 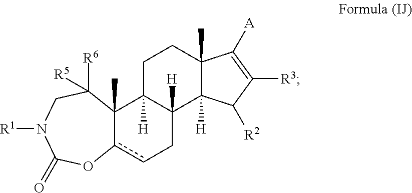
or a single stereoisomer or single tautomer or mixture thereof, or a pharmaceutically acceptable salt or solvate thereof.

17. The compound of claim 1 having the structure of Formula (IK): 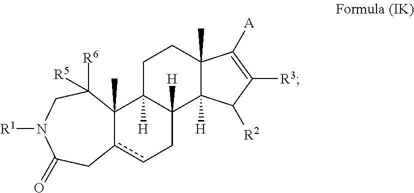
or a single stereoisomer or single tautomer or mixture thereof, or a pharmaceutically acceptable salt or solvate thereof.

18. The compound of claim 1 having the structure of Formula (IL): 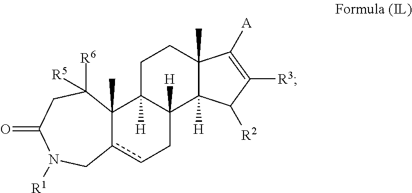
or a single stereoisomer or single tautomer or mixture thereof, or a pharmaceutically acceptable salt or solvate thereof.

19. The compound of claim 1 having the structure of Formula (IM): 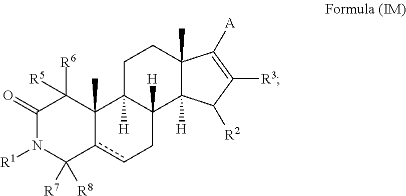
or a single stereoisomer or single tautomer or mixture thereof, or a pharmaceutically acceptable salt or solvate thereof.

20. The compound of claim 1 having the structure of Formula (IN): 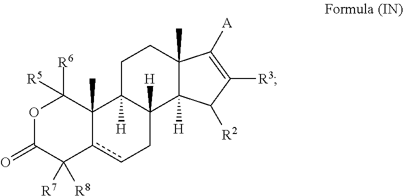
or a single stereoisomer or single tautomer or mixture thereof, or a pharmaceutically acceptable salt or solvate thereof.

21. The compound of claim 6 having the structure of Formula (IIA): 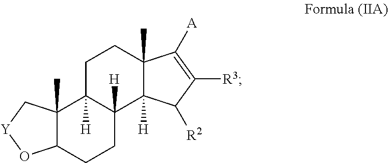
or a single stereoisomer or single tautomer or mixture thereof, or a pharmaceutically acceptable salt or solvate thereof.

22. The compound of claim 6 having the structure of Formula (IIB): 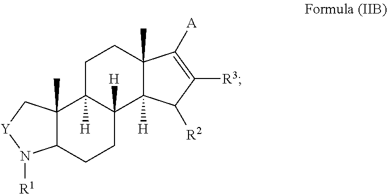
or a single stereoisomer or single tautomer or mixture thereof, or a pharmaceutically acceptable salt or solvate thereof.

23. The compound of claim 6 having the structure of Formula (IIC): 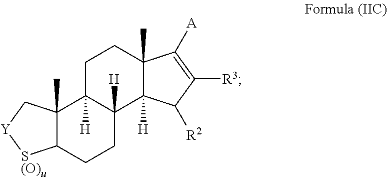
or a single stereoisomer or single tautomer or mixture thereof, or a pharmaceutically acceptable salt or solvate thereof.

24.-47. (canceled)

48. A compound having a structure selected from the group consisting of: 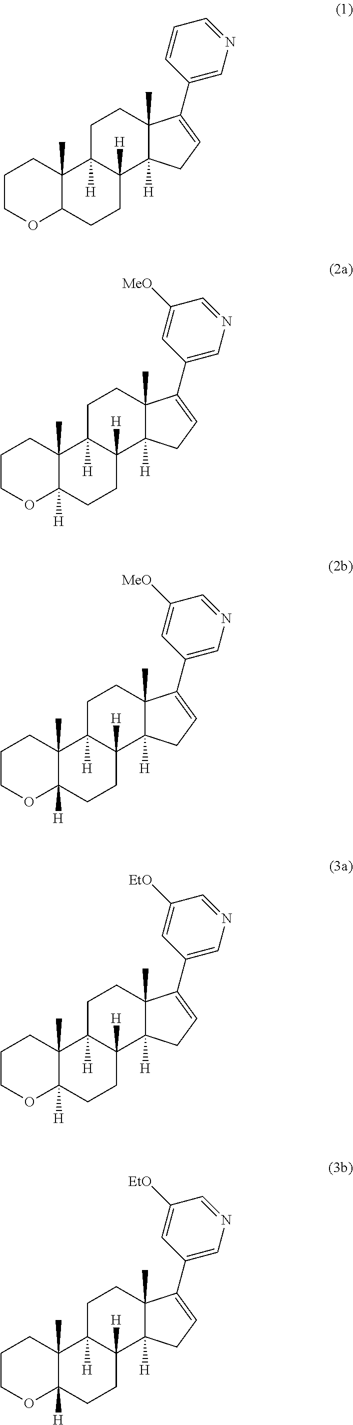 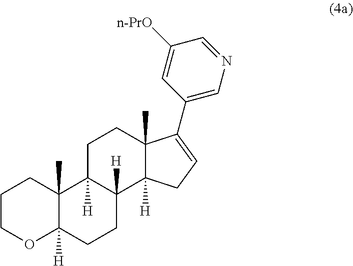 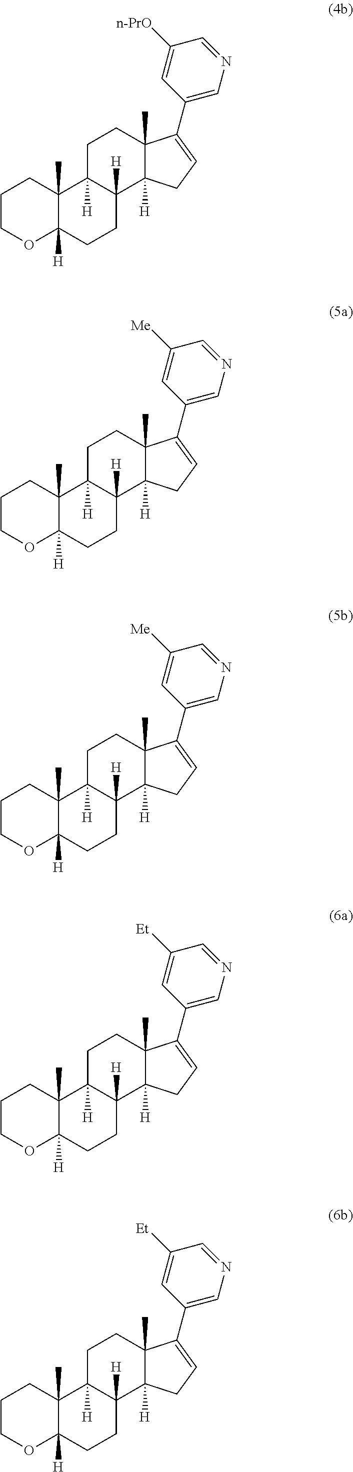 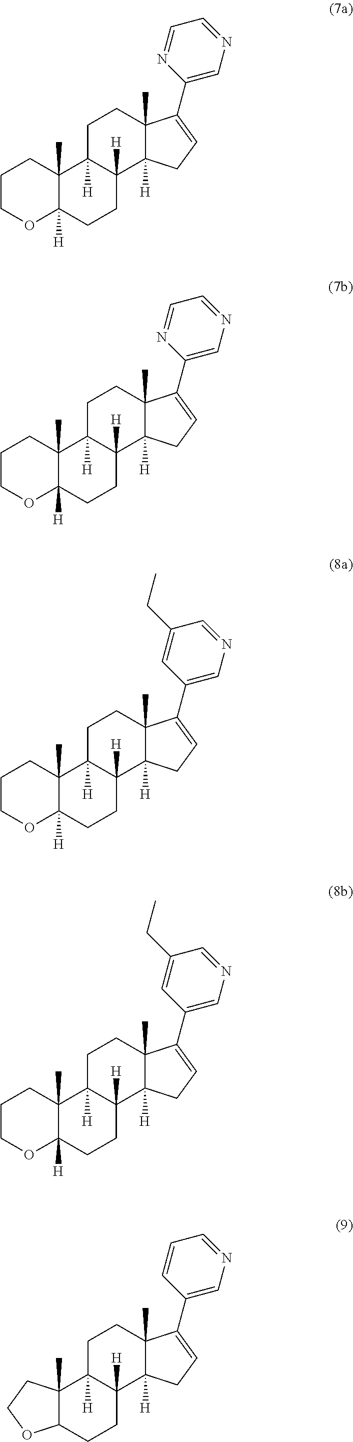 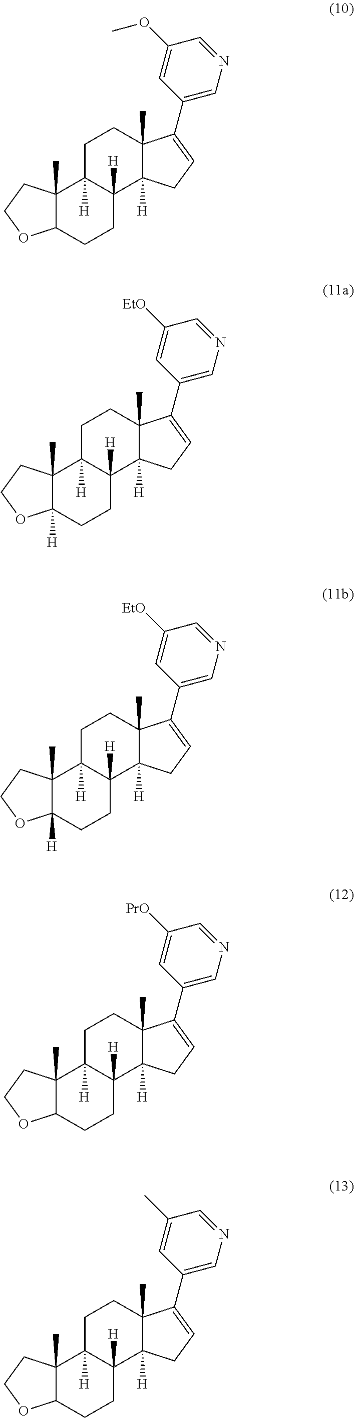 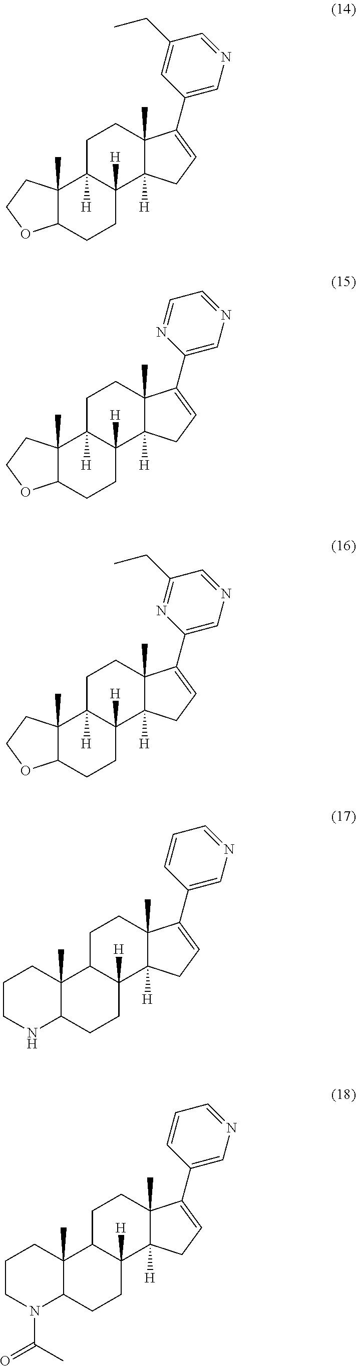 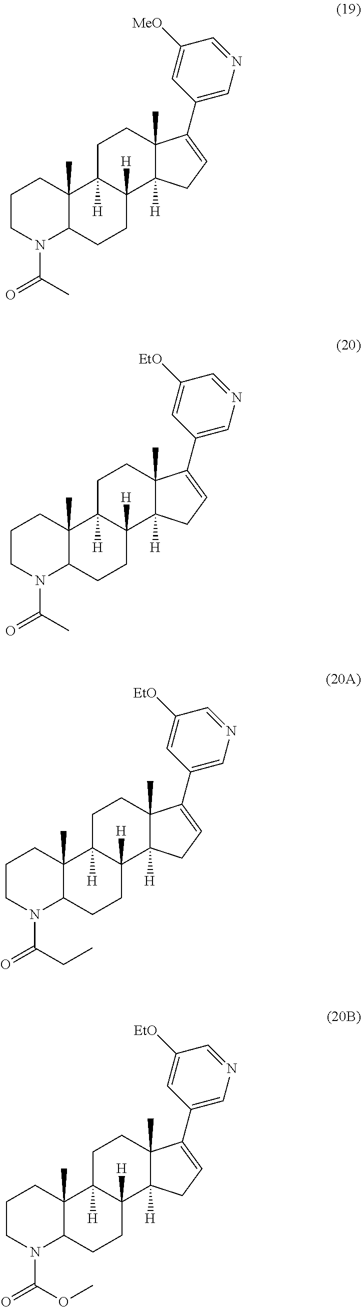 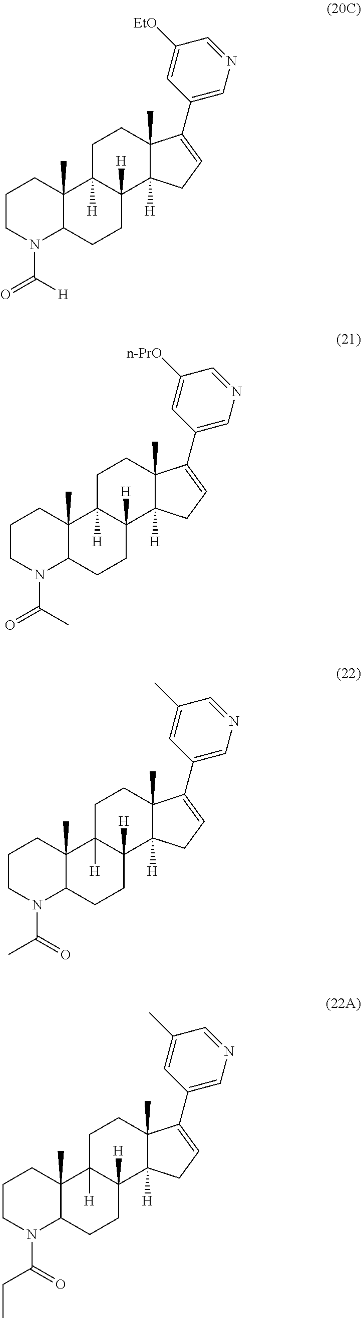 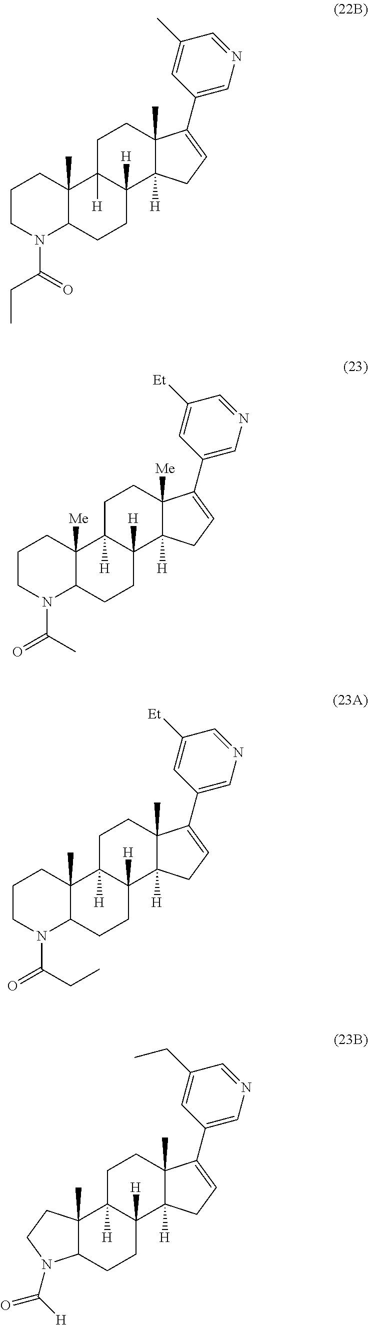 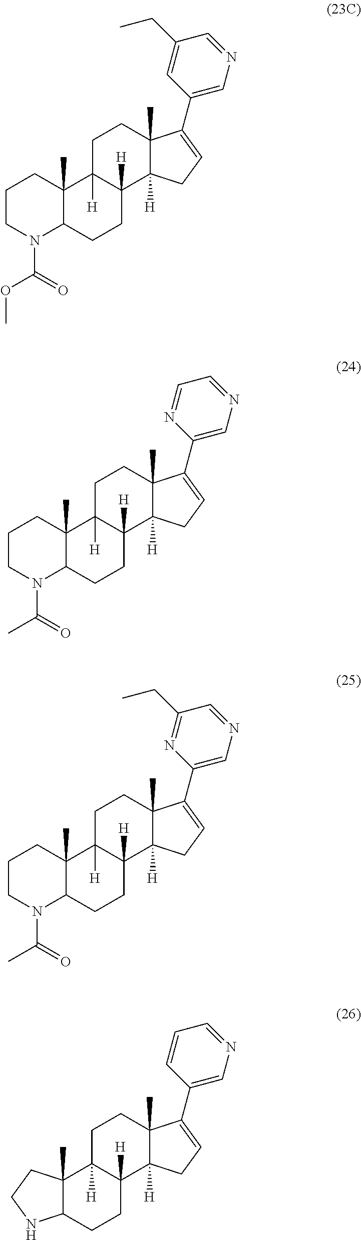 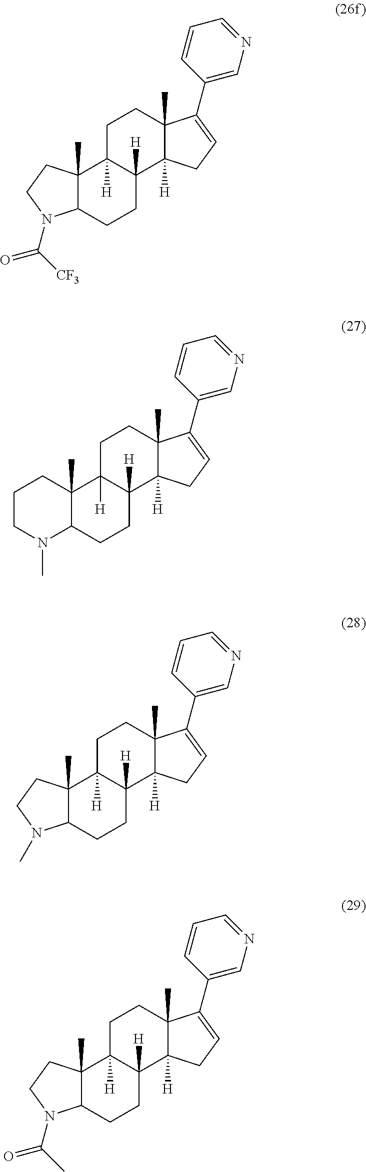 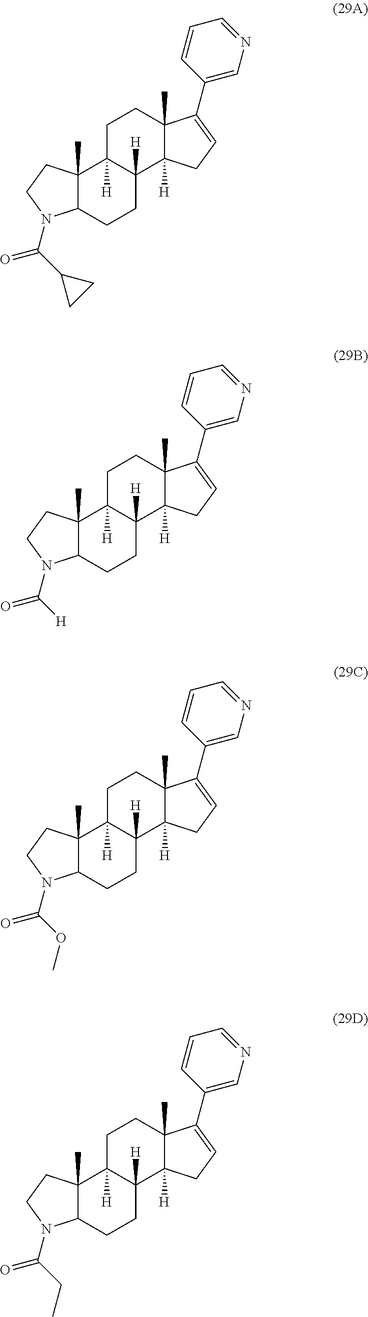 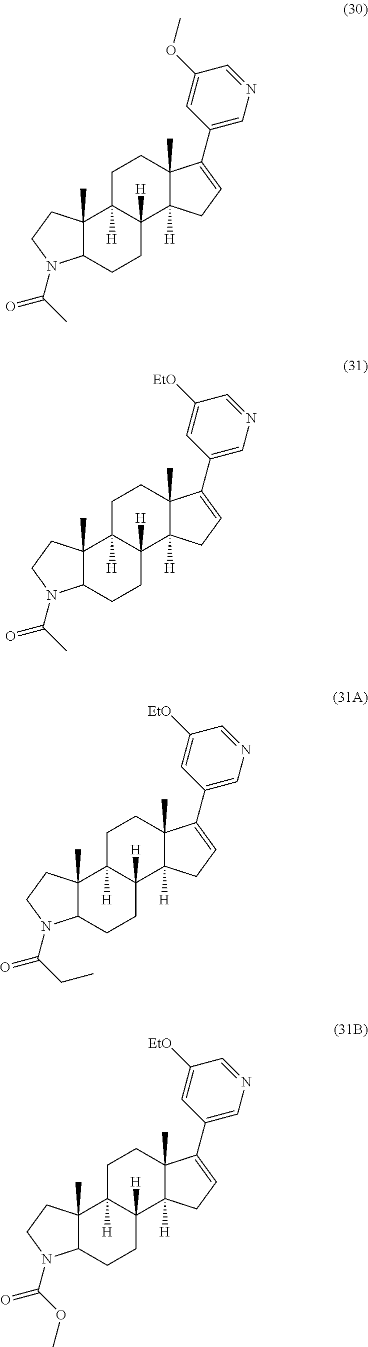 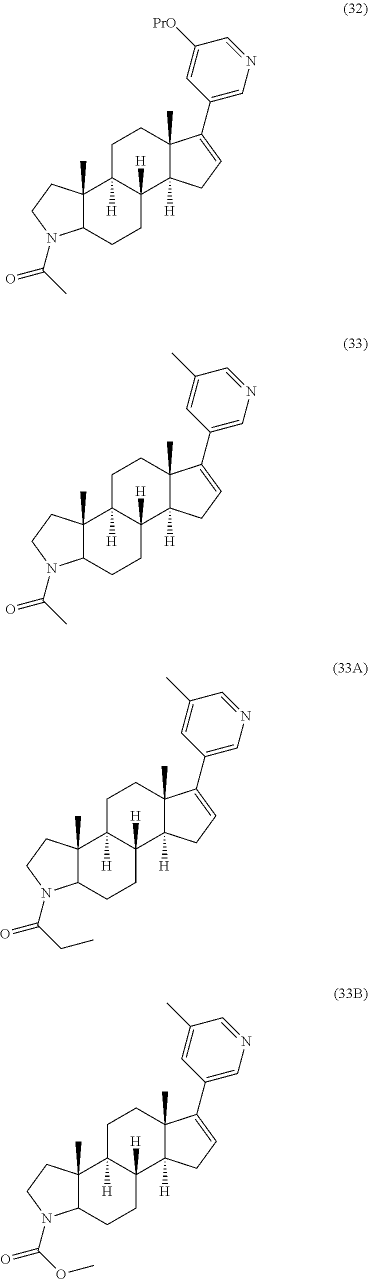 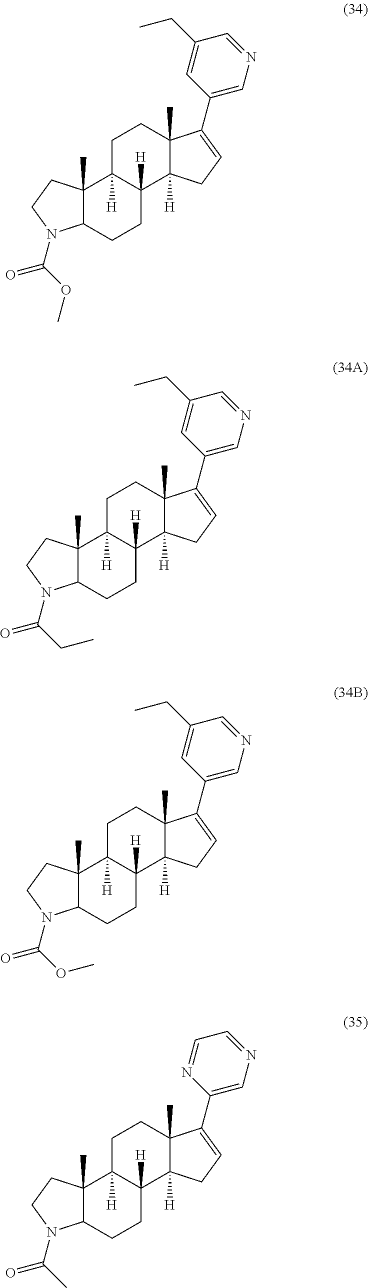 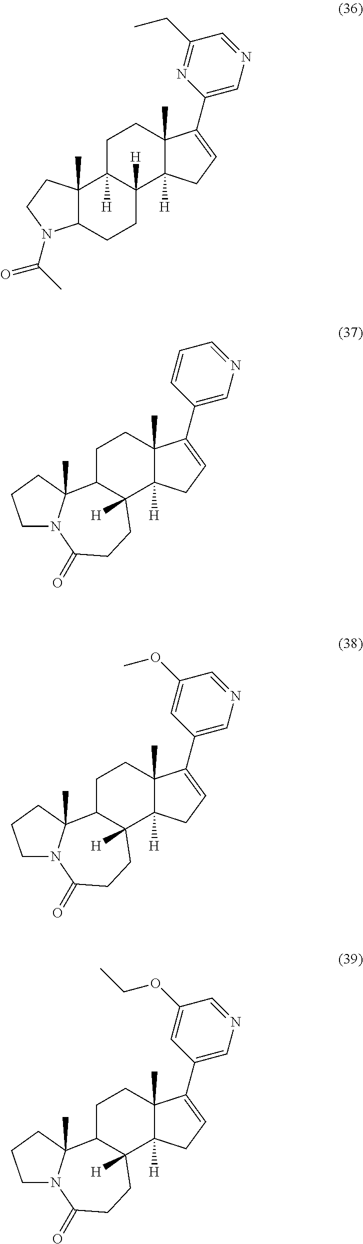 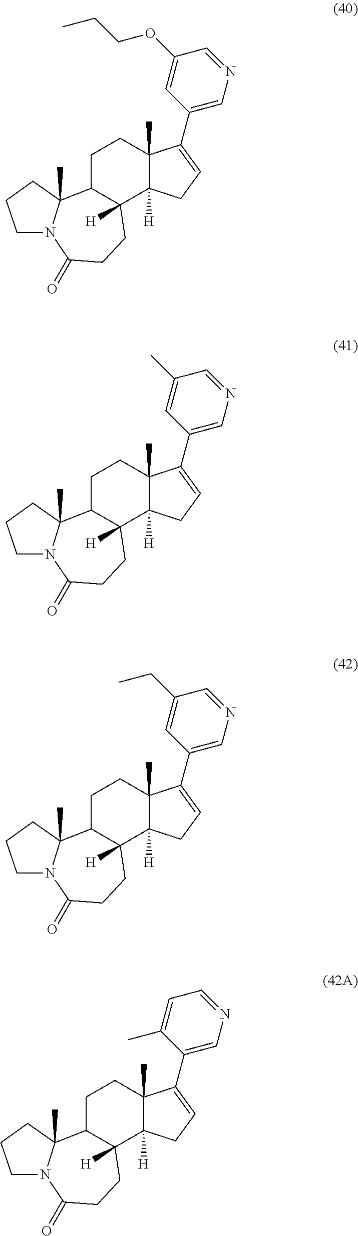 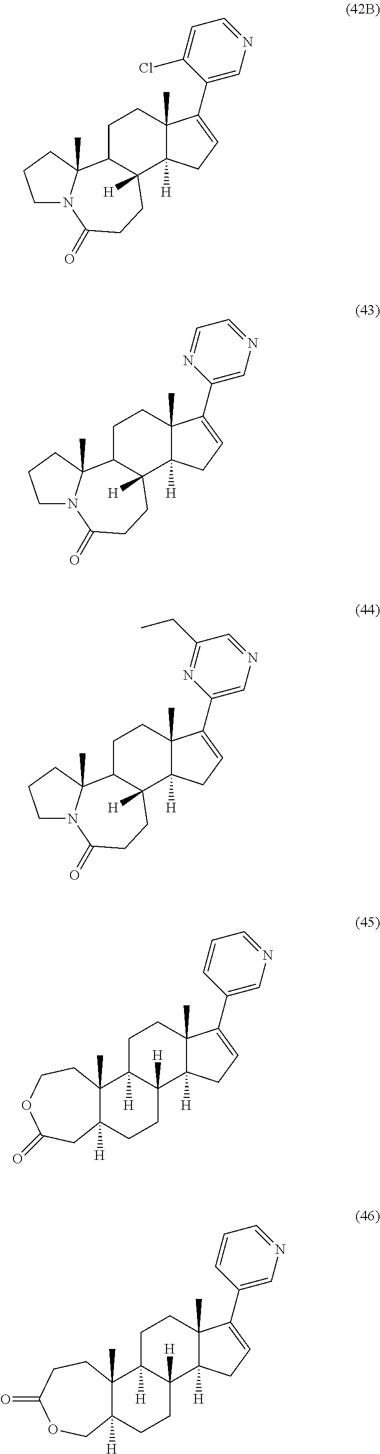 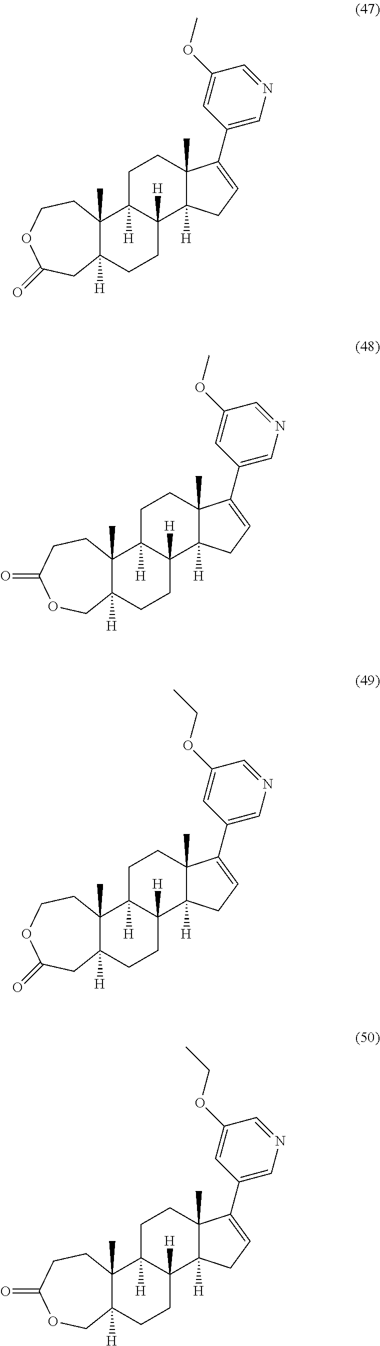 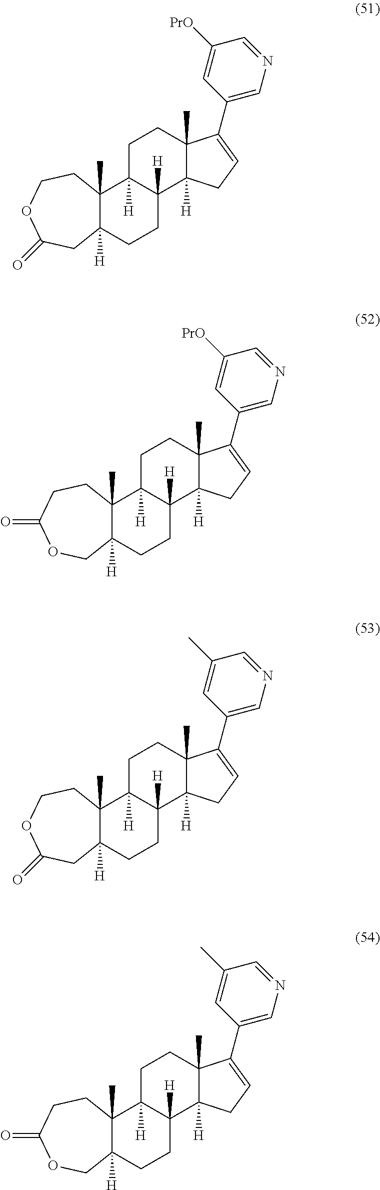 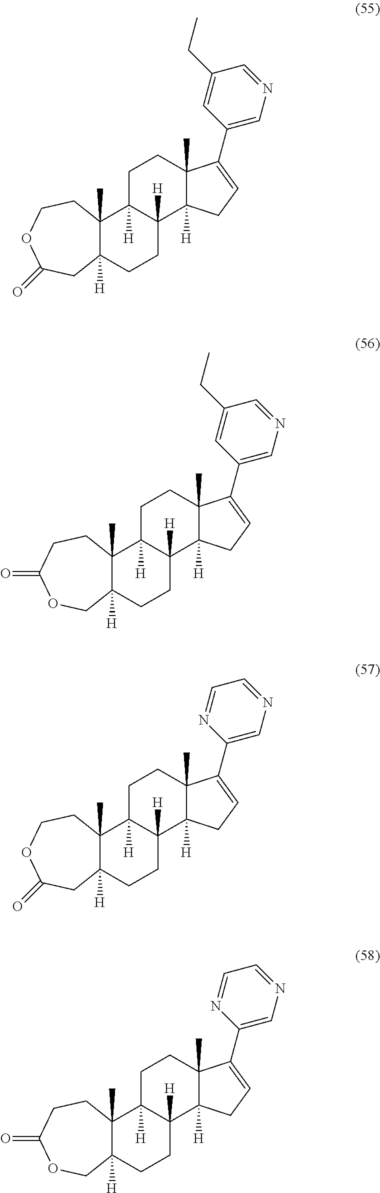 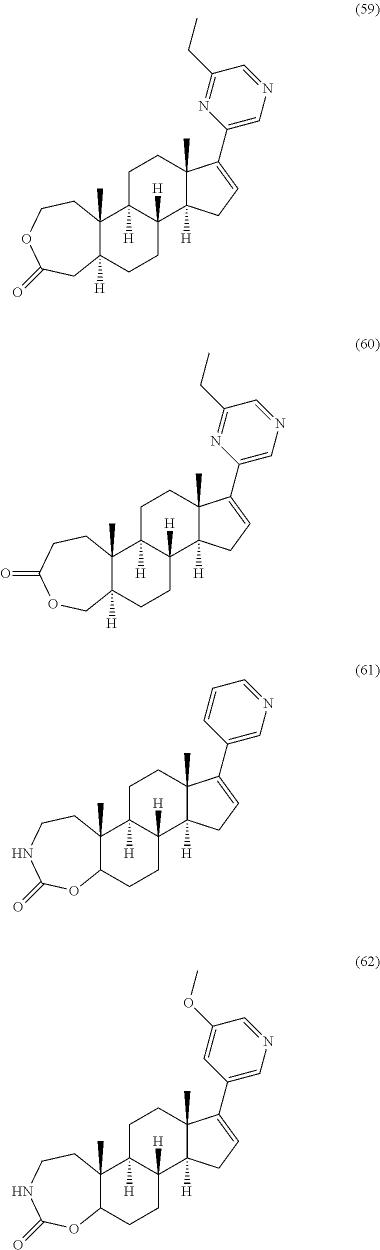 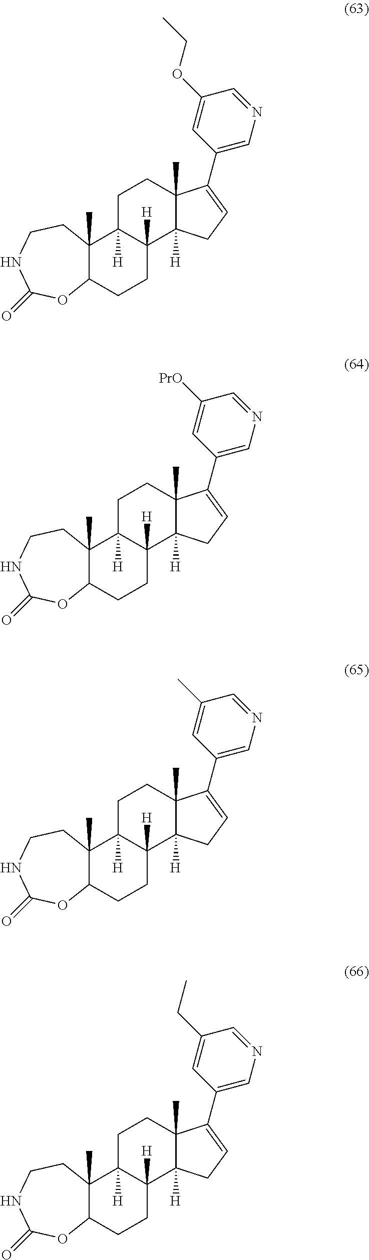 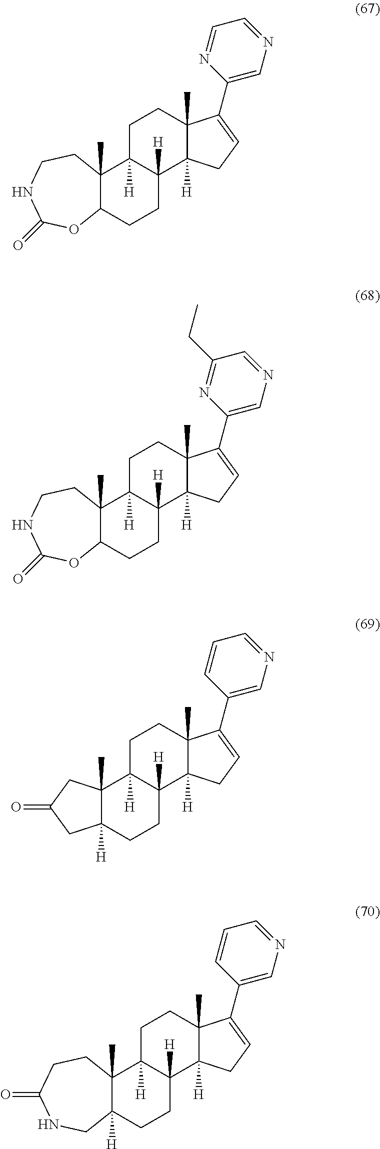 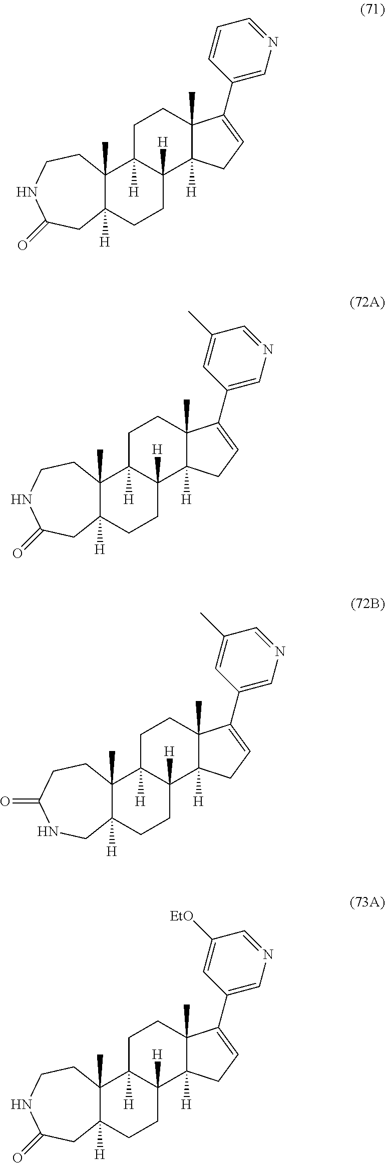 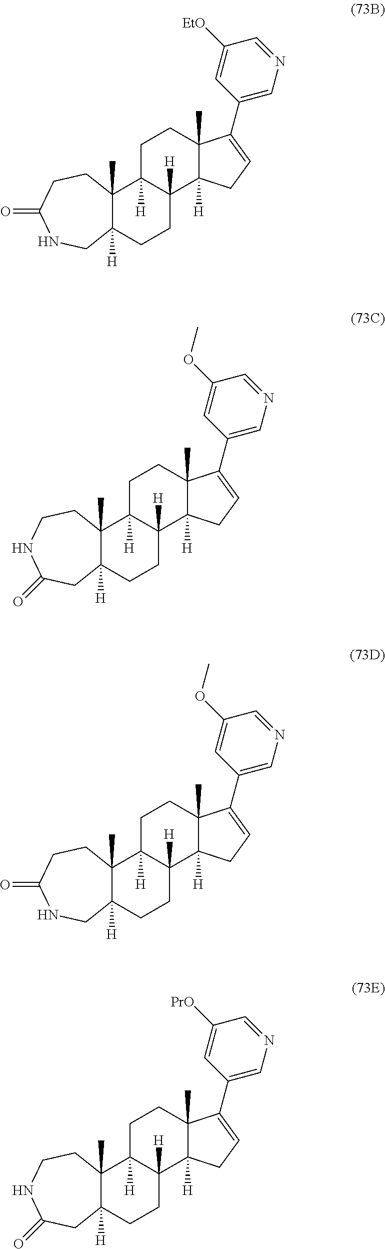 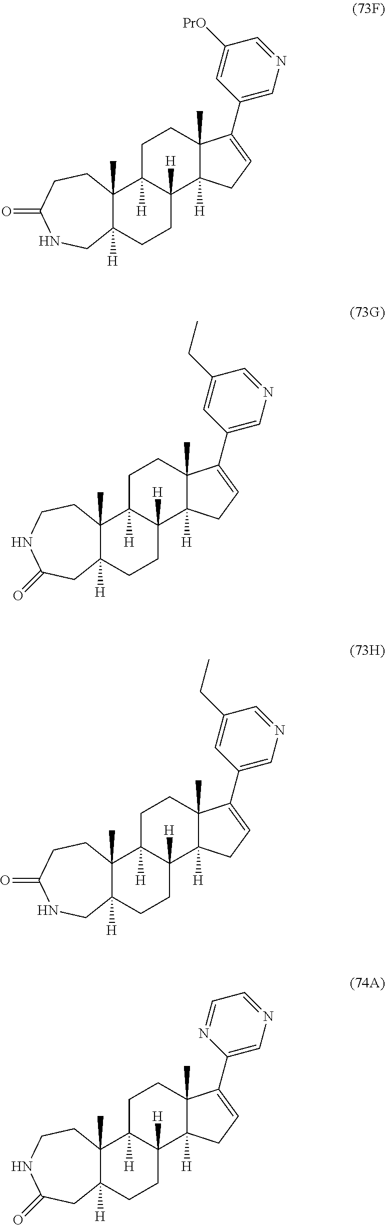 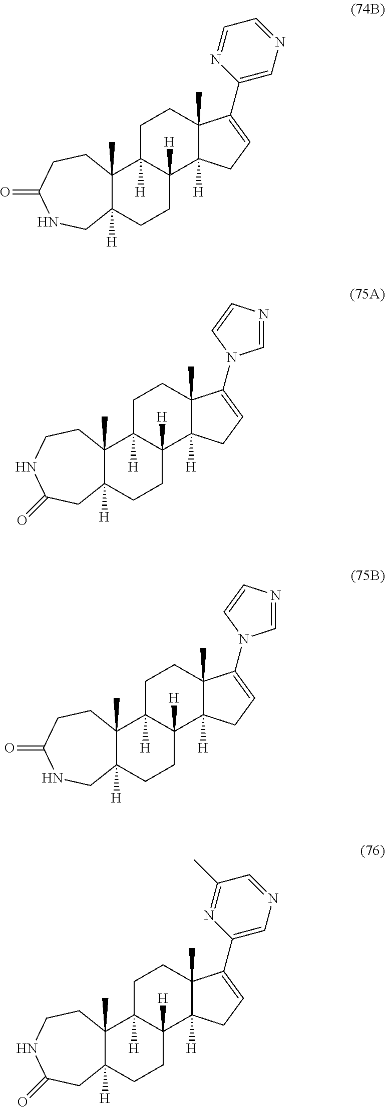 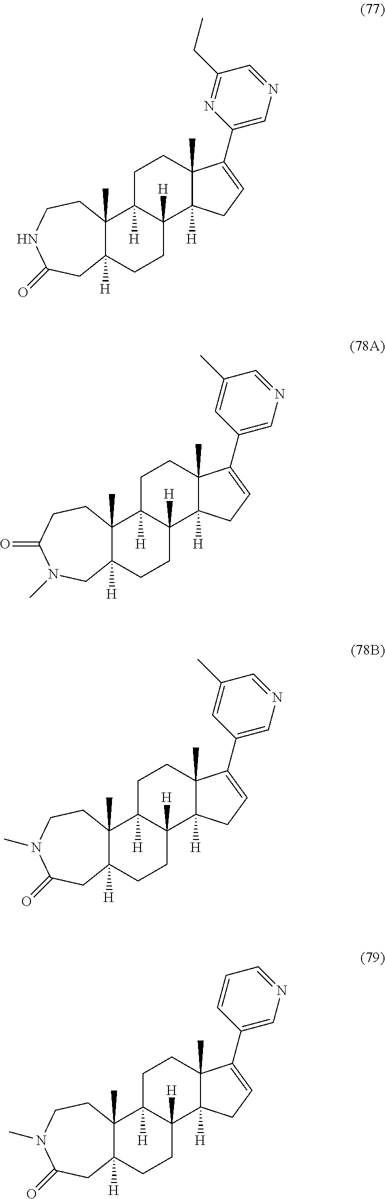 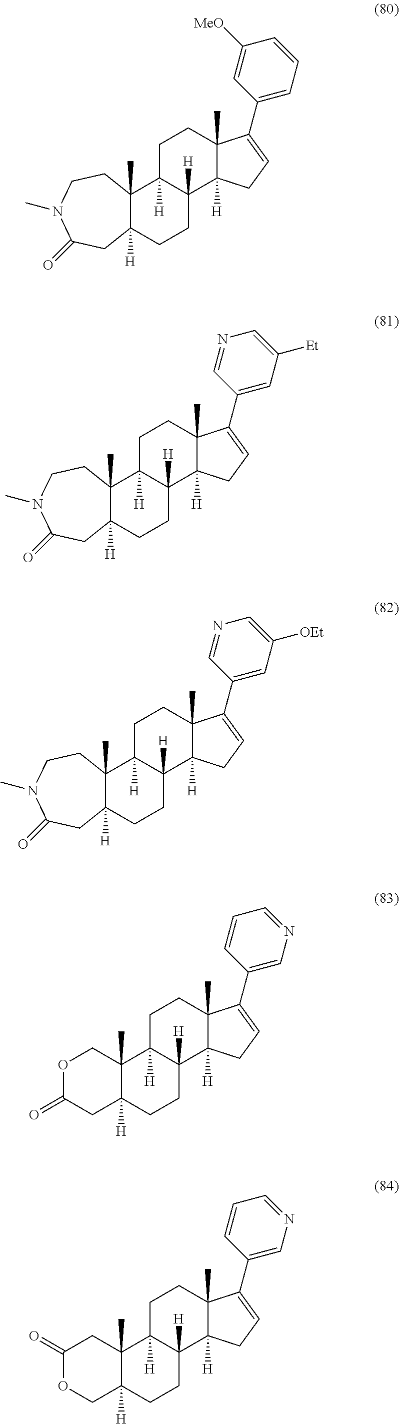 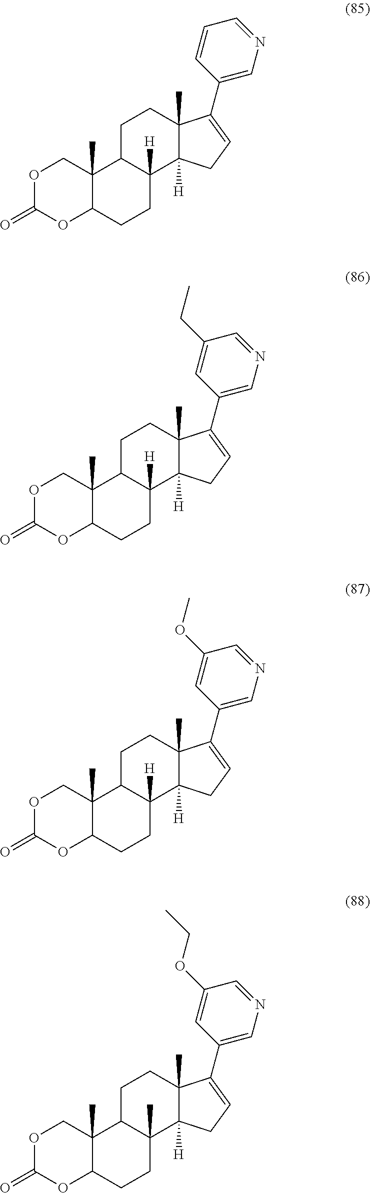 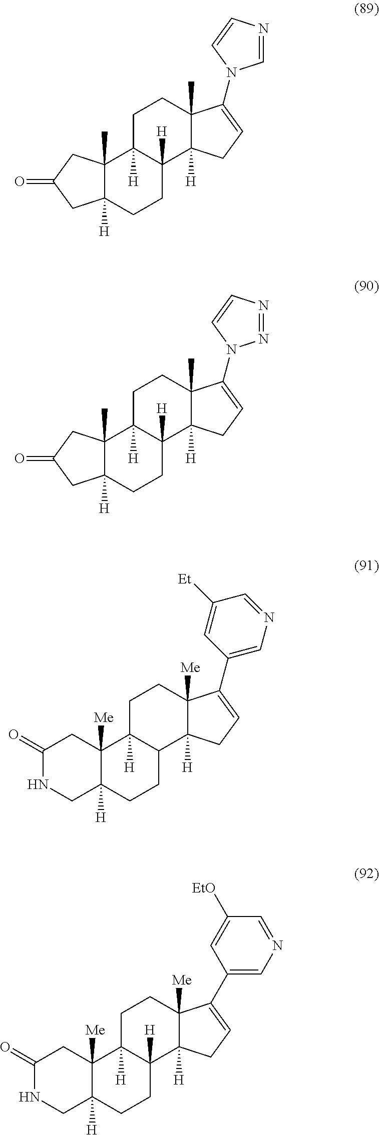 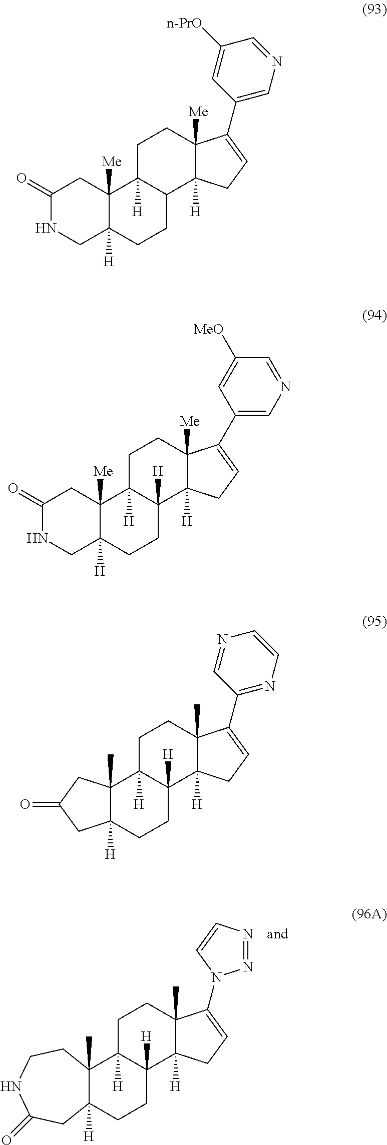 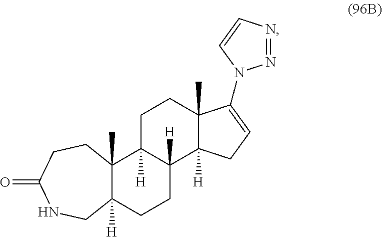
or a single stereoisomer or single tautomer or mixture thereof, or a pharmaceutically acceptable salt or solvate thereof.

49. (canceled)

50. A method for treating cancer in a subject comprising administering to a subject in need a therapeutically effective amount of a compound of claim 1 or a pharmaceutically acceptable salt or solvate thereof.

51.-56. (canceled)

57. A method of inhibiting CYP17 enzyme comprising contacting a compound of claim 1 or a pharmaceutically acceptable salt or solvate thereof with a CYP17 enzyme.

58. (canceled)

59. A method of treating an androgen-dependent disorder in a subject comprising administering to a subject in need a therapeutically effective amount of a compound of claim 1 or a pharmaceutically acceptable salt or solvate thereof.

60.-61. (canceled)

62. A method of treating a proliferative disease comprising administering to a subject in need a therapeutically effective amount of a compound of claim 1 or a pharmaceutically acceptable salt or solvate thereof.

63.-68. (canceled)
